(19) United States
(12) Patent Application Publication
(10) Pub. No.: US 2023/0059368 A1
(43) Pub. Date: Feb. 23, 2023

(54) POLYNUCLEOTIDE EDITORS AND METHODS OF USING THE SAME

(71) Applicant: PRIME MEDICINE, INC., Cambridge, MA (US)

(72) Inventors: Brian CAFFERTY, Cambridge, MA (US); John EVANS, Cambridge, MA (US)

(21) Appl. No.: 17/841,029

(22) Filed: Jun. 15, 2022

Related U.S. Application Data
(60) Provisional application No. 63/210,786, filed on Jun. 15, 2021.

(51) Int. Cl. C12N 15/10; C12N 9/00

(52) U.S. Cl. CPC C12N 15/102; C12N 9/93; C12N 2310/20; C12N 2320/53

(57) ABSTRACT

Provided herein are prime editing systems featuring prime editors complexed with a chimeric prime editing (PE) guide polynucleotide. Systems, prime editor fusion proteins and methods of using such editors for editing a double-stranded DNA target sequence are also provided.

### CROSS-REFERENCE

The present application claims the benefit of U.S. provisional Patent Application No. 63/210,786 filed Jun. 15, 2021, which is hereby incorporated by reference in its entirety.

### SEQUENCE LISTING

The instant application contains a Sequence Listing which has been submitted electronically in ASCII format and is hereby incorporated by reference in its entirety. Said ASCII copy, created on Aug. 29, 2022, is named 59761-739_201_SL.txt and is 365,777 bytes in size.

### BACKGROUND OF THE DISCLOSURE

Programmable nucleases (e.g., CRISPR-Cas9) can be used to form double-strand breaks (DSB) at precise locations in the genome. By changing the sequence of the variable region of the guide RNA (gRNA), these nucleases can target any sequence located proximal to their protospacer adjacent motif (PAM). Therapeutics based on these endonucleases are being developed due to their ability to allow insertion and/or deletion of genomic DNA. However, double-stranded breaks (DSB) are associated with unfavorable outcomes including translocations of chromosomes, p53 activation, and undesirable insertion/deletion (indel) events.

New approaches have been developed to enable genome editing that does not require DSB. One approach involves using partially inactivated endonucleases, termed nickases, that have been covalently joined to nucleobase deaminases to enable precision base editing. However, base editors are currently limited to four transition mutations and are unable to perform the eight transversion mutations necessary to treat all human genetic diseases that result from point mutations. Both programmable endonucleases and base editors are also constrained to editing within a narrow region of DNA that is proximal to the PAM. Thus, there remains a need for more versatile gene editing technology.

### SUMMARY OF THE DISCLOSURE

The disclosure provides gene editors featuring a nucleic acid programmable DNA binding protein (napDNAbp) with nickase activity (e.g., nCas9) and a DNA polymerase. The disclosure provides gene editors that are complexed with a chimeric prime editing (PE) guide polynucleotide. Systems, prime editor fusion proteins and methods of using such editors for editing a double-stranded DNA target sequence are also provided.

In some embodiments, the disclosure provides a method of editing a double-stranded target polynucleotide comprising contacting the double-stranded target polynucleotide with a chimeric prime editing guide polynucleotide (chimeric PEg polynucleotide), a nucleic acid programmable DNA binding protein (napDNAbp) having nickase activity, and a DNA polymerase; wherein the double-stranded target polynucleotide comprises a target strand and an edit strand; wherein the chimeric PEg polynucleotide comprises i) a deoxyribonucleic acid (DNA) segment comprising one or more intended nucleotide edits to be incorporated into the double-stranded target polynucleotide and is at least partially complementary to a portion of the edit strand of the double-stranded target polynucleotide, and ii) a ribonucleic acid (RNA) segment capable of binding to the napDNAbp and is at least partially complementary to a portion of the target strand of the double-stranded target polynucleotide; wherein the napDNAbp results in a nick in the edit strand of the double-stranded target polynucleotide; wherein the RNA segment hybridizes to the nicked edit strand of the double-stranded target polynucleotide; and wherein the DNA polymerase synthesizes a single stranded DNA that replaces an editing target sequence in the edit strand of the double-stranded target polynucleotide, thereby editing the double-stranded target polynucleotide.

In some embodiments, the disclosure provides a method of editing a double-stranded target polynucleotide comprising (a) contacting the double-stranded target polynucleotide with a chimeric prime editing guide polynucleotide (chimeric PEg polynucleotide), a nucleic acid programmable DNA binding protein (napDNAbp) having nickase activity, and a DNA polymerase; wherein the double-stranded target polynucleotide comprises a target strand and an edit strand; wherein the chimeric PEg polynucleotide comprises a ribonucleic acid (RNA) segment comprising a variable region and an invariable region, and a deoxyribonucleic acid (DNA) segment comprising an editing template and a primer binding site (PBS); wherein the variable region is at least partially complementary to a portion of the target strand of the double-stranded target polynucleotide; wherein the invariable region is capable of binding to the napDNAbp; wherein the primer binding site is at least partially complementary to a portion of the edit strand of the double-stranded target polynucleotide; wherein the editing template comprises one or more intended nucleotide edits to be incorporated into the double-stranded target polynucleotide; wherein the napDNAbp results in a nick in the edit strand of the double-stranded target polynucleotide; wherein the PBS hybridizes to the nicked edit strand of the double-stranded target polynucleotide; and wherein the DNA polymerase synthesizes a single stranded DNA encoded by the editing template; wherein the single stranded DNA replaces an editing target sequence in the edit strand of the double-stranded target polynucleotide, thereby altering the double-stranded target polynucleotide. In some embodiments, editing a double-stranded target polynucleotide comprises altering the double-stranded target polynucleotide.

In some embodiments, the nicking the edit strand of the double-stranded target polynucleotide with the napDNAbp forms 5′ and 3′ ends. In some embodiments, the PBS hybridizes to the 3′ end of the nicked edit strand of the double-stranded target polynucleotide. In some embodiments, the DNA polymerase extends the 3′ end of the nicked edit strand, thereby altering the double-stranded target polynucleotide. In some embodiments, the method further comprises repairing the double-stranded target polynucleotide with a DNA repair protein to. In some embodiments, the DNA repair protein is FEN1 or a DNA ligase. In some embodiments, the method further comprises nicking the target strand of the double-stranded target polynucleotide with a nickase to promote incorporation of the one or more intended nucleotide edits. In some embodiments, the RNA segment is at the 5′ end of the chimeric PE guide polynucleotide and the DNA segment is at the 3′ end of the chimeric PE guide polynucleotide. In some embodiments, the DNA segment is at the 5′ end of the chimeric PE guide polynucleotide and the RNA segment is at the 3′ end of the chimeric PE guide polynucleotide.

In some embodiments, the chimeric PEg polynucleotide comprises from 5′ to 3′: i) the RNA segment comprising a) the variable region; and b) the invariable region; and ii) the DNA segment comprising a) the editing template; and b) the primer binding site.

In some embodiments, the chimeric PEg polynucleotide comprises from 5′ to 3′: i) the DNA segment comprising a) the editing template; and b) the primer binding site; and ii) the RNA segment comprising a) the variable region; and b) the invariable region.

In some embodiments, one or more intended nucleotide edits comprises a substitution, insertion, deletion, and/or modification. In some embodiments, the altered double-stranded target polynucleotide differs by one or more nucleotides compared to the unaltered double-stranded target polynucleotide. In some embodiments, the one or more adenine bases of the unaltered double-stranded target polynucleotide are changed to a cytosine, guanine, or thymine in the altered double-stranded target polynucleotide. In some embodiments, the one or more cytosine bases of the unaltered double-stranded target polynucleotide are changed to an adenine, guanine, or thymine in the altered double-stranded target polynucleotide. In some embodiments, the one or more guanine bases of the unaltered double-stranded target polynucleotide are changed to an adenine, cytosine, or thymine in the altered double-stranded target polynucleotide. In some embodiments, the one or more thymine bases of the unaltered double-stranded target polynucleotide are changed to an adenine, guanine, or cytosine in the altered double-stranded target polynucleotide. In some embodiments, the altered double-stranded target polynucleotide comprises 1, 2, 3, 4, 5, 6, 7, 8, 9, 10, or more nucleotides that differ from the unaltered double-stranded target polynucleotide. In some embodiments, the altered double-stranded target polynucleotide comprises two or more nucleotides that differ from the unaltered double-stranded target polynucleotide. In some embodiments, any of the two or more nucleotides of the altered double-stranded target polynucleotide are consecutive nucleotides. In some embodiments, any of the two or more nucleotides of the altered double-stranded target polynucleotide are non-consecutive nucleotides. In some embodiments, the DNA segment comprises 1, 2, 3, 4, 5, 6, 7, 8, 9, 10, 11, 12, 13, 14, 15, 16, 17, 18, 19, 20, 30, 40, 50, 60, 70, 80, 90, 100 or more nucleotides to be inserted into the double-stranded target polynucleotide. In some embodiments, the altered double-stranded target polynucleotide comprises a deletion of one or more nucleotides compared to the unaltered double-stranded target polynucleotide. In some embodiments, the napDNAbp or the DNA polymerase is associated with at least one nuclear localization sequence (NLS). In some embodiments, the napDNAbp and the DNA polymerase are each bound to at least one nuclear localization sequence (NLS). In some embodiments, the at least one nuclear localization sequence (NLS) comprises an amino acid sequence selected from the group consisting of KRTADGSEFESPKKKRKV (SEQ ID NO: 7), KRPAATKKAGQAKKKK (SEQ ID NO: 8), KKTELQTTNAENKTKKL (SEQ ID NO: 9), KRGINDRNFWRGENGRKTR (SEQ ID NO: 10), RKSGKIAAIVVKRPRK (SEQ ID NO: 11), PKKKRKV (SEQ ID NO: 12), or MDSLLMNRRKFLYQFKNVRWAKGRRETYLC (SEQ ID NO: 13). In some embodiments, the at least one nuclear localization sequence (NLS) is a bipartite NLS. In some embodiments, the double-stranded target polynucleotide is in the genome of a cell. In some embodiments, the cell is a bacterial cell, plant cell, insect cell, or mammalian cell. In some embodiments, the cell is a human cell. In some embodiments, the DNA polymerase is an endogenous DNA polymerase. In some embodiments, the napDNAbp is fused to the DNA polymerase. In some embodiments, the napDNAbp is attached to the DNA polymerase by a peptide linker. In some embodiments, the linker is at least about 5-50 amino acids in length. In some embodiments, the linker is at least about 10-30 amino acids in length. In some embodiments, the linker comprises a sequence selected from the group consisting of (SGGS)ₙ (SEQ ID NO: 14), (GGGS)ₙ (SEQ ID NO: 15), (GGGGS)ₙ (SEQ ID NO: 16), (G)ₙ, (EAAAK)ₙ (SEQ ID NO: 17), (GGS)ₙ, SGSETPGTSESATPES (SEQ ID NO: 18), and (XP)ₙ motif. In some embodiments, the linker comprises the amino acid sequence: SGGSSGGSSGSETPGTSESATPES (SEQ ID NO: 19). In some embodiments, the DNA segment is fused to the RNA segment. In some embodiments, the DNA segment is covalently linked to the RNA segment. In some embodiments, the DNA segment and the RNA segment are linked by a linker. In some embodiments, the linker comprises a sequence selected from the group consisting of AAAUUAACAAACUAA (SEQ ID NO: 20), AACAAACUAA (SEQ ID NO: 21), UUUUUUUUUUUUUUU (SEQ ID NO: 22), UUUUUUUUUUU (SEQ ID NO: 23), and UUUUU. In some embodiments, the DNA polymerase is a bacterial, eukaryotic, insect or plant DNA polymerase. In some embodiments, the DNA polymerase is a eukaryotic DNA polymerase and is selected from the group consisting of DNA polymerase α, β, γ, δ, and ε, or a corresponding DNA polymerase thereof. In some embodiments, the DNA polymerase is a bacterial DNA polymerase and is selected from the group consisting of DNA polymerase I, II, and III, or a corresponding DNA polymerase thereof. In some embodiments, the napDNAbp comprises a Cas9 nickase (nCas9), a Cpf1 nickase (nCpf1), or a variant thereof. In some embodiments, the napDNAbp comprises a Cas9 nickase (nCas9). In some embodiments, the Cas9 is a Streptococcus pyogenes Cas9 (SpCas9), a Staphylococcus aureus Cas9 (SaCas9), a Streptococcus thermophilus 1 Cas9 (St1Cas9), a Campylobacter jejuni Cas9 (CjCas9), or variants thereof. In some embodiments, the Cas9 is a modified Cas9. In some embodiments, a Cas protein, e.g., Cas9, can comprise an amino acid change such as a deletion, insertion, substitution, fusion, chimera, or any combination thereof relative to a corresponding wild-type version of the Cas protein. In some embodiments, a Cas protein can be a polypeptide with at least about 50%, 60%, 70%, 80%, 90%, 91%, 92%, 93%, 94%, 95%, 96%, 97%, 98%, 99%, or 100% sequence identity or sequence similarity to a wild type exemplary Cas protein. In some embodiments, the modified Cas9 comprises amino acid substitution D10A or H840A, or a corresponding amino acid substitution thereof.

In some embodiments, the disclosure provides a chimeric prime editing chimeric guide polynucleotide (chimeric PEg polynucleotide) comprising a deoxyribonucleic acid (DNA) segment and a ribonucleic acid (RNA) segment; wherein the DNA segment comprises i) an editing template comprising one or more intended nucleotide edits to be incorporated into a double-stranded target polynucleotide comprising a target strand and an edit strand, and ii) a primer binding site (PBS) that is at least partially complementary to a portion of the edit strand of the double-stranded target polynucleotide, and wherein the RNA segment comprises i) a variable region that is at least partially complementary to a portion of the target strand of the double-stranded target polynucleotide, and ii) an invariable region that is capable of binding to a nucleic acid programmable DNA binding protein (napDNAbp).

In some embodiments, the RNA segment is at the 5′ end of the chimeric PEg polynucleotide and the DNA segment is at the 3′ end of the chimeric PEg polynucleotide. In some embodiments, the DNA segment is at the 5′ end of the chimeric PE guide polynucleotide and the RNA segment is at the 3′ end of the chimeric PE guide polynucleotide.

In some embodiments, the disclosure provides a chimeric prime editing guide polynucleotide (chimeric PEg polynucleotide) capable of binding to a double-stranded target polynucleotide comprising a target strand and an editstrand, the guide comprising from 5′ to 3′: i) a ribonucleic acid (RNA) segment comprising a) a variable region that is at least partially complementary to a portion of the target strand of a double-stranded target polynucleotide; and b) an invariable region that is capable of binding to a nucleic acid programmable DNA binding protein (napDNAbp); and ii) a deoxyribonucleic acid (DNA) segment comprising a) an editing template comprising one or more intended nucleotide edits to be incorporated into the double-stranded target polynucleotide; and b) a primer binding site (PBS) that is at least partially complementary to a portion of the edit strand of the double-stranded target polynucleotide.

In some embodiments, the invention provides a chimeric prime editing guide polynucleotide (chimeric PEg polynucleotide) capable of binding to a double-stranded target polynucleotide comprising a target strand and an editstrand, the guide comprising from 5′ to 3′: i) a deoxyribonucleic acid (DNA) segment comprising a) an editing template comprising one or more intended nucleotide edits to be incorporated into the double-stranded target polynucleotide; and b) a primer binding site (PBS) that is at least partially complementary to a portion of the edit strand of the double-stranded target polynucleotide; and ii) a ribonucleic acid (RNA) segment comprising a) a variable region that is at least partially complementary to a portion of the target strand of a double-stranded target polynucleotide; and b) an invariable region that is capable of binding to a nucleic acid programmable DNA binding protein (napDNAbp).

In some embodiments, the one or more intended nucleotide edits comprises a substitution, insertion, deletion, and/or modification. In some embodiments, the chimeric PEg polynucleotide further comprises a linker. In some embodiments, the linker comprises a sequence selected from the group consisting of AAAUUAACAAACUAA (SEQ ID NO: 20), AACAAACUAA (SEQ ID NO: 21), UUUUUUUUUUUUUUU (SEQ ID NO: 22), UUUUUUUUUUU (SEQ ID NO: 23), and UUUUU. In some embodiments, the edit strand of the double-stranded target polynucleotide comprises a portion of genomic DNA in proximity to a nick site wherein a portion of the nucleic acid segment binds. In some embodiments, the chimeric PEg polynucleotide mediates binding of a napDNAbp to a double-stranded target polynucleotide. In some embodiments, the napDNAbp has nickase activity. In some embodiments, the napDNAbp comprises a Cas9 nickase (nCas9), a Cpf1 nickase (nCpf1), or a variant thereof. In some embodiments, the napDNAbp is a Streptococcus pyogenes Cas9 (SpCas9), a Staphylococcus aureus Cas9 (SaCas9), a Streptococcus thermophilus 1 Cas9 (St1Cas9), a Campylobacter jejuni Cas9 (CjCas9), or variants thereof. In some embodiments, the Cas9 is a modified Cas9. In some embodiments, the modified Cas9 comprises amino acid substitution D10A or H840A, or a corresponding amino acid substitution thereof.

In some embodiments, the disclosure provides a prime editor fusion protein comprising (i) a nucleic acid programmable DNA binding protein (napDNAbp) with nickase activity; and (ii) a DNA polymerase.

In some embodiments, the napDNAbp is capable of nicking a strand of a double-stranded target polynucleotide, and wherein the DNA polymerase is capable of extending the nicked strand when the nicked strand is bound to a chimeric prime editing guide polynucleotide. In some embodiments, the chimeric prime editing guide polynucleotide comprises a deoxyribonucleic acid (DNA) and a ribonucleic acid (RNA) segment. In some embodiments, the DNA segment is covalently linked or fused to the RNA segment. In some embodiments, the DNA segment and the RNA segment are linked by a linker. In some embodiments, the linker comprises a sequence selected from the group consisting of AAAUUAACAAACUAA (SEQ ID NO: 20), AACAAACUAA (SEQ ID NO: 21), UUUUUUUUUUUUUUU (SEQ ID NO: 22), UUUUUUUUUUU (SEQ ID NO: 23), and UUUUU. In some embodiments, the DNA polymerase is a bacterial, eukaryotic, insect or plant DNA polymerase. In some embodiments, the DNA polymerase is a eukaryotic DNA polymerase and is selected from the group consisting of DNA polymerase α, β, γ, δ, and ε, or a corresponding DNA polymerase thereof. In some embodiments, the DNA polymerase is a bacterial DNA polymerase and is selected from the group consisting of DNA polymerase I, II, and III, or a corresponding DNA polymerase thereof. In some embodiments, the napDNAbp comprises a Cas9 nickase (nCas9), a Cpf1 nickase (nCpf1), or a variant thereof. In some embodiments, the napDNAbp comprises a Cas9 nickase (nCas9). In some embodiments, the Cas9 is a Streptococcus pyogenes Cas9 (SpCas9), a Staphylococcus aureus Cas9 (SaCas9), a Streptococcus thermophilus 1 Cas9 (St1Cas9), a Campylobacter jejuni Cas9 (CjCas9), or variants thereof. In some embodiments, the Cas9 is a modified Cas9. In some embodiments, the modified Cas9 comprises amino acid substitution D10A or H840A, or a corresponding amino acid substitution thereof. In some embodiments, the wherein the napDNAbp is fused to the DNA polymerase via a peptide linker. In some embodiments, the linker is at least about 5-50 amino acids in length. In some embodiments, the linker is at least about 10-30 amino acids in length. In some embodiments, the linker comprises a sequence selected from the group consisting of (SGGS)ₙ (SEQ ID NO: 14), (GGGS)ₙ (SEQ ID NO: 15), (GGGGS)ₙ (SEQ ID NO: 16), (G)ₙ (EAAAK)ₙ (SEQ ID NO: 17), (GGS)ₙ, SGSETPGTSESATPES (SEQ ID NO: 18), and (XP)ₙ motif. In some embodiments, the linker comprises the amino acid sequence: SGGSSGGSSGSETPGTSESATPES (SEQ ID NO: 19).

In some embodiments, the disclosure provides a nucleic acid encoding the prime editor fusion protein of the disclosure.

In some embodiments, the disclosure a vector comprising the nucleic acid of this disclosure.

In some embodiments, the disclosure provides a prime editing system comprising (i) a prime editor fusion protein of the disclosure; and (ii) the guide of this disclosure.

In some embodiments, the disclosure provides a prime editing system comprising (i) a nucleic acid programmable DNA binding protein (napDNAbp) with nicking activity; (ii) a DNA polymerase; and (ii) the chimeric prime editing guide polynucleotide of this disclosure.

In some embodiments, the disclosure provides a prime editing system for editing a double-stranded target polynucleotide comprising a target strand and an edit strand, wherein the prime editing system comprises comprising a chimeric prime editing guide polynucleotide (chimeric PEg polynucleotide), a nucleic acid programmable DNA binding protein (napDNAbp) with nickase activity, and a DNA polymerase; wherein the chimeric PEg polynucleotide comprises a deoxyribonucleic acid (DNA) segment and a ribonucleic acid (RNA) segment, wherein the DNA segment comprises i) a primer binding site (PBS) that is at least partially complementary to a portion of the edit strand of the double-stranded target polynucleotide, and ii) an editing template comprising one or more intended nucleotide edits to be incorporated into the double-stranded target polynucleotide, and wherein the RNA segment comprises i) a variable region that is at least partially complementary to a portion of the target strand of the double-stranded target polynucleotide, and ii) an invariable region that is capable of binding to the napDNAbp.

In some embodiments, the RNA segment is at the 5′ end of the chimeric PE guide polynucleotide and the DNA segment is at the 3′ end of the chimeric PE guide polynucleotide. In some embodiments, the DNA segment is at the 5′ end of the chimeric PE guide polynucleotide and the RNA segment is at the 3′ end of the chimeric PE guide polynucleotide.

In some embodiments, the disclosure provides a prime editing system for editing a double-stranded target polynucleotide comprising a target strand and a edit strand, wherein the prime editor system comprises a chimeric prime editing guide polynucleotide (chimeric PEg polynucleotide), a nucleic acid programmable DNA binding protein (napDNAbp) with nickase activity, and a DNA polymerase; wherein the chimeric PEg polynucleotide comprises from 5′ to 3′: i) a ribonucleic acid (RNA) segment comprising a) a variable region that is at least partially complementary to a portion of the target strand of a double-stranded target polynucleotide; and b) an invariable region that is capable of binding to the napDNAbp; and ii) a deoxyribonucleic acid (DNA) segment comprising a) an editing template comprising one or more intended nucleotide edits to be incorporated into the double-stranded target polynucleotide; and b) a primer binding site (PBS) that is at least partially complementary to a portion of the edit strand of the double-stranded target polynucleotide.

In some embodiments, the disclosure provides a prime editing system for editing a double-stranded target polynucleotide comprising a target strand and an edit strand, wherein the prime editor system comprises a chimeric prime editing guide polynucleotide (chimeric PEg polynucleotide), a nucleic acid programmable DNA binding protein (napDNAbp) with nickase activity, and a DNA polymerase; wherein the chimeric PE guide polynucleotide comprises from 5′ to 3′: i) a deoxyribonucleic acid (DNA) segment comprising a) an editing diting template comprising one or more intended nucleotide edits to be incorporated into the double-stranded target polynucleotide; and b) a primer binding site (PBS) that is at least partially complementary to a portion of the edit strand of the double-stranded target polynucleotide; and ii) a ribonucleic acid (RNA) segment comprising a) a variable region that is at least partially complementary to a portion of the target strand of a double-stranded target polynucleotide; and b) an invariable region that is capable of binding to the napDNAbp.

In some embodiments, the one or more intended nucleotide edits comprises a substitution, insertion, deletion, and/or modification. In some embodiments, the altered double-stranded target polynucleotide differs by one or more nucleotides compared to the unaltered double-stranded target polynucleotide. In some embodiments, the one or more adenine bases of the unaltered double-stranded target polynucleotide are changed to a cytosine, guanine, or thymine in the altered double-stranded target polynucleotide. In some embodiments, the one or more cytosine bases of the unaltered double-stranded target polynucleotide are changed to an adenine, guanine, or thymine in the altered double-stranded target polynucleotide. In some embodiments, the one or more guanine bases of the unaltered double-stranded target polynucleotide are changed to an adenine, cytosine, or thymine in the altered double-stranded target polynucleotide. In some embodiments, the one or more thymine bases of the unaltered double-stranded target polynucleotide are changed to an adenine, guanine, or cytosine in the altered double-stranded target polynucleotide. In some embodiments, the altered double-stranded target polynucleotide comprises 1, 2, 3, 4, 5, 6, 7, 8, 9, 10, or more nucleotides that differ from the unaltered double-stranded target polynucleotide. In some embodiments, the altered double-stranded target polynucleotide comprises two or more nucleotides that differ from the unaltered double-stranded target polynucleotide. In some embodiments, any of the two or more nucleotides of the altered double-stranded target polynucleotide are consecutive nucleotides. In some embodiments, any of the two or more nucleotides of the altered double-stranded target polynucleotide are non-consecutive nucleotides. In some embodiments, the DNA segment comprises 1, 2, 3, 4, 5, 6, 7, 8, 9, 10, 11, 12, 13, 14, 15, 16, 17, 18, 19, 20, 30, 40, 50, 60, 70, 80, 90, 100 or more nucleotides to be inserted into the double-stranded target polynucleotide. In some embodiments, the altered double-stranded target polynucleotide comprises a deletion of one or more nucleotides compared to the unaltered double-stranded target polynucleotide. In some embodiments, the DNA polymerase is a bacterial, eukaryotic, insect or plant DNA polymerase. In some embodiments, the DNA polymerase is a eukaryotic DNA polymerase and is selected from the group consisting of DNA polymerase α, β, γ, δ, and ε, or a corresponding DNA polymerase thereof. In some embodiments, the DNA polymerase is a bacterial DNA polymerase and is selected from the group consisting of DNA polymerase I, II, and III, or a corresponding DNA polymerase thereof. In some embodiments, the napDNAbp comprises a Cas9 nickase (nCas9), a Cpf1 nickase (nCpf1), or a variant thereof. In some embodiments, the napDNAbp comprises a Cas9 nickase (nCas9). In some embodiments, the Cas9 is a Streptococcus pyogenes Cas9 (SpCas9), a Staphylococcus aureus Cas9 (SaCas9), a Streptococcus thermophilus 1 Cas9 (St1Cas9), a Campylobacter jejuni Cas9 (CjCas9), or variants thereof. In some embodiments, the Cas9 is a modified Cas9. In some embodiments, the modified Cas9 comprises amino acid substitution D10A or H840A, or a corresponding amino acid substitution thereof. In some embodiments, the napDNAbp comprises a non-functional catalytic domain that is not capable of cleaving the first strand of the double-stranded target polynucleotide. In some embodiments, the napDNAbp does not comprise a catalytic domain capable of cleaving the double-stranded target polynucleotide. In some embodiments, the catalytic domain is an HNH domain. In some embodiments, the DNA polymerase is an endogenous DNA polymerase. In some embodiments, the napDNAbp is fused to the DNA polymerase. In some embodiments, the DNA polymerase is covalently or non-covalently attached to said napDNAbp. In some embodiments, the DNA polymerase is attached to the napDNAbp by a linker. In some embodiments, the linker is at least about 5-50 amino acids in length. In some embodiments, the linker is at least about 10-30 amino acids in length. In some embodiments, the linker comprises a sequence selected from the group consisting of (SGGS)ₙ (SEQ ID NO: 14), (GGGS)ₙ (SEQ ID NO: 15), (GGGGS)ₙ (SEQ ID NO: 16), (G)ₙ, (EAAAK)ₙ (SEQ ID NO: 17), (GGS)ₙ, SGSETPGTSESATPES (SEQ ID NO: 18), and (XP)ₙ motif. In some embodiments, the linker comprises the amino acid sequence: SGGSSGGSSGSETPGTSESATPES (SEQ ID NO: 19). In some embodiments, the napDNAbp nicks the second strand of the double-stranded target polynucleotide at a target locus of interest. In some embodiments, the DNA polymerase is a modified DNA polymerase that does not occur in nature. In some embodiments, the prime editing system further comprising at least one nuclear localization signals (NLS). In some embodiments, the napDNAbp and/or the DNA polymerase comprise an NLS. In some embodiments, the at least one nuclear localization sequence (NLS) comprises an amino acid sequence selected from the group consisting of KRTADGSEFESPKKKRKV (SEQ ID NO: 7), KRPAATKKAGQAKKKK (SEQ ID NO: 8), KKTELQTTNAENKTKKL (SEQ ID NO: 9), KRGINDRNFWRGENGRKTR (SEQ ID NO: 10), RKSGKIAAIVVKRPRK (SEQ ID NO: 11), PKKKRKV (SEQ ID NO: 12), or MDSLLMNRRKFLYQFKNVRWAKGRRETYLC (SEQ ID NO: 13). In some embodiments, the at least one NLS is a bipartite NLS.

In some embodiments, the disclosure provides a method of modifying a double-stranded target polynucleotide, comprising contacting the double-stranded target polynucleotide with the prime editing composition or the prime editor system of this disclosure. In some embodiments, the method is carried out in a cell. In some embodiments, the cell is in vitro or in vivo.

In some embodiments, the disclosure provides a cell comprising the prime editor system or the prime editing composition of this disclosure.

In some embodiments, the disclosure provides a cell comprising the vector of this disclosure. In some embodiments, the cell is a bacterial cell, plant cell, insect cell, or mammalian cell. In some embodiments, the cell is a human cell.

In some embodiments, the disclosure provides a pharmaceutical composition comprising the chimeric PEg polynucleotide of any this disclosure, the prime editor fusion protein of this disclosure, the nucleic acid of this disclosure, the vector of this disclosure, the prime editor system of this disclosure, and/or the cell of this disclosure.

In some embodiments, the pharmaceutical composition further comprises a pharmaceutically acceptable excipient. In some embodiments, the pharmaceutical composition further comprises a lipid. In some embodiments, the lipid is a cationic lipid. In some embodiments, the pharmaceutical composition further comprises a lipid nanoparticle (LNP).

In some embodiments, the disclosure provides a kit comprising the chimeric PEg polynucleotide of this disclosure, the prime editor fusion protein of this disclosure, the nucleic acid of this disclosure, the vector of this disclosure, the prime editor system of this disclosure, the cell of this disclosure, and/or the pharmaceutical composition of this disclosure.

In some embodiments, the kit further comprises instructions for using the kit to edit one or more polynucleotides.

### BRIEF DESCRIPTION OF THE DRAWINGS

FIG. 1 provides a schematic diagram illustrating incorporating an intended nucleotide edit in genomic DNA using a prime editor containing a nucleic acid programmable DNA binding protein (e.g., Cas9 nickase) fused to a DNA polymerase and a chimeric DNA/RNA prime editing guide polynucleotide.

FIG. 2 provides a schematic diagram illustrating incorporating an intended nucleotide edit in genomic DNA using a prime editor containing a nucleic acid programmable DNA binding protein (e.g., a Cas9 nickase), a DNA polymerase that is not fused to the programmable nickase, and a chimeric DNA/RNA prime editing guide polynucleotide.

FIG. 3 provides a schematic illustrating DNA/RNA hybrid constructs and approximate functional regions for 5′ and 3′ extended chimeric DNA/RNA prime editing guide polynucleotide.

### DETAILED DESCRIPTION OF THE INVENTION

DNA editing has emerged as a viable means to modify disease states by correcting pathogenic mutations at the genetic level. Until recently, all DNA editing platforms have functioned by inducing a DNA double strand break (DSB) at a specified genomic site and relying on endogenous DNA repair pathways to determine the product outcome in a semi-stochastic manner, resulting in complex populations of genetic products. Though precise, user-defined repair outcomes can be achieved through the homology directed repair (HDR) pathway, a number of challenges can prevent high efficiency repair using HDR in therapeutically-relevant cell types. In practice, this pathway is inefficient relative to the competing, error-prone non-homologous end joining pathway. Further, HDR is tightly restricted to the G1 and S phases of the cell cycle, preventing precise repair of DSBs in post-mitotic cells.

The disclosure provides compositions comprising prime editors featuring a nucleic acid programmable DNA binding protein (napDNAbp) (e.g., a napDNAbp having nickase activity), a DNA polymerase (e.g., DNA polymerase α, β, γ, δ, or ε), and chimeric PE guide polynucleotides (e.g., DNA-RNA or RNA-DNA guide). Also provided herein are methods of using such prime editors and chimeric PE guide polynucleotides for editing a double-stranded target polynucleotide.

### Definitions

The following definitions supplement those in the art and are directed to the current application and are not to be imputed to any related or unrelated case, e.g., to any commonly owned patent or application. Although any methods and materials similar or equivalent to those described herein can be used in the practice for testing of the present disclosure, the preferred materials and methods are described herein. Accordingly, the terminology used herein is for the purpose of describing particular embodiments only and is not intended to be limiting.

Unless defined otherwise, all technical and scientific terms used herein have the meaning commonly understood by a person skilled in the art to which this disclosure belongs. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991).

In this application, the use of the singular includes the plural unless specifically stated otherwise. It must be noted that, as used in the specification, the singular forms “a,” “an,” and “the” include plural references unless the context clearly dictates otherwise. In this application, the use of “or” means “and/or,” unless stated otherwise, and is understood to be inclusive. Furthermore, use of the term “including” as well as other forms, such as “include,” “includes,” and “included,” is not limiting.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, such as within 5-fold or within 2-fold, of a value. Where particular values are described in the application and claims, unless otherwise stated the term “about” meaning within an acceptable error range for the particular value should be assumed.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures.

As used herein, a “cell” can generally refer to a biological cell. A cell can be the basic structural, functional and/or biological unit of a living organism. A cell can originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant, an animal cell, a cell from an invertebrate animal (e.g. fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), et cetera. Sometimes a cell may not originate from a natural organism (e.g., a cell can be synthetically made, sometimes termed an artificial cell). In some embodiments, the cell is a human cell. A cell can be of or derived from different tissues, organs, and/or cell types.

“Administering” is referred to herein as providing one or more compositions described herein to a patient or a subject. By way of example and without limitation, composition administration, e.g., injection, can be performed by intravenous (i.v.) injection, sub-cutaneous (s.c.) injection, intradermal (i.d.) injection, intraperitoneal (i.p.) injection, or intramuscular (i.m.) injection. One or more such routes can be employed. Parenteral administration can be, for example, by bolus injection or by gradual perfusion over time. Alternatively, or concurrently, administration can be by the oral route.

By “agent” is meant any small molecule chemical compound, antibody, nucleic acid molecule, or polypeptide, or fragments thereof.

By “ameliorate” is meant decrease, suppress, attenuate, diminish, arrest, or stabilize the development or progression of a disease.

By “analog” is meant a molecule that is not identical but has analogous functional or structural features. For example, a polynucleotide or polypeptide analog retains the biological activity of a corresponding naturally occurring polynucleotide or polypeptide, while having certain modifications that enhance the analog's function relative to a naturally occurring polynucleotide or polypeptide. Such modifications could increase the analog's affinity for DNA, efficiency, specificity, protease or nuclease resistance, membrane permeability, and/or half-life, without altering, for example, ligand binding. An analog may include an unnatural nucleotide or amino acid.

The term “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz, G. E. and Schirmer, R. H., Principles of Protein Structure, Springer-Verlag, New York (1979)). According to such analyses, groups of amino acids can be defined where amino acids within a group exchange preferentially with each other, and therefore resemble each other most in their impact on the overall protein structure (Schulz, G. E. and Schirmer, R. H., supra). Non-limiting examples of conservative mutations include amino acid substitutions of amino acids, for example, lysine for arginine and vice versa such that a positive charge can be maintained; glutamic acid for aspartic acid and vice versa such that a negative charge can be maintained; serine for threonine such that a free —OH can be maintained; and glutamine for asparagine such that a free —NH₂ can be maintained.

The term “coding sequence” or “protein coding sequence” as used interchangeably herein refers to a segment of a polynucleotide that codes for a protein. The region or sequence is bounded nearer the 5′ end by a start codon and nearer the 3′ end with a stop codon. Coding sequences can also be referred to as open reading frames.

The term “complement”, “complementary”, or “complementarity” as used herein, refers to the ability of two polynucleotide molecules to base pair with each other. Complementary polynucleotides may base pair via hydrogen bonding, which may be Watson-Crick, Hoogsteen or reversed Hoogsteen hydrogen bonding. For example, an adenine on one polynucleotide molecule will base pair to a guanine on a second polynucleotide molecule and a cytosine on one polynucleotide molecule will base pair to a thymine or uracil on a second polynucleotide molecule. Two polynucleotide molecules are complementary to each other when a first polynucleotide molecule comprising a first nucleotide sequence can base pair with a second polynucleotide molecule comprising a second nucleotide sequence. For instance, the two DNA molecules 5′-ATGC-3′ and 5′-GCAT-3′ are complementary, and the complement of the DNA molecule 5′-ATGC-3′ is 5′-GCAT-3′. A percentage of complementarity indicates the percentage of nucleotides in a polynucleotide molecule which can base pair with a second polynucleotide molecule (e.g., 5, 6, 7, 8, 9, 10 out of 10 being 50%, 60%, 70%, 80%, 90%, and 100% complementary, respectively). “Perfectly complementary” means that all the contiguous nucleotides of a polynucleotide molecule will base pair with the same number of contiguous nucleotides in a second polynucleotide molecule. “Substantially complementary” as used herein refers to a degree of complementarity that can be 70%, 75%, 80%, 85%, 90%, 95%, 97%, 98%, or 99% over all or a portion of two polynucleotide molecules. In some embodiments, the portion of complementarity may be a region of 10, 15, 20, 25, 30, 35, 40, 45, 50, or more nucleotides. “Substantial complementary” can also refer to a 100% complementarity over a portion of two polynucleotide molecules. In some embodiments, the portion of complementarity between the two polynucleotide molecules is at least 60%, 65%, 70%, 75%, 80%, 85%, 90%, 95%, 97%, 98%, or 99% of the length of at least one of the two polynucleotide molecules or a functional or defined portion thereof.

“Detect” refers to identifying the presence, absence or amount of the analyte to be detected. In one embodiment, a sequence alteration in a polynucleotide or polypeptide is detected. In another embodiment, the presence of indels is detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition or disorder that damages or interferes with the normal function of a cell, tissue, or organ.

The term “effective amount,” as used herein, refers to an amount of a biologically active agent that is sufficient to elicit a desired biological response. The effective amount of an active agent(s) used to practice the present disclosure for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount. In one embodiment, an effective amount is the amount of prime editing composition of the disclosure (e.g., a complex of a nucleic acid programmable DNA binding protein (napDNAbp) with nickase activity (e.g., nCas9), a DNA polymerase (e.g., DNA polymerase α, β, γ, δ, or ε), and chimeric PE guide polynucleotide) sufficient to introduce one or more intended nucleotide edits in a gene of interest in a cell (e.g., a cell in vitro or in vivo). In one embodiment, an effective amount is the amount of a prime editing composition required to achieve a therapeutic effect (e.g., to reduce or control a disease or a symptom or condition thereof). Such therapeutic effect need not be sufficient to alter a gene of interest in all cells of a subject, tissue or organ, but only to alter a gene of interest in about 1%, 5%, 10%, 25%, 50%, 75% or more of the cells present in a subject, tissue or organ.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507). For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In one embodiment, hybridization will occur at 30° C. in 750 mM NaCl, 75 mM trisodium citrate, and 1% SDS. In another embodiment, hybridization will occur at 37° C. in 500 mM NaCl, 50 mM trisodium citrate, 1% SDS, 35% formamide, and 100 μg/ml denatured salmon sperm DNA (ssDNA). In another embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In an embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “increases” or “decreases” is meant a positive or negative, respectively, alteration of at least 10%, 25%, 50%, 75%, or 100%.

The term “linker” as used herein can refer to a covalent linker (e.g., covalent bond), a non-covalent linker, a chemical group, or a molecule linking two molecules or moieties, e.g., two components of a protein complex or a ribonucleocomplex, or two domains of a prime editor fusion protein, such as, for example, a nucleic acid programmable DNA binding protein (napDNAbp) having nickase activity (e.g., nCas9) and a DNA polymerase (e.g., DNA polymerase α, β, γ, δ, and ε). A linker can join different components of, or different portions of components of, a prime editing composition. For example, in some embodiments, a linker can join a napDNAbp (e.g., nCas9) and a DNA polymerase (e.g., DNA polymerase α, β, γ, δ, and ε). In some embodiments, a linker can join a Cas9 and a DNA polymerase. In some embodiments, a linker can join a nCas9 and a DNA polymerase. In some embodiments, a linker can join a chimeric PE guide polynucleotide (e.g., DNA-RNA or RNA-DNA guide) and a DNA polymerase. In some embodiments, a linker can join a DNA polymerase component and a napDNAbp component of a prime editing composition. In some embodiments, a linker can join a DNA segment and an RNA segment of a chimeric PE guide polynucleotide.

A linker can be positioned between, or flanked by, two groups, molecules, or other moieties and connected to each one via a covalent bond or non-covalent interaction, thus connecting the two. In some embodiments, the linker can be an organic molecule, group, polymer, or chemical moiety. In some embodiments, the linker can be a polynucleotide. In some embodiments, the linker can be a DNA linker. In some embodiments, the linker can be an RNA linker.

The linker may be as simple as a covalent bond, or it may be a polymeric linker many atoms in length. In some embodiments, the linker can be an amino acid or a polypeptide comprising a plurality of amino acids. In some embodiments, the linker can be about 5-100 amino acids in length, for example, about 5, 6, 7, 8, 9, 10, 11, 12, 13, 14, 15, 16, 17, 18, 19, 20, 20-30, 30-40, 40-50, 50-60, 60-70, 70-80, 80-90, or 90-100 amino acids in length. In some embodiments, the linker can be about 100-150, 150-200, 200-250, 250-300, 300-350, 350-400, 400-450, or 450-500 amino acids in length. In some embodiments, the linker is 5-200 amino acids in length, for example, 5, 6, 7, 8, 9, 10, 11, 12, 13, 14, 15, 16, 17, 18, 19, 20, 25, 35, 45, 50, 55, 60, 60, 65, 70, 70, 75, 80, 85, 90, 90, 95, 100, 101, 102, 103, 104, 105, 110, 120, 130, 140, 150, 160, 175, 180, 190, or 200 amino acids in length. Longer or shorter linkers can be also contemplated.

In other embodiments, the linker is not a polypeptide. In certain embodiments, the linker is a covalent bond (e.g., a carbon-carbon bond, disulfide bond, carbon-heteroatom bond, etc.). In certain embodiments, the linker is a carbon-nitrogen bond of an amide linkage. In certain embodiments, the linker is a cyclic or acyclic, substituted or unsubstituted, branched or unbranched aliphatic or heteroaliphatic linker. In certain embodiments, the linker is polymeric (e.g., polyethylene, polyethylene glycol, polyamide, polyester, etc.). In certain embodiments, the linker comprises a monomer, dimer, or polymer of aminoalkanoic acid. In certain embodiments, the linker comprises an aminoalkanoic acid (e.g., glycine, ethanoic acid, alanine, beta-alanine, 3-aminopropanoic acid, 4-aminobutanoic acid, 5-pentanoic acid, etc.). In certain embodiments, the linker comprises a monomer, dimer, or polymer of aminohexanoic acid (Ahx). In certain embodiments, the linker is based on a carbocyclic moiety (e.g., cyclopentane, cyclohexane). In other embodiments, the linker comprises a polyethylene glycol moiety (PEG). In other embodiments, the linker comprises amino acids. In certain embodiments, the linker comprises a peptide. In certain embodiments, the linker comprises an aryl or heteroaryl moiety. In certain embodiments, the linker is based on a phenyl ring. The linker may include functionalized moieties to facilitate attachment of a nucleophile (e.g., thiol, amino) from the peptide to the linker. Any electrophile may be used as part of the linker. Exemplary electrophiles include, but are not limited to, activated esters, activated amides, Michael acceptors, alkyl halides, aryl halides, acyl halides, and isothiocyanates.

The terms “isolated,” “purified,” or “biologically pure” refer to material that is free to varying degrees from components which normally accompany it as found in its native state. “Isolated” denotes a degree of separation from the material's original source or surroundings while “isolate” refers to the process of obtaining an isolated material. “Purified” denotes a degree of separation that is higher than isolation while “purify” refers to the process of obtaining a purified material. A “purified” or “biologically pure” material is sufficiently free of other materials such that any impurities do not materially affect the biological properties of the material. For example, a nucleic acid or polypeptide of this disclosure is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis, high-performance liquid chromatography (HPLC), ultra HPLC (UHPLC), liquid chromatography/mass spectrometry (LC/MS) or gas chromatography/mass spectrometry (GC/MS).

By “isolated polynucleotide” is meant a nucleic acid molecule (e.g., a DNA molecule or an RNA molecule) that is separated from other nucleic acid or cellular components that normally accompany it as found in its native state. For example, an isolated DNA molecule may be one that is substantially free of the chromosomal DNA that flank the DNA molecule in the genome of the organism from which the DNA molecule of the disclosure is derived. The term therefore includes, for example, a DNA molecule (e.g., a recombinant DNA molecule) that is incorporated into a vector such as an autonomously replicating plasmid or virus; the genomic DNA of a prokaryote or eukaryote with which the recombinant DNA is not normally associated or at a different location within the genomic DNA than it is normally located; a prokaryotic cell or eukaryotic cell or tissue, wherein the cell or tissue does not normally comprise the recombinant DNA; or that exists as a separate molecule (for example, a cDNA or a genomic or cDNA fragment produced by PCR or restriction endonuclease digestion) independent of other sequences. An isolated RNA molecule may be one that is separated from the cellular materials from which the RNA molecule is derived. The term includes, for example, an RNA molecule that is incorporated into a vector such as a plasmid or virus; a prokaryotic cell or eukaryotic cell or tissue, wherein the cell or tissue does not normally comprise the RNA molecule; or that exists as a separate molecule (e.g., a synthetic or recombinantly-produced RNA molecule). In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the disclosure that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the disclosure. An isolated polypeptide of the disclosure may be obtained, for example, by extraction from a natural source, by expression of a recombinant nucleic acid encoding such a polypeptide; or by chemically synthesizing the protein.

The term “encode” as it is applied to polynucleotides refers to a polynucleotide which is said to “encode” another polynucleotide, a polypeptide, or an amino acid if, in its native state or when manipulated by methods well known to those skilled in the art, it can be used as polynucleotide synthesis template, e.g., transcribed into an RNA, reverse transcribed into a DNA or cDNA, and/or translated to produce an amino acid, or a polypeptide or fragment thereof. In some embodiments, a polynucleotide comprising three contiguous nucleotides form a codon that encodes a specific amino acid. In some embodiments, a polynucleotide comprises one or more codons that encode an amino acid or a polypeptide. In some embodiments, a polynucleotide comprising one or more codons comprises a mutation in a codon compared to a wild-type polynucleotide or a reference polynucleotide. In some embodiments, the mutation in the codon encodes an amino acid substitution in a polypeptide encoded by the polynucleotide as compared to a wild-type polypeptide or a reference polypeptide.

The term “mutation” as used herein refers to a change and/or alteration in an amino acid sequence of a protein or nucleic acid sequence of a polynucleotide. Such changes and/or alterations may comprise the substitution, insertion, deletion and/or truncation of one or more amino acids, in the case of an amino acid sequence, and/or nucleotides, in the case of nucleic acid sequence, compared to a reference amino acid or nucleic acid sequence. In some embodiments, the reference sequence is a wild-type sequence. In some embodiments, a mutation in a nucleic acid sequence of a polynucleotide encodes a mutation in the amino acid sequence of a polypeptide. In some embodiments, the mutation in the amino acid sequence of the polypeptide or the mutation in the nucleic acid sequence of the polynucleotide is a mutation associated with a disease state.

In general, mutations made or identified in a sequence (e.g., an amino acid sequence as described herein) are numbered in relation to a reference (or wild-type) sequence, i.e., a sequence that does not contain the mutations. The skilled practitioner in the art would readily understand how to determine the position of mutations in amino acid and nucleic acid sequences relative to a reference sequence.

The term “polynucleotide” or “nucleic acid molecule” can be any polymeric form of nucleotides, including DNA, RNA, a hybridization thereof, or RNA-DNA chimeric molecules. In some embodiments, a polynucleotide comprises cDNA, genomic DNA, mRNA, tRNA, rRNA, or microRNA. In some embodiments, a polynucleotide is double stranded, e.g., a double-stranded DNA in a gene. In some embodiments, a polynucleotide is single-stranded or substantially single-stranded, e.g., single-stranded DNA or an mRNA. In some embodiments, a polynucleotide is a cell-free nucleic acid molecule. In some embodiments, a polynucleotide circulates in blood. In some embodiments, a polynucleotide is a cellular nucleic acid molecule. In some embodiments, a polynucleotide is a cellular nucleic acid molecule in a cell circulating in blood.

Polynucleotides can have any three-dimensional structure. The following are nonlimiting examples of polynucleotides: a gene or gene fragment (for example, a probe, primer, EST or SAGE tag), an exon, an intron, intergenic DNA (including, without limitation, heterochromatic DNA), messenger RNA (mRNA), transfer RNA (tRNA), ribosomal RNA (rRNA), a ribozyme, cDNA, a recombinant polynucleotide, a branched polynucleotide, a plasmid, a vector, isolated DNA, isolated RNA, sgRNA, guide RNA, a nucleic acid probe, a primer, an snRNA, a long non-coding RNA, a snoRNA, a siRNA, a miRNA, a tRNA-derived small RNA (tsRNA), an antisense RNA, an shRNA, or a small rDNA-derived RNA (srRNA).

In some embodiments, a polynucleotide comprises deoxyribonucleotides, ribonucleotides or analogs thereof. In some embodiments, a polynucleotide comprises modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component.

In some embodiments, a polynucleotide is composed of a specific sequence of four nucleotide bases: adenine (A); cytosine (C); guanine (G); thymine (T); and uracil (U) for thymine when the polynucleotide is RNA. In some embodiments, the polynucleotide may comprise one or more other nucleotide bases, such as inosine (I), which is read by the translation machinery as guanine (G).

In some embodiments, a polynucleotide may be modified. As used herein, the terms “modified” or “modification” refers to chemical modification with respect to the A, C, G, T and U nucleotides. In some embodiments, modifications may be on the nucleoside base and/or sugar portion of the nucleosides that comprise the polynucleotide. In some embodiments, the modification may be on the internucleoside linkage (e.g., phosphate backbone). In some embodiments, multiple modifications are included in the modified nucleic acid molecule. In some embodiments, a single modification is included in the modified nucleic acid molecule.

Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, and backbone modifications. In some embodiments, a nucleic acid is or comprises natural nucleosides (e.g., adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (2′—e.g., fluororibose, ribose, 2′-deoxyribose, arabinose, and hexose); and/or modified phosphate groups (e.g., phosphorothioates and 5′-N-phosphoramidite linkages). Non-limiting exemplary modified bases include hypoxanthine, xanthine, 7-methylguanine, 5,6-dihydrouracil, 5-methylcytosine (m5C), and 5-hydromethylcytosine.

A “patient” or “subject” as used herein refers to a mammalian subject or individual diagnosed with, at risk of having or developing, or suspected of having or developing a disease or a disorder. In some embodiments, the term “patient” refers to a mammalian subject with a higher than average likelihood of developing a disease or a disorder. Exemplary patients can be humans, non-human primates, cats, dogs, pigs, cattle, cats, horses, camels, llamas, goats, sheep, rodents (e.g., mice, rabbits, rats, or guinea pigs) and other mammalians that can benefit from the therapies disclosed herein. Exemplary human patients can be male or female. In some embodiments, a “subject” refers to a mammal, including, but not limited to, a human or non-human mammal, such as a bovine, equine, canine, ovine, or feline. Subjects include livestock, domesticated animals raised to produce labor and to provide commodities, such as food, including without limitation, cattle, goats, chickens, horses, pigs, rabbits, and sheep.

“Patient in need thereof” or “subject in need thereof” is referred to herein as a patient diagnosed with, at risk or having, predetermined to have, or suspected of having a disease or disorder.

The terms “pathogenic mutation,” “pathogenic variant,” “disease casing mutation,” “disease causing variant,” “deleterious mutation,” or “predisposing mutation” refers to a genetic alteration or mutation that increases an individual's susceptibility or predisposition to a certain disease or disorder.

The term “recombinant” as used herein in the context of proteins or nucleic acids refers to proteins or nucleic acids that do not occur in nature but are the product of human engineering.

The terms “protein” and “polypeptide” can be used interchangeably to refer to a polymer of two or more amino acids joined by covalent bonds (e.g., an amide bond) that can adopt a three-dimensional conformation. In some embodiments, a protein comprises at least two amide bonds. In some embodiments, a protein comprises multiple amide bonds. In some embodiments, a protein or polypeptide comprises at least 10 amino acids, 15 amino acids, 20 amino acids, 30 amino acids or 50 amino acids joined by covalent bonds (e.g., amide bonds). In some embodiments, a protein comprises an enzyme, enzyme precursor proteins, regulatory protein, structural protein, receptor, nucleic acid binding protein, a biomarker, a member of a specific binding pair (e.g., a ligand or aptamer), or an antibody. In some embodiments, a protein includes a full-length protein (e.g., a fully processed protein having certain biological function). In some embodiments, a protein includes a variant or a fragment of a full-length protein. For example, in some embodiments, a Cas9 protein domain comprises a H840A amino acid substitution compared to a naturally occurring S. pyogenes Cas9 protein. A variant of a protein or enzyme, for example a variant DNA polymerase (e.g., a DNA-dependent DNA polymerase), or a variant reverse transcriptase, may include polypeptides having about 60% identical, about 70% identical, about 80% identical, about 90% identical, about 95% identical, about 96% identical, about 97% identical, about 98% identical, about 99% identical, about 99.5% identical, or about 99.9% identical to a reference protein.

In some embodiments, a protein comprises one or more protein domains or subdomains. As used herein, the term “polypeptide domain”, “domain”, or “protein domain” refers to a polypeptide chain that has one or more biological functions, e.g., a catalytic function, a protein-protein binding function, or a protein-DNA function. In some embodiments, a protein comprises multiple protein domains. In some embodiments, a protein comprises multiple protein domains that are naturally occurring. In some embodiments, a protein comprises multiple protein domains from different naturally occurring proteins. For example, in some embodiments, a prime editor fusion protein comprises a Cas9 protein domain of S. pyogenes and a DNA polymerase domain. A protein that comprises amino acid sequences from different origins or naturally occurring proteins may be referred to as a prime editor fusion protein or chimeric protein, for example, a prime editor fusion protein.

In some embodiments, a protein comprises a functional variant or functional fragment of a full-length wild-type protein. A “functional fragment” or “functional portion”, as used herein, refers to any portion of a reference protein (e.g., a wild-type protein) that encompasses less than the entire amino acid sequence of the reference protein while retaining one or more of the functions, e.g., catalytic or binding functions. For example, a functional fragment of a DNA polymerase (e.g., a DNA-dependent DNA polymerase) may encompass less than the entire amino acid sequence of a wild-type DNA polymerase (e.g., a wild-type DNA-dependent DNA polymerase), but retains the ability under at least one set of conditions to catalyze the polymerization of a polynucleotide. For example, a functional fragment of a reverse transcriptase may encompass less than the entire amino acid sequence of a wild-type reverse transcriptase, but retains the ability under at least one set of conditions to catalyze the polymerization of a polynucleotide. When the reference protein is a fusion of multiple functional domains, a functional fragment thereof may retain one or more of the functions of at least one of the functional domains. For example, a functional fragment of a Cas9 may encompass less than the entire amino acid sequence of a wild-type Cas9, but retains its DNA binding ability and lacks its nuclease activity partially or completely.

A “functional variant” or “functional mutant”, as used herein, refers to any variant or mutant of a reference protein (e.g., a wild-type protein) that encompasses one or more alterations to the amino acid sequence of the reference protein while retaining one or more of the functions, e.g., catalytic or binding functions. In some embodiments, the one or more alterations to the amino acid sequence comprises amino acid substitutions, insertions or deletions, or any combination thereof. In some embodiments, the one or more alterations to the amino acid sequence comprises amino acid substitutions. For example, a functional variant of a DNA polymerase (e.g., a DNA-dependent DNA polymerase) may encompass less than the entire amino acid sequence of a wild-type DNA polymerase (e.g., a wild-type DNA-dependent DNA polymerase, but retains the ability under at least one set of conditions to catalyze the polymerization of a polynucleotide. For example, a functional variant of a reverse transcriptase may comprise one or more amino acid substitutions compared to the amino acid sequence of a wild-type reverse transcriptase, but retains the ability under at least one set of conditions to catalyze the polymerization of a polynucleotide. When the reference protein is a fusion of multiple functional domains, a functional variant thereof may retain one or more of the functions of at least one of the functional domains. For example, in some embodiments, a functional variant fragment of a Cas9 may comprise one or more amino acid substitutions in a nuclease domain, e.g., an H840A amino acid substitution, compared to the amino acid sequence of a wild-type Cas9, but retains the DNA binding ability and lacks the nuclease activity partially or completely. A variant of a protein or enzyme, for example a variant DNA polymerase, or a variant Cas9 or a variant Cpf1 comprises a polypeptide having an amino acid sequence that is about 60% identical, about 70% identical, about 80% identical, about 90% identical, about 95% identical, about 96% identical, about 97% identical, about 98% identical, about 99% identical, about 99.5% identical, or about 99.9% identical to the amino acid sequence of a reference protein.

For example, in some embodiments, a functional fragment of a Cas9 may comprise one or more amino acid substitutions in a nuclease domain, e.g., an H840A amino acid substitution, compared to the amino acid sequence of a wild-type Cas9, but retains the DNA binding ability and lacks the nuclease activity partially or completely.

The term “function” and its grammatical equivalents as used herein may refer to a capability of operating, having, or serving an intended purpose. Functional may comprise any percent from baseline to 100% of an intended purpose. For example, functional may comprise or comprise about 5%, 10%, 15%, 20%, 25%, 30%, 35%, 40%, 45%, 50%, 55%, 60%, 65%, 70%, 75%, 80%, 85%, 90%, 95%, or up to about 100% of an intended purpose. In some embodiments, the term functional may mean over or over about 100% of normal function, for example, 125%, 150%, 175%, 200%, 250%, 300%, 400%, 500%, 600%, 700% or up to about 1000% of an intended purpose.

Any of the proteins provided herein can be produced by any method known in the art. For example, the proteins provided herein can be produced via recombinant protein expression and purification, which is especially suited for prime editor fusion proteins comprising a peptide linker. Methods for recombinant protein expression and purification are well known, and include those described by Green and Sambrook, Molecular Cloning: A Laboratory Manual (4th ed., Cold Spring Harbor Laboratory Press, Cold Spring Harbor, N.Y. (2012)), the entire contents of which are incorporated herein by reference.

In some embodiments, a protein or polypeptide includes naturally occurring amino acids (e.g., one of the twenty amino acids commonly found in peptides synthesized in nature, and known by the one letter abbreviations A, R, N, C, D, Q, E, G, H, I, L, K, M, F, P, S, T, W, Y and V). In some embodiments, a protein or polypeptides includes non-naturally occurring amino acids (e.g., amino acids which is not one of the twenty amino acids commonly found in peptides synthesized in nature, including synthetic amino acids, amino acid analogs, and amino acid mimetics). In some embodiments, a protein or polypeptide is modified.

Polypeptides and proteins disclosed herein (including functional fragments and functional variants thereof) can comprise synthetic amino acids in place of one or more naturally-occurring amino acids. Such synthetic amino acids are known in the art, and include, for example, aminocyclohexane carboxylic acid, norleucine, α-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, α-naphthylalanine, cyclohexylalanine, cyclohexylglycine, indoline-2-carboxylic acid, 1,2,3,4-tetrahydroisoquinoline carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, α-aminocyclohexane carboxylic acid, α-aminocycloheptane carboxylic acid, α-(2-amino-2-norbornane)-carboxylic acid, α,γ-diaminobutyric acid, α,β-diaminopropionic acid, homophenylalanine, and α-tert-butylglycine. The polypeptides and proteins can be associated with post-translational modifications of one or more amino acids of the polypeptide constructs. Non-limiting examples of post-translational modifications include phosphorylation, acylation including acetylation and formylation, glycosylation (including N-linked and O-linked), amidation, hydroxylation, alkylation including methylation and ethylation, ubiquitylation, addition of pyrrolidone carboxylic acid, formation of disulfide bridges, sulfation, myristoylation, palmitoylation, isoprenylation, farnesylation, geranylation, glypiation, lipoylation and iodination.

By “reference” is meant a standard or control condition. In one embodiment, the reference is a wild-type or healthy cell. In other embodiments and without limitation, a reference is an untreated cell that is not subjected to a test condition, or is subjected to placebo or normal saline, medium, buffer, and/or a control vector that does not harbor a polynucleotide of interest.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, at least about 20 amino acids, at least about 25 amino acids, about 35 amino acids, about 50 amino acids, or about 100 amino acids. For nucleic acids, the length of the reference nucleic acid sequence will generally be at least about 50 nucleotides, at least about 60 nucleotides, at least about 75 nucleotides, about 100 nucleotides or about 300 nucleotides or any integer thereabout or therebetween. In some embodiments, a reference sequence is a wild-type sequence of a protein of interest. In other embodiments, a reference sequence is a polynucleotide sequence encoding a wild-type protein.

By “specifically binds” is meant a nucleic acid molecule, polypeptide, or complex thereof (e.g., a nucleic acid programmable DNA binding domain, DNA polymerase and chimeric PE guide polynucleotide), compound, or molecule that recognizes and binds a polypeptide and/or nucleic acid molecule of the disclosure, but which does not substantially recognize and bind other molecules in a sample, for example, a biological sample.

By “split” is meant divided into two or more fragments.

By “substantially identical” is meant a polypeptide or nucleic acid molecule exhibiting at least 70% identity to a reference amino acid sequence (for example, any one of the amino acid sequences described herein) or nucleic acid sequence (for example, any one of the nucleic acid sequences described herein). In one embodiment, such a sequence is at least 70%, 80% or 85%, 90%, 95% or 99% identical to the amino acid sequence or nucleic acid sequence of the sequence used for comparison. Polynucleotides having “substantial identity” to an endogenous sequence are typically capable of hybridizing with at least one strand of a double-stranded nucleic acid molecule.

The terms “homologous,” “homology,” or “percent homology” as used herein refer to the degree of sequence identity between an amino acid and a corresponding reference amino acid sequence, or a polynucleotide sequence and a corresponding reference polynucleotide sequence. “Homology” can refer to polymeric sequences, e.g., polypeptide or DNA sequences that are similar. Homology can mean, for example, nucleic acid sequences with at least about: 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity. In other embodiments, a “homologous sequence” of nucleic acid sequences can exhibit 93%, 95% or 98% sequence identity to the reference nucleic acid sequence. For example, a “region of homology to a genomic region” can be a region of DNA that has a similar sequence to a given genomic region in the genome. A region of homology can be of any length that is sufficient to promote binding of a spacer, a primer binding site, or a protospacer sequence to the genomic region. For example, the region of homology can comprise at least 5, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100 or more bases in length such that the region of homology has sufficient homology to undergo binding with the corresponding genomic region.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence. COBALT is used, for example, with the following parameters: a) alignment parameters: Gap penalties−11,−1 and End-Gap penalties-5,−1, b) CDD Parameters: Use RPS BLAST on; Blast E-value 0.003; Find Conserved columns and Recompute on, and c) Query Clustering Parameters: Use query clusters on; Word Size 4; Max cluster distance 0.8; Alphabet Regular. EMBOSS Needle is used, for example, with the following parameters: a) Matrix: BLOSUM62; b) GAP OPEN: 10; c) GAP EXTEND: 0.5; d) OUTPUT FORMAT: pair; e) END GAP PENALTY: false; f) END GAP OPEN: 10; and g) END GAP EXTEND: 0.5.

When a percentage of sequence homology or identity is specified, in the context of two nucleic acid sequences or two polypeptide sequences, the percentage of homology or identity generally refers to the alignment of two or more sequences across a portion of their length when compared and aligned for maximum correspondence. When a position in the compared sequence can be occupied by the same base or amino acid, then the molecules can be homologous at that position. A skilled person understands that amino acid (or nucleotide) positions can be determined in homologous sequences based on alignment, for example, “H840” in a reference Cas9 sequence can correspond to H839, or another position in a Cas9 homolog. Unless stated otherwise, sequence homology or identity is assessed over the specified length of the nucleic acid, polypeptide or portion thereof. In some embodiments, the homology or identity is assessed over a functional portion or specified portion of the length. Alignment of sequences for assessment of sequence homology can be conducted by algorithms known in the art, such as the Basic Local Alignment Search Tool (BLAST) algorithm, which is described in Altschul et al, J. Mol. Biol. 215:403-410, 1990. A publicly available, internet interface, for performing BLAST analyses is accessible through the National Center for Biotechnology Information. Additional known algorithms include those published in: Smith & Waterman, “Comparison of Biosequences”, Adv. Appl. Math. 2:482, 1981; Needleman & Wunsch, “A general method applicable to the search for similarities in the amino acid sequence of two proteins” J. Mol. Biol. 48:443, 1970; Pearson & Lipman “Improved tools for biological sequence comparison”, Proc. Natl. Acad. Sci. USA 85:2444, 1988; or by automated implementation of these or similar algorithms. Global alignment programs can also be used to align similar sequences of roughly equal size. Examples of global alignment programs include NEEDLE (available at www.ebi.ac.uk/Tools/psa/emboss_needle/) which is part of the EMBOSS package (Rice P et al., Trends Genet., 2000; 16: 276-277), and the GGSEARCH program https://fasta.bioch.virginia.edu/fasta_www2/, which is part of the FASTA package (Pearson W and Lipman D, 1988, Proc. Natl. Acad. Sci. USA, 85: 2444-2448). Both of these programs are based on the Needleman-Wunsch algorithm which is used to find the optimum alignment (including gaps) of two sequences along their entire length. A detailed discussion of sequence analysis can also be found in Unit 19.3 of Ausubel et al (“Current Protocols in Molecular Biology” John Wiley & Sons Inc, 1994-1998, Chapter 15, 1998). In some embodiments, alignment between a query sequence and a reference sequence is performed with Needleman-Wunsch alignment with Gap Costs set to Existence: 11 Extension: 1 where percent identity is calculated by dividing the number of identities by the length of the alignment, as further described in Altschul et al. (“Gapped BLAST and PSIBLAST: a new generation of protein database search programs”, Nucleic Acids Res. 25:3389-3402, 1997) and Altschul et al, (“Protein database searches using compositionally adjusted substitution matrices”, FEBS J. 272:5101-5109, 2005).

The terms “treatment” or “treating” and their grammatical equivalents may refer to the medical management of a subject with an intent to cure, stabilize the progression of, or ameliorate a symptom of, a disease, condition, or disorder. Treatment may include active treatment, that is, treatment directed specifically toward the improvement of a disease, condition, or disorder. Treatment may include causal treatment, that is, treatment directed toward removal of the cause of the associated disease, condition, or disorder. In addition, this treatment may include palliative treatment, that is, treatment designed for the relief of symptoms rather than the curing of the disease, condition, or disorder. Treatment may include supportive treatment, that is, treatment employed to supplement another specific therapy directed toward the improvement of the disease, condition, or disorder. In some embodiments, a condition may be pathological. In some embodiments, a treatment may not completely cure or prevent a disease, condition, or disorder. In some embodiments, a treatment ameliorates or partially prevents, but not completely cure or prevent a disease, condition, or disorder. In some embodiments, a subject may be treated for 12 hours, 24 hours, 2 days, 3 days, 4 days, 5 days, 6 days, 7 days, 2 weeks, 3 weeks, 4 weeks, 2 months, 3 months, 4 months, 5 months, 6 months, 1 year, 2 years, 3 years, 4 years, 5 years, 6 years, indefinitely, or life of the subject.

The terms “prevent” or “preventing” means delaying, forestalling, or avoiding the onset or development of a disease, condition, or disorder for a period of time. Prevent also means reducing risk of developing a disease, disorder, or condition. Prevention includes minimizing or partially or completely inhibiting the development of a disease, condition, or disorder. In some embodiments, a composition, e.g., a pharmaceutical composition, prevents a disorder by delaying the onset of the disorder for 12 hours, 24 hours, 2 days, 3 days, 4 days, 5 days, 6 days, 7 days, 2 weeks, 3 weeks, 4 weeks, 2 months, 3 months, 4 months, 5 months, 6 months, 1 year, 2 years, 3 years, 4 years, 5 years, 6 years, indefinitely, or life of a subject.

By “upstream” and “downstream” it is intended to define relevant positions at least two regions or sequences in a nucleic acid molecule orientated in a 5′-to-3′ direction. For example, a first sequence is upstream of a second sequence in a DNA molecule where the first sequence is positioned 5′ to the second sequence. Accordingly, the second sequence is downstream of the first sequence.

### Prime Editing

The term “prime editing” refers to programmable editing of a target DNA using a prime editing composition comprising a prime editor and a PEg polynucleotide to incorporate an intended nucleotide edit into the target DNA through target-primed DNA synthesis. A target gene of prime editing may comprise a double stranded DNA molecule having two complementary strands: a first strand that may be referred to as a “target strand” or a “non-edit strand”, and a second strand that may be referred to as a “non-target strand,” or an “edit strand.” In some embodiments, in a prime editing guide polynucleotide, e.g., a PEgRNA or a chimeric prime editor (PE) guide polynucleotide, a spacer sequence is complementary or substantially complementary to a specific sequence on the target strand, which may be referred to as a “search target sequence”. In some embodiments, the spacer sequence is annealed to the target strand at the search target sequence. In some embodiments, the spacer sequence is capable of annealing to the target strand at the search target sequence. The target strand may also be referred to as the “non-Protospacer Adjacent Motif (non-PAM) strand.” In some embodiments, the non-target strand may also be referred to as the “PAM strand”. In some embodiments, the PAM strand comprises a protospacer sequence and optionally a protospacer adjacent motif (PAM) sequence. A protospacer sequence refers to a specific sequence in the PAM strand of the target gene that is complementary to the search target sequence. In a PE guide polynucleotide, a spacer sequence may have substantially identical sequence as the protospacer sequence on the edit strand of a target gene, except that, in some embodiments wherein the spacer sequence comprises RNA, the spacer sequence may comprise Uracil (U) and the protospacer sequence may comprise Thymine (T).

In some embodiments, the double stranded target polynucleotide comprises a nick site on the PAM strand (or non-target strand). As used herein, a “nick site” refers to a specific position in between two nucleotides or two base pairs of the double stranded target polynucleotide. In some embodiments, the position of a nick site is determined relative to the position of a specific PAM sequence. In some embodiments, the nick site is the particular position where a nick will occur when the double stranded target DNA is contacted with a nickase, for example, a Cas nickase, that recognizes a specific PAM sequence. In some embodiments, the nick site is upstream of a specific PAM sequence on the PAM strand of the double stranded target DNA. In some embodiments, the nick site is downstream of a specific PAM sequence on the PAM strand of the double stranded target DNA. In some embodiments, the nick site is 3 nucleotides upstream of the PAM sequence, and the PAM sequence is recognized by a Streptococcus pyogenes Cas9 nickase, a P. lavamentivorans Cas9 nickase, a C. diphtheriae Cas9 nickase, a N. cinerea Cas9, a S. aureus Cas9, or a N. lari Cas9 nickase. In some embodiments, the nick site is 3 nucleotides upstream of the PAM sequence, and the PAM sequence is recognized by a Cas9 nickase, wherein the Cas9 nickase comprises a nuclease inactive HNH domain and a nuclease active RuvC domain. In some embodiments, the nick site is 3 nucleotides upstream of the PAM sequence, and the PAM sequence is recognized by a Cas9 nickase, wherein the Cas9 nickase comprises a nuclease active HNH domain and a nuclease inactive RuvC domain. In some embodiments, the nick site is 2 nucleotides upstream of the PAM sequence, and the PAM sequence is recognized by a S. thermophilus Cas9 nickase.

In some embodiments, a PEg polynucleotide complexes with and directs a prime editor to bind to the search target sequence of the double stranded target DNA, e.g., a target gene. In some embodiments, the bound prime editor generates a nick at a nick site on the edit strand (PAM strand) of the target gene. In some embodiments, a primer binding site (PBS) of the PEg polynucleotide anneals with a free 3′ end formed at the nick site, and the prime editor initiates DNA synthesis from the nick site, using the free 3′ end as a primer. Subsequently, a single-stranded DNA encoded by the editing template of the PEg polynucleotide is synthesized by a DNA polymerase component of the prime editor. In some embodiments, the newly synthesized single-stranded DNA comprises one or more intended nucleotide edits compared to the endogenous target gene sequence. Accordingly, in some embodiments, the editing template of a PEg polynucleotide is complementary to a sequence in the edit strand except for one or more mismatches at the intended nucleotide edit positions in the editing template. The endogenous, e.g., genomic, sequence that is partially complementary to the editing template may be referred to as an “editing target sequence”.

In some embodiments, the newly synthesized single-stranded DNA equilibrates with the editing target sequence on the edit strand of the double stranded target DNA for pairing with the target strand of the double stranded target DNA. In some embodiments, the editing target sequence of the target gene is excised by a flap endonuclease (FEN), for example, FEN1. In some embodiments, the FEN is an endogenous FEN, for example, in a cell comprising the double stranded target DNA, e.g., a target gene. In some embodiments, the FEN is provided as part of the prime editor, either linked to other components of the prime editor or provided in trans. In some embodiments, the newly synthesized single stranded DNA, which comprises the intended nucleotide edit, replaces the endogenous single stranded editing target sequence on the edit strand of the target gene. In some embodiments, the newly synthesized single stranded DNA and the endogenous DNA on the target strand form a heteroduplex DNA structure at the region corresponding to the editing target sequence of the double stranded target DNA. In some embodiments, the newly synthesized single-stranded DNA comprising the nucleotide edit is paired in the heteroduplex with the target strand of the target DNA that does not comprise the nucleotide edit, thereby creating a mismatch. In some embodiments, the mismatch is recognized by DNA repair machinery, e.g., an endogenous DNA repair machinery. In some embodiments, through DNA repair, the intended nucleotide edit is incorporated into the double stranded target DNA.

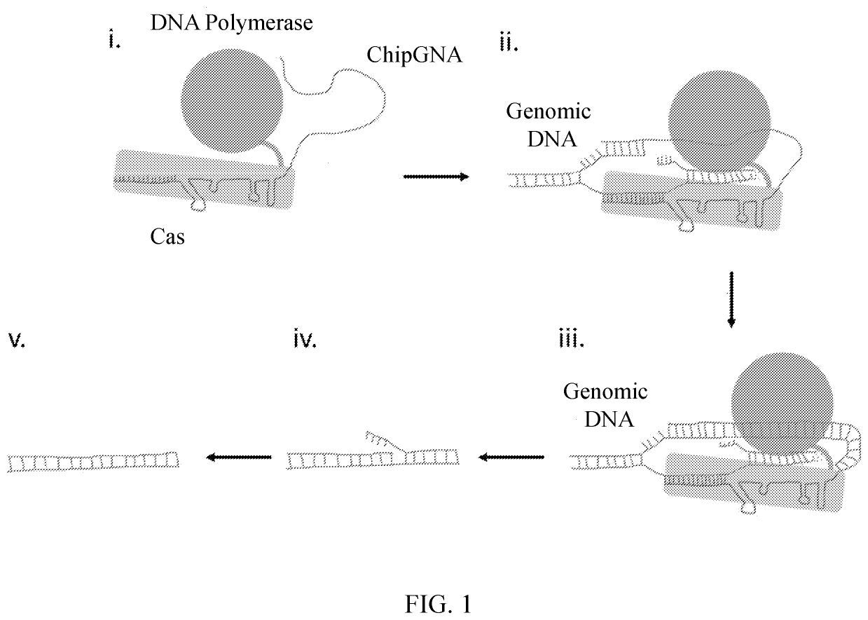

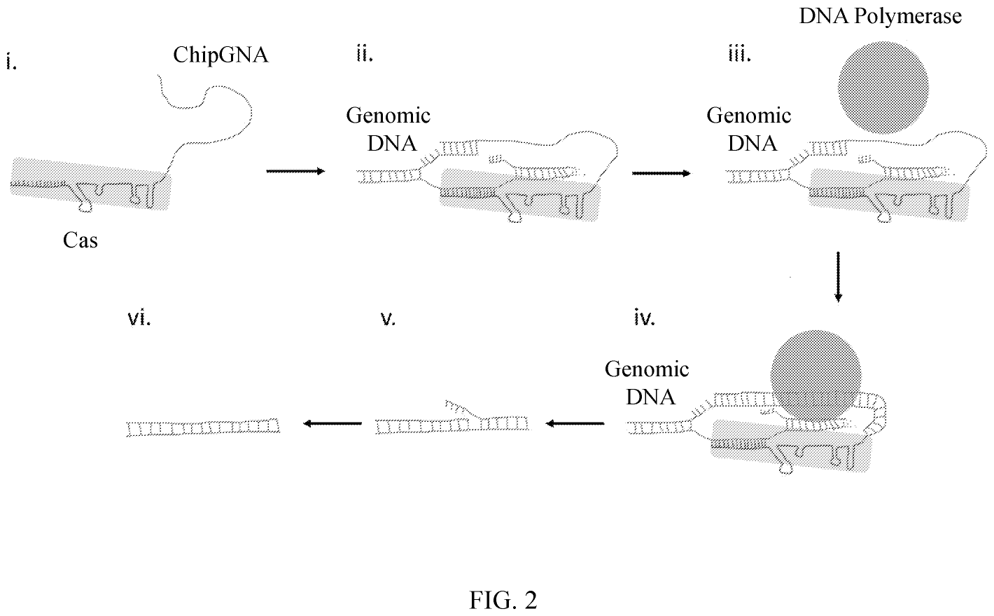

Exemplary prime editing schematics are shown in FIG. 1 and FIG. 2. In some embodiments, a PE guide polynucleotide complexes with and directs a prime editor to bind to the search target sequence of the target gene. In some embodiments, the bound prime editor generates a nick on the edit strand (PAM strand) of the target gene. In some embodiments, a primer binding site (PBS) of the PEgRNA anneals with a free 3′ end formed at the nick site, and the prime editor initiates DNA synthesis from the nick site, using the free 3′ end as a primer. Subsequently, a single stranded DNA encoded by the editing template of the PE guide polynucleotide is synthesized. In some embodiments, the newly synthesized single stranded DNA comprises one or more intended nucleotide edits compared to the endogenous target gene sequence. Accordingly, in some embodiments, the editing template is complementary to a sequence in the edit strand except for one or more mismatches at the intended nucleotide edit positions in the editing template. The endogenous, e.g., genomic, sequence that is partially complementary to the editing template may be referred to as an “editing target sequence”.

The nick on the edit strand generated by the prime editor may depend on the particular napDNAbp (e.g., Cas protein) used in the prime editor. Accordingly, the position of initiation of DNA polymerization may depend on the particular napDNAbp (e.g., Cas protein) used in the prime editor. In some embodiments, a nick generated by the prime editor can be upstream of a PAM. In some embodiments, a nick generated by the prime editor can be about 3 base pairs upstream of a PAM. In some embodiments, an adjacent cut, in some embodiments, a nick generated by the prime editor can be or can be about 10 base pairs upstream of a PAM. In some embodiments, a nick generated by the prime editor can be about 0-20 base pairs upstream of a PAM. In some embodiments, a nick generated by the prime editor can be 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 base pairs upstream of a PAM. In some embodiments, a nick generated by the prime editor can be downstream of a PAM. In some embodiments, a nick generated by the prime editor can be downstream of a PAM by 1 to 30 base pairs.

### Prime Editors

The term “prime editor” (PE) refers to the polypeptide or polypeptide components involved in prime editing. In various embodiments, a prime editor includes i) a polypeptide domain having DNA binding activity, e.g., a napDNAbp, and ii) a polypeptide domain having DNA polymerase activity.

In some embodiments, the prime editor can comprise multiple domains, or a functional fragment or functional variant thereof. For example, the prime editor comprising a polynucleotide programmable nucleotide binding domain derived from Cas9 can comprise an REC lobe and an NUC lobe corresponding to the REC lobe and NUC lobe of a wild-type or natural Cas9. In another example, the prime editor can comprise one or more of a RuvCI domain, BH domain, REC1 domain, REC2 domain, RuvCII domain, L1 domain, HNH domain, L2 domain, RuvCIII domain, WED domain, TOPO domain or CTD domain. In some embodiments, one or more domains of the prime editor comprise a mutation (e.g., substitution, insertion, deletion) relative to a wild-type version of a polypeptide comprising the domain. Different domains of the prime editor can be associated with each other covalently or non-covalently, directly fused, or connected with one or more linkers described herein or known in the art.

In some embodiments, a prime editor comprises a polypeptide domain having DNA binding activity, e.g., a napDNAbp and a polypeptide domain having DNA polymerase activity wherein the amino acid sequences of the polypeptide domain having DNA binding activity and the polypeptide domain having DNA polymerase activity comprise a N terminus methionine. In some embodiments, a prime editor comprises a polypeptide domain having DNA binding activity and a polypeptide domain having DNA polymerase activity wherein the amino acid sequences of the polypeptide domain having DNA binding activity and the polypeptide domain having DNA polymerase activity do not comprise a N terminus methionine. In some embodiments, a prime editor comprises a polypeptide domain having DNA binding activity and a polypeptide domain having DNA polymerase activity wherein the amino acid sequence of the polypeptide domain having DNA binding activity comprises a N terminus methionine and the amino acid sequence of the polypeptide domain having DNA polymerase activity does not comprise a N terminus methionine. In some embodiments, a prime editor comprises a polypeptide domain having DNA binding activity and a polypeptide domain having DNA polymerase activity wherein the amino acid sequence of the polypeptide domain having DNA binding activity does not comprise a N terminus methionine and the amino acid sequence of the polypeptide domain having DNA polymerase activity comprises a N terminus methionine.

In some embodiments, the prime editor comprises a polypeptide domain having nuclease activity. In some embodiments, the polypeptide domain having DNA binding activity comprises a nuclease domain. In some embodiments, the polypeptide domain having nuclease activity comprises a nickase, or a fully active nuclease. As used herein, the term “nickase” refers to a nuclease capable of cleaving only one strand of a double-stranded DNA target. In some embodiments, a nickase can be derived from a fully catalytically active (e.g., natural) form of a polynucleotide programmable nucleotide binding domain by introducing one or more mutations into the active polynucleotide programmable nucleotide binding domain. In some embodiments, the prime editor comprises a polypeptide domain that is an inactive nuclease. In some embodiments, the polypeptide domain having DNA binding activity comprises a napDNAbp, for example, a CRISPR-Cas protein, for example, a Cas9 nickase (referred to as “nCas9”), a Cpf1 nickase (referred to as “nCpf1”), or another CRISPR-Cas nuclease. In some embodiments, the polypeptide domain having DNA polymerase activity comprises a template dependent DNA polymerase, for example, a DNA-dependent DNA polymerase or a RNA-dependent DNA polymerase. In some embodiments, the DNA polymerase is a reverse transcriptase. In some embodiments, the DNA polymerase is DNA polymerase α, β, γ, δ, or ε. In some embodiments, the DNA polymerase is DNA polymerase I, II, III, IV, DNA polymerase α, β, γ, δ, or ε, a T4 DNA polymerase, DNA polymerase θ, v, or any functional variant or fragment thereof.

In some embodiments, a prime editor comprises a nucleic acid programmable DNA binding protein (napDNAbp) fused to a DNA polymerase (e.g., DNA polymerase α, β, γ, δ, or ε). In some embodiments, a prime editor comprises a nucleic acid programmable DNA binding protein (napDNAbp) with nickase activity (e.g., nCas9 or nCpf1) fused to a DNA polymerase (e.g., DNA polymerase α, β, γ, δ, or ε). In some embodiments, a prime editor comprises a napDNAbp with nickase activity (e.g., nCas9 or nCpf1) fused to a DNA polymerase (e.g., DNA polymerase α, β, γ, δ, or ε) and is used to edit a double-stranded target polynucleotide as shown in FIG. 1. In some embodiments, a prime editor comprises a nucleic acid programmable DNA binding protein (napDNAbp) associated with a DNA polymerase (e.g., DNA polymerase α, β, γ, δ, or ε). In some embodiments, a prime editor comprises a nucleic acid programmable DNA binding protein (napDNAbp) with nickase activity (e.g., nCas9 or nCpf1) associated with a DNA polymerase (e.g., DNA polymerase α, β, γ, δ, or ε). In some embodiments, a prime editor comprises a nucleic acid programmable DNA binding protein (napDNAbp) with nickase activity (e.g., nCas9 or nCpf1) associated with a DNA polymerase (e.g., DNA polymerase α, β, γ, δ, or ε) to edit an edit strand of a double-stranded target polynucleotide as shown in FIG. 2.

In some embodiments, prime editing compositions and prime editors of the present disclosure contain a programmable nucleotide binding domain which may guide the prime editor to a specific target nucleic acid (e.g., DNA or RNA) sequence. In some embodiments, prime editing compositions and prime editors of the present disclosure contain a polynucleotide programmable nucleotide binding domain which may guide the prime editor to a specific target nucleic acid (e.g., DNA or RNA) sequence.

In some embodiments, a programmable nucleotide binding domain is a programmable DNA binding domain. Non-limiting examples of programmable DNA binding domains which can be incorporated into a prime editor include a CRISPR-associated protein (Cas protein) domain, a restriction nuclease, a meganuclease, TAL nuclease (TALEN), and a zinc finger nuclease (ZFN).

A polynucleotide programmable nucleotide binding domain of a prime editor can itself comprise one or more protein domains. A polynucleotide programmable nucleotide binding domain can comprise one or more nuclease domains. In some embodiments, the nuclease domain of a polynucleotide programmable nucleotide binding domain can comprise an endonuclease or an exonuclease. Herein the term “exonuclease” refers to a protein or polypeptide capable of digesting a nucleic acid (e.g., RNA or DNA) from free ends, and the term “endonuclease” refers to a protein or polypeptide capable of catalyzing (e.g., cleaving) internal regions in a nucleic acid (e.g., DNA or RNA). In some embodiments, a polynucleotide programmable nucleotide binding domain can be a deoxyribonuclease. In some embodiments a polynucleotide programmable nucleotide binding domain can be a ribonuclease.

As used herein, a programmable DNA binding domain refers to a protein domain that is designed to bind a specific nucleic acid sequence, e.g., a target DNA or a target RNA. In some embodiments, the DNA-binding domain is a polynucleotide programmable DNA-binding domain (napDNAbp) that can associate with a guide polynucleotide (e.g., a chimeric PEg polynucleotide) that guides the DNA-binding domain to a specific DNA sequence, e.g., a search target sequence in a target gene. In some embodiments, the polynucleotide programmable nucleotide binding domain is a polynucleotide programmable RNA binding domain (napRNAbp). For example, the polynucleotide programmable nucleotide binding domain can be associated with a guide polynucleotide that guides the polynucleotide programmable nucleotide binding domain to an RNA. Other nucleic acid programmable DNA binding proteins are also within the scope of this disclosure, though they are not specifically listed in this disclosure.

The term “nucleic acid programmable DNA binding protein” or “napDNAbp” refers to a protein domain that can associate with a guide polynucleotide (e.g., a chimeric PE guide polynucleotide) that guides the DNA-binding domain to a specific DNA sequence, e.g., a search target sequence in a double stranded target gene. In some embodiments, a napDNAbp of a prime editor associates with a prime editing guide polynucleotide (e.g., chimeric PE guide polynucleotide (e.g., DNA-RNA or RNA-DNA guide) to incorporate one or more intended nucleotide edits in a specific nucleic acid sequence (e.g., double-stranded DNA target sequence).

In some embodiments, the napDNAbp is an active nuclease. In some embodiments, the napDNAbp is a nickase. In some embodiments, the napDNAbp is a nuclease having reduced or abolished nuclease activity. In some embodiments, the napDNAbp is nuclease inactive. In some embodiments, a napDNAbp is a nickase that cuts a first strand but not the second strand of a double-stranded target polynucleotide. In some embodiments, the napDNAbp comprises a catalytic domain capable of cleaving the edit strand of the double-stranded target polynucleotide. In some embodiments, the napDNAbp comprises a non-functional catalytic domain that is not capable of cleaving the target strand of the double-stranded target polynucleotide. In some embodiments, the catalytic domain is an HNH domain. In some embodiments, the catalytic domain is an RuvC domain. In some embodiments, the napDNAbp nicks an edit strand of the double-stranded target polynucleotide at a target locus of interest. In some embodiments, the napDNAbp nicks the target strand of the double-stranded target polynucleotide at a target locus of interest. In some embodiments, the napDNAbp nicks both strands of the double-stranded target polynucleotide at a target locus of interest for each strand. In some embodiments, a prime editor comprising a napDNAbp nickase has improve the editing activity as compared to a reference prime editor having a nuclease active napDNAbp.

In some embodiments, the napDNAbp protein associates with a chimeric PE guide polynucleotide (e.g., DNA-RNA or RNA-DNA guide). In some embodiments, a napDNAbp is configured to bind to at least a portion of a PE guide polynucleotide (e.g., chimeric PE guide polynucleotide (e.g., DNA-RNA or RNA-DNA guide). In some embodiments, the napDNAbp protein is configured to bind to at least a portion of a chimeric PE guide polynucleotide. In some embodiments, the napDNAbp is configured to bind to at least a portion of a DNA-RNA chimeric PE guide polynucleotide. In some embodiments, the napDNAbp is configured to bind to at least a portion of a RNA-DNA chimeric PE guide polynucleotide. In some embodiments, the chimeric PE guide polynucleotide targets a napDNAbp to a specific DNA sequence, e.g., a search target sequence, that is complementary to a spacer of the chimeric PE guide polynucleotide (e.g., DNA-RNA or RNA-DNA guide).

In some embodiments, a prime editor comprises a napDNAbp domain that is a CRISPR associated (Cas) protein domain. Accordingly, disclosed herein is a prime editor comprising a Cas protein domain, or a functional fragment or functional variant thereof.

A Cas protein domain incorporated into a prime editing composition can be modified compared to a wild-type or natural version of the CRISPR protein. For example, as described below a Cas protein-derived domain can comprise one or more mutations, insertions, deletions, rearrangements and/or recombinations relative to a wild-type or natural version of the Cas protein.

In some embodiments, a Cas protein domain incorporated into a prime editor is an endonuclease (e.g., deoxyribonuclease or ribonuclease) capable of binding a target polynucleotide when in conjunction with a bound prime editing guide polynucleotide. In some embodiments, a Cas protein domain incorporated into a prime editor is a nickase capable of binding a target polynucleotide when in conjunction with a bound guide nucleic acid. In some embodiments, a Cas protein domain incorporated into a prime editor is a catalytically dead nuclease domain capable of binding a target polynucleotide when in conjunction with a bound guide nucleic acid. In some embodiments, a target polynucleotide bound by a Cas protein derived domain of a prime editor is DNA. In some embodiments, a target polynucleotide bound by a Cas protein domain of a prime editor is RNA.

Cas proteins that can be used in a prime editor provided herein include class 1 and class 2 Cas proteins. Non-limiting examples of Cas proteins include, Cas9 (e.g., dCas9 and nCas9), Cas12a/Cpf1, Cas12b/C2c1, Cas12c/C2c3, Cas12d/CasY, Cas12e/CasX, Cas12g, Cas12h, and Cas12i, and homologues, functional fragments, or functional variants thereof. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at ease 99.5% identical to Cas12a, Cas12b/C2c1, Cas12c, Cas12d, Cas12e, Cas12f, Cas12g, Cas12h, Cas12i.

In some embodiments, the Cas protein of a prime editor is a Class 2 Cas protein. In some embodiments, the Cas protein is a type II Cas protein. In some embodiments, the Cas protein is a Cas9 protein, a modified version of a Cas9 protein, a Cas9 protein homolog, mutant, variant, or a functional fragment thereof. As used herein, a Cas9, Cas9 protein, Cas9 polypeptide or a Cas9 nuclease refers to an RNA guided nuclease comprising one or more Cas9 nuclease domains and a Cas9 gRNA binding domain having the ability to bind a guide polynucleotide, e.g., a chimeric PEg polynucleotide.

In some embodiments, a prime editor as provided herein comprise the full-length amino acid sequence of a Cas9 protein, e.g., one of the Cas9 sequences provided herein. In some embodiments, a prime editor comprises a functional fragment of Cas9. Exemplary amino acid sequences of suitable Cas9 domains and Cas9 fragments are provided herein, and additional suitable sequences of Cas9 domains and fragments will be apparent to those of skill in the art.

For example, in some embodiments, a prime editor protein comprises (1) the guide polynucleotide binding domain of Cas9; or (2) the DNA cleavage domain of Cas9. In some embodiments, a prime editor protein comprises one of two Cas9 domains: (1) the guide polynucleotide binding domain of Cas9; or (2) the DNA cleavage domain of Cas9. In some embodiments, a wild-type Cas9 nuclease has two functional endonuclease domains: RuvC and HNH. In some embodiments, a prime editor protein comprises a guide polynucleotide binding domain of Cas9 and a HNH domain of Cas9, but not the RuvC domain. In some embodiments, a prime editor protein comprises a guide polynucleotide binding domain of Cas9 and a RuvC domain of Cas9, but not the HNH domain.

In some embodiments, the Cas9 domain is a functional variant of a wild-type Cas9 that comprises any one of the Cas9 amino acid sequences as set forth herein. In some embodiments, the Cas9 domain is a functional variant of a wild-type Cas9 that consists of any one of the Cas9 amino acid sequences as set forth herein. In some embodiments, the Cas9 domains is a functional variant of a wild-type Cas9 that comprise an amino acid sequence that is at least 50%, at least 60%, at least 65%, at least 70%, at least 75%, at least 80%, at least 85%, at least 90%, at least 95%, at least 96%, at least 97%, at least 98%, at least 99%, or at least 99.5% identical to a Cas9 polypeptide described herein. In some embodiments, the Cas9 domain is a functional variant of a wild-type Cas9 that comprises an amino acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more or more mutations (e.g., amino acid insertions, deletions or substitutions) compared to any one of the amino acid sequences set forth herein. In some embodiments, the Cas9 domain is a functional variant of a wild-type Cas9 that comprises any one of the amino acid sequences as set forth herein. comprises an amino acid sequence that has at least 10, at least 15, at least 20, at least 30, at least 40, at least 50, at least 60, at least 70, at least 80, at least 90, at least 100, at least 150, at least 200, at least 250, at least 300, at least 350, at least 400, at least 500, at least 600, at least 700, at least 800, at least 900, at least 1000, at least 1100, or at least 1200 identical contiguous amino acid residues as compared to any one of the amino acid sequences set forth herein.

In some embodiments, the Cas9 domain is a Streptococcus pyogenes Cas9 (SpCas9), a Staphylococcus aureus Cas9 (SaCas9), a Streptococcus thermophilus 1 Cas9 (St1Cas9), a Campylobacter jejuni Cas9 (Cj Cas9), or a functional fragment or functional variant thereof. In some embodiments, the Cas9 of a prime editing composition as provided herein can include Cas9 from Corynebacterium ulcerans (NCBI Refs: NC_015683.1, NC_017317.1); Corynebacterium diphtheria (NCBI Refs: NC_016782.1, NC_016786.1); Spiroplasma syrphidicola (NCBI Ref: NC_021284.1); Prevotella intermedia (NCBI Ref: NC_017861.1); Spiroplasma taiwanense (NCBI Ref: NC_021846.1); Streptococcus iniae (NCBI Ref: NC_021314.1); Belliella baltica (NCBI Ref: NC_018010.1); Psychrojlexus torquis (NCBI Ref: NC_018721.1); Streptococcus thermophilus (NCBI Ref: YP_820832.1); Listeria innocua (NCBI Ref: NP_472073.1); Campylobacter jejuni (NCBI Ref: YP_002344900.1); Neisseria meningitidis (NCBI Ref: YP_002342100.1), or to a Cas9 from any other organism, or functional fragments or functional variants thereof.

Non-limiting, exemplary Cas9 domains are provided herein. In some embodiments, the napDNAbp is a wild-type Cas9.

In some embodiments, a wild-type Cas9 corresponds to Cas9 from Streptococcus pyogenes (NCBI Reference Sequence: NC_017053.1, nucleotide and amino acid sequences as follows).

(SEQ ID NO: 24)
ATGGATAAGAAATACTCAATAGGCTTAGATATCGGCACAAATAGCGTCGGATGGGCGGTGATCACTGATG
ATTATAAGGTTCCGTCTAAAAAGTTCAAGGTTCTGGGAAATACAGACCGCCACAGTATCAAAAAAAATCT
TATAGGGGCTCTTTTATTTGGCAGTGGAGAGACAGCGGAAGCGACTCGTCTCAAACGGACAGCTCGTAGA
AGGTATACACGTCGGAAGAATCGTATTTGTTATCTACAGGAGATTTTTTCAAATGAGATGGCGAAAGTAG
ATGATAGTTTCTTTCATCGACTTGAAGAGTCTTTTTTGGTGGAAGAAGACAAGAAGCATGAACGTCATCC
TATTTTTGGAAATATAGTAGATGAAGTTGCTTATCATGAGAAATATCCAACTATCTATCATCTGCGAAAA
AAATTGGCAGATTCTACTGATAAAGCGGATTTGCGCTTAATCTATTTGGCCTTAGCGCATATGATTAAGT
TTCGTGGTCATTTTTTGATTGAGGGAGATTTAAATCCTGATAATAGTGATGTGGACAAACTATTTATCCA
GTTGGTACAAATCTACAATCAATTATTTGAAGAAAACCCTATTAACGCAAGTAGAGTAGATGCTAAAGCG
ATTCTTTCTGCACGATTGAGTAAATCAAGACGATTAGAAAATCTCATTGCTCAGCTCCCCGGTGAGAAGA
GAAATGGCTTGTTTGGGAATCTCATTGCTTTGTCATTGGGATTGACCCCTAATTTTAAATCAAATTTTGA
TTTGGCAGAAGATGCTAAATTACAGCTTTCAAAAGATACTTACGATGATGATTTAGATAATTTATTGGCG
CAAATTGGAGATCAATATGCTGATTTGTTTTTGGCAGCTAAGAATTTATCAGATGCTATTTTACTTTCAG
ATATCCTAAGAGTAAATAGTGAAATAACTAAGGCTCCCCTATCAGCTTCAATGATTAAGCGCTACGATGA
ACATCATCAAGACTTGACTCTTTTAAAAGCTTTAGTTCGACAACAACTTCCAGAAAAGTATAAAGAAATC
TTTTTTGATGAATGAAAAAAGGGATATGGAGGTTATATTGATGGGGGAGGTAGGGAAGAAGAATTTTATA
AATTTATCAAACCAATTTTAGAAAAAATGGATGGTACTGAGGAATTATTGGTGAAACTAAATCGTGAAGA
TTTGCTGCGCAAGCAACGGACCTTTGACAACGGCTCTATTCCCCATCAAATTCACTTGGGTGAGCTGCAT
GCTATTTTGAGAAGACAAGAAGACTTTTATCCATTTTTAAAAGACAATCGTGAGAAGATTGAAAAAATCT
TGACTTTTCGAATTCCTTATTATGTTGGTCCATTGGCGCGTGGCAATAGTCGTTTTGCATGGATGACTCG
GAAGTCTGAAGAAACAATTACCCCATGGAATTTTGAAGAAGTTGTCGATAAAGGTGCTTCAGCTCAATCA
TTTATTGAACGCATGACAAACTTTGATAAAAATCTTCCAAATGAAAAAGTACTACCAAAACATAGTTTGC
TTTATGAGTATTTTACGGTTTATAACGAATTGACAAAGGTCAAATATGTTACTGAGGGAATGCGAAAACC
AGCATTTCTTTCAGGTGAACAGAAGAAAGCCATTGTTGATTTACTCTTCAAAACAAATCGAAAAGTAACC
GTTAAGCAATTAAAAGAAGATTATTTCAAAAAAATAGAATGTTTTGATAGTGTTGAAATTTCAGGAGTTG
AAGATAGATTTAATGCTTCATTAGGCGCCTACCATGATTTGCTAAAAATTATTAAAGATAAAGATTTTTT
GGATAATGAAGAAAATGAAGATATCTTAGAGGATATTGTTTTAACATTGACCTTATTTGAAGATAGGGGG
ATGATTGAGGAAAGACTTAAAACATATGCTCACCTCTTTGATGATAAGGTGATGAAACAGCTTAAACGTC
GCCGTTATACTGGTTGGGGACGTTTGTCTCGAAAATTGATTAATGGTATTAGGGATAAGCAATCTGGCAA
AACAATATTAGATTTTTTGAAATCAGATGGTTTTGCCAATCGCAATTTTATGCAGCTGATCCATGATGAT
AGTTTGACATTTAAAGAAGATATTCAAAAAGCACAGGTGTCTGGACAAGGCCATAGTTTACATGAACAGA
TTGCTAACTTAGCTGGCAGTCCTGCTATTAAAAAAGGTATTTTACAGACTGTAAAAATTGTTGATGAACT
GGTCAAAGTAATGGGGCATAAGCCAGAAAATATCGTTATTGAAATGGCACGTGAAAATCAGACAACTCAA
AAGGGCCAGAAAAATTCGCGAGAGCGTATGAAACGAATCGAAGAAGGTATCAAAGAATTAGGAAGTCAGA
TTCTTAAAGAGCATCCTGTTGAAAATACTCAATTGCAAAATGAAAAGCTCTATCTCTATTATCTACAAAA
TGGAAGAGACATGTATGTGGACCAAGAATTAGATATTAATCGTTTAAGTGATTATGATGTCGATCACATT
GTTCCACAAAGTTTCATTAAAGACGATTCAATAGACAATAAGGTACTAACGCGTTCTGATAAAAATCGTG
GTAAATCGGATAACGTTCCAAGTGAAGAAGTAGTCAAAAAGATGAAAAACTATTGGAGACAACTTCTAAA
CGCCAAGTTAATCACTCAACGTAAGTTTGATAATTTAACGAAAGCTGAACGTGGAGGTTTGAGTGAACTT
GATAAAGCTGGTTTTATCAAACGCCAATTGGTTGAAACTCGCCAAATCACTAAGCATGTGGCACAAATTT
TGGATAGTCGCATGAATACTAAATACGATGAAAATGATAAACTTATTCGAGAGGTTAAAGTGATTACCTT
AAAATCTAAATTAGTTTCTGACTTCCGAAAAGATTTCCAATTCTATAAAGTACGTGAGATTAACAATTAC
CATCATGCCCATGATGCGTATCTAAATGCCGTCGTTGGAACTGCTTTGATTAAGAAATATCCAAAACTTG
AATCGGAGTTTGTCTATGGTGATTATAAAGTTTATGATGTTCGTAAAATGATTGCTAAGTCTGAGCAAGA
AATAGGCAAAGCAACCGCAAAATATTTCTTTTACTCTAATATCATGAACTTCTTCAAAACAGAAATTACA
CTTGCAAATGGAGAGATTCGCAAACGCCCTCTAATCGAAACTAATGGGGAAACTGGAGAAATTGTCTGGG
ATAAAGGGCGAGATTTTGCCACAGTGCGCAAAGTATTGTCCATGCCCCAAGTCAATATTGTCAAGAAAAC
AGAAGTACAGACAGGCGGATTCTCCAAGGAGTCAATTTTACCAAAAAGAAATTCGGACAAGCTTATTGCT
CGTAAAAAAGACTGGGATCCAAAAAAATATGGTGGTTTTGATAGTCCAACGGTAGCTTATTCAGTCCTAG
TGGTTGCTAAGGTGGAAAAAGGGAAATCGAAGAAGTTAAAATCCGTTAAAGAGTTACTAGGGATCACAAT
TATGGAAAGAAGTTCCTTTGAAAAAAATCCGATTGACTTTTTAGAAGCTAAAGGATATAAGGAAGTTAAA
AAAGACTTAATCATTAAACTACCTAAATATAGTCTTTTTGAGTTAGAAAACGGTCGTAAACGGATGCTGG
CTAGTGCCGGAGAATTACAAAAAGGAAATGAGCTGGCTCTGCCAAGCAAATATGTGAATTTTTTATATTT
AGCTAGTCATTATGAAAAGTTGAAGGGTAGTCCAGAAGATAACGAACAAAAACAATTGTTTGTGGAGCAG
CATAAGCATTATTTAGATGAGATTATTGAGCAAATCAGTGAATTTTCTAAGCGTGTTATTTTAGCAGATG
CCAATTTAGATAAAGTTCTTAGTGCATATAACAAACATAGAGACAAACCAATACGTGAACAAGCAGAAAA
TATTATTCATTTATTTACGTTGACGAATCTTGGAGCTCCCGCTGCTTTTAAATATTTTGATACAACAATT
GATCGTAAACGATATACGTCTACAAAAGAAGTTTTAGATGCCACTCTTATCCATCAATCCATCACTGGTC
TTTATGAAACACGCATTGATTTGAGTCAGCTAGGAGGTGACTGA
(SEQ ID NO: 25)
MDKKYSIGLDIGTNSVGWAVITDDYKVPSKKFKVLGNTDRHSIKKNLIGALLFGSGETAEATRLKRTARR
RYTRRKNRICYLQEIFSNEMAKVDDSFFHRLEESFLVEEDKKHERHPIFGNIVDEVAYHEKYPTIYHLRK
KLADSTDKADLRLIYLALAHMIKFRGHFLIEGDLNPDNSDVDKLFIQLVQIYNQLFEENPINASRVDAKA
ILSARLSKSRRLENLIAQLPGEKRNGLFGNLIALSLGLTPNFKSNFDLAEDAKLQLSKDTYDDDLDNLLA
QIGDQYADLFLAAKNLSDAILLSDILRVNSEITKAPLSASMIKRYDEHHQDLTLLKALVRQQLPEKYKEI
FFDQSKNGYAGYIDGGASQEEFYKFIKPILEKMDGTEELLVKLNREDLLRKQRTFDNGSIPHQIHLGELH
AILRRQEDFYPFLKDNREKIEKILTFRIPYYVGPLARGNSRFAWMTRKSEETITPWNFEEVVDKGASAQS
FIERMTNFDKNLPNEKVLPKHSLLYEYFTVYNELTKVKYVTEGMRKPAFLSGEQKKAIVDLLFKTNRKVT
VKQLKEDYFKKIECFDSVEISGVEDRFNASLGAYHDLLKIIKDKDFLDNEENEDILEDIVLTLTLFEDRG
MIEERLKTYAHLFDDKVMKQLKRRRYTGWGRLSRKLINGIRDKQSGKTILDFLKSDGFANRNFMQLIHDD
SLTFKEDIQKAQVSGQGHSLHEQIANLAGSPAIKKGILQTVKIVDELVKVMGHKPENIVIEMARENQTTQ
KGQKNSRERMKRIEEGIKELGSQILKEHPVENTQLQNEKLYLYYLQNGRDMYVDQELDINRLSDYDVDHI
VPQSFIKDDSIDNKVLTRSDKNRGKSDNVPSEEVVKKMKNYWRQLLNAKLITQRKFDNLTKAERGGLSEL
DKAGFIKRQLVETRQITKHVAQILDSRMNTKYDENDKLIREVKVITLKSKLVSDFRKDFQFYKVREINNY
HHAHDAYLNAVVGTALIKKYPKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEVQTGGFSKESILPKRNSDKLIA
RKKDWDPKKYGGFDSPTVAYSVLVVAKVEKGKSKKLKSVKELLGITIMERSSFEKNPIDFLEAKGYKEVK
KDLIIKLPKYSLFELENGRKRMLASAGELQKGNELALPSKYVNFLYLASHYEKLKGSPEDNEQKQLFVEQ
HKHYLDEIIEQISEFSKRVILADANLDKVLSAYNKHRDKPIREQAENIIHLFTLTNLGAPAAFKYFDTTI
DRKRYTSTKEVLDATLIHQSITGLYETRIDLSQLGGD
(single underline: HNH domain; double underline: RuvC domain)

In some embodiments, wild-type Cas9 corresponds to Cas9 from Streptococcus pyogenes (NCBI Reference Sequence: NC_002737.2 (nucleotide sequence as follows); and Uniprot Reference Sequence: Q99ZW2 (amino acid sequence as follows):

(SEQ ID NO: 26)
ATGGATAAGAAATACTCAATAGGCTTAGATATCGGCACAAATAGCGTCGGATGGGCGGTGATCACTGATG
AATATAAGGTTCCGTCTAAAAAGTTCAAGGTTCTGGGAAATACAGACCGCCACAGTATCAAAAAAAATCT
TATAGGGGCTCTTTTATTTGACAGTGGAGAGACAGCGGAAGCGACTCGTCTCAAACGGACAGCTCGTAGA
AGGTATACACGTCGGAAGAATCGTATTTGTTATCTACAGGAGATTTTTTCAAATGAGATGGCGAAAGTAG
ATGATAGTTTCTTTCATCGACTTGAAGAGTCTTTTTTGGTGGAAGAAGACAAGAAGCATGAACGTCATCC
TATTTTTGGAAATATAGTAGATGAAGTTGCTTATCATGAGAAATATCCAACTATCTATCATCTGCGAAAA
AAATTGGTAGATTCTACTGATAAAGCGGATTTGCGCTTAATCTATTTGGCCTTAGCGCATATGATTAAGT
TTCGTGGTCATTTTTTGATTGAGGGAGATTTAAATCCTGATAATAGTGATGTGGACAAACTATTTATCCA
GTTGGTACAAACCTACAATCAATTATTTGAAGAAAACCCTATTAACGCAAGTGGAGTAGATGCTAAAGCG
ATTCTTTCTGCACGATTGAGTAAATCAAGACGATTAGAAAATCTCATTGCTCAGCTCCCCGGTGAGAAGA
AAAATGGCTTATTTGGGAATCTCATTGCTTTGTCATTGGGTTTGACCCCTAATTTTAAATCAAATTTTGA
TTTGGCAGAAGATGCTAAATTACAGCTTTCAAAAGATACTTACGATGATGATTTAGATAATTTATTGGCG
CAAATTGGAGATCAATATGCTGATTTGTTTTTGGCAGCTAAGAATTTATCAGATGCTATTTTACTTTCAG
ATATCCTAAGAGTAAATACTGAAATAACTAAGGCTCCCCTATCAGCTTCAATGATTAAACGCTACGATGA
ACATCATCAAGACTTGACTCTTTTAAAAGCTTTAGTTCGACAACAACTTCCAGAAAAGTATAAAGAAATC
TTTTTTGATGAATGAAAAAAGGGATATGGAGGTTATATTGATGGGGGAGGTAGGGAAGAAGAATTTTATA
AATTTATCAAACCAATTTTAGAAAAAATGGATGGTACTGAGGAATTATTGGTGAAACTAAATCGTGAAGA
TTTGCTGCGCAAGCAACGGACCTTTGACAACGGCTCTATTCCCCATCAAATTCACTTGGGTGAGCTGCAT
GCTATTTTGAGAAGACAAGAAGACTTTTATCCATTTTTAAAAGACAATCGTGAGAAGATTGAAAAAATCT
TGACTTTTCGAATTCCTTATTATGTTGGTCCATTGGCGCGTGGCAATAGTCGTTTTGCATGGATGACTCG
GAAGTCTGAAGAAACAATTACCCCATGGAATTTTGAAGAAGTTGTCGATAAAGGTGCTTCAGCTCAATCA
TTTATTGAACGCATGACAAACTTTGATAAAAATCTTCCAAATGAAAAAGTACTACCAAAACATAGTTTGC
TTTATGAGTATTTTACGGTTTATAACGAATTGACAAAGGTCAAATATGTTACTGAAGGAATGCGAAAACC
AGCATTTCTTTCAGGTGAACAGAAGAAAGCCATTGTTGATTTACTCTTCAAAACAAATCGAAAAGTAACC
GTTAAGCAATTAAAAGAAGATTATTTCAAAAAAATAGAATGTTTTGATAGTGTTGAAATTTCAGGAGTTG
AAGATAGATTTAATGCTTCATTAGGTACCTACCATGATTTGCTAAAAATTATTAAAGATAAAGATTTTTT
GGATAATGAAGAAAATGAAGATATCTTAGAGGATATTGTTTTAACATTGACCTTATTTGAAGATAGGGAG
ATGATTGAGGAAAGACTTAAAACATATGCTCACCTCTTTGATGATAAGGTGATGAAACAGCTTAAACGTC
GCCGTTATACTGGTTGGGGACGTTTGTCTCGAAAATTGATTAATGGTATTAGGGATAAGCAATCTGGCAA
AACAATATTAGATTTTTTGAAATCAGATGGTTTTGCCAATCGCAATTTTATGCAGCTGATCCATGATGAT
AGTTTGACATTTAAAGAAGACATTCAAAAAGCACAAGTGTCTGGACAAGGCGATAGTTTACATGAACATA
TTGCAAATTTAGCTGGTAGCCCTGCTATTAAAAAAGGTATTTTACAGACTGTAAAAGTTGTTGATGAATT
GGTCAAAGTAATGGGGCGGCATAAGCCAGAAAATATCGTTATTGAAATGGCACGTGAAAATCAGACAACT
CAAAAGGGCCAGAAAAATTCGCGAGAGCGTATGAAACGAATCGAAGAAGGTATCAAAGAATTAGGAAGTC
AGATTCTTAAAGAGCATCCTGTTGAAAATACTCAATTGCAAAATGAAAAGCTCTATCTCTATTATCTCCA
AAATGGAAGAGACATGTATGTGGACCAAGAATTAGATATTAATCGTTTAAGTGATTATGATGTCGATCAC
ATTGTTCCACAAAGTTTCCTTAAAGACGATTCAATAGACAATAAGGTCTTAACGCGTTCTGATAAAAATC
GTGGTAAATCGGATAACGTTCCAAGTGAAGAAGTAGTCAAAAAGATGAAAAACTATTGGAGACAACTTCT
AAACGCCAAGTTAATCACTCAACGTAAGTTTGATAATTTAACGAAAGCTGAACGTGGAGGTTTGAGTGAA
CTTGATAAAGCTGGTTTTATCAAACGCCAATTGGTTGAAACTCGCCAAATCACTAAGCATGTGGCACAAA
TTTTGGATAGTCGCATGAATACTAAATACGATGAAAATGATAAACTTATTCGAGAGGTTAAAGTGATTAC
CTTAAAATCTAAATTAGTTTCTGACTTCCGAAAAGATTTCCAATTCTATAAAGTACGTGAGATTAACAAT
TACCATCATGCCCATGATGCGTATCTAAATGCCGTCGTTGGAACTGCTTTGATTAAGAAATATCCAAAAC
TTGAATCGGAGTTTGTCTATGGTGATTATAAAGTTTATGATGTTCGTAAAATGATTGCTAAGTCTGAGCA
AGAAATAGGCAAAGCAACCGCAAAATATTTCTTTTACTCTAATATCATGAACTTCTTCAAAACAGAAATT
ACACTTGCAAATGGAGAGATTCGCAAACGCCCTCTAATCGAAACTAATGGGGAAACTGGAGAAATTGTCT
GGGATAAAGGGCGAGATTTTGCCACAGTGCGCAAAGTATTGTCCATGCCCCAAGTCAATATTGTCAAGAA
AACAGAAGTACAGACAGGCGGATTCTCCAAGGAGTCAATTTTACCAAAAAGAAATTCGGACAAGCTTATT
GCTCGTAAAAAAGACTGGGATCCAAAAAAATATGGTGGTTTTGATAGTCCAACGGTAGCTTATTCAGTCC
TAGTGGTTGCTAAGGTGGAAAAAGGGAAATCGAAGAAGTTAAAATCCGTTAAAGAGTTACTAGGGATCAC
AATTATGGAAAGAAGTTCCTTTGAAAAAAATCCGATTGACTTTTTAGAAGCTAAAGGATATAAGGAAGTT
AAAAAAGACTTAATCATTAAACTACCTAAATATAGTCTTTTTGAGTTAGAAAACGGTCGTAAACGGATGC
TGGCTAGTGCCGGAGAATTACAAAAAGGAAATGAGCTGGCTCTGCCAAGCAAATATGTGAATTTTTTATA
TTTAGCTAGTCATTATGAAAAGTTGAAGGGTAGTCCAGAAGATAACGAACAAAAACAATTGTTTGTGGAG
CAGCATAAGCATTATTTAGATGAGATTATTGAGCAAATCAGTGAATTTTCTAAGCGTGTTATTTTAGCAG
ATGCCAATTTAGATAAAGTTCTTAGTGCATATAACAAACATAGAGACAAACCAATACGTGAACAAGCAGA
AAATATTATTCATTTATTTACGTTGACGAATCTTGGAGCTCCCGCTGCTTTTAAATATTTTGATACAACA
ATTGATCGTAAACGATATACGTCTACAAAAGAAGTTTTAGATGCCACTCTTATCCATCAATCCATCACTG
GTCTTTATGAAACACGCATTGATTTGAGTCAGCTAGGAGGTGACTGA
(SEQ ID NO: 27)
MDKKYSIGLDIGTNSVGWAVITDEYKVPSKKFKVLGNTDRHSIKKNLIGALLFDSGETAEATRLKRTARR
RYTRRKNRICYLQEIFSNEMAKVDDSFFHRLEESFLVEEDKKHERHPIFGNIVDEVAYHEKYPTIYHLRK
KLVDSTDKADLRLIYLALAHMIKFRGHFLIEGDLNPDNSDVDKLFIQLVQTYNQLFEENPINASGVDAKA
ILSARLSKSRRLENLIAQLPGEKKNGLFGNLIALSLGLTPNFKSNFDLAEDAKLQLSKDTYDDDLDNLLA
QIGDQYADLFLAAKNLSDAILLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLRKQRTFDNGSIPHQIHLGELH
AILRRQEDEYPFLKDNREKIEKILTFRIPYYVGPLARGNSRFAWMTRKSEETITPWNFEEVVDKGASAQS
FIERMTNFDKNLPNEKVLPKHSLLYEYFTVYNELTKVKYVTEGMRKPAFLSGEQKKAIVDLLFKTNRKVT
VKQLKEDYFKKIECFDSVEISGVEDRFNASLGTYHDLLKIIKDKDFLDNEENEDILEDIVLTLTLFEDRE
MIEERLKTYAHLFDDKVMKQLKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKVMGRHKPENIVIEMARENQTT
QKGQKNSRERMKRIEEGIKELGSQILKEHPVENTQLQNEKLYLYYLQNGRDMYVDQELDINRLSDYDVDH
IVPQSFLKDDSIDNKVLTRSDKNRGKSDNVPSEEVVKKMKNYWRQLLNAKLITQRKFDNLTKAERGGLSE
LDKAGFIKRQLVETRQITKHVAQILDSRMNTKYDENDKLIREVKVITLKSKLVSDFRKDFQFYKVREINN
YHHAHDAYLNAVVGTALIKKYPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEVQTGGFSKESILPKRNSDKLI
ARKKDWDPKKYGGFDSPTVAYSVLVVAKVEKGKSKKLKSVKELLGITIMERSSFEKNPIDFLEAKGYKEV
KKDLIIKLPKYSLFELENGRKRMLASAGELQKGNELALPSKYVNFLYLASHYEKLKGSPEDNEQKQLFVE
QHKHYLDEIIEQISEFSKRVILADANLDKVLSAYNKHRDKPIREQAENIIHLFTLTNLGAPAAFKYFDTT
IDRKRYTSTKEVLDATLIHQSITGLYETRIDLSQLGGD
(single underline: HNH
domain; double underline: RuvC domain)

In some embodiments, the napDNAbp is a SaCas9 or SpCas9. An exemplary Streptococcus pyogenes Cas9 (spCas9) amino acid sequence is provided below:

(SEQ ID NO: 25)
MDKKYSIGLDIGTNSVGWAVITDDYKVPSKKFKVLGNTDRHSIKKNLIGALLFGSGETAEATRLKRTARR
RYTRRKNRICYLQEIFSNEMAKVDDSFFHRLEESFLVEEDKKHERHPIFGNIVDEVAYHEKYPTIYHLRK
KLADSTDKADLRLIYLALAHMIKFRGHFLIEGDLNPDNSDVDKLFIQLVQIYNQLFEENPINASRVDAKA
ILSARLSKSRRLENLIAQLPGEKRNGLFGNLIALSLGLTPNFKSNFDLAEDAKLQLSKDTYDDDLDNLLA
QIGDQYADLFLAAKNLSDAILLSDILRVNSEITKAPLSASMIKRYDEHHQDLTLLKALVRQQLPEKYKEI
FFDQSKNGYAGYIDGGASQEEFYKFIKPILEKMDGTEELLVKLNREDLLRKQRTFDNGSIPHQIHLGELH
AILRRQEDFYPFLKDNREKIEKILTFRIPYYVGPLARGNSRFAWMTRKSEETITPWNFEEVVDKGASAQS
FIERMTNFDKNLPNEKVLPKHSLLYEYFTVYNELTKVKYVTEGMRKPAFLSGEQKKAIVDLLFKTNRKVT
VKQLKEDYFKKIECFDSVEISGVEDRFNASLGAYHDLLKIIKDKDFLDNEENEDILEDIVLTLTLFEDRG
MIEERLKTYAHLFDDKVMKQLKRRRYTGWGRLSRKLINGIRDKQSGKTILDFLKSDGFANRNFMQLIHDD
SLTFKEDIQKAQVSGQGHSLHEQIANLAGSPAIKKGILQTVKIVDELVKVMGHKPENIVIEMARENQTTQ
KGQKNSRERMKRIEEGIKELGSQILKEHPVENTQLQNEKLYLYYLQNGRDMYVDQELDINRLSDYDVDHI
VPQSFIKDDSIDNKVLTRSDKNRGKSDNVPSEEVVKKMKNYWRQLLNAKLITQRKFDNLTKAERGGLSEL
DKAGFIKRQLVETRQITKHVAQILDSRMNTKYDENDKLIREVKVITLKSKLVSDFRKDFQFYKVREINNY
HHAHDAYLNAVVGTALIKKYPKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEVQTGGFSKESILPKRNSDKLIA
RKKDWDPKKYGGFDSPTVAYSVLVVAKVEKGKSKKLKSVKELLGITIMERSSFEKNPIDFLEAKGYKEVK
KDLIIKLPKYSLFELENGRKRMLASAGELQKGNELALPSKYVNELYLASHYEKLKGSPEDNEQKQLFVEQ
HKHYLDEIIEQISEFSKRVILADANLDKVLSAYNKHRDKPIREQAENIIHLFTLTNLGAPAAFKYFDTTI
DRKRYTSTKEVLDATLIHQSITGLYETRIDLSQLGGD
(single underline: HNH domain; double underline: RuvC domain)

In some embodiments, a prime editor comprises a functional variant of a wild-type Cas9. In some instances, the functional variant Cas9 has one or more amino acid deletion, insertion, or substitution that reduces the nuclease activity of the Cas9 polypeptide. For example, in some instances, the functional Cas9 variant has less than 50%, less than 40%, less than 30%, less than 20%, less than about 15%, less than about 10%, less than about 5%, less than about 1%, or less than about 0.1% of the nuclease activity of the corresponding wild-type Cas9 protein. In some embodiments, a functional Cas9 variant a can associate with a guide polynucleotide, e.g., a chimeric PE guide polynucleotide and bind target polynucleotide, but has reduced or abolished nuclease activity. In some embodiments, the Cas9 domain may be a nuclease active Cas9, a nuclease inactive Cas9 (dCas9), or a Cas9 nickase (nCas9). In some embodiments, the Cas9 domain is a nuclease active domain. For example, the Cas9 domain may be a Cas9 domain that cuts both strands of a double stranded DNA molecule).

In some embodiments, the Cas9 is a Cas9 nickase (nCas9) that nicks an edit strand but not the target strand of a double-stranded target polynucleotide. In some embodiments, the Cas9 nickase comprises a nuclease inactive HNH domain and a nuclease active RuvC domain. In some embodiments the Cas9 nickase cleaves the edit strand and not the target strand of a double stranded target polynucleotide. In some embodiments, a Cas9 nickase comprises a nuclease inactive RuvC domain and an active HNH domain. In some embodiments the Cas9 nickase cleaves the target strand and not the edit strand of a double stranded target polynucleotide. In some embodiments, the Cas9 nickase comprises an amino acid substation, deletion, or insertion in a HNH domain compared to a wild-type Cas9 that reduces or abolishes nuclease activity of the HNH domain. In some embodiments, the Cas9 nickase comprises an amino acid substation in a RuvC domain compared to a wild-type Cas9 that reduces or abolishes nuclease activity of the RuvC domain.

In some embodiments, the Cas9 nickase comprises amino acid substitution D10A or H840A, or a corresponding amino acid substitution thereof compared to the wild-type SpCas9. In some embodiments, the Cas9 nickase comprises amino acid substitution D10A, or a corresponding amino acid substitution thereof compared to the wild-type SpCas9. In some embodiments, the Cas9 nickase comprises amino acid substitution H840A, or a corresponding amino acid substitution thereof compared to the wild-type SpCas9. In some embodiments, the Cas9 nickase comprises amino acid substitution H840A, while the residue at position 10 remains a aspartic acid as set forth in the wild-type SpCas9 amino acid sequence or at a corresponding position thereof. In some embodiments, the Cas9 nickase comprises amino acid substitution D10A, while the residue at position 840 remains a aspartic acid as set forth in the wild-type SpCas9 amino acid sequence or at a corresponding position thereof.

In some embodiments, the functional Cas9 variant retains the ability to bind a double-stranded target polynucleotide can cleave the PAM strand (the edit strand) of the double-stranded target polynucleotide but has reduced ability to cleave the target strand of the double-stranded target polynucleotide. Non-limiting examples of amino acid residues that can be altered to reduce Cas9 nuclease activity include D10, G12, G17, E762, H840, N854, N863, H982, H983, A984, D986, and A987, as compared to a wild-type Cas9. In some embodiments, a functional Cas9 variant that has reduced catalytic activity (e.g., when a Cas9 protein has a E762, H840, N854, N863, H982, H983, A984, D986, and/or a A987 mutation, e.g., E762A, H840A, N854A, N863A, H982A, H983A, A984A, and/or D986A), the functional Cas9 variant retains the ability to bind the guide polynucleotide and can still bind to target DNA in a site-specific manner. Mutations other than alanine substitutions known in the art are also suitable.

In some embodiments, the functional Cas9 variant can have a mutation (amino acid substitution) that reduces the nuclease function of the RuvC domain such that the polypeptide has a reduced ability to cleave a target DNA. In some embodiments, the functional Cas9 variant harbors W476A and/or W1126A mutations compared to a wild-type Cas9. In some embodiments, the functional Cas9 variant harbors P475A, W476A, N477A, D1125A, W1126A, and/or D1127A mutations compared to a wild-type Cas9. In some embodiments, the functional Cas9 variant harbors H840A, W476A, and/or W1126A mutations compared to a wild-type Cas9. In some embodiments, the functional Cas9 variant harbors H840A, D10A, W476A, and/or W1126A mutations compared to a wild-type Cas9. In some embodiments, functional Cas9 variant has restored catalytic His residue at position 840 in the Cas9 HNH domain (A840H). In some embodiments, the functional Cas9 variant harbors, H840A, P475A, W476A, N477A, D1125A, W1126A, and/or D1127A mutations compared to a wild-type Cas9.

In some embodiments, the SpCas9 comprises a D9A mutation, or a corresponding mutation compared to a wild-type Cas9. In some embodiments, the functional Cas9 variant harbors D10A, G12A, and/or G17A mutations compared to a wild-type Cas9.

In some embodiments, the SpCas9 comprises a H840A mutation, or a corresponding mutation compared to a wild type SpCas9. In some embodiments, a functional Cas9 variant comprise E762A, H840A, N854A, N863A, H982A, H983A, A984A, and/or D986A mutations compared to a wild type SpCas9. In some embodiments, the napDNAbp is a SpCas9 nickase as according to Accession No. AWD73737.1.

An exemplary amino acid sequence of SpCas9 H840A nickase is provided below:

(SEQ ID NO: 28)
MDKKYSIGLAIGTNSVGWAVITDEYKVPSKKFKVLGNTDRHSIKKNLIGALLFDSGETAEATRLKRTARR
RYTRRKNRICYLQEIFSNEMAKVDDSFFHRLEESFLVEEDKKHERHPIFGNIVDEVAYHEKYPTIYHLRK
KLVDSTDKADLRLIYLALAHMIKFRGHFLIEGDLNPDNSDVDKLFIQLVQTYNQLFEENPINASGVDAKA
ILSARLSKSRRLENLIAQLPGEKKNGLFGNLIALSLGLTPNFKSNFDLAEDAKLQLSKDTYDDDLDNLLA
QIGDQYADLFLAAKNLSDAILLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLRKQRTFDNGSIPHQIHLGELH
AILRRQEDFYPFLKDNREKIEKILTFRIPYYVGPLARGNSRFAWMTRKSEETITPWNFEEVVDKGASAQS
FIERMTNFDKNLPNEKVLPKHSLLYEYFTVYNELTKVKYVTEGMRKPAFLSGEQKKAIVDLLFKTNRKVT
VKQLKEDYFKKIECFDSVEISGVEDRFNASLGTYHDLLKIIKDKDFLDNEENEDILEDIVLTLTLFEDRE
MIEERLKTYAHLFDDKVMKQLKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKVMGRHKPENIVIEMARENQTT
QKGQKNSRERMKRIEEGIKELGSQILKEHPVENTQLQNEKLYLYYLQNGRDMYVDQELDINRLSDYDVDH
IVPQSFLKDDSIDNKVLTRSDKNRGKSDNVPSEEVVKKMKNYWRQLLNAKLITQRKFDNLTKAERGGLSE
LDKAGFIKRQLVETRQITKHVAQILDSRMNTKYDENDKLIREVKVITLKSKLVSDFRKDFQFYKVREINN
YHHAHDAYLNAVVGTALIKKYPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEVQTGGFSKESILPKRNSDKLI
ARKKDWDPKKYGGFDSPTVAYSVLVVAKVEKGKSKKLKSVKELLGITIMERSSFEKNPIDFLEAKGYKEV
KKDLIIKLPKYSLFELENGRKRMLASAGELQKGNELALPSKYVNFLYLASHYEKLKGSPEDNEQKQLFVE
QHKHYLDEIIEQISEFSKRVILADANLDKVLSAYNKHRDKPIREQAENIIHLFTLTNLGAPAAFKYFDTT
IDRKRYTSTKEVLDATLIHQSITGLYETRIDLSQLGGD
(single underline: HNH domain; double underline: RuvC domain).

In some embodiments an exemplary amino acid sequence of SpCas9 H840A nickase lacks N-terminal methionine as provided below:

(SEQ ID NO: 29)
DKKYSIGLAIGTNSVGWAVITDEYKVPSKKFKVLGNTDRHSIKKNLIGALLFDSGETAEATRLKRTARRR
YTRRKNRICYLQEIFSNEMAKVDDSFFHRLEESFLVEEDKKHERHPIFGNIVDEVAYHEKYPTIYHLRKK
LVDSTDKADLRLIYLALAHMIKFRGHFLIEGDLNPDNSDVDKLFIQLVQTYNQLFEENPINASGVDAKAI
LSARLSKSRRLENLIAQLPGEKKNGLFGNLIALSLGLTPNFKSNFDLAEDAKLQLSKDTYDDDLDNLLAQ
IGDQYADLFLAAKNLSDAILLSDILRVNTEITKAPLSASMIKRYDEHHQDLTLLKALVRQQLPEKYKEIF
FDQSKNGYAGYIDGGASQEEFYKFIKPILEKMDGTEELLVKLNREDLLRKQRTFDNGSIPHQIHLGELHA
ILRRQEDFYPFLKDNREKIEKILTFRIPYYVGPLARGNSRFAWMTRKSEETITPWNFEEVVDKGASAQSF
IERMTNFDKNLPNEKVLPKHSLLYEYFTVYNELTKVKYVTEGMRKPAFLSGEQKKAIVDLLFKTNRKVTV
KQLKEDYFKKIECFDSVEISGVEDRFNASLGTYHDLLKIIKDKDFLDNEENEDILEDIVLTLTLFEDREM
IEERLKTYAHLFDDKVMKQLKRRRYTGWGRLSRKLINGIRDKQSGKTILDFLKSDGFANRNFMQLIHDDS
LTFKEDIQKAQVSGQGDSLHEHIANLAGSPAIKKGILQTVKVVDELVKVMGRHKPENIVIEMARENQTTQ
KGQKNSRERMKRIEEGIKELGSQILKEHPVENTQLQNEKLYLYYLQNGRDMYVDQELDINRLSDYDVDHI
VPQSFLKDDSIDNKVLTRSDKNRGKSDNVPSEEVVKKMKNYWRQLLNAKLITQRKFDNLTKAERGGLSEL
DKAGFIKRQLVETRQITKHVAQILDSRMNTKYDENDKLIREVKVITLKSKLVSDFRKDFQFYKVREINNY
HHAHDAYLNAVVGTALIKKYPKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEVQTGGFSKESILPKRNSDKLIA
RKKDWDPKKYGGFDSPTVAYSVLVVAKVEKGKSKKLKSVKELLGITIMERSSFEKNPIDFLEAKGYKEVK
KDLIIKLPKYSLFELENGRKRMLASAGELQKGNELALPSKYVNFLYLASHYEKLKGSPEDNEQKQLFVEQ
HKHYLDEIIEQISEFSKRVILADANLDKVLSAYNKHRDKPIREQAENIIHLFTLTNLGAPAAFKYFDTTI
DRKRYTSTKEVLDATLIHQSITGLYETRIDLSQLGGD
(single underline: HNH domain; double underline: RuvC domain).

In some embodiments, the Cas9 nickase is a SaCas9 that comprises an amino acid substitution at position N579 as compared to the wild-type SaCas9, or a corresponding amino acid substitution thereof. In some embodiments, the SaCas9 nickase comprises a N579A amino acid substitution as compared to the wild-type SaCas9.

An exemplary amino acid sequence of wild-type SaCas9 comprises the amino acid sequence:

(SEQ ID NO: 30)
MKRNYILGLDIGITSVGYGIIDYETRDVIDAGVRL
FKEANVENNEGRRSKRGARRLKRRRRHRIQRVKKL
LFDYNLLTDHSELSGINPYEARVKGLSQKLSEEEF
SAALLHLAKRRGVHNVNEVEEDTGNELSTKEQISR
NSKALEEKYVAELQLERLKKDGEVRGSINRFKTSD
YVKEAKQLLKVQKAYHQLDQSFIDTYIDLLETRRT
YYEGPGEGSPFGWKDIKEWYEMLMGHCTYFPEELR
SVKYAYNADLYNALNDLNNLVITRDENEKLEYYEK
FQIIENVFKQKKKPTLKQIAKEILVNEEDIKGYRV
TSTGKPEFTNLKVYHDIKDITARKEIIENAELLDQ
IAKILTIYQSSEDIQEELTNLNSELTQEEIEQISN
LKGYTGTHNLSLKAINLILDELWHTNDNQIAIFNR
LKLVPKKVDLSQQKEIPTTLVDDFILSPVVKRSFI
QSIKVINAIIKKYGLPNDIIIELAREKNSKDAQKM
INEMQKRNRQTNERIEEIIRTTGKENAKYLIEKIK
LHDMQEGKCLYSLEAIPLEDLLNNPFNYEVDHIIP
RSVSFDNSFNNKVLVKQEENSKKGNRTPFQYLSSS
DSKISYETFKKHILNLAKGKGRISKTKKEYLLEER
DINRFSVQKDFINRNLVDTRYATRGLMNLLRSYFR
VNNLDVKVKSINGGFTSFLRRKWKFKKERNKGYKH
HAEDALIIANADFIFKEWKKLDKAKKVMENQMFEE
KQAESMPEIETEQEYKEIFITPHQIKHIKDFKDYK
YSHRVDKKPNRELINDTLYSTRKDDKGNTLIVNNL
NGLYDKDNDKLKKLINKSPEKLLMYHHDPQTYQKL
KLIMEQYGDEKNPLYKYYEETGNYLTKYSKKDNGP
VIKKIKYYGNKLNAHLDITDDYPNSRNKVVKLSLK
PYRFDVYLDNGVYKFVTVKNLDVIKKENYYEVNSK
CYEEAKKLKKISNQAEFIASFYNNDLIKINGELYR
VIGVNNDLLNRIEVNMIDITYREYLENMNDKRPPR
IIKTIASKTQSIKKYSTDILGNLYEVKSKKHPQII
KKG

In some embodiments an exemplary amino acid sequence of wild-type SaCas9 does not comprise a N-terminal Methionine as below:

(SEQ ID NO: 31)
KRNYILGLDIGITSVGYGIIDYETRDVIDAGVRLF
KEANVENNEGRRSKRGARRLKRRRRHRIQRVKKLL
FDYNLLTDHSELSGINPYEARVKGLSQKLSEEEFS
AALLHLAKRRGVHNVNEVEEDTGNELSTKEQISRN
SKALEEKYVAELQLERLKKDGEVRGSINRFKTSDY
VKEAKQLLKVQKAYHQLDQSFIDTYIDLLETRRTY
YEGPGEGSPFGWKDIKEWYEMLMGHCTYFPEELRS
VKYAYNADLYNALNDLNNLVITRDENEKLEYYEKF
QIIENVFKQKKKPTLKQIAKEILVNEEDIKGYRVT
STGKPEFTNLKVYHDIKDITARKEIIENAELLDQI
AKILTIYQSSEDIQEELTNLNSELTQEEIEQISNL
KGYTGTHNLSLKAINLILDELWHTNDNQIAIFNRL
KLVPKKVDLSQQKEIPTTLVDDFILSPVVKRSFIQ
SIKVINAIIKKYGLPNDIIIELAREKNSKDAQKMI
NEMQKRNRQTNERIEEIIRTTGKENAKYLIEKIKL
HDMQEGKCLYSLEAIPLEDLLNNPFNYEVDHIIPR
SVSFDNSFNNKVLVKQEENSKKGNRTPFQYLSSSD
SKISYETFKKHILNLAKGKGRISKTKKEYLLEERD
INRFSVQKDFINRNLVDTRYATRGLMNLLRSYFRV
NNLDVKVKSINGGFTSFLRRKWKFKKERNKGYKHH
AEDALIIANADFIFKEWKKLDKAKKVMENQMFEEK
QAESMPEIETEQEYKEIFITPHQIKHIKDFKDYKY
SHRVDKKPNRELINDTLYSTRKDDKGNTLIVNNLN
GLYDKDNDKLKKLINKSPEKLLMYHHDPQTYQKLK
LIMEQYGDEKNPLYKYYEETGNYLTKYSKKDNGPV
IKKIKYYGNKLNAHLDITDDYPNSRNKVVKLSLKP
YRFDVYLDNGVYKFVTVKNLDVIKKENYYEVNSKC
YEEAKKLKKISNQAEFIASFYNNDLIKINGELYRV
IGVNNDLLNRIEVNMIDITYREYLENMNDKRPPRI
IKTIASKTQSIKKYSTDILGNLYEVKSKKHPQIIK
KG
Residue N579 above, which is
underlined and in bold, may be
mutated (e.g., to a A579)
to yield a SaCas9 nickase.

Additional suitable Cas9 nickases will be apparent to those of skill in the art based on this disclosure and knowledge in the field and are within the scope of this disclosure.

In some embodiments, the Cas9 is a nuclease inactive Cas9 (dCas9) that cannot cleave either strand of a double-stranded target polynucleotide. In some embodiments, the dCas9 comprises an amino acid substation, deletion, or insertion in a HNH domain and a RuvC domain compared to a wild-type Cas9 that reduces or abolishes nuclease activity. In further embodiments, a catalytically dead Cas9 comprises a point mutation (e.g., D10A or H840A) as well as a deletion of all or a portion of a nuclease domain. In some embodiments, the dCas9 comprises both a D10X mutation and an H840X mutation compared to a wild-type Cas9, wherein X is any amino acid other than the wild-type amino acid. In some embodiments, the dCas9 comprises both a D10A mutation and an H840A mutation compared to a wild-type Cas9.

As one example, a nuclease-inactive Cas9 domain (D10A and H840A) comprises the amino acid sequence of an exemplary catalytically inactive Cas9 (dCas9) is as follows:

(SEQ ID NO: 32)
MDKKYSIGLAIGTNSVGWAVITDEYKVPSKKFKVL
GNTDRHSIKKNLIGALLFDSGETAEATRLKRTARR
RYTRRKNRICYLQEIFSNEMAKVDDSFFHRLEESF
LVEEDKKHERHPIFGNIVDEVAYHEKYPTIYHLRK
KLVDSTDKADLRLIYLALAHMIKFRGHFLIEGDLN
PDNSDVDKLFIQLVQTYNQLFEENPINASGVDAKA
ILSARLSKSRRLENLIAQLPGEKKNGLFGNLIALS
LGLTPNFKSNFDLAEDAKLQLSKDTYDDDLDNLLA
QIGDQYADLFLAAKNLSDAILLSDILRVNTEITKA
PLSASMIKRYDEHHQDLTLLKALVRQQLPEKYKEI
FFDQSKNGYAGYIDGGASQEEFYKFIKPILEKMDG
TEELLVKLNREDLLRKQRTFDNGSIPHQIHLGELH
AILRRQEDFYPFLKDNREKIEKILTFRIPYYVGPL
ARGNSRFAWMTRKSEETITPWNFEEVVDKGASAQS
FIERMTNFDKNLPNEKVLPKHSLLYEYFTVYNELT
KVKYVTEGMRKPAFLSGEQKKAIVDLLFKTNRKVT
VKQLKEDYFKKIECFDSVEISGVEDRFNASLGTYH
DLLKIIKDKDFLDNEENEDILEDIVLTLTLFEDRE
MIEERLKTYAHLFDDKVMKQLKRRRYTGWGRLSRK
LINGIRDKQSGKTILDFLKSDGFANRNFMQLIHDD
SLTFKEDIQKAQVSGQGDSLHEHIANLAGSPAIKK
GILQTVKVVDELVKVMGRHKPENIVIEMARENQTT
QKGQKNSRERMKRIEEGIKELGSQILKEHPVENTQ
LQNEKLYLYYLQNGRDMYVDQELDINRLSDYDVDA
IVPQSFLKDDSIDNKVLTRSDKNRGKSDNVPSEEV
VKKMKNYWRQLLNAKLITQRKFDNLTKAERGGLSE
LDKAGFIKRQLVETRQITKHVAQILDSRMNTKYDE
NDKLIREVKVITLKSKLVSDFRKDFQFYKVREINN
YHHAHDAYLNAVVGTALIKKYPKLESEFVYGDYKV
YDVRKMIAKSEQEIGKATAKYFFYSNIMNFFKTE1
TLANGEIRKRPLIETNGETGEIVWDKGRDFATVRK
VLSMPQVNIVKKTEVQTGGFSKESILPKRNSDKLI
ARKKDWDPKKYGGFDSPTVAYSVLVVAKVEKGKSK
KLKSVKELLGITIMERSSFEKNPIDFLEAKGYKEV
KKDLIIKLPKYSLFELENGRKRMLASAGELQKGNE
LALPSKYVNFLYLASHYEKLKGSPEDNEQKQLFVE
QHKHYLDEIIEQISEFSKRVILADANLDKVLSAYN
KHRDKPIREQAENIIHLFTLTNLGAPAAFKYFDTT
IDRKRYTSTKEVLDATLIHQSITGLYETRIDLSQL
GGD
(see, e.g., Qi etal.,
“Repurposing CRISPR as an RNA-guided
platform for sequence-specific control
of gene expression.”
Cell. 2013; 152(5): 1173-83, the entire
contents of which are incorporated
herein by reference).

In some embodiments, a nuclease-inactive Cas9 domain (D10A and H840A) does not comprise an N terminal methionine, the amino acid sequence of an exemplary catalytically inactive Cas9 (dCas9) is as follows:

(SEQ ID NO: 32)
MDKKYSIGLAIGTNSVGWAVITDEYKVPSKKFKVL
GNTDRHSIKKNLIGALLFDSGETAEATRLKRTARR
RYTRRKNRICYLQEIFSNEMAKVDDSFFHRLEESF
LVEEDKKHERHPIFGNIVDEVAYHEKYPTIYHLRK
KLVDSTDKADLRLIYLALAHMIKFRGHFLIEGDLN
PDNSDVDKLFIQLVQTYNQLFEENPINASGVDAKA
ILSARLSKSRRLENLIAQLPGEKKNGLFGNLIALS
LGLTPNFKSNFDLAEDAKLQLSKDTYDDDLDNLLA
QIGDQYADLFLAAKNLSDAILLSDILRVNTEITKA
PLSASMIKRYDEHHQDLTLLKALVRQQLPEKYKEI
FFDQSKNGYAGYIDGGASQEEFYKFIKPILEKMDG
TEELLVKLNREDLLRKQRTFDNGSIPHQIHLGELH
AILRRQEDFYPFLKDNREKIEKILTFRIPYYVGPL
ARGNSRFAWMTRKSEETITPWNFEEVVDKGASAQS
FIERMTNFDKNLPNEKVLPKHSLLYEYFTVYNELT
KVKYVTEGMRKPAFLSGEQKKAIVDLLFKTNRKVT
VKQLKEDYFKKIECFDSVEISGVEDRFNASLGTYH
DLLKIIKDKDFLDNEENEDILEDIVLTLTLFEDRE
MIEERLKTYAHLEDDKVMKQLKRRRYTGWGRLSRK
LINGIRDKQSGKTILDFLKSDGFANRNEMQLIHDD
SLTFKEDIQKAQVSGQGDSLHEHIANLAGSPAIKK
GILQTVKVVDELVKVMGRHKPENIVIEMARENQTT
QKGQKNSRERMKRIEEGIKELGSQILKEHPVENTQ
LQNEKLYLYYLQNGRDMYVDQELDINRLSDYDVDA
IVPQSFLKDDSIDNKVLTRSDKNRGKSDNVPSEEV
VKKMKNYWRQLLNAKLITQRKFDNLTKAERGGLSE
LDKAGFIKRQLVETRQITKHVAQILDSRMNTKYDE
NDKLIREVKVITLKSKLVSDFRKDFQFYKVREINN
YHHAHDAYLNAVVGTALIKKYPKLESEFVYGDYKV
YDVRKMIAKSEQEIGKATAKYFFYSNIMNFFKTEI
TLANGEIRKRPLIETNGETGEIVWDKGRDFATVRK
VLSMPQVNIVKKTEVQTGGFSKESILPKRNSDKLI
ARKKDWDPKKYGGFDSPTVAYSVLVVAKVEKGKSK
KLKSVKELLGITIMERSSFEKNPIDFLEAKGYKEV
KKDLIIKLPKYSLFELENGRKRMLASAGELQKGNE
LALPSKYVNFLYLASHYEKLKGSPEDNEQKQLFVE
QHKHYLDEIIEQISEFSKRVILADANLDKVLSAYN
KHRDKPIREQAENIIHLFTLTNLGAPAAFKYFDTT
IDRKRYTSTKEVLDATLIHQSITGLYETRIDLSQL
GGD

Additional suitable nuclease-inactive dCas9 domains will be apparent to those of skill in the art based on this disclosure and knowledge in the field and are within the scope of this disclosure. Such additional exemplary suitable nuclease-inactive Cas9 domains include, but are not limited to, D10A/H840A, D10A/D839A/H840A, and D10A/D839A/H840A/N863A mutant domains (See, e.g., Prashant et al., CAS9 transcriptional activators for target specificity screening and paired nickases for cooperative genome engineering. Nature Biotechnology. 2013; 31(9): 833-838, the entire contents of which are incorporated herein by reference).

In some embodiments, the Cas9 is a circular permutant Cas9.Circular permutant Cas9s are known in the art and described, for example, in Oakes et al., Cell 176, 254-267, 2019. An exemplary circular permutant Cas9 amino acid sequence is provided below, wherein the bold sequence indicates sequence derived from Cas9, the italics sequence denotes a linker sequence, and the underlined sequence denotes a bipartite nuclear localization sequence (BPNLS).

CP5 (with MSP “NGC=Pam Variant with mutations Regular Cas9 likes NGG” PID=Protein Interacting Domain and “D10A” nickase):

(SEQ ID NO: 33)
EIGKATAKYFFYSNIMNFFKTEITLANGEIRKRPL
IETNGETGEIVWDKGRDFATVRKVLSMPQVNIVKK
TEVQTGGFSKESILPKRNSDKLIARKKDWDPKKYG
GFMQPTVAYSVLVVAKVEKGKSKKLKSVKELLGIT
IMERSSFEKNPIDFLEAKGYKEVKKDLIIKLPKYS
LFELENGRKRMLASAKFLQKGNELALPSKYVNFLY
LASHYEKLKGSPEDNEQKQLFVEQHKHYLDEIIEQ
ISEFSKRVILADANLDKVLSAYNKHRDKPIREQAE
NIIHLFTLTNLGAPRAFKYFDTTIARKEYRSTKEV
LDATLIHQSITGLYETRIDLSQLGGDGGSGGSGGS
GGSGGSGGSGGMDKKYSIGLAIGTNSVGWAVITDE
YKVPSKKFKVLGNTDRHSIKKNLIGALLFDSGETA
EATRLKRTARRRYTRRKNRICYLQEIFSNEMAKVD
DSFFHRLEESFLVEEDKKHERHPIFGNIVDEVAYH
EKYPTIYHLRKKLVDSTDKADLRLIYLALAHMIKF
RGHFLIEGDLNPDNSDVDKLFIQLVQTYNQLFEEN
PINASGVDAKAILSARLSKSRRLENLIAQLPGEKK
NGLFGNLIALSLGLTPNFKSNFDLAEDAKLQLSKD
TYDDDLDNLLAQIGDQYADLFLAAKNLSDAILLSD
ILRVNTEITKAPLSASMIKRYDEHHQDLTLLKALV
RQQLPEKYKEIFFDQSKNGYAGYIDGGASQEEFYK
FIKPILEKMDGTEELLVKLNREDLLRKQRTFDNGS
IPHQIHLGELHAILRRQEDFYPFLKDNREKIEKIL
TFRIPYYVGPLARGNSRFAWMTRKSEETITPWNFE
EVVDKGASAQSFIERMTNFDKNLPNEKVLPKHSLL
YEYFTVYNELTKVKYVTEGMRKPAFLSGEQKKAIV
DLLFKTNRKVTVKQLKEDYFKKIECFDSVEISGVE
DRFNASLGTYHDLLKIIKDKDFLDNEENEDILEDI
VLTLTLFEDREMIEERLKTYAHLFDDKVMKQLKRR
RYTGWGRLSRKLINGIRDKQSGKTILDFLKSDGFA
NRNFMQLIHDDSLTFKEDIQKAQVSGQGDSLHEHI
ANLAGSPAIKKGILQTVKVVDELVKVMGRHKPENI
VIEMARENQTTQKGQKNSRERMKRIEEGIKELGSQ
ILKEHPVENTQLQNEKLYLYYLQNGRDMYVDQELD
INRLSDYDVDHIVPQSFLKDDSIDNKVLTRSDKNR
GKSDNVPSEEVVKKMKNYWRQLLNAKLITQRKFDN
LTKAERGGLSELDKAGFIKRQLVETRQITKHVAQI
LDSRMNTKYDENDKLIREVKVITLKSKLVSDFRKD
FQFYKVREINNYHHAHDAYLNAVVGTALIKKYPKL
ESEFVYGDYKVYDVRKMIAKSEQEGADKRTADGSE
FESPKKKRKV*

Some aspects of the disclosure provide high fidelity Cas9 domains for use in prime editors. In some embodiments, high fidelity Cas9 domains are engineered Cas9 domains comprising one or more mutations that decrease electrostatic interactions between the Cas9 domain and a sugar-phosphate backbone of a DNA, as compared to a corresponding wild-type Cas9 domain. Without wishing to be bound by any particular theory, high fidelity Cas9 domains that have decreased electrostatic interactions with a sugar-phosphate backbone of DNA may have less off-target effects. In some embodiments, a Cas9 domain (e.g., a wild-type Cas9 domain) comprises one or more mutations that decreases the association between the Cas9 domain and a sugar-phosphate backbone of a DNA. In some embodiments, a Cas9 domain comprises one or more mutations that decreases the association between the Cas9 domain and a sugar-phosphate backbone of a DNA by at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, or at least 70%.

In some embodiments, any of the prime editors provided herein comprise a Cas9 with one or more of a N497X, a R661X, a Q695X, and/or a Q926X amino acid substitutions as compared to a wild-type SpCas9, or a corresponding amino acid substitution thereof, wherein X is any amino acid other than the original amino acid. In some embodiments, any of the prime editors provided herein comprise a Cas9 with one or more of a N497A, a R661A, a Q695A, and/or a Q926A amino acid substitutions as compared to a wild-type SpCas9, or a corresponding amino acid substitution thereof. Cas9 domains with high fidelity are known in the art and would be apparent to the skilled artisan. For example, Cas9 domains with high fidelity have been described in Kleinstiver, B. P., et al. “High-fidelity CRISPR-Cas9 nucleases with no detectable genome-wide off-target effects.” Nature 529, 490-495 (2016); and Slaymaker, I. M., et al. “Rationally engineered Cas9 nucleases with improved specificity.” Science 351, 84-88 (2015); the entire contents of each are incorporated herein by reference.

In some embodiments, the high fidelity Cas9 enzyme is SpCas9(K855A), eSpCas9(1.1), SpCas9-HF1, or hyper accurate Cas9 variant (HypaCas9). In some embodiments, the modified Cas9 eSpCas9(1.1) contains alanine substitutions that may weaken the interactions between the HNH/RuvC groove and the target DNA strand, preventing strand separation and cutting at off-target sites. Similarly, SpCas9-HF1 may lower off-target editing through alanine substitutions that disrupt Cas9's interactions with the DNA phosphate backbone. HypaCas9 contains mutations (SpCas9 N692A/M694A/Q695A/H698A) in the REC3 domain that may increase Cas9 proofreading and target discrimination.

An exemplary high fidelity Cas9 is provided below.

High Fidelity Cas9 domain mutations relative to Cas9 are shown in bold and underlined.

(SEQ ID NO: 34)
DKKYSIGLAIGTNSVGWAVITDEYKVPSKKFKVLG
NTDRHSIKKNLIGALLFDSGETAEATRLKRTARRR
YTRRKNRICYLQEIFSNEMAKVDDSFFHRLEESFL
VEEDKKHERHPIFGNIVDEVAYHEKYPTIYHLRKK
LVDSTDKADLRLIYLALAHMIKFRGHFLIEGDLNP
DNSDVDKLFIQLVQTYNQLFEENPINASGVDAKAI
LSARLSKSRRLENLIAQLPGEKKNGLFGNLIALSL
GLTPNFKSNFDLAEDAKLQLSKDTYDDDLDNLLAQ
IGDQYADLFLAAKNLSDAILLSDILRVNTEITKAP
LSASMIKRYDEHHQDLTLLKALVRQQLPEKYKEIF
FDQSKNGYAGYIDGGASQEEFYKFIKPILEKMDGT
EELLVKLNREDLLRKQRTFDNGSIPHQIHLGELHA
ILRRQEDFYPFLKDNREKIEKILTFRIPYYVGPLA
RGNSRFAWMTRKSEETITPWNFEEVVDKGASAQSF
IERMTAFDKNLPNEKVLPKHSLLYEYFTVYNELTK
VKYVTEGMRKPAFLSGEQKKAIVDLLFKTNRKVTV
KQLKEDYFKKIECFDSVEISGVEDRFNASLGTYHD
LLKIIKDKDFLDNEENEDILEDIVLTLTLFEDREM
IEERLKTYAHLFDDKVMKQLKRRRYTGWGALSRKL
INGIRDKQSGKTILDFLKSDGFANRNFMALIHDDS
LTFKEDIQKAQVSGQGDSLHEHIANLAGSPAIKKG
ILQTVKVVDELVKVMGRHKPENIVIEMARENQTTQ
KGQKNSRERMKRIEEGIKELGSQILKEHPVENTQL
QNEKLYLYYLQNGRDMYVDQELDINRLSDYDVDHI
VPQSFLKDDSIDNKVLTRSDKNRGKSDNVPSEEVV
KKMKNYWRQLLNAKLITQRKFDNLTKAERGGLSEL
DKAGFIKRQLVETRAITKHVAQILDSRMNTKYDEN
DKLIREVKVITLKSKLVSDFRKDFQFYKVREINNY
HHAHDAYLNAVVGTALIKKYPKLESEFVYGDYKVY
DVRKMIAKSEQEIGKATAKYFFYSNIMNFFKTEIT
LANGEIRKRPLIETNGETGEIVWDKGRDFATVRKV
LSMPQVNIVKKTEVQTGGFSKESILPKRNSDKLIA
RKKDWDPKKYGGFDSPTVAYSVLVVAKVEKGKSKK
LKSVKELLGITIMERSSFEKNPIDFLEAKGYKEVK
KDLIIKLPKYSLFELENGRKRMLASAGELQKGNEL
ALPSKYVNFLYLASHYEKLKGSPEDNEQKQLFVEQ
HKHYLDEIIEQISEFSKRVILADANLDKVLSAYNK
HRDKPIREQAENIIHLFTLTNLGAPAAFKYFDTTI
DRKRYTSTKEVLDATLIHQSITGLYETRIDLSQLG
GD

In some embodiments, the napDNAbp is a Cpf1 protein or a functional fragment or functional variant thereof.

In some embodiments, the Cpf1 in a prime editor is from Prevotella. In some embodiments, the Cpf1 in a prime editor is from Francisella 1. In some embodiments, the Cpf1 in a prime editor is from an organism from a genus comprising Streptococcus, Campylobacter, Nitratifr actor, Staphylococcus, Parvibaculum, Roseburia, Neisseria, Gluconacetobacier, Azospirillum, Sphaerochaeta, Lactobacillus, Eubacterium, Corynebacter, Carnobacterium, Rhodobacter, Listeria, Paludibacter, Clostridium, Lachnospiraceae, Clostridiaridium, Leptotrichia, Francisella, Legionella, Methanomethyophilus, Porphyromonas, Prevotella, Bacteroidetes, Helcococcus, Leptospira, Desulfovibrio, Desulfonatronum, Opitutaceae, Tuberibacillus, Bacillus, Brevibacilus, Methylobacterium, Butyvibrio, Perigrinibacterium, Pareubacterium, Moraxella, Thiomicrospira or Acidaminococcus. In some embodiments, the Cpf1 in a prime editor is from an organism from a genus selected from Eubacterium, Lachnospiraceae, Leptotrichia, Francisella, Methanomethyophilus, Porphymmonas, Prevotella, Leptospira, Butyvibrio, Perigrinibacterium, Pareubacterium, Moraxella, Thiomicrospira or Acidaminococcus. In some embodiments, the Cpf1 in a prime editor is from an organism selected from S. mutans, S. agalactiae, S. equisimilis, S. sanguinis, S. pneumonia; C. jejuni, C. coli; N. salsuginis, N. tergarcus; S. auricularis, S. carnosus; N. meningitides, N. gonorrhoeae; L. monocytogenes, L. ivanovii; C. botulinum, C. difficile, C. tetani, C. sordellii, L. inadai, F tularensis 1, P. albensis, L. bacterium, B. proteoclasticus, P. bacterium, P. crevioricanis, P. disiens and P. macacae. In other embodiments, a Cpf1 in a prime editor is from organism selected from Streptococcus, Campylobacter, Nitratifractor, Staphylococcus, Parvibaculum, Roseburia, Neisseria, Gluconacetobacter, Azospirillum, Sphaerochaeta, Lactobacillus, Eubacterium, Corynebacter, Carnobacterium, Rhodobacter, Listeria, Paludibacter, Clostridium, Lachnospiraceae, Clostridiaridium, Leptotrichia, Francisella, Legionella, Alicyclobacillus, Methanomethyophilus, Porphyromonas, Prevotella, Bacteroidetes, Helcococcus, Letospira, Desulfovibrio, Desulfonatronum, Opitutaceae, Tuberibacillus, Bacillus, Brevibacilus, Methylobacterium, Butyvibrio, Perigrinibacterium, Pareubacterium, Moraxella, Thiomicrospira or Acidaminococcus. In some embodiments, the Cpf1 in a prime editor is derived from a bacterial species selected from Francisella tularensis 1, Prevotella albensis, Lachnospiraceae bacterium MC2017 1, Butyrivibrio proteoclasticus, Peregrinibacteria bacterium Gih2011 GRA2 33 10, Parcubacteria bacterium GW2011 GWC2 44 17, Smithella sp. SCADC, Acidaminococcus sp. BV3L6, Lachnospiraceae bacterium M42020, Candidatus methanoplasma termitum, Eubacterium eligens, Moraxella bovoculi 237, Moraxella bovoculi AAX08 00205, Moraxella bovoculi AAX11 00205, Butyrivibrio sp. NC3005, Thiomicrospira sp. XS5, Leptospira inadai, Lachnospiraceae bacterium ND2006, Porphyromonas crevioricanis 3, Prevotella disiens and Porphyromonas macacae. In certain embodiments, the Cpf1 in a prime editor is derived from a bacterial species selected from Acidaminococcus sp. BV3L6, Lachnospiraceae bacterium MA2020. In certain embodiments, the in a prime editor is derived from a subspecies of Francsella tularensis 1, including but not limited to Francisella tularensis subsp. novicida. In certain preferred embodiments, the Cpf1 in a prime editor is derived from a bacterial species selected from Acidaminococcus sp. BV3L6, Lachnospiraceae bacterium ND2006, Lachnospiraceae bacterium MA2020, Moraxella bovoculi AAX08 00205, Moraxella bovoculi AAX11 00205, Butyrivibrio sp. NC3005, or Thiomicrospira sp. XS5. In some embodiments, the Cpf1 in a prime editor is derived from Francisella novicida UI 12 (FnCpf1), Acidaminococcus sp. BV3L6 (AsCpf1), Lachnospiraceae bacterium ND2006 (LbCpf1) or Moraxella bovoculi 237 (MbCpf1). It should be appreciated that Cpf1 from other bacterial species may also be used in accordance with the present disclosure.

In some embodiments, a prime editor comprises a wild-type Cpf1. An exemplary wild-type Francisella novicida Cpf1 amino acid sequence is provided below (D917, E1006, and D1255 are bolded and underlined):

(SEQ ID NO: 35)
MSIYQEFVNKYSLSKTLRFELIPQGKTLENIKARG
LILDDEKRAKDYKKAKQIIDKYHQFFIEEILSSVC
ISEDLLQNYSDVYFKLKKSDDDNLQKDFKSAKDTI
KKQISEYIKDSEKFKNLFNQNLIDAKKGQESDLIL
WLKQSKDNGIELFKANSDITDIDEALEIIKSFKGW
TTYFKGFHENRKNVYSSNDIPTSIIYRIVDDNLPK
FLENKAKYESLKDKAPEAINYEQIKKDLAEELTFD
IDYKTSEVNQRVFSLDEVFEIANFNNYLNQSGITK
FNTIIGGKFVNGENTKRKGINEYINLYSQQINDKT
LKKYKMSVLFKQILSDTESKSFVIDKLEDDSDVVT
TMQSFYEQIAAFKTVEEKSIKETLSLLFDDLKAQK
LDLSKIYFKNDKSLTDLSQQVFDDYSVIGTAVLEY
ITQQIAPKNLDNPSKKEQELIAKKTEKAKYLSLE
TIKLAL
EEFNKHRDIDKQCRFEEILANFAAIPMIFDEIAQN
KDNLAQISIKYQNQGKKDLLQASAEDDVKAIKDLL
DQTNNLLHKLKIFHISQSEDKANILDKDEHFYLVF
EECYFELANIVPLYNKIRNYITQKPYSDEKFKLNF
ENSTLANGWDKNKEPDNTAILFIKDDKYYLGVMNK
KNNKIFDDKAIKENKGEGYKKIVYKLLPGANKMLP
KVFFSAKSIKFYNPSEDILRIRNHSTHTKNGSPQK
GYEKFEFNIEDCRKFIDFYKQSISKHPEWKDFGFR
FSDTQRYNSIDEFYREVENQGYKLTFENISESYID
SVVNQGKLYLFQIYNKDFSAYSKGRPNLHTLYWKA
LFDERNLQDVVYKLNGEAELFYRKQSIPKKITHPA
KEAIANKNKDNPKKESVFEYDLIKDKRFTEDKFFF
HCPITINFKSSGANKFNDEINLLLKEKANDVHILS
IDRGERHLAYYTLVDGKGNIIKQDTFNIIGNDRMK
TNYHDKLAAIEKDRDSARKDWKKINNIKEMKEGYL
SQVVHEIAKLVIEYNAIVVFEDLNFGFKRGRFKVE
KQVYQKLEKMLIEKLNYLVFKDNEFDKTGGVLRAY
QLTAPFETFKKMGKQTGIIYYVPAGFTSKICPVTG
FVNQLYPKYESVSKSQEFFSKFDKICYNLDKGYFE
FSFDYKNFGDKAAKGKWTIASFGSRLINFRNSDKN
HNWDTREVYPTKELEKLLKDYSIEYGHGECIKAAI
CGESDKKFFAKLTSVLNTILQMRNSKTGTELDYLI
SPVADVNGNFFDSRQAPKNMPQDADANGAYHIGLK
GLMLLGRIKNNQEGKKLNLVIKNEEYFEEVQNRNN

In some embodiments, a prime editor comprises a wild-type Cpf1 lacking an N terminal methionine residue. An exemplary amino acid sequence of a wild-type Francisella novicida Cpf1 lacking an N terminal methionine is provided below (D917, E1006, and D1255 are bolded and underlined):

(SEQ ID NO: 36)
SIYQEFVNKYSLSKTLRFELIPQGKTLENIKARGL
ILDDEKRAKDYKKAKQIIDKYHQFEIEEILSSVCI
SEDLLQNYSDVYFKLKKSDDDNLQKDFKSAKDTIK
KQISEYIKDSEKFKNLFNQNLIDAKKGQESDLILW
LKQSKDNGIELFKANSDITDIDEALEIIKSFKGWT
TYFKGFHENRKNVYSSNDIPTSIIYRIVDDNLPKE
LENKAKYESLKDKAPEAINYEQIKKDLAEELTFDI
DYKTSEVNQRVFSLDEVFEIANFNNYLNQSGITKE
NTIIGGKFVNGENTKRKGINEYINLYSQQINDKTL
KKYKMSVLFKQILSDTESKSFVIDKLEDDSDVVTT
MQSFYEQIAAFKTVEEKSIKETLSLLFDDLKAQKL
DLSKIYFKNDKSLTDLSQQVFDDYSVIGTAVLEYI
TQQIAPKNLDNPSKKEQELIAKKTEKAKYLSLETI
KLALEEFNKHRDIDKQCRFEEILANFAAIPMIFDE
IAQNKDNLAQISIKYQNQGKKDLLQASAEDDVKAI
KDLLDQTNNLLHKLKIFHISQSEDKANILDKDEHF
YLVFEECYFELANIVPLYNKIRNYITQKPYSDEKF
KLNFENSTLANGWDKNKEPDNTAILFIKDDKYYLG
VMNKKNNKIFDDKAIKENKGEGYKKIVYKLLPGAN
KMLPKVFFSAKSIKFYNPSEDILRIRNHSTHTKNG
SPQKGYEKFEFNIEDCRKFIDFYKQSISKHPEWKD
FGFRFSDTQRYNSIDEFYREVENQGYKLTFENISE
SYIDSVVNQGKLYLFQIYNKDFSAYSKGRPNLHTL
YWKALFDERNLQDVVYKLNGEAELFYRKQSIPKKI
THPAKEAIANKNKDNPKKESVFEYDLIKDKRFTED
KFFFHCPITINFKSSGANKFNDEINLLLKEKANDV
HILSIDRGERHLAYYTLVDGKGNIIKQDTFNIIGN
DRMKTNYHDKLAAIEKDRDSARKDWKKINNIKEMK
EGYLSQVVHEIAKLVIEYNAIVVFEDLNFGFKRGR
FKVEKQVYQKLEKMLIEKLNYLVFKDNEFDKTGGV
LRAYQLTAPFETFKKMGKQTGIIYYVPAGFTSKIC
PVTGFVNQLYPKYESVSKSQEFFSKFDKICYNLDK
GYFEFSFDYKNFGDKAAKGKWTIASFGSRLINFRN
SDKNHNWDTREVYPTKELEKLLKDYSIEYGHGECI
KAAICGESDKKFFAKLTSVLNTILQMRNSKTGTEL
DYLISPVADVNGNFFDSRQAPKNMPQDADANGAYH
IGLKGLMLLGRIKNNQEGKKLNLVIKNEEYFEFVQ
NRNN

In some embodiments, a prime editor comprises a functional variant of a wild-type Cpf1 that comprises one or more amino acid substitutions, insertions, and/or deletions compared to a wild-type Cpf1. In some embodiments, the Cpf1 domain is a functional variant of a wild-type Cpf1 that comprises any one of the Cpf1 amino acid sequences as set forth herein. In some embodiments, the Cpf1 domain is a functional variant of a wild-type Cpf1 that consists of any one of the Cpf1 amino acid sequences as set forth herein.

In some embodiments, the Cpf1 domains is a functional variant of a wild-type Cpf1 that comprise an amino acid sequence that is at least 50%, at least 60%, at least 65%, at least 70%, at least 75%, at least 80%, at least 85%, at least 90%, at least 95%, at least 96%, at least 97%, at least 98%, at least 99%, or at least 99.5% identical to a Cpf1 polypeptide described herein. In some embodiments, the Cpf1 domain is a functional variant of a wild-type Cpf1 that comprises an amino acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more or more mutations compared to any one of the amino acid sequences set forth herein. In some embodiments, the Cpf1 domain is a functional variant of a wild-type Cpf1 that comprises any one of the amino acid sequences as set forth herein. comprises an amino acid sequence that has at least 10, at least 15, at least 20, at least 30, at least 40, at least 50, at least 60, at least 70, at least 80, at least 90, at least 100, at least 150, at least 200, at least 250, at least 300, at least 350, at least 400, at least 500, at least 600, at least 700, at least 800, at least 900, at least 1000, at least 1100, or at least 1200 identical contiguous amino acid residues as compared to any one of the amino acid sequences set forth herein.

In some embodiments, a prime editor as provided herein comprise the full-length amino acid sequence of a Cpf1 protein, e.g., one of the Cpf1 sequences provided herein. In some embodiments, a prime editor comprises a functional fragment of Cpf1. Exemplary amino acid sequences of suitable Cpf1 domains and Cpf1 fragments are provided herein, and additional suitable sequences of Cpf1 domains and fragments will be apparent to those of skill in the art.

The Cpf1 domain may be a nuclease active Cpf1, a nuclease inactive Cpf1 (dCpf1), or a Cpf1 nickase (nCpf1). In some embodiments, the Cpf1 domain is a nuclease active domain. For example, the Cpf1 domain may be a Cpf1 domain that cuts both strands of a double stranded DNA molecule).

In some embodiments, a prime editor comprises a functional variant of a wild-type Cpf1 that has one or more amino acid deletion, insertion, or substitution that reduces the nuclease activity of the Cpf1 polypeptide. For example, in some embodiments, the functional Cpf1 has less than 50%, less than 40%, less than 30%, less than 20%, less than about 15%, less than about 10%, less than about 5%, less than about 1%, or less than about 0.1% of the nuclease activity of the corresponding wild-type Cpf1 protein.

In some embodiments, a prime editor comprises a functional variant of a wild-type Cpf1 that is a Cpf1 nickase (nCpf1). In some embodiments, the nCpf1 cleaves only the edit strand, and not the target strand, of a double stranded target polynucleotide, e.g., a target DNA molecule. in some embodiments, the nCpf1 cleaves only the target strand, and not the edit strand, of a double stranded target polynucleotide, e.g., a target DNA molecule.

In some embodiments, the nCpf1 comprises a mutation in a Nuc domain. In some embodiments, the nCpf1 comprises a mutation at position 1226A in the Nuc domain of as compared to a wild-type Cpf1 from Acidaminococcus (AsCpf1) or a corresponding position thereof. In some embodiments, the nCpf1 comprises an arginine-to-alanine substitution or an R1226A mutation as compared to a wild-type AsCpf1 or a corresponding position thereof. It will be understood by the skilled person that where the enzyme is not AsCpf1, a mutation may be made at a residue in a corresponding position. In particular embodiments, nCpf1. is FnCpf1 functional variant and the mutation is at position 81218 as set forth in a wild-type Francisella novicida Cpf1 (FnCpf1). In some embodiments, the nCpf1 is LbCpf1 functional variant and the mutation is at position R1138 as set forth in a wild-type LbCpf1. In particular embodiments, the nCpf1 is MbCpf1 functional variant and the mutation is at position R1293.

In some embodiments, a prime editor comprises a functional variant of a wild-type Cpf1 that is a nuclease inactive Cpf1 (dCpf1). In some embodiments, the dCpf1 of a prime editor retains the ability to associate with a PE guide polynucleotide and bind to a double stranded target polynucleotide but lacks the ability to cleave either strand of the target polynucleotide. In some embodiments, the dCpf1 comprises one or more amino acid substitutions, deletions or insertions in a RuvC-like domain that reduces or abolishes nuclease activity of the RuvC-like domain. In some embodiments, the dCpf1 comprises an amino acid substitution at position D917, E1006, or D1255 as compared to a wild-type Francisella novicida Cpf1 (FnCpf1) or a corresponding substitution thereof. In some embodiments, the dCpf1 comprises a D917A, E1006A, or D1255A amino acid substitution as compared to a wild-type Francisella novicida Cpf1 (FnCpf1) or a corresponding position thereof.

In some embodiments, the dCpf1 of comprises amino acid substitutions D917A, E1006A, D1255A, D917A/E1006A, D917A/D1255A, E1006A/D1255A, or D917A/E1006A/D1255A as compared to a wild-type Francisella novicida Cpf1 (FnCpf1) or corresponding substitutions thereof. In some embodiments, the dCpf1 comprises an amino acid sequence that is at least 85%, at least 90%, at least 91%, at least 92%, at least 93%, at least 94%, at least 95%, at least 96%, at least 97%, at least 98%, at least 99%, or at ease 99.5% identical to a Cpf1 sequence disclosed herein, and comprises mutations corresponding to D917A, E1006A, D1255A, D917A/E1006A, D917A/D1255A, E1006A/D1255A, or D917A/E1006A/D1255A as compared to a wild-type Francisella novicida Cpf1 (FnCpf1) or corresponding substitutions thereof. In some embodiments, the dCpf1 of comprises amino acid substitution D917A or E1006A as compared to a wild-type FnCpf1 or corresponding substitutions thereof, wherein the D917A or E1006A amino acid substitution completely inactivates the DNA cleavage activity. In some embodiments, the Cpf1 functional variant comprises amino acid substitution D1255A as compared to a wild-type FnCpf1 or a corresponding substitution thereof, wherein the Cpf1 functional variant has significantly reduced nucleolytic activity.

Additional non-limiting examples of amino acid substitutions that can be used to reduce or abolish Cpf1 nuclease activity include D917A, E1006A, E1028A, D1227A, D1255A, N1257A, D917A E1006A, E1028A, D1227A, D1255A and N1257A as compared to a wild-type FnCpf1. In some embodiments, a dCpf1 comprises an amino acid substitution at position D908, E993, and/or D1263 as compared to a wild-type AsCpf1 or corresponding positions thereof. In some embodiments, the dCpf1 comprises an amino acid substitution D908A, E993A, and/or D1263A as compared to a wild-type AsCpf1 or corresponding positions thereof. In some embodiments, a dCpf1 comprises an amino acid substitution at position R909, R912, 8930, R947, K949, R951, R955, K965, K968, K1000, K1002, R1003, K1009, K1017, K1022, K1029, K1035, K1054, K1072, K1086, R1094, K1095, K1109, K1118, K1142, K1150, K1158, K1159, R1220, R1226, R1242, and/or R1252 as compared to a wild-type AsCpf1 or corresponding positions thereof (Acidaminococcus sp. BV3L6).

In some embodiments, a dCpf1 comprises an amino acid substitution at position D832, E925, D947 and/or D1180 as compared to a wild-type LbCpf1 or corresponding positions thereof. In some embodiments, a dCpf1 comprises amino acid substitution D832A, E925A, D947A and/or D1180A as compared to a wild-type LbCpf1 or corresponding positions thereof.

In some embodiments, a Cpf1 functional variant of a prime editor comprises one or more amino acid substitutions in a putative second nuclease domain similar to PD-(D/E)XK nuclease superfamily and is Hinc III endonuclease like. In some embodiments, the Cpf1 functional variant comprises an amino acid substitution in the Hindi endonuclease like domain that reduces or abolishes Cpf1 nuclease activity. in some embodiments, the Cpf1 functional variant comprises a N580A, N584A, T587A, W609A, D610A, K613A, E614A, D616A, K624A, D625A, K627A and Y629A as compared to a wild-type FnCpf1.

Mutations can also be made at neighboring residues, e.g., at amino acids near those indicated above that participate in the nuclease activity. In some embodiments, only the RuvC domain of a Cpf1 is inactivated. In other embodiments, another putative nuclease domain is inactivated. In some embodiments, the other putative nuclease domain is a HincII-like endonuclease domain. In some embodiments, mutation(s) in the HincII-like endonuclease domain and not the RuvC domain results in a Cpf1 nickase.

Francisella novicida Cpf1 D917A (A917, E1006, and D1255 are bolded and
underlined)
(SEQ ID NO: 37)
MSIYQEFVNKYSLSKTLRFELIPQGKTLENIKARGLILDDEKRAKDYKKAKQIIDKYHQFFIEEILSSVC
ISEDLLQNYSDVYFKLKKSDDDNLQKDFKSAKDTIKKQISEYIKDSEKFKNLFNQNLIDAKKGQESDLIL
WLKQSKDNGIELFKANSDITDIDEALEIIKSFKGWTTYFKGFHENRKNVYSSNDIPTSIIYRIVDDNLPK
FLENKAKYESLKDKAPEAINYEQIKKDLAEELTFDIDYKTSEVNQRVFSLDEVFEIANFNNYLNQSGITK
FNTIIGGKFVNGENTKRKGINEYINLYSQQINDKTLKKYKMSVLFKQILSDTESKSFVIDKLEDDSDVVT
TMQSFYEQIAAFKTVEEKSIKETLSLLFDDLKAQKLDLSKIYFKNDKSLTDLSQQVFDDYSVIGTAVLEY
ITQQIAPKNLDNPSKKEQELIAKKTEKAKYLSLETIKLALEEFNKHRDIDKQCRFEEILANFAAIPMIFD
EIAQNKDNLAQISIKYQNQGKKDLLQASAEDDVKAIKDLLDQTNNLLHKLKIFHISQSEDKANILDKDEH
FYLVFEECYFELANIVPLYNKIRNYITQKPYSDEKFKLNFENSTLANGWDKNKEPDNTAILFIKDDKYYL
GVMNKKNNKIFDDKAIKENKGEGYKKIVYKLLPGANKMLPKVFFSAKSIKFYNPSEDILRIRNHSTHTKN
GSPQKGYEKFEFNIEDCRKFIDFYKQSISKHPEWKDFGFRFSDTQRYNSIDEFYREVENQGYKLTFENIS
ESYIDSVVNQGKLYLFQIYNKDFSAYSKGRPNLHTLYWKALFDERNLQDVVYKLNGEAELFYRKQSIPKK
ITHPAKEAIANKNKDNPKKESVFEYDLIKDKRFTEDKFFFHCPITINFKSSGANKFNDEINLLLKEKAND
VHILSIARGERHLAYYTLVDGKGNIIKQDTFNIIGNDRMKTNYHDKLAAIEKDRDSARKDWKKINNIKEM
KEGYLSQVVHEIAKLVIEYNAIVVFEDLNFGFKRGRFKVEKQVYQKLEKMLIEKLNYLVFKDNEFDKTGG
VLRAYQLTAPFETFKKMGKQTGIIYYVPAGFTSKICPVTGFVNQLYPKYESVSKSQEFFSKFDKICYNLD
KGYFEFSFDYKNFGDKAAKGKWTIASFGSRLINFRNSDKNHNWDTREVYPTKELEKLLKDYSIEYGHGEC
IKAAICGESDKKFFAKLTSVLNTILQMRNSKTGTELDYLISPVADVNGNFFDSRQAPKNMPQDADANGAY
HIGLKGLMLLGRIKNNQEGKKLNLVIKNEEYFEFVQNRNN
Francisella novicida Cpf1 E1006A (D917, A1006, and D1255 are bolded and
underlined)
(SEQ ID NO: 38)
MSIYQEFVNKYSLSKTLRFELIPQGKTLENIKARGLILDDEKRAKDYKKAKQIIDKYHQFFIEEILSSVC
ISEDLLQNYSDVYFKLKKSDDDNLQKDFKSAKDTIKKQISEYIKDSEKFKNLFNQNLIDAKKGQESDLIL
WLKQSKDNGIELFKANSDITDIDEALEIIKSFKGWTTYFKGFHENRKNVYSSNDIPTSIIYRIVDDNLPK
FLENKAKYESLKDKAPEAINYEQIKKDLAEELTFDIDYKTSEVNQRVFSLDEVFEIANFNNYLNQSGITK
FNTIIGGKFVNGENTKRKGINEYINLYSQQINDKTLKKYKMSVLFKQILSDTESKSFVIDKLEDDSDVVT
TMQSFYEQIAAFKTVEEKSIKETLSLLFDDLKAQKLDLSKIYFKNDKSLTDLSQQVFDDYSVIGTAVLEY
ITQQIAPKNLDNPSKKEQELIAKKTEKAKYLSLETIKLALEEFNKHRDIDKQCRFEEILANFAAIPMIFD
EIAQNKDNLAQISIKYQNQGKKDLLQASAEDDVKAIKDLLDQTNNLLHKLKIFHISQSEDKANILDKDEH
FYLVFEECYFELANIVPLYNKIRNYITQKPYSDEKFKLNFENSTLANGWDKNKEPDNTAILFIKDDKYYL
GVMNKKNNKIFDDKAIKENKGEGYKKIVYKLLPGANKMLPKVFFSAKSIKFYNPSEDILRIRNHSTHTKN
GSPQKGYEKFEFNIEDCRKFIDFYKQSISKHPEWKDFGFRFSDTQRYNSIDEFYREVENQGYKLTFENIS
ESYIDSVVNQGKLYLFQIYNKDFSAYSKGRPNLHTLYWKALFDERNLQDVVYKLNGEAELFYRKQSIPKK
ITHPAKEAIANKNKDNPKKESVFEYDLIKDKRFTEDKFFFHCPITINFKSSGANKFNDEINLLLKEKAND
VHILSIDRGERHLAYYTLVDGKGNIIKQDTFNIIGNDRMKTNYHDKLAAIEKDRDSARKDWKKINNIKEM
KEGYLSQVVHEIAKLVIEYNAIVVFADLNFGFKRGRFKVEKQVYQKLEKMLIEKLNYLVFKDNEFDKTGG
VLRAYQLTAPFETFKKMGKQTGIIYYVPAGFTSKICPVTGFVNQLYPKYESVSKSQEFFSKFDKICYNLD
KGYFEFSFDYKNFGDKAAKGKWTIASFGSRLINFRNSDKNHNWDTREVYPTKELEKLLKDYSIEYGHGEC
IKAAICGESDKKFFAKLTSVLNTILQMRNSKTGTELDYLISPVADVNGNFFDSRQAPKNMPQDADANGAY
HIGLKGLMLLGRIKNNQEGKKLNLVIKNEEYFEFVQNRNN
Francisella novicida Cpf1 D1255A (D917, E1006, and A1255 are bolded and
underlined)
(SEQ ID NO: 39)
MSIYQEFVNKYSLSKTLRFELIPQGKTLENIKARGLILDDEKRAKDYKKAKQIIDKYHQFFIEEILSSVC
ISEDLLQNYSDVYFKLKKSDDDNLQKDFKSAKDTIKKQISEYIKDSEKFKNLFNQNLIDAKKGQESDLIL
WLKQSKDNGIELFKANSDITDIDEALEIIKSFKGWTTYFKGFHENRKNVYSSNDIPTSIIYRIVDDNLPK
FLENKAKYESLKDKAPEAINYEQIKKDLAEELTFDIDYKTSEVNQRVFSLDEVFEIANFNNYLNQSGITK
FNTIIGGKFVNGENTKRKGINEYINLYSQQINDKTLKKYKMSVLFKQILSDTESKSFVIDKLEDDSDVVT
TMQSFYEQIAAFKTVEEKSIKETLSLLFDDLKAQKLDLSKIYFKNDKSLTDLSQQVFDDYSVIGTAVLEY
ITQQIAPKNLDNPSKKEQELIAKKTEKAKYLSLETIKLALEEFNKHRDIDKQCRFEEILANFAAIPMIFD
EIAQNKDNLAQISIKYONQGKKDLLQASAEDDVKAIKDLLDQTNNLLHKLKIFHISQSEDKANILDKDEH
EYLVFEECYFELANIVPLYNKIRNYITQKPYSDEKFKLNFENSTLANGWDKNKEPDNTAILFIKDDKYYL
GVMNKKNNKIFDDKAIKENKGEGYKKIVYKLLPGANKMLPKVFFSAKSIKFYNPSEDILRIRNHSTHTKN
GSPQKGYEKFEFNIEDCRKFIDFYKQSISKHPEWKDFGFRFSDTQRYNSIDEFYREVENQGYKLTFENIS
ESYIDSVVNQGKLYLFQIYNKDFSAYSKGRPNLHTLYWKALFDERNLQDVVYKLNGEAELFYRKQSIPKK
ITHPAKEAIANKNKDNPKKESVFEYDLIKDKRFTEDKFFFHCPITINFKSSGANKFNDEINLLLKEKAND
VHILSIDRGERHLAYYTLVDGKGNIIKQDTFNIIGNDRMKTNYHDKLAAIEKDRDSARKDWKKINNIKEM
KEGYLSQVVHEIAKLVIEYNAIVVFEDLNFGFKRGRFKVEKQVYQKLEKMLIEKLNYLVFKDNEFDKTGG
VLRAYQLTAPFETFKKMGKQTGIIYYVPAGFTSKICPVTGFVNQLYPKYESVSKSQEFFSKFDKICYNLD
KGYFEFSFDYKNFGDKAAKGKWTIASFGSRLINFRNSDKNHNWDTREVYPTKELEKLLKDYSIEYGHGEC
IKAAICGESDKKFFAKLTSVLNTILQMRNSKTGTELDYLISPVADVNGNFFDSRQAPKNMPQDAAANGAY
HIGLKGLMLLGRIKNNQEGKKLNLVIKNEEYFEFVQNRNN
Francisella novicida Cpf1 D917A/E1006A (A917, A1006, and D1255 are bolded
and underlined)
(SEQ ID NO: 40)
MSIYQEFVNKYSLSKTLRFELIPQGKTLENIKARGLILDDEKRAKDYKKAKQIIDKYHQFFIEEILSSVC
ISEDLLQNYSDVYFKLKKSDDDNLQKDFKSAKDTIKKQISEYIKDSEKFKNLFNQNLIDAKKGQESDLIL
WLKQSKDNGIELFKANSDITDIDEALEIIKSFKGWTTYFKGFHENRKNVYSSNDIPTSIIYRIVDDNLPK
FLENKAKYESLKDKAPEAINYEQIKKDLAEELTFDIDYKTSEVNQRVFSLDEVFEIANFNNYLNQSGITK
FNTIIGGKFVNGENTKRKGINEYINLYSQQINDKTLKKYKMSVLFKQILSDTESKSFVIDKLEDDSDVVT
TMQSFYEQIAAFKTVEEKSIKETLSLLFDDLKAQKLDLSKIYFKNDKSLTDLSQQVFDDYSVIGTAVLEY
ITQQIAPKNLDNPSKKEQELIAKKTEKAKYLSLETIKLALEEFNKHRDIDKQCRFEEILANFAAIPMIFD
EIAQNKDNLAQISIKYQNQGKKDLLQASAEDDVKAIKDLLDQTNNLLHKLKIFHISQSEDKANILDKDEH
FYLVFEECYFELANIVPLYNKIRNYITQKPYSDEKFKLNFENSTLANGWDKNKEPDNTAILFIKDDKYYL
GVMNKKNNKIFDDKAIKENKGEGYKKIVYKLLPGANKMLPKVFFSAKSIKFYNPSEDILRIRNHSTHTKN
GSPQKGYEKFEFNIEDCRKFIDFYKQSISKHPEWKDFGFRFSDTQRYNSIDEFYREVENQGYKLTFENIS
ESYIDSVVNQGKLYLFQIYNKDFSAYSKGRPNLHTLYWKALFDERNLQDVVYKLNGEAELFYRKQSIPKK
ITHPAKEAIANKNKDNPKKESVFEYDLIKDKRFTEDKFFFHCPITINFKSSGANKFNDEINLLLKEKAND
VHILSIARGERHLAYYTLVDGKGNIIKQDTFNIIGNDRMKTNYHDKLAAIEKDRDSARKDWKKINNIKEM
KEGYLSQVVHEIAKLVIEYNAIVVFADLNFGFKRGRFKVEKQVYQKLEKMLIEKLNYLVFKDNEFDKTGG
VLRAYQLTAPFETFKKMGKQTGIIYYVPAGFTSKICPVTGFVNQLYPKYESVSKSQEFFSKFDKICYNLD
KGYFEFSFDYKNFGDKAAKGKWTIASFGSRLINFRNSDKNHNWDTREVYPTKELEKLLKDYSIEYGHGEC
IKAAICGESDKKFFAKLTSVLNTILQMRNSKTGTELDYLISPVADVNGNFFDSRQAPKNMPQDADANGAY
HIGLKGLMLLGRIKNNQEGKKLNLVIKNEEYFEFVQNRNN
Francisella novicida Cpf1 D917A/D1255A (A917, E1006, and A1255 are bolded
and underlined)
(SEQ ID NO: 41)
MSIYQEFVNKYSLSKTLRFELIPQGKTLENIKARGLILDDEKRAKDYKKAKQIIDKYHQFFIEEILSSVC
ISEDLLQNYSDVYFKLKKSDDDNLQKDFKSAKDTIKKQISEYIKDSEKFKNLFNQNLIDAKKGQESDLIL
WLKQSKDNGIELFKANSDITDIDEALEIIKSFKGWTTYFKGFHENRKNVYSSNDIPTSIIYRIVDDNLPK
FLENKAKYESLKDKAPEAINYEQIKKDLAEELTFDIDYKTSEVNQRVFSLDEVFEIANFNNYLNQSGITK
FNTIIGGKFVNGENTKRKGINEYINLYSQQINDKTLKKYKMSVLFKQILSDTESKSFVIDKLEDDSDVVT
TMQSFYEQIAAFKTVEEKSIKETLSLLFDDLKAQKLDLSKIYFKNDKSLTDLSQQVFDDYSVIGTAVLEY
ITQQ1APKNLDNPSKKEQELIAKKTEKAKYLSLETIKLALEEFNKHRDIDKQCRFEEILANFAAIPMIFD
EIAQNKDNLAQISIKYQNQGKKDLLQASAEDDVKAIKDLLDQTNNLLHKLKIFHISQSEDKANILDKDEH
FYLVFEECYFELANIVPLYNKIRNYITQKPYSDEKFKLNFENSTLANGWDKNKEPDNTAILFIKDDKYYL
GVMNKKNNKIFDDKAIKENKGEGYKKIVYKLLPGANKMLPKVFFSAKSIKFYNPSEDILRIRNHSTHTKN
GSPQKGYEKFEFNIEDCRKFIDFYKQSISKHPEWKDFGFRFSDTQRYNSIDEFYREVENQGYKLTFENIS
ESYIDSVVNQGKLYLFQIYNKDFSAYSKGRPNLHTLYWKALFDERNLQDVVYKLNGEAELFYRKQSIPKK
ITHPAKEAIANKNKDNPKKESVFEYDLIKDKRFTEDKFFFHCPITINFKSSGANKFNDEINLLLKEKAND
VHILSIARGERHLAYYTLVDGKGNIIKQDTFNIIGNDRMKTNYHDKLAAIEKDRDSARKDWKKINNIKEM
KEGYLSQVVHEIAKLVIEYNAIVVFEDLNFGFKRGRFKVEKQVYQKLEKMLIEKLNYLVFKDNEFDKTGG
VLRAYQLTAPFETFKKMGKQTGIIYYVPAGFTSKICPVTGFVNQLYPKYESVSKSQEFFSKFDKICYNLD
KGYFEFSFDYKNFGDKAAKGKWTIASFGSRLINFRNSDKNHNWDTREVYPTKELEKLLKDYSIEYGHGEC
IKAAICGESDKKFFAKLTSVLNTILQMRNSKTGTELDYLISPVADVNGNFFDSRQAPKNMPQDAAANGAY
HIGLKGLMLLGRIKNNQEGKKLNLVIKNEEYFEFVQNRNN
Francisella novicida Cpf1 E1006A/D1255A (D917, A1006, and A1255 are bolded
and underlined)
(SEQ ID NO: 42)
MSIYQEFVNKYSLSKTLRFELIPQGKTLENIKARGLILDDEKRAKDYKKAKQIIDKYHQFFIEEILSSVC
ISEDLLQNYSDVYFKLKKSDDDNLQKDFKSAKDTIKKQISEYIKDSEKFKNLFNQNLIDAKKGQESDLIL
WLKQSKDNGIELFKANSDITDIDEALEIIKSFKGWTTYFKGFHENRKNVYSSNDIPTSIIYRIVDDNLPK
FLENKAKYESLKDKAPEAINYEQIKKDLAEELTFDIDYKTSEVNQRVFSLDEVFEIANFNNYLNQSGITK
FNTIIGGKFVNGENTKRKGINEYINLYSQQINDKTLKKYKMSVLFKQILSDTESKSFVIDKLEDDSDVVT
TMQSFYEQIAAFKTVEEKSIKETLSLLFDDLKAQKLDLSKIYFKNDKSLTDLSQQVFDDYSVIGTAVLEY
ITQQIAPKNLDNPSKKEQELIAKKTEKAKYLSLETIKLALEEFNKHRDIDKQCRFEEILANFAAIPMIFD
EIAQNKDNLAQISIKYQNQGKKDLLQASAEDDVKAIKDLLDQTNNLLHKLKIFHISQSEDKANILDKDEH
FYLVFEECYFELANIVPLYNKIRNYITQKPYSDEKFKLNFENSTLANGWDKNKEPDNTAILFIKDDKYYL
GVMNKKNNKIFDDKAIKENKGEGYKKIVYKLLPGANKMLPKVFFSAKSIKFYNPSEDILRIRNHSTHTKN
GSPQKGYEKFEFNIEDCRKFIDFYKQSISKHPEWKDFGFRFSDTQRYNSIDEFYREVENQGYKLTFENIS
ESYIDSVVNQGKLYLFQIYNKDFSAYSKGRPNLHTLYWKALFDERNLQDVVYKLNGEAELFYRKQSIPKK
ITHPAKEAIANKNKDNPKKESVFEYDLIKDKRFTEDKFFFHCPITINFKSSGANKFNDEINLLLKEKAND
VHILSIDRGERHLAYYTLVDGKGNIIKQDTENIIGNDRMKTNYHDKLAAIEKDRDSARKDWKKINNIKEM
KEGYLSQVVHEIAKLVIEYNAIVVFADLNFGFKRGRFKVEKQVYQKLEKMLIEKLNYLVFKDNEFDKTGG
VLRAYQLTAPFETFKKMGKQTGIIYYVPAGFTSKICPVTGFVNQLYPKYESVSKSQEFFSKFDKICYNLD
KGYFEFSFDYKNFGDKAAKGKWTIASFGSRLINFRNSDKNHNWDTREVYPTKELEKLLKDYSIEYGHGEC
IKAAICGESDKKFFAKLTSVLNTILQMRNSKTGTELDYLISPVADVNGNFFDSRQAPKNMPQDAAANGAY
HIGLKGLMLLGRIKNNQEGKKLNLVIKNEEYFEFVQNRNN
Francisella novicida Cpf1 D917A/E1006A/D1255A (A917, A1006, and A1255 are
bolded and underlined)
(SEQ ID NO: 43)
MSIYQEFVNKYSLSKTLRFELIPQGKTLENIKARGLILDDEKRAKDYKKAKQIIDKYHQFFIEEILSSVC
ISEDLLQNYSDVYFKLKKSDDDNLQKDFKSAKDTIKKQISEYIKDSEKFKNLFNQNLIDAKKGQESDLIL
WLKQSKDNGIELFKANSDITDIDEALEIIKSFKGWTTYFKGFHENRKNVYSSNDIPTSIIYRIVDDNLPK
FLENKAKYESLKDKAPEAINYEQIKKDLAEELTFDIDYKTSEVNQRVFSLDEVFEIANFNNYLNQSGITK
FNTIIGGKFVNGENTKRKGINEYINLYSQQINDKTLKKYKMSVLFKQILSDTESKSFVIDKLEDDSDVVT
TMQSFYEQIAAFKTVEEKSIKETLSLLFDDLKAQKLDLSKIYFKNDKSLTDLSQQVFDDYSVIGTAVLEY
ITQQIAPKNLDNPSKKEQELIAKKTEKAKYLSLETIKLALEEFNKHRDIDKQCRFEEILANFAAIPMIFD
EIAQNKDNLAQISIKYQNQGKKDLLQASAEDDVKAIKDLLDQTNNLLHKLKIFHISQSEDKANILDKDEH
FYLVFEECYFELANIVPLYNKIRNYITQKPYSDEKFKLNFENSTLANGWDKNKEPDNTAILFIKDDKYYL
GVMNKKNNKIFDDKAIKENKGEGYKKIVYKLLPGANKMLPKVFFSAKSIKFYNPSEDILRIRNHSTHTKN
GSPQKGYEKFEFNIEDCRKFIDFYKQSISKHPEWKDFGFRFSDTQRYNSIDEFYREVENQGYKLTFENIS
ESYIDSVVNQGKLYLFQIYNKDFSAYSKGRPNLHTLYWKALFDERNLQDVVYKLNGEAELFYRKQSIPKK
ITHPAKEAIANKNKDNPKKESVFEYDLIKDKRFTEDKFFFHCPITINFKSSGANKFNDEINLLLKEKAND
VHILSIARGERHLAYYTLVDGKGNIIKQDTFNIIGNDRMKTNYHDKLAAIEKDRDSARKDWKKINNIKEM
KEGYLSQVVHEIAKLVIEYNAIVVFADLNFGFKRGRFKVEKQVYQKLEKMLIEKLNYLVFKDNEFDKTGG
VLRAYQLTAPFETFKKMGKQTGIIYYVPAGFTSKICPVTGFVNQLYPKYESVSKSQEFFSKFDKICYNLD
KGYFEFSFDYKNFGDKAAKGKWTIASFGSRLINFRNSDKNHNWDTREVYPTKELEKLLKDYSIEYGHGEC
IKAAICGESDKKFFAKLTSVLNTILQMRNSKTGTELDYLISPVADVNGNFFDSRQAPKNMPQDAAANGAY
HIGLKGLMLLGRIKNNQEGKKLNLVIKNEEYFEFVQNRNN

In certain embodiments, a prime editor comprises a Cpf1 functional variant comprising one or more amino acid substitutions, insertions, and/or deletions to have modified, more preferably increased specificity for the target polynucleotide.

In some embodiments, the Cpf1 functional variant comprises an amino acid substitution in a RAD50 domain as compared to a wild-type Cpf1. In some embodiments, the Cpf1 functional variant comprises an amino acid substitution at position K324, K335, K337, R331, K369, K370, R386, 8392, R393, K400, K404, K406, K408, K414, K429, K436, K438, K459, K460, K464, R670, K675, 8681, K686, K689, R699, K705, R725, K729, K739, K748, and/or K752 with reference to amino acid position numbering of AsCpf1 (Acidaminococcus sp. BV3L6). In certain embodiments, the Cpf1 functional variants comprising said one or more amino acid substitutions have modified, more preferably increased specificity for the target polynucleotide.

In some embodiments, the Cpf1 functional variant comprises an amino acid substitution at position R912, T923, 8947, K949, 8951, 8955, K965, K968, K1000, 81003, K1009, K1017, K1022, K1029, K1072, K1086, F1103, R1226, and/or R1252 with reference to amino acid position numbering of AsCpf1 (Acidaminococcus sp. BV3L6). In certain embodiments, the Cpf1 functional variants comprising said one or more mutations have modified, more preferably increased specificity for the target polynucleotide.

In some embodiments, the Cpf1 functional variant comprises an amino acid substitution at position 8833. R836, K847, K879, K881, R883, 8887, K897, K900, K932, R935, K940, K948, K953, K960, K984, K1003, K1017, R1033, R1138, R1165, and/or R1252 with reference to amino acid position numbering of LbCpf1 (Lachnospiraceae bacterium D2006). In certain embodiments, the Cpf1 functional variants comprising said one or more mutations have modified, more preferably increased specificity for the target polynucleotide.

In some embodiments, the Cpf1 functional variant comprises an amino acid substitution at position K15, R18, K26, Q34, R43, K48, K51, R56, R84, K85, K87, N93, R103, N104, T118, K123, K134, R176, K177, R192, K200, K226, K273, K275, T291, 8301, K307, K369, 5404, V409, K414, K436, K438, K468, D482, K516, R518, K524, K530, K532, K548, K559, K570, 8574, K592, D596, K603, K607, K613, C647, R681, K686, H720, K739, K748, K757, T766, K780, R790, P791, K796, K809, K815, T816, K860, R862, R863, K868, K897, R909, R912, T923, 8947, K949, R951, 8955, K965, K968, K1000, R1003, K1009, K1017, K1022, K1029, A1053, K1072, K1086, F1103, 51209, R1226, R1252, K1273, K1282, and/or K1288 with reference to amino acid position numbering of AsCpf1 (Acidaminococcus sp. BV3L6). In certain embodiments, the Cpf1 functional variants comprising said one or more mutations have modified, more preferably increased specificity for the target polynucleotide.

In some embodiments, the Cpf1 functional variant comprises an amino acid substitution at position K15, R18, K26, R34, R43, K48, K51, K56, K87, K88, D90, K96, K106, K107, K120, Q125, K143, R186, K187, R202, K210, K235, K296, K298, K314, K320, K326, K397, K444, K449, E454, A483, E491, K527, K541, K581, 8583, K589, K595, K597, K613, K624, K635, K639, K656, K660, K667, K671, K677, K719, K725, K730, K763, K782, K791, R800, K809, K823, R833, K834, K839, K852, K858, K859, K869, K871, R872, K877, K905, 8918, R921, K932, 1960, K962, R964, R968, K978, K981, K1013, R1016, K1021, K1029, K1034, K1041, K1065, K1084, and/or K1098 with reference to amino acid position numbering of FnCpf1 (Francisella novicida U112). In certain embodiments, the Cpf1 functional variants comprising said one or more mutations have modified, more preferably increased specificity for the target polynucleotide.

In some embodiments. the Cpf1 functional variant comprises an amino acid substitution at position K15, R18, K26, K34, R43, K48, K51, R56, K83, K84, R86, K92, R102, K103, K116, K121, R158, E159, R174, R182, K206, K251, K253, K269, K271, K278, P342, K380, R385, K390, K415, K421, K457, K471, A506, R508, K514, K520, K522, K538, Y548, K560, K564, K580, K584, K591, K595, K601, K634, K640, R645, K679, K689, K707, T716, K725, 8737, 8747, R748, K753, K768, K774, K775, K785, K787, R788, Q793, K821, R833, R836, K847, K879, K881, R883, R887, K897, K900, K932, R935, K940, K948, K953, K960, K984, K1003, K1017, R1033, K1121, R1138, R1165, K1190, K1199, and/or K1208 with reference to amino acid position numbering of LbCpf1 (Lachnospiraceae bacterium D2006). In certain embodiments, the Cpf1 functional variants comprising said one or more mutations have modified, more preferably increased specificity for the target polynucleotide.

In some embodiments, the Cpf1 functional variant comprises an amino acid substitution at position K14, R17, R25, K33, M42, Q47, K50, D55, K85, N86, K88, K94, R104, K105, K118, K123, K131, R174, K175, R190, R198, I221, K267, Q269, K285, K291, K297, K357, K403, K409, K414, K448, K460, K501, K515, K550, R552, K558, K564, K566, K582, K593, K604, K608, K623, K627, K633, K637, E643, K780, Y787, K792, K830, Q846, K858, K867, K876, K890, R900, K901, M906, K921, K927, K928, K937, K939, R940, K945, Q975, R987, R990, K1001, R1034, I1036, R1038, R1042, K1052, K1055, K1087, R1090, K1095, N1103, K1108, K1115, K1139, K1158, R1172, K1188, K1276, R1293, A1319, K1340, K1349, and/or K1356 with reference to amino acid position numbering of MbCpf1 (Moraxella bovoculi 237). In one embodiment, the Cpf1 functional variant comprises an amino acid substitution at position S1228 (e.g., S1228A) with reference to amino acid position numbering of AsCpf1. See Yamano et al., Cell 165:949-962 (2016), which is incorporated herein by reference in its entirety.

In some embodiments, a prime editor comprises a napDNAbp that is a Cas12b/C2c1. In some embodiments, a prime editor comprises a napDNAbp that is a Cas12c/C2c3. Cas12 proteins have been described by Shmakov et al., “Discovery and Functional Characterization of Diverse Class 2 CRISPR Cas Systems”, Mol. Cell, 2015 Nov. 5; 60(3): 385-397, the entire contents of which is hereby incorporated by reference.

In some embodiments, the Cas12b/C2c1 of a prime editor contain a RuvC-like endonuclease domain. The crystal structure of Alicyclobaccillus acidoterrastris Cas12b/C2c1 (AacC2c1) has been reported in complex with a single-molecule guide RNA (sgRNA). See e.g., Liu et al., “C2c1-sgRNA Complex Structure Reveals RNA-Guided DNA Cleavage Mechanism”, Mol. Cell, 2017 Jan. 19; 65(2):310-322, the entire contents of which are hereby incorporated by reference. The crystal structure has also been reported in Alicyclobacillus acidoterrestris C2c1 bound to target DNAs as ternary complexes. See e.g., Yang et al., “PAM-dependent Target DNA Recognition and Cleavage by C2C1 CRISPR-Cas endonuclease”, Cell, 2016 Dec. 15; 167(7):1814-1828, the entire contents of which are hereby incorporated by reference.

In some embodiments, the napDNAbp of a prime editor is a Cas12b/C2c1 protein or a functional fragment or functional variant thereof. In some embodiments, a Cas12b is a BhCas12b protein. In some embodiments, the BhCas12b comprises one or more gain-of-function mutations that enhance the protein's activity at 37 degrees C. as described by Strecker et al., Nature Communications volume 10, Article number: 212 (2019), the entirety of which is incorporated herein by reference.

An exemplary Cas12b/C2c1 amino acid sequence is provided below:

Alicyclobacillus acidoterrestris Cas12b|
Casuniprot.org/uniprot/T0D7A2#2) sp|T0D7A2|C2C1_ALIAG CRISPR-associated endo-nuclease
C2c1 OS = Alicyclobacillus acidoterrestris (strain ATCC 49025/DSM 3922/CIP 106132/
NCIMB 13137/GD3B) GN = c2c1 PE = 1 SV = 1
(SEQ ID NO: 44)
MAVKSIKVKLRLDDMPEIRAGLWKLHKEVNAGVRYYTEWLSLLRQENLYRRSPNGDGEQECDKTAEECKA
ELLERLRARQVENGHRGPAGSDDELLQLARQLYELLVPQAIGAKGDAQQIARKFLSPLADKDAVGGLGIA
KAGNKPRWVRMREAGEPGWEEEKEKAETRKSADRTADVLRALADFGLKPLMRVYTDSEMSSVEWKPLRKG
QAVRTWDRDMFQQAIERMMSWESWNQRVGQEYAKLVEQKNRFEQKNFVGQEHLVHLVNQLQQDMKEASPG
LESKEQTAHYVTGRALRGSDKVFEKWGKLAPDAPFDLYDAEIKNVQRRNTRRFGSHDLFAKLAEPEYQAL
WREDASFLTRYAVYNSILRKLNHAKMFATFTLPDATAHPIWTRFDKLGGNLHQYTFLFNEFGERRHAIRF
HKLLKVENGVAREVDDVTVPISMSEQLDNLLPRDPNEPIALYFRDYGAEQHFTGEFGGAKIQCRRDQLAH
MHRRRGARDVYLNVSVRVQSQSEARGERRPPYAAVFRLVGDNHRAFVHFDKLSDYLAEHPDDGKLGSEGL
LSGLRVMSVDLGLRTSASISVFRVARKDELKPNSKGRVPFFFPIKGNDNLVAVHERSQLLKLPGETESKD
LRAIREERQRTLRQLRTQLAYLRLLVRCGSEDVGRRERSWAKLIEQPVDAANHMTPDWREAFENELQKLK
SLHGICSDKEWMDAVYESVRRVWRHMGKQVRDWRKDVRSGERPKIRGYAKDVVGGNSIEQIEYLERQYKF
LKSWSFFGKVSGQVIRAEKGSRFAITLREHIDHAKEDRLKKLADRIIMEALGYVYALDERGKGKWVAKYP
PCQLILLEELSEYQFNNDRPPSENNQLMQWSHRGVFQELINQAQVHDLLVGTMYAAFSSRFDARTGAPGI
RCRRVPARCTQEHNPEPFPWWLNKFVVEHTLDACPLRADDLIPTGEGEIFVSPFSAEEGDFHQIHADLNA
AQNLQQRLWSDFDISQIRLRCDWGEVDGELVLIPRLTGKRTADSYSNKVFYTNTGVTYYERERGKKRRKV
FAQEKLSEEEAELLVEADEAREKSVVLMRDPSGIINRGNWTRQKEFWSMVNQRIEGYLVKQIRSRVPLQ
DSACENTGDI
BhCas12b (Bacillus hisashii) NCBI Reference Sequence: WP_095142515
(SEQ ID NO: 45)
MATRSFILKIEPNEEVKKGLWKTHEVLNHGIAYYMNILKLIRQEAIYEHHEQDPKNPKKV
SKAEIQAELWDFVLKMQKCNSFTHEVDKDEVFNILRELYEELVPSSVEKKGEANQLSNKF
LYPLVDPNSQSGKGTASSGRKPRWYNLKIAGDPSWEEEKKKWEEDKKKDPLAKILGKLAE
YGLIPLFIPYTDSNEPIVKEIKWMEKSRNQSVRRLDKDMFIQALERFLSWESWNLKVKEE
YEKVEKEYKTLEERIKEDIQALKALEQYEKERQEQLLRDTLNTNEYRLSKRGLRGWREII
QKWLKMDENEPSEKYLEVFKDYQRKHPREAGDYSVYEFLSKKENHFIWRNHPEYPYLYAT
FCEIDKKKKDAKQQATFTLADPINHPLWVRFEERSGSNLNKYRILTEQLHTEKLKKKLTV
QLDRLIYPTESGGWEEKGKVDIVLLPSRQFYNQIFLDIEEKGKHAFTYKDESIKFPLKGT
LGGARVQFDRDHLRRYPHKVESGNVGRIYFNMTVNIEPTESPVSKSLKIHRDDFPKVVNF
KPKELTEWIKDSKGKKLKSGIESLEIGLRVMSIDLGQRQAAAASIFEVVDQKPDIEGKLE
FPIKGTELYAVHRASFNIKLPGETLVKSREVLRKAREDNLKLMNQKLNFLRNVLHFQQFE
DITEREKRVTKWISRQENSDVPLVYQDELIQIRELMYKPYKDWVAFLKQLHKRLEVEIGK
EVKHWRKSLSDGRKGLYGISLKNIDEIDRTRKFLLRWSLRPTEPGEVRRLEPGQRFAIDQ
LNHLNALKEDRLKKMANTIIMHALGYCYDVRKKKWQAKNPACQIILFEDLSNYNPYEERS
RFENSKLMKWSRREIPRQVALQGEIYGLQVGEVGAQFSSRFHAKTGSPGIRCSVVTKEKL
QDNRFFKNLQREGRLTLDKIAVLKEGDLYPDKGGEKFISLSKDRKCVTTHADINAAQNLQ
KRFWTRTHGFYKVYCKAYQVDGQTVYIPESKDQKQKIIEEFGEGYFILKDGVYEWVNAGK
LKIKKGSSKQSSSELVDSDILKDSFDLASELKGEKLMLYRDPSGNVFPSDKWMAAGVFFG
KLERILISKLTNQYSISTIEDDSSKQSM

In some embodiments, the Cas12b is BvCas12B, which is a variant of BhCas12b and comprises the following changes relative to BhCas12B: S893R, K846R, and E837G.

BvCas12b (Bacillus sp. V3-13) NCBI Reference Sequence: WP_101661451.1
(SEQ ID NO: 46)
MAIRSIKLKMKTNSGTDSIYLRKALWRTHQLINEGIAYYMNLLTLYRQEAIGDKTKEAYQAELINIIRNQ
QRNNGSSEEHGSDQEILALLRQLYELIIPSSIGESGDANQLGNKFLYPLVDPNSQSGKGTSNAGRKPRWK
RLKEEGNPDWELEKKKDEERKAKDPTVKIFDNLNKYGLLPLFPLFTNIQKDIEWLPLGKRQSVRKWDKDM
FIQAIERLLSWESWNRRVADEYKQLKEKTESYYKEHLTGGEEWIEKIRKFEKERNMELEKNAFAPNDGYF
ITSRQIRGWDRVYEKWSKLPESASPEELWKVVAEQQNKMSEGFGDPKVFSFLANRENRDIWRGHSERIYH
IAAYNGLQKKLSRTKEQATFTLPDAIEHPLWIRYESPGGTNLNLFKLEEKQKKNYYVTLSKIIWPSEEKW
IEKENIEIPLAPSIQFNRQIKLKQHVKGKQEISFSDYSSRISLDGVLGGSRIQFNRKYIKNHKELLGEGD
IGPVFFNLVVDVAPLQETRNGRLQSPIGKALKVISSDFSKVIDYKPKELMDWMNTGSASNSFGVASLLEG
MRVMSIDMGQRTSASVSIFEVVKELPKDQEQKLFYSINDTELFAIHKRSFLLNLPGEVVTKNNKQQRQER
RKKRQFVRSQIRMLANVLRLETKKTPDERKKAIHKLMEIVQSYDSWTASQKEVWEKELNLLTNMAAFNDE
IWKESLVELHHRIEPYVGQIVSKWRKGLSEGRKNLAGISMWNIDELEDTRRLLISWSKRSRTPGEANRIE
TDEPFGSSLLQHIQNVKDDRLKQMANLIIMTALGFKYDKEEKDRYKRWKETYPACQIILFENLNRYLFNL
DRSRRENSRLMKWAHRSIPRTVSMQGEMFGLQVGDVRSEYSSRFHAKTGAPGIRCHALTEEDLKAGSNTL
KRLIEDGFINESELAYLKKGDIIPSQGGELFVTLSKRYKKDSDNNELTVIHADINAAQNLQKRFWQQNSE
VYRVPCQLARMGEDKLYIPKSQTETIKKYFGKGSFVKNNTEQEVYKWEKSEKMKIKTDTTFDLQDLDGFE
DISKTIELAQEQQKKYLTMFRDPSGYFFNNETWRPQKEYWSIVNNIIKSCLKKKILSNKVEL

In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at ease 99.5% identical to a naturally-occurring Cas12b/C2c1 protein. In some embodiments, the napDNAbp is a naturally-occurring Cas12b/C2c1 protein. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at ease 99.5% identical to any one of the Cas12b amino acid sequences provided herein. It should be appreciated that Cas12b/C2c1 from other bacterial species may also be used in accordance with the present disclosure.

In some embodiments, the napDNAbp is a Cas12c/C2c3 protein or a functional fragment or a functional variant thereof. In some embodiments, the Cas12c/C2c3 of a prime editor contain a RuvC-like endonuclease domain. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at ease 99.5% identical to a naturally-occurring Cas12c/C2c3 protein. In some embodiments, the napDNAbp is a naturally-occurring Cas12c/C2c3 protein. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at ease 99.5% identical to any one of the Cas12c amino acid sequences provided herein. It should be appreciated that Cas12c/C2c3 from other bacterial species may also be used in accordance with the present disclosure.

In some embodiments, a prime editor comprises a Cas protein domain that requires recognition of a protospacer adjacent motif (PAM) for target binding. In some embodiments, a prime editor comprises a Cas domain that recognizes a PAM in a naturally occurring Cas-PAM recognition patter, or a “canonical” PAM. For example, in some embodiments, a prime editor comprises a Cas9 domain that recognizes a 5′-NGG-3′ PAM (a canonical PAM), where the “N” in “NGG” is adenine (A), thymine (T), guanine (G), or cytosine (C), and the G is guanine. A PAM can be Cas protein-specific and can be different between different Cas proteins, and accordingly between different prime editors comprising different Cas protein domains. In some embodiments, a prime editor comprises a Cas domain that recognizes a PAM different from a naturally occurring Cas-PAM recognition patter, non-canonical PAM. A PAM can be 5′ or 3′ of protospacer sequence. A PAM can be upstream or downstream of a protospacer sequence. A PAM can be 1, 2, 3, 4, 5, 6, 7, 8, 9, 10 or more nucleotides in length. In some embodiments, a PAM is between 2-6 nucleotides in length.

For example, in some embodiments, a prime editor comprises a Cas9 domain that recognizes a 5′-NGG-3′ PAM. In some embodiments, the Cas9 domain is a Streptococcus pyogenes Cas9 (SpCas9) domain. In some embodiments, a prime editor comprises a Cas9 functional variant that recognize non-NGG PAM sequences. Additionally, other Cas9 orthologues from various species have been identified and these “non-SpCas9s” can bind a variety of PAM sequences that can also be useful for the present disclosure.

Exemplary non-canonical PAM sequences and corresponding Cas proteins are provided in Table 1:

TABLE 1
Cas9 proteins and corresponding PAM sequences

| Variant | PAM |
|---|---|
| spCas9 (wild-type) | NGG, NGA, NAG |
| spCas9-VRVRFRR | NG |
| R1335V/L1111R/D1135V/G1218R/E1219F/ | |
| A1322R/T1337R | |
| spCas9-VQR (D1135V/R1335Q/T1337R) | NGA |
| spCas9-EQR (D1135E/R1335Q/T1337R) | NGA |
| spCas9-VRER | NGCG |
| (D1135V/G1218R/R1335E/T1337R) | |
| xCas9 (E480K, E543D, E1219V, K294R, | NGN |
| Q1256K, A262T, S409I, M694I) | |
| SluCas9 | NNGG |
| saCas9 | NNGRRT |
| | (SEQ ID NO: 1), |
| | NNGRRN |
| | (SEQ ID NO: 2) |
| saCas9-KKH (E782K, N968K, R1015H) | NNNRRT |
| | (SEQ ID NO: 3) |
| spCas9-MQKSER (D1135M, S1136Q, | NGCG/NGCN |
| G1218K, E1219S, R1335E, T1337R) | |
| spCas9-LRKIQK (D1135L, S1136R, | NGTN |
| G1218K, E1219I, R1335Q, T1337K) | |
| spCas9-LRVSQK (D1135L, S1136R, | NGTN |
| G1218V, E1219S, R1335Q, T1337K) | |
| spCas9-LRVSQL (D1135L, S1136R, | NGTN |
| G1218V, E1219S, R1335Q, T1337L) | |
| Cpf1 | TTTV |
| Spy-Mac | NAA |
| NmCas9 | NNNNGATT |
| | (SEQ ID NO: 4) |
| StCas9 | NNAGAAW |
| | (SEQ ID NO: 5) |
| TdCas9 | NAAAAC |
| | (SEQ ID NO: 6) |

In some embodiments, the PAM is NGC. In some embodiments, the NGC PAM is recognized by a Cas9 variant. In some embodiments, the NGC PAM variant includes one or more amino acid substitutions selected from D1135M, S1136Q, G1218K, E1219F, A1322R, D1332A, R1335E, and T1337R (collectively termed “MQKFRAER”). In some embodiments, the prime editing composition comprises a modified SpCas9 including amino acid substitutions D1135M, S1136Q, G1218K, E1219F, A1322R, D1332A, R1335E, and T1337R (SpCas9-MQKFRAER) and having specificity for the altered PAM 5′-NGC-3′.

In some embodiments, the PAM is NGT. In some embodiments, the NGT PAM is recognized by a Cas9 variant. In some embodiments, the NGT PAM variant is generated through targeted mutations at one or more residues 1335, 1337, 1135, 1136, 1218, and/or 1219. In some embodiments, the NGT PAM variant is created through targeted mutations at one or more residues 1219, 1335, 1337, 1218. In some embodiments, the NGT PAM variant is created through targeted mutations at one or more residues 1135, 1136, 1218, 1219, and 1335. In some embodiments, the NGT PAM variant is selected from the set of targeted mutations provided in Table 2 and Table 3 below.

TABLE 2
NGT PAM Variant Mutations at residues 1219, 1335, 1337, 1218

| Variant | E1219V | R1335Q | T1337 | G1218 |
|---|---|---|---|---|
| 1 | F | V | T | |
| 2 | F | V | R | |
| 3 | F | V | Q | |
| 4 | F | V | L | |
| 5 | F | V | T | R |
| 6 | F | V | R | R |
| 7 | F | V | Q | R |
| 8 | F | V | L | R |
| 9 | L | L | T | |
| 10 | L | L | R | |
| 11 | L | L | Q | |
| 12 | L | L | L | |
| 13 | F | I | T | |
| 14 | F | I | R | |
| 15 | F | I | Q | |
| 16 | F | I | L | |
| 17 | F | G | C | |
| 18 | H | L | N | |
| 19 | F | G | C | A |
| 20 | H | L | N | V |
| 21 | L | A | W | |
| 22 | L | A | F | |
| 23 | L | A | Y | |
| 24 | I | A | W | |
| 25 | I | A | F | |
| 26 | I | A | Y | |

TABLE 3
NGT PAM Variant Mutations at residues 1135, 1136, 1218,
1219, and 1335

| Variant | D1135L | S1136R | G1218S | E1219V | R1335Q |
|---|---|---|---|---|---|
| 27 | G | | | | |
| 28 | V | | | | |
| 29 | I | | | | |
| 30 | | A | | | |
| 31 | | W | | | |
| 32 | | H | | | |
| 33 | | K | | | |
| 34 | | | K | | |
| 35 | | | R | | |
| 36 | | | Q | | |
| 37 | | | T | | |
| 38 | | | N | | |
| 39 | | | | I | |
| 40 | | | | A | |
| 41 | | | | N | |
| 42 | | | | Q | |
| 43 | | | | G | |
| 44 | | | | L | |
| 45 | | | | S | |
| 46 | | | | T | |
| 47 | | | | | L |
| 48 | | | | | I |
| 49 | | | | | V |
| 50 | | | | | N |
| 51 | | | | | S |
| 52 | | | | | T |
| 53 | | | | | F |
| 54 | | | | | Y |
| 55 | N1286Q | I1331F | | | |

In some embodiments, the NGT PAM variant is selected from variant 5, 7, 28, 31, or 36 in Tables 2 and 3. In some embodiments, the variants have improved NGT PAM recognition.

In some embodiments, the NGT PAM variants have mutations at residues 1219, 1335, 1337, and/or 1218. In some embodiments, the NGT PAM variant is selected with mutations for improved recognition from the variants provided in Table 4 below.

TABLE 4
NGT PAM Variant Mutations at residues 1219, 1335, 1337, and 1218

| Variant | E1219V | R1335Q | T1337 | G1218 |
|---|---|---|---|---|
| 1 | F | V | T | |
| 2 | F | V | R | |
| 3 | F | V | Q | |
| 4 | F | V | L | |
| 5 | F | V | T | R |
| 6 | F | V | R | R |
| 7 | F | V | Q | R |
| 8 | F | V | L | R |

In some embodiments, a prime editor comprises a Cas9 domain that comprises one or more amino acid substitutions, insertions, or deletions compared to a wild-type Cas9 that alter the PAM recognition specificity of the Cas9 domain. In some embodiments, the Cas9 comprises one or more of a D1134X, a R1334X, and a T1336X mutation, or a corresponding mutation compared to a wild-type SpCas9, wherein X is any amino acid other than the original amino acid. In some embodiments, the Cas9 domain comprises one or more of a D1134E, R1334Q, and T1336R mutation, or a corresponding mutation compared to a wild-type SpCas9. In some embodiments, the Cas9 domain comprises a D1134E, a R1334Q, and a T1336R mutation, or corresponding mutations compared to a wild-type SpCas9. In some embodiments, the Cas9 domain comprises one or more of a D1134X, a R1334X, and a T1336X mutation, or a corresponding mutation compared to a wild-type SpCas9, wherein X is any amino acid other than the original amino acid. In some embodiments, the Cas9 domain comprises one or more of a D1134V, a R1334Q, and a T1336R mutation, or a corresponding mutation compared to a wild-type SpCas9. In some embodiments, the Cas9 domain comprises a D1134V, a R1334Q, and a T1336R mutation, or corresponding mutations compared to a wild-type SpCas9. In some embodiments, the Cas9 domain comprises one or more of a D1134X, a G1217X, a R1334X, and a T1336X mutation, compared to a wild-type SpCas9, wherein X is any amino acid other than the original amino acid. In some embodiments, the Cas9 domain comprises one or more of a D1134V, a G1217R, a R1334Q, and a T1336R mutation, or a corresponding mutation compared to a wild-type SpCas9. In some embodiments, the Cas9 domain comprises a D1134V, a G1217R, a R1334Q, and a T1336R mutation, or corresponding mutations compared to a wild-type SpCas9.

The amino acid sequence of an exemplary PAM-binding SpEQR Cas9 is as follows:

(SEQ ID NO: 47)
MDKKYSIGLAIGTNSVGWAVITDEYKVPSKKFKVLGNTDRHSIKKNLIGALLFDSGETAEATRLKRTARR
RYTRRKNRICYLQEIFSNEMAKVDDSFFHRLEESFVEEDKKHERHPIFGNIVDEVAYHEKYPTIYHLRKK
LVDSTDKADLRLIYLALAHMIKFRGHFLIEGDLNPDNSDVDKLFIQLVQTYNQLFEENPINASGVDAKAI
LSARLSKSRRLENLIAQLPGEKKNGLFGNLIALSLGLTPNFKSNFDLAEDAKLQLSKDTYDDDLDNLLAQ
IGDQYADLFLAAKNLSDAILLSDILRVNTEITKAPLSASMIKRYDEHHQDLTLLKALVRQQLPEKYKEIF
FDQSKNGYAGYIDGGASQEEFYKFIKPILEKMDGTEELLVKLNREDLLRKQRTFDNGSIPHQIHLGELHA
ILRRQEDFYPFLKDNREKIEKILTFRIPYYVGPLARGNSRFAWMTRKSEETITPWNFEEVVDKGASAQSF
IERMTNFDKNLPNEKVLPKHSLLYEYFTVYNELTKVKYVTEGMRKPAFLSGEQKKAIVDLLFKTNRKVTV
KQLKEDYFKKIECFDSVEISGVEDRFNASLGTYHDLLKIIKDKDFLDNEENEDILEDIVLTLTLFEDREM
IEERLKTYAHLFDDKVMKQLKRRRYTGWGRLSRKLINGIRDKQSGKTILDFLKSDGFANRNEMQLIHDDS
LTFKEDIQKAQVSGQGDSLHEHIANLAGSPAIKKGILQTVKVVDELVKVMGRHKPENIVIEMARENQTTQ
KGQKNSRERMKRIEEGIKELGSQILKEHPVENTQLQNEKLYLYYLQNGRDMYVDQELDINRLSDYDVDHI
VPQSFLKDDSIDNKVLTRSDKNRGKSDNVPSEEVVKKMKNYWRQLLNAKLITQRKFDNLTKAERGGLSEL
DKAGFIKRQLVETRQITKHVAQILDSRMNTKYDENDKLIREVKVITLKSKLVSDFRKDFQFYKVREINNY
HHAHDAYLNAVVGTALIKKYPKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEVQTGGFSKESILPKRNSDKLIA
RKKDWDPKKYGGFESPTVAYSVLVVAKVEKGKSKKLKSVKELLGITIMERSSFEKNPIDFLEAKGYKEVK
KDLIIKLPKYSLFELENGRKRMLASAGELQKGNELALPSKYVNFLYLASHYEKLKGSPEDNEQKQLFVEQ
HKHYLDEIIEQISEFSKRVILADANLDKVLSAYNKHRDKPIREQAENIIHLFTLTNLGAPAAFKYFDTTI
DRKQYRSTKEVLDATLIHQSITGLYETRIDLSQLGGD

In the above sequence, residues E1134, Q1334, and R1336, which can be mutated from D1134, R1334, and T1336 to yield a SpEQR Cas9, are underlined and in bold.

The amino acid sequence of an exemplary PAM-binding SpVQR Cas9 is as follows:

(SEQ ID NO: 48)
MDKKYSIGLAIGTNSVGWAVITDEYKVPSKKFKVLGNTDRHSIKKNLIGALLFDSGETAEATRLKRTARR
RYTRRKNRICYLQEIFSNEMAKVDDSFFHRLEESFLVEEDKKHERHPIFGNIVDEVAYHEKYPTIYHLRK
KLVDSTDKADLRLIYLALAHMIKFRGHFLIEGDLNPDNSDVDKLFIQLVQTYNQLFEENPINASGVDAKA
ILSARLSKSRRLENLIAQLPGEKKNGLFGNLIALSLGLTPNFKSNFDLAEDAKLQLSKDTYDDDLDNLLA
QIGDQYADLFLAAKNLSDAILLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLRKQRTFDNGSIPHQIHLGELH
AILRRQEDFYPFLKDNREKIEKILTFRIPYYVGPLARGNSRFAWMTRKSEETITPWNFEEVVDKGASAQS
FIERMTNFDKNLPNEKVLPKHSLLYEYFTVYNELTKVKYVTEGMRKPAFLSGEQKKAIVDLLFKTNRKVT
VKQLKEDYFKKIECFDSVEISGVEDRFNASLGTYHDLLKIIKDKDFLDNEENEDILEDIVLTLTLFEDRE
MIEERLKTYAHLFDDKVMKQLKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKVMGRHKPENIVIEMARENQTT
QKGQKNSRERMKRIEEGIKELGSQILKEHPVENTQLQNEKLYLYYLQNGRDMYVDQELDINRLSDYDVDH
IVPQSFLKDDSIDNKVLTRSDKNRGKSDNVPSEEVVKKMKNYWRQLLNAKLITQRKFDNLTKAERGGLSE
LDKAGFIKRQLVETRQITKHVAQILDSRMNTKYDENDKLIREVKVITLKSKLVSDFRKDFQFYKVREINN
YHHAHDAYLNAVVGTALIKKYPKLESEFVYGDYKVYDVRKMIAKSEQEIGKATAKYFFYSNIMNFFKTE1
TLANGEIRKRPLIETNGETGEIVWDKGRDFATVRKVLSMPQVNIVKKTEVQTGGFSKESILPKRNSDKLI
ARKKDWDPKKYGGFVSPTVAYSVLVVAKVEKGKSKKLKSVKELLGITIMERSSFEKNPIDFLEAKGYKEV
KKDLIIKLPKYSLFELENGRKRMLASAGELQKGNELALPSKYVNFLYLASHYEKLKGSPEDNEQKQLFVE
QHKHYLDEIIEQISEFSKRVILADANLDKVLSAYNKHRDKPIREQAENIIHLFTLTNLGAPAAFKYFDTT
IDRKQYRSTKEVLDATLIHQSITGLYETRIDLSQLGGD

In the above sequence, residues V1134, Q1334, and R1336, which can be mutated from D1134, R1334, and T1336 to yield a SpVQR Cas9, are underlined and in bold.

The amino acid sequence of an exemplary PAM-binding SpVRER Cas9 is as follows:

(SEQ ID NO: 49)
MDKKYSIGLAIGTNSVGWAVITDEYKVPSKKFKVLGNTDRHSIKKNLIGALLFDSGETAEATRLKRTARR
RYTRRKNRICYLQEIFSNEMAKVDDSFFHRLEESFLVEEDKKHERHPIFGNIVDEVAYHEKYPTIYHLRK
KLVDSTDKADLRLIYLALAHMIKFRGHFLIEGDLNPDNSDVDKLFIQLVQTYNQLFEENPINASGVDAKA
ILSARLSKSRRLENLIAQLPGEKKNGLFGNLIALSLGLTPNFKSNFDLAEDAKLQLSKDTYDDDLDNLLA
QIGDQYADLFLAAKNLSDAILLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLRKQRTFDNGSIPHQIHLGELH
AILRRQEDFYPFLKDNREKIEKILTFRIPYYVGPLARGNSRFAWMTRKSEETITPWNFEEVVDKGASAQS
FIERMTNFDKNLPNEKVLPKHSLLYEYFTVYNELTKVKYVTEGMRKPAFLSGEQKKAIVDLLFKTNRKVT
VKQLKEDYFKKIECFDSVEISGVEDRFNASLGTYHDLLKIIKDKDFLDNEENEDILEDIVLTLTLFEDRE
MIEERLKTYAHLFDDKVMKQLKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKVMGRHKPENIVIEMARENQTT
QKGQKNSRERMKRIEEGIKELGSQILKEHPVENTQLQNEKLYLYYLQNGRDMYVDQELDINRLSDYDVDH
IVPQSFLKDDSIDNKVLTRSDKNRGKSDNVPSEEVVKKMKNYWRQLLNAKLITQRKFDNLTKAERGGLSE
LDKAGFIKRQLVETRQITKHVAQILDSRMNTKYDENDKLIREVKVITLKSKLVSDFRKDFQFYKVREINN
YHHAHDAYLNAVVGTALIKKYPKLESEFVYGDYKVYDVRKMIAKSEQEIGKATAKYFFYSNIMNFFKTE1
TLANGEIRKRPLIETNGETGEIVWDKGRDFATVRKVLSMPQVNIVKKTEVQTGGFSKESILPKRNSDKLI
ARKKDWDPKKYGGFVSPTVAYSVLVVAKVEKGKSKKLKSVKELLGITIMERSSFEKNPIDFLEAKGYKEV
KKDLIIKLPKYSLFELENGRKRMLASARELQKGNELALPSKYVNFLYLASHYEKLKGSPEDNEQKQLFVE
QHKHYLDEIIEQISEFSKRVILADANLDKVLSAYNKHRDKPIREQAENIIHLFTLTNLGAPAAFKYFDTT
IDRKEYRSTKEVLDATLIHQSITGLYETRIDLSQLGGD.

In the above sequence, residues V1134, R1217, Q1334, and R1336, which can be mutated from D1134, G1217, R1334, and T1336 to yield a SpVRER Cas9, are underlined and in bold.

In some embodiments, the Cas9 domain is a Cas9 domain from Staphylococcus aureus (SaCas9). In some embodiments, the SaCas9 domain is a nuclease active SaCas9, a nuclease inactive SaCas9 (SaCas9d), or a SaCas9 nickase (SaCas9n). In some embodiments, the SaCas9 domain, the SaCas9d domain, or the SaCas9n domain can bind to a nucleic acid sequence having a non-canonical PAM. In some embodiments, the SaCas9 domain, the SaCas9d domain, or the SaCas9n domain can bind to a nucleic acid sequence having a NNGRRT (SEQ ID NO: 1) or a NNGRRT (SEQ ID NO: 1) PAM sequence. In some embodiments, the SaCas9 domain comprises one or more of a E781X, a N967X, and a R1014X mutation, or a corresponding mutation compared to a wild-type SaCas9, wherein X is any amino acid other than the original amino acid. In some embodiments, the SaCas9 domain comprises one or more of a E781K, a N967K, and a R1014H mutation, or one or more corresponding mutation compared to a wild-type SaCas9. In some embodiments, the SaCas9 domain comprises a E781K, a N967K, or a R1014H mutation, or corresponding mutations compared to a wild-type SaCas9.

An exemplary SaCas9 sequence is provided below: (Exemplary SaKKH Cas9)

(SEQ ID NO: 50)
KRNYILGLDIGITSVGYGIIDYETRDVIDAGVRLFKEANVENNEGRRSKRGARRLKRRRRHRIQRVKKLL
FDYNLLTDHSELSGINPYEARVKGLSQKLSEEEFSAALLHLAKRRGVHNVNEVEEDTGNELSTKEQISRN
SKALEEKYVAELQLERLKKDGEVRGSINRFKTSDYVKEAKQLLKVQKAYHQLDQSFIDTYIDLLETRRTY
YEGPGEGSPFGWKDIKEWYEMLMGHCTYFPEELRSVKYAYNADLYNALNDLNNLVITRDENEKLEYYEKF
QIIENVFKQKKKPTLKQIAKEILVNEEDIKGYRVTSTGKPEFTNLKVYHDIKDITARKEIIENAELLDQI
AKILTIYQSSEDIQEELTNLNSELTQEEIEQISNLKGYTGTHNLSLKAINLILDELWHTNDNQIAIFNRL
KLVPKKVDLSQQKEIPTTLVDDFILSPVVKRSFIQSIKVINAIIKKYGLPNDIIIELAREKNSKDAQKMI
NEMQKRNRQTNERIEEIIRTTGKENAKYLIEKIKLHDMQEGKCLYSLEAIPLEDLLNNPFNYEVDHIIPR
SVSFDNSFNNKVLVKQEEASKKGNRTPFQYLSSSDSKISYETFKKHILNLAKGKGRISKTKKEYLLEERD
INRFSVQKDFINRNLVDTRYATRGLMNLLRSYFRVNNLDVKVKSINGGFTSFLRRKWKFKKERNKGYKHH
AEDALIIANADFIFKEWKKLDKAKKVMENQMFEEKQAESMPEIETEQEYKEIFITPHQIKHIKDFKDYKY
SHRVDKKPNRKLINDTLYSTRKDDKGNTLIVNNLNGLYDKDNDKLKKLINKSPEKLLMYHHDPQTYQKLK
LIMEQYGDEKNPLYKYYEETGNYLTKYSKKDNGPVIKKIKYYGNKLNAHLDITDDYPNSRNKVVKLSLKP
YRFDVYLDNGVYKFVTVKNLDVIKKENYYEVNSKCYEEAKKLKKISNQAEFIASFYKNDLIKINGELYRV
IGVNNDLLNRIEVNMIDITYREYLENMNDKRPPHIIKTIASKTQSIKKYSTDILGNLYEVKSKKHPQIIK
KG.

Residue A579 above, which can be mutated from N579 to yield a SaCas9 nickase, is underlined and in bold. Residues K781, K967, and H1014 above, which can be mutated from E781, N967, and R1014 to yield a SaKKH Cas9 are underlined and in italics.

In some embodiments, other Cas9 orthologs can have different PAM requirements. For example, other PAMs such as those of S. thermophilus (5′-NNAGAA for CRISPR1 and 5′-NGGNG for CRISPR3) and Neisseria meningiditis (5′-NNNNGATT (SEQ ID NO: 4)) can also be found adjacent to a target gene. In some embodiments, the PAM sequence is AGC, GAG, TTT, GTG, or CAA. In some embodiments, the PAM sequence is NGA, NGCG, NGN, NNGRRT (SEQ ID NO: 1), NNNRRT (SEQ ID NO: 3), NGCG, NGCN, NGTN, NGTN, NGTN, TTTV.

In some embodiments, the Cas9 domain is a recombinant Cas9 domain. In some embodiments, the recombinant Cas9 domain is a SpyMacCas9 domain. In some embodiments, the SpyMacCas9 domain is a SpyMacCas9 nickase (SpyMacCas9n). In some embodiments, the SaCas9n domain can bind to a nucleic acid sequence having a non-canonical PAM. In some embodiments, the SpCas9n domain can bind to a nucleic acid sequence having a NAA PAM sequence.

The sequence of an exemplary Cas9 A homolog of Spy Cas9 in Streptococcus macacae with native 5′-NAAN-3′ PAM specificity is known in the art and described, for example, by Jakimo et al., (www.biorxiv.org/content/biorxiv/early/2018/09/27/429654.full.pdf), and is provided below.

SpyMacCas9
(SEQ ID NO: 51)
MDKKYSIGLDIGTNSVGWAVITDDYKVPSKKFKVLGNTDRHSIKKNLIGALLFGSGETAE
ATRLKRTARRRYTRRKNRICYLQEIFSNEMAKVDDSFFHRLEESFLVEEDKKHERHPIFG
NIVDEVAYHEKYPTIYHLRKKLADSTDKADLRLIYLALAHMIKFRGHFLIEGDLNPDNSD
VDKLFIQLVQIYNQLFEENPINASRVDAKAILSARLSKSRRLENLIAQLPGEKRNGLFGN
LIALSLGLTPNFKSNFDLAEDAKLQLSKDTYDDDLDNLLAQIGDQYADLFLAAKNLSDAI
LLSDILRVNSEITKAPLSASMIKRYDEHHQDLTLLKALVRQQLPEKYKEIFFDQSKNGYA
GYIDGGASQEEFYKFIKPILEKMDGTEELLVKLNREDLLRKQRTFDNGSIPHQIHLGELH
AILRRQEDFYPFLKDNREKIEKILTFRIPYYVGPLARGNSRFAWMTRKSEETITPWNFEE
VVDKGASAQSFIERMTNFDKNLPNEKVLPKHSLLYEYFTVYNELTKVKYVTEGMRKPAFL
SGEQKKAIVDLLFKTNRKVTVKQLKEDYFKKIECFDSVEISGVEDRFNASLGAYHDLLKI
IKDKDFLDNEENEDILEDIVLTLTLFEDRGMIEERLKTYAHLFDDKVMKQLKRRRYTGWG
RLSRKLINGIRDKQSGKTILDFLKSDGFANRNFMQLIHDDSLTFKEDIQKAQVSGQGHSL
HEQIANLAGSPAIKKGILQTVKIVDELVKVMGHKPENIVIEMARENQTTQKGQKNSRERM
KRIEEGIKELGSQILKEHPVENTQLQNEKLYLYYLQNGRDMYVDQELDINRLSDYDVDHI
VPQSFIKDDSIDNKVLTRSDKNRGKSDNVPSEEVVKKMKNYWRQLLNAKLITQRKFDNLT
KAERGGLSELDKAGFIKRQLVETRQITKHVAQILDSRMNTKYDENDKLIREVKVITLKSK
LVSDFRKDFQFYKVREINNYHHAHDAYLNAVVGTALIKKYPKLESEFVYGDYKVYDVRKM
IAKSEQEIGKATAKYFFYSNIMNFFKTEITLANGEIRKRPLIETNGETGEIVWDKGRDFA
TVRKVLSMPQVNIVKKTEIQTVGQNGGLFDDNPKSPLEVTPSKLVPLKKELNPKKYGGYQ
KPTTAYPVLLITDTKQLIPISVMNKKQFEQNPVKFLRDRGYQQVGKNDFIKLPKYTLVDI
GDGIKRLWASSKEIHKGNQLVVSKKSQILLYHAHHLDSDLSNDYLQNHNQQFDVLFNEII
SFSKKCKLGKEHIQKIENVYSNKKNSASIEELAESFIKLLGFTQLGATSPFNFLGVKLNQ
KQYKGKKDYILPCTEGTLIRQSITGLYETRVDLSKIGED.

Cas9 domains that bind non-canonical PAM sequences have been described in Kleinstiver, B. P., et al., “Engineered CRISPR-Cas9 nucleases with altered PAM specificities” Nature 523, 481-485 (2015); Kleinstiver, B. P., et al., “Broadening the targeting range of Staphylococcus aureus CRISPR-Cas9 by modifying PAM recognition” Nature Biotechnology 33, 1293-1298 (2015); and Walton et al., Unconstrained genome targeting with near-PAMless engineered CRISPR-Cas9 variants; the entire contents of each are hereby incorporated by reference.

In some embodiments, one of the Cas9 domains present in the prime editor fusion protein may be replaced with a guide nucleotide sequence-programmable DNA-binding protein domain that has no requirements for a PAM sequence.

In some embodiments, a functional Cas9 variant comprises W476A and/or W1126A mutations compared to a wild-type SpCas9. In some embodiments, a functional Cas9 variant comprises P475A, W476A, N477A, D1125A, W1126A, and/or D1127A mutations compared to a wild-type SpCas9. In some embodiments, the functional Cas9 variant does not bind efficiently to a PAM sequence. Thus, in some such embodiments, when such a functional Cas9 variant is used in a method of binding, the method does not require a PAM sequence. In other words, in some embodiments, when such a functional Cas9 variant is used in a method of binding, the method can include a guide RNA, but the method can be performed in the absence of a PAM sequence (and the specificity of binding is therefore provided by the targeting segment of the guide RNA).

In certain embodiments, the Cpf1 functional variant has been modified to recognize a non-naturally occurring PAM, such as recognizing a PAM having a sequence or comprising a sequence YCN, YCV, AYV, TYV, RYN, RCN, TGYV, NTTN, TTN, TRTN, TYTV, TYCT, TYCN, TRTN, NTTN, TACT, TYCC, TRTC, TATV, NTTV, TTV, TSTG, TVTS, TYYS, TCYS, TBYS, TCYS, TNYS, TYYS, TNTN, TSTG, TTCC, TCCC, TATC, TGTG, TCTG, TYCV, or TCTC. In some embodiments, the Cpf1 functional variant comprises one or more amino acid substitutions at position 11, 12, 13, 14, 15, 16, 17, 34, 36, 39, 40, 43, 46, 47, 50, 54, 57, 58, 111, 126, 127, 128, 129, 130, 131, 132, 133, 134, 135, 136, 157, 158, 159, 160, 161, 162, 163, 164, 165, 166, 167, 168, 169, 170, 171, 172, 173, 174, 175, 176, 177, 178, 532, 533, 534, 535, 536, 537, 538, 539, 540, 541, 542, 543, 544, 545, 546, 547, 548, 549, 550, 551, 552, 553, 5M, 555, 556, 565, 566, 567, 568, 569, 570, 571, 572, 573, 574, 575, 592, 593, 594, 595, 596, 597, 598, 599, 600, 601, 602, 603, 604, 605, 606, 607, 608, 609, 610, 611, 612, 613, 614, 615, 616, 617, 618, 619, 620, 626, 627, 628, 629, 630, 631, 632, 633, 634, 635, 636, 637, 638, 6′42, 643, 644, 645, 646, 647, 648, 649, 651, 652, 653, 654, 655, 656, 676, 679, 680, 682, 683, 684, 685, 686, 687, 688, 689, 690, 691, 692, 693, 707, 711, 714, 715, 716, 717, 718, 719, 720, 721, 722, 739, 765, 768, 769, 773, 777, 778, 779, 780, 781, 782, 783, 784, 785, 786, 871, 872, 873, 874, 875, 876, 877, 878, 879, 880, 881, 882, 883, 884, and/or 1048 as compared to a wild-type AsCpf1 or a position corresponding thereof in some embodiments, the Cpf1 functional variant comprises one or more amino acid substitutions at position 130, 131, 132, 133, 134, 135, 136, 162, 163, 164, 165, 166, 167, 168, 169, 170, 171, 172, 173, 174, 175, 176, 177, 536, 537, 538, 539, 540, 541, 542, 543, 544, 545, 546, 547, 548, 549, 550, 551, 552, 570, 571, 572, 573, 595, 596, 597, 598, 599, 600, 601, 602, 603, 604, 605, 606, 607, 608, 609, 610, 611, 612, 613, 614, 615, 630, 631, 632, 646, 647, 648, 649, 650, 651, 652, 653, 683, 684, 685, 686, 687, 688, 689, or 690 as compared to a wild-type AsCpf1 or a position corresponding thereof.

In certain embodiments, the Cpf1 functional variant is modified to have increased activity, i.e. wider PAM specificity. In particular embodiments, the Cpf1 functional variant is modified by mutation of one or more residues including but not limited positions 539, 542, 547, 548, 550, 551, 552, 167, 604, and/or 607 as compared to a wild-type AsCpf1, or a corresponding position thereof. In some embodiments, the Cpf1 functional variant comprises one or more amino acid substitutions at positions 542 or 542 and 607, wherein said amino acid substitutions preferably are 542R and 607R, such as S542R and K607R as compared to a wild-type AsCpf1, or a corresponding position thereof. In some embodiments, the Cpf1 functional variant comprises one or more amino acid substitutions at positions 542 and 548 (and optionally 552), wherein said mutations preferably are 542R and 548V (and optionally 552R), such as S542R and K548V (and optionally N552R); or at position 532, 538, 542, and/or 595, preferably mutated amino acid residues at positions 532 or 532 and 595, wherein said mutations preferably are 532R and 595R, such as G532R and K595R as compared to a wild-type LbCpf1, or a corresponding position thereof. In some embodiments, the Cpf1 functional variant comprises one or more amino acid substitutions at positions positions 532 and 538 (and optionally 542), wherein said mutations preferably are 532R and 538V (and optionally 542R), such as G532R and K538V (and optionally Y542R), or preferably wherein said mutations are S542R and K607R, S542R and K548V, or S542R, K548V and N552R as compared to a wild-type AsCpf1, or a corresponding position thereof.

### DNA Polymerase

In some embodiments, a prime editor comprises a DNA polymerase. In some embodiments, the polypeptide domain having DNA polymerase activity comprises a template-dependent DNA polymerase, for example, a DNA-dependent DNA polymerase or an RNA-dependent DNA polymerase. In some embodiments, the DNA polymerase is a reverse transcriptase. The DNA polymerase domain may be a wild-type DNA polymerase domain, a full-length DNA polymerase protein domain, or may be a functional mutant, a functional variant, or a functional fragment thereof. In some embodiments, the polymerase domain is a template dependent polymerase domain. In some embodiments, the DNA polymerase is a DNA-dependent DNA polymerase, which uses an editing template comprising DNA to synthesize a DNA strand. In some embodiments, the DNA-dependent DNA polymerase is a DNA polymerase α, β, γ, δ, or ε. In some embodiments, the DNA polymerase is an RNA-dependent DNA polymerase, which uses an editing template comprising RNA to synthesize a DNA strand. In some embodiments, the RNA-dependent DNA polymerase is a reverse transcriptase. In some embodiments, a DNA polymerase can be targeted to a double-stranded target polynucleotide (e.g., double-stranded target DNA) by a nucleic acid programmable DNA binding protein (napDNAbp) having nickase activity (e.g., nCas9) and a chimeric PE guide polynucleotide. In some embodiments, the DNA polymerase (e.g., DNA polymerase α, β, γ, δ, or ε) is capable of binding to a portion of the chimeric PE guide polynucleotide. In some embodiments, the DNA polymerase (e.g., DNA polymerase α, β, γ, δ, or ε) is capable of binding to an extension arm of the chimeric PE guide polynucleotide.

In some embodiments, the DNA polymerase (e.g., DNA polymerase α, β, γ, δ, and ε) is an endogenously expressed DNA polymerase. In some embodiments, the DNA polymerase (e.g., DNA polymerase α, β, γ, δ, and ε) is a DNA polymerase exogenously expressed in trans. In some embodiments, the DNA polymerase is a bacterial, eukaryotic, insect or plant DNA polymerase. In some embodiments, the DNA polymerase is a modified DNA polymerase that does not occur in nature.

In some embodiments, the DNA polymerase is a eukaryotic DNA polymerase. In some embodiments, the DNA polymerase is selected from the group consisting of DNA polymerase α, β, γ, δ, and ε, or a corresponding DNA polymerase thereof. In some embodiments, the DNA polymerase is a bacterial DNA polymerase. In some embodiments, the DNA polymerase is selected from the group consisting of DNA polymerase I, II, and III, or a corresponding DNA polymerase thereof. In some embodiments, the DNA polymerase is a viral DNA polymerase. DNA polymerases from other species may also be used in accordance with the present disclosure. In some embodiments, the DNA polymerase is a modified DNA polymerase that does not occur in nature. Exemplary sequences of DNA polymerase are provided below:

Exemplary amino acid sequence of DNA polymerase α (Accession No. AAA16459)
(SEQ ID NO: 52)
MSASAQQLAEELQIFGLDCEEALIEKLVELCVQYGQNEEGMVGELIAFCTSTHKVGLTSE
ILNSFEHEFLSKRLSKARHSTCKDSGHAGARDIVSIQELIEVEEEEEILLNSYTTPSKGSQKRAISTP
ETPLTKRSVSTRSPHQLLSPSSFSPSATPSQKYNSRSNRGEVVTSFGLAQGVSWSGRGGAGNISLK
VLGCPEALTGSYKSMFQKLPDIREVLTCKIEELGSELKEHYKIEAFTPLLAPAQEPVTLLGQIGCDS
NGKLNNKSVILEGDREHSSGAQIPVDLSELKEYSLFPGQVVIMEGINTTGRKLVATKLYEGVPLPF
YQPTEEDADFEQSMVLVACGPYTTSDSITYDPLLDLIAVINHDRPDVCILFGPFLESKHEQVENCL
LTSPFEDIFKQCLRTIIEGTRSSGSHLVFVPSLRDVHHEPVYPQPPFSYSDLSREDKKQVQFVSEPCS
LSINGVIFGLTSTDLLFHLGAEEISSSSGTSDRFSRILKHILTQRSYYPLYPPQEDMAIDYESFYVYA
QLPVTPDVLIIPSELRYFVKDVLGCVCVNPGRLTKGQVGGTFARLYLRRPAADGAERQSPCIAVQ
WRI
Exemplary amino acid sequence of DNA polymerase β (Accession No. NP_002681)
(SEQ ID NO: 53)
MSKRKAPQETLNGGITDMLTELANFEKNVSQAIHKYNAYRKAASVIAKYPHKIKSGAEA
KKLPGVGTKIAEKIDEFLATGKLRKLEKIRQDDTSSSINFLTRVSGIGPSAARKFVDEGIKTLEDLR
KNEDKLNHHQRIGLKYFGDFEKRIPREEMLQMQDIVLNEVKKVDSEYIATVCGSFRRGAESSGD
MDVLLTHPSFTSESTKQPKLLHQVVEQLQKVHFITDTLSKGETKFMGVCQLPSKNDEKEYPHRRI
DIRLIPKDQYYCGVLYFTGSDIFNKNMRAHALEKGFTINEYTIRPLGVTGVAGEPLPVDSEKDIFD
YIQWKYREPKDRSE
Exemplary amino acid sequence of DNA polymerase δ (Accession No. AAA35768)
(SEQ ID NO: 54)
MDGKRRPGPGPGVPPKRARGGLWDDDDAPRPSQFEEDLALMEEMEAEHRLQEQEEEEL
QSVLEGVADGQVPPSAIDPRWLRPTPPALDPQTEPLIFQQLEIDHYVGPAQPVPGGPPPSHGSVPV
LRAFGVTDEGFSVCCHIHGFAPYFYTPAPPGFGPEHMGDLQRELNLAINRDSRGGRELTGPAVLA
VELCSRESMFGYHGHGPSPFLRITVALPRLVAPARRLLEQGIRVAGLGTPSFAPYEANVDFEIRFM
VDTDIVGCNWLELPAGKYALRLKEKATQCQLEADVLWSDVVSHPPEGPWQRIAPLRVLSFDIEC
AGRKGIFPEPERDPVIQICSLGLRWGEPEPFLRLALTLRPCAPILGAKVQSYEKEEDLLQAWSTFIR
IMDPDVITGYNIQNFDLPYLISRAQTLKVQTFPFLGRVAGLCSNIRDSSFQSKQTGRRDTKVVSMV
GRVQMDMLQVLLREYKLRSYTLNAVSFHFLGEQKEDVQHSIITDLQNGNDQTRRRLAVYCLKD
AYLPLRLLERLMVLVNAVEMARVTGVPLSYLLSRGQQVKVVSQLLRQAMHEGLLMPVVKSEG
GEDYTGATVIEPLKGYYDVPIATLDFSSLYPSIMMAHNLCYTTLLRPGTAQKLGLTEDQFIRTPTG
DEFVKTSVRKGLLPQILENLLSARKRAKAELAKETDPLRRQVLDGRQLALKVSANSVYGFTGAQ
VGKLPCLEISQSVTGFGRQMIEKTKQLVESKYTVENGYSTSAKVVYGDTDSVMCRFGVSSVAEA
MALGGEAADWVSGHFPSPIRLEFEKVYFPYLLISKKRYAGLLFSSRPDAHDRMDCKGLEAVRRD
NCPLVANLVTASLRRLLIDRDPEGAVAHAQDVISDLLCNRIDISQLVITKELTRAASDYAGKQAH
VELAERMRKRDPGSAPSLGDRVPYVIISAAKGVAAYMKSEDPLFVLEHSLPIDTQYYLEQQLAKP
LLRIFEPILGEGRAEAVLLRGDHTRCKTVLTGKVGGLLAFAKRRNCCIGCRTVLSHQGAVCEFCQ
PRESELYQKEVSHLNALEERFSRLWTQCQRCQGSLHEDVICTSRDCPIFYMRKKVRKDLEDQEQ
LLRRFGPPGPEAW
Exemplary amino acid sequence of DNA polymerase ϵ (Accession No. KAI4069032)
(SEQ ID NO: 55)
MSLRSGGRRRADPGADGEASRDDGATSSVSALKRLERSQWTDKMDLRFGFERLKEPGE
KTGWLINMHPVALPYKPYFYIATRKGCEREVSSFLSKKFQGKIAKVETVPKEDLDLPNHLVGLK
RNYIRLSFHTVEDLVKVRKEISPAVKKNREQDHASDAYTALLSSVLQRGGVITDEEETSKKIADQ
LDNIVDMREYDVPYHIRLSIDLKIHVAHWYNVRYRGNAFPVEITRRDDLVERPDPVVLAFDIETT
KLPLKFPDAETDQIMMISYMIDGQGYLITNREIVSEDIEDFEFTPKPEYEGPFCVFNEPDEAHLIQR
WFEHVQETKPTIMVTYNGDFFDWPFVEARAAVHGLSMQQEIGFQKDSQGEYKAPQCIHMDCLR
WVKRDSYLPVGSHNLKAAAKAKLGYDPVELDPEDMCRMATEQPQTLATYSVSDAVATYYLY
MKYVHPFIFALCTIIPMEPDEVLRKGSGTLCEALLMVQAFHANIIFPNKQEQEFNKLTDDGHVLD
SETYVGGHVEALESGVFRSDIPCRFRMNPAAFDFLLQRVEKTLRHALEEEEKVPVEQVTNFEEVC
DEIKSKLASLKDVPSRIECPLIYHLDVGAMYPNIILTNRLQPSAMVDEATCAACDFNKPGANCQR
KMAWQWRGEFMPASRSEYHRIQHQLESEKFPPLFPEGPARAFHELSREEQAKYEKRRLADYCRK
AYKKIHITKVEERLTTICQRENSFYVDTVRAFRDRRYEFKGLHKVWKKKLSAAVEVGDAAEVK
RCKNMEVLYDSLQLAHKCILNSFYGYVMRKGARWYSMEMAGIVCFTGANIITQARELIEQIGRP
LELDTDGIWCVLPNSFPENFVFKTTNVKKPKVTISYPGAMLNIMVKEGFTNDQYQELAEPSSLTY
VTRSENSIFFEVDGPYLAMILPASKEEGKKLKKRYAVFNEDGSLAELKGFEVKRRGELQLIKIFQS
SVFEAFLKGSTLEEVYGSVAKVADYWLDVLYSKAANMPDSELFELISENRSMSRKLEDYGEQKS
TSISTAKRLAEFLGDQMVKDAGLSCRYIISRKPEGSPVTERAIPLAIFQAEPTVRKHFLRKWLKSSS
LQDFDIRAILDWDYYIERLGSAIQKIITIPAALQQVKNPVPRVKHPDWLHKKLLEKNDVYKQKKI
SELFTLEGRRQVTMAEASEDSPRPSAPDMEDFGLVKLPHPAAPVTVKRKRVLWESQEESQDLTP
TVPWQEILGQPPALGTSQEEWLVWLRFHKKKWQLQARQRLARRKRQRLESAEGVLRPGAIRDG
PATGLGSFLRRTARSILDLPWQIVQISETSQAGLFRLWALVGSDLHCIRLSIPRVFYVNQRVAKAE
EGASYRKVNRVLPRSNMVYNLYEYSVPEDMYQEHINEINAELSAPDIEGVYETQVPLLFRALVH
LGCVCVVNKQLVRHLSGWEAETFALEHLEMRSLAQFSYLEPGSIRHIYLYHHAQAHKALFGIFIP
SQRRASVFVLDTVRSNQMPSLGALYSAEHGLLLEKVGPELLPPPKHTFEVRAETDLKTICRAIQRF
LLAYKEERRGPTLIAVQSSWELKRLASEIPVLEEFPLVPICVADKINYGVLDWQRHGARRMIRHY
LNLDTCLSQAFEMSRYFHIPIGNLPEDISTFGSDLFFARHLQRHNHLLWLSPTARPDLGGKEADDN
CLVMEFDDQATVEINSSGCYSTVCVELDLQNLAVNTILQSHHVNDMEGADSMGISFDVIQQASL
EDMITGGQAASAPASYDETALCSNTFRILKSMVVGWVKEITQYHNIYADNQVMHFYRWLRSPSS
LLHDPALHRTLHNMMKKLFLQLIAEFKRLGSSVIYANFNRIILCTKKRRVEDAIAYVEYITSSIHSK
ETFHSLTISFSRCWEFLLWMDPSNYGGIKGKVSSRIHCGLQDSQKAGGAEDEQENEDDEEERDGE
EEEEAEESNVEDLLENNWNILQFLPQAASCQNYFLMIVSAYIVAVYHCMKDGLRRSAPGSTPVR
RRGASQLSQEAEGAVGALPGMITFSQDYVANELTQSFFTITQKIQKKVTGSRNSTELSEMFPVLP
GSHLLLNNPALEFIKYVCKVLSLDTNITNQVNKLNRDLLRLVDVGEFSEEAQFRDPCRSYVLPEVI
CRSCNFCRDLDLCKDSSFSEDGAVLPQWLCSNCQAPYDSSAIEMTLVEVLQKKLMAFTLQDLVC
LKCRGVKETSMPVYCSCAGDFALTIHTQVFMEQIGIFRNIAQHYGMSYLLETLEWLLQKNPQLG
H

In some embodiments, a prime editor further comprises an RNase enzyme. In some embodiments, the RNase enzyme cleaves double-stranded RNA. In some embodiments, the RNase enzyme cleaves the RNA strand of an RNA-DNA hybrid. In some embodiments, the RNase enzyme is an RNase H. In some embodiments, the RNase enzyme is RNaseH II or RNase H III. In some embodiments, the RNase is RNase H II (198 amino acids) from E. coli or RNase H III (257 amino acids) from Aquifex aeolicus. In some embodiments, the RNase comprises an amino acid sequence that is at least 70%, 75%, 80%, 85%, 90, 95%, 96%, 97%, 98%, 99%, or 99.5% identical to the amino acid sequence

(SEQ ID NO: 56)
MPSLKISPSEAEKIQNYLVSSGFRKINAPYTLWALEGNGVKVYYYKTGS
LLIQGKNSEKVLKEVLNLLEKKKLPGCDESGKGDIFGSLVLCCVCIPEE
NYLKVSSLNPRDTKRLSDKRVERLYLALKPLVKAYCYEIKPEEYNKLYR
KFRNLNKMMTHFYKLLIERVKEECGVSEVVVDKYQPSNPFGEDVIFETE
AERNLAVAVASIFARYKFLQSLKEVERELGIKIPKGTSKEVKELAKSLK
NPERFIKLNFNV.

In some embodiments, the RNase comprises the amino acid sequence

(SEQ ID NO: 56)
MPSLKISPSEAEKIQNYLVSSGFRKINAPYTLWALEGNGVKVYYYKTGS
LLIQGKNSEKVLKEVLNLLEKKKLPGCDESGKGDIFGSLVLCCVCIPEE
NYLKVSSLNPRDTKRLSDKRVERLYLALKPLVKAYCYEIKPEEYNKLYR
KFRNLNKMMTHFYKLLIERVKEECGVSEVVVDKYQPSNPFGEDVIFETE
AERNLAVAVASIFARYKFLQSLKEVERELGIKIPKGTSKEVKELAKSLK
NPERFIKLNFNV.

In some embodiments, the RNase comprises an amino acid sequence that is at least 70%, 75%, 80%, 85%, 90, 95%, 96%, 97%, 98%, 99%, or 99.5% identical to the amino acid sequence

(SEQ ID NO: 57)
MIEEVYPHTQLVAGVDEVGRGPLVGAVVTAAVILDPARPIAGLNDSKKL
SEKRRLALYEEIKEKALSWSLGRAEPHEIDELNILHATMLAMQRAVAGL
HIAPEYVLIDGNRCPKLPMPAMAVVKGDSRVPEISAASILAKVTRDAEM
AALDIVFPQYGFAQHKGYPTAFHLEKLAEHGATEHHRRSFGPVKRALGL
AS.

In some embodiments, the RNase comprises the amino acid sequence

(SEQ ID NO: 57)
MIEEVYPHTQLVAGVDEVGRGPLVGAVVTAAVILDPARPIAGLNDSKKL
SEKRRLALYEEIKEKALSWSLGRAEPHEIDELNILHATMLAMQRAVAGL
HIAPEYVLIDGNRCPKLPMPAMAVVKGDSRVPEISAASILAKVTRDAEM
AALDIVFPQYGFAQHKGYPTAFHLEKLAEHGATEHHRRSFGPVKRALGL
AS.

In some embodiments, the prime editor fusion protein further comprises an RNase inhibitor. In some embodiments, the RNase inhibitor is a catalytically inactive RNase. In some embodiments, the RNase inhibitor is a catalytically inactive RNase H. In some embodiments, the RNase is tethered to the prime editing composition with a linker. In some embodiments, the prime editing composition further comprises a zinc finger domain.

The prime editors of the present disclosure can comprise any domain, feature, or amino acid sequence which facilitates the editing of a target polynucleotide sequence. In various embodiments, the DNA polymerase and the napDNAbp of a prime editor are associated with each other covalently, non-covalently, or both covalently and non-covalently. In some embodiments, a napDNAbp with nickase activity (e.g., nCas9) is covalently or non-covalently attached to a DNA polymerase (e.g., DNA polymerase α, β, γ, δ, or ε) in a prime editor. In some embodiments, a prime editor comprises a Cas9 with nickase activity (nCas9) covalently or non-covalently attached to a DNA polymerase (e.g., DNA polymerase α, β, γ, δ, or ε). In some embodiments, a prime editor comprises a Cpf1 nickase (nCpf1) covalently or non-covalently attached to a DNA polymerase (e.g., DNA polymerase α, β, γ, δ, or ε).

In some embodiments, a prime editor does not comprise a peptide linker. In some embodiments, a prime editor comprises a napDNAbp with nickase activity (e.g., nCas9) and a DNA polymerase (e.g., DNA polymerase α, β, γ, δ, and ε) directly fused to each other to form a prime editor fusion protein.

In some embodiments, the protein domains of a prime editor are attached to each other via a linker. In some embodiments, the linker is a peptide linker. In some embodiments, a prime editor comprises a napDNAbp with nickase activity (e.g., nCas9) and a DNA polymerase (e.g., DNA polymerase α, β, γ, δ, and ε) fused via a peptide linker to form a prime editor fusion protein. In some embodiments, a napDNAbp with nickase activity (e.g., nCas9) is attached to DNA polymerase (e.g., DNA polymerase α, β, γ, δ, or ε) by a linker in a prime editing composition with a chimeric PE guide polynucleotide (e.g., DNA-RNA or RNA-DNA guide). In some embodiments, the a napDNAbp with nickase activity (e.g., nCas9) attached to the DNA polymerase (e.g., DNA polymerase α, β, γ, δ, or ε) and the chimeric PE guide polynucleotide form a complex. In some embodiments, a Cas9 with nickase activity (nCas9) is attached to a DNA polymerase (e.g., DNA polymerase α, β, γ, δ, or ε) by a linker. In some embodiments, a Cpf1 nickase (nCpf1) is attached to a DNA polymerase (e.g., DNA polymerase α, β, γ, δ, or ε) by a linker. In some embodiments, the linker is at least about 5-50 amino acids in length. In some embodiments, the linker is at least about 10-30 amino acids in length.

In some embodiments, the linker comprises a sequence selected from the group consisting of (SGGS)ₙ (SEQ ID NO: 58), (GGGS)ₙ (SEQ ID NO: 59), (GGGGS)ₙ (SEQ ID NO: 60), (G)ₙ (SEQ ID NO: 61), (EAAAK)ₙ (SEQ ID NO: 62), (GGS)ₙ (SEQ ID NO: 63), SGSETPGTSESATPES (SEQ ID NO: 18), (XP)ₙ (SEQ ID NO: 64) motif, or any combination thereof, wherein n is independently an integer between 1 and 30, and wherein X is any amino acid. In some embodiments, n is 1, 2, 3, 4, 5, 6, 7, 8, 9, 10, 11, 12, 13, 14, or 15. In some embodiments, the linker comprises the amino acid sequence: SGGSSGGSSGSETPGTSESATPES (SEQ ID NO: 19), SGGSSGSETPGTSESATPESSGGS (SEQ ID NO: 65), SGGSSGGSSGSETPGTSESATPESSGGSSGGS (SEQ ID NO: 66), or GGSGGSPGSPAGSPTSTEEGTSESATPESGPGTSTEPSEGSAPGSPAGSPTSTEEGTSTEPSEGSAPG TSTEPSEGSAPGTSESATPESGPGSEPATSGGSGGS (SEQ ID NO: 67). In some embodiments, the linker comprises the amino acid sequence SGSETPGTSESATPES (SEQ ID NO: 18), which may also be referred to as the XTEN linker. In some embodiments, a linker comprises the amino acid sequence SGGS (SEQ ID NO: 68). In some embodiments, the linker comprises a (GGS). motif, wherein n is 1, 3, or 7 (SEQ ID NO: 69).

In some embodiments, the linker is 24 amino acids in length. In some embodiments, the linker comprises the amino acid sequence SGGSSGGSSGSETPGTSESATPES (SEQ ID NO: 19). In some embodiments, the linker is 40 amino acids in length. In some embodiments, the linker comprises the amino acid sequence SGGSSGGSSGSETPGTSESATPESSGGSSGGSSGGSSGGS (SEQ ID NO: 70). In some embodiments, the linker is 64 amino acids in length. In some embodiments, the linker comprises the amino acid sequence SGGSSGGSSGSETPGTSESATPESSGGSSGGSSGGSSGGSSGSETPGTSESATPESSGGS SGGS (SEQ ID NO: 71). In some embodiments, the linker is 92 amino acids in length. In some embodiments, the linker comprises the amino acid sequence

(SEQ ID NO: 72)
PGSPAGSPTSTEEGTSESATPESGPGTSTEPSEGSAPGSPAGSPTSTEE
GTSTEPSEGSAPGTSTEPSEGSAPGTSESATPESGPGSEPATS.

In some embodiments, the domains of the prime editing composition are fused via a linker that comprises the amino acid sequence of SGGSSGSETPGTSESATPESSGGS (SEQ ID NO: 65), SGGSSGGSSGSETPGTSESATPESSGGSSGGS (SEQ ID NO: 66), or GGSGGSPGSPAGSPTSTEEGTSESATPESGPGTSTEPSEGSAPGSPAGSPTSTEEGTSTE PSEGSAPGTSTEPSEGSAPGTSESATPESGPGSEPATSGGSGGS (SEQ ID NO: 67).

In some embodiments, a linker comprises a plurality of proline residues and is 5-21, 5-14, 5-9, 5-7 amino acids in length, e.g., PAPAP (SEQ ID NO: 74), PAPAPA (SEQ ID NO: 75), PAPAPAP (SEQ ID NO: 76), PAPAPAPA (SEQ ID NO: 77), P(AP)₄ (SEQ ID NO: 78), P(AP)₇ (SEQ ID NO: 79), P(AP)₁₀ (SEQ ID NO: 80). Such proline-rich linkers are also termed “rigid” linkers.

Other exemplary features that can be present in a prime editing composition as disclosed herein are localization sequences, such as nuclear localization sequences, cytoplasmic localization sequences, export sequences, such as nuclear export sequences, or other localization sequences, as well as sequence tags that are useful for solubilization, purification, or detection of the prime editors. Suitable protein tags provided herein include, but are not limited to, biotin carboxylase carrier protein (BCCP) tags, myc-tags, calmodulin-tags, FLAG-tags, hemagglutinin (HA)-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FlAsH tags, V5 tags, and SBP-tags. Additional suitable sequences will be apparent to those of skill in the art. In some embodiments, the prime editor fusion protein comprises one or more His tags.

Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). Additional protein sequences can include amino acid sequences that bind DNA molecules or bind other cellular molecules, including, but not limited to, maltose binding protein (MBP), S-tag, Lex A DNA binding domain (DBD) fusions, GAL4 DNA binding domain fusions, and herpes simplex virus (HSV) BP16 protein fusions.

In some embodiments, a prime editor comprises one or more nuclear targeting sequences, for example, nuclear localization sequences (NLSs). In some embodiments, a prime editor comprises a fusion protein comprising a DNA polymerase (e.g., DNA polymerase α, β, γ, δ, and ε), a napDNAbp with nickase activity (e.g., nCas9), and one or more NLSs. In some embodiments, the prime editor comprises a DNA polymerase (e.g., DNA polymerase α, β, γ, δ, and ε) bound to at least one nuclear localization sequence (NLS). In some embodiments, the prime editor comprises a napDNAbp with nickase activity (e.g., nCas9) bound to at least one nuclear localization sequence (NLS). In some embodiments, the prime editor comprises a napDNAbp with nickase activity (e.g., nCas9) and a DNA polymerase bound to at least one nuclear localization sequence (NLS).

In some embodiments, a prime editor comprises two or more (e.g., 2, 3, 4, 5) nuclear targeting sequences, for example NLSs. The term “nuclear localization signal,” or “NLS” refers to an amino acid sequence that promotes import of a protein into the cell nucleus. In some embodiments, a prime editor comprises 1, 2, 3, 4, 5, 6, 7, 8, 9, 10 or more NLSs. In some embodiments, a prime editor comprises a fusion protein comprising a napDNAbp (e.g., nCas9), a DNA polymerase (e.g., DNA polymerase α, β, γ, δ, and ε), and 1, 2, 3, 4, 5, 6, 7, 8, 9, 10 or more NLSs. The NLSs may be at fused or linked at the N-terminus, the C-terminus, or anywhere in the prime editor fusion protein. When more than one NLS is present, each can be selected independently of others, such that a single NLS can be present in more than one copy and/or in combination with one or more other NLSs present in one or more copies. An NLS is considered near the N- or C-terminus when the nearest amino acid to the NLS is within about 50 amino acids along a polypeptide chain from the N- or C-terminus, e.g., within 1, 2, 3, 4, 5, 10, 15, 20, 25, 30, 40, or 50 amino acids.

In some embodiments, the NLS comprises an amino acid sequence selected from the group consisting of KRTADGSEFESPKKKRKV (SEQ ID NO: 7), KRPAATKKAGQAKKKK (SEQ ID NO: 8), KKTELQTTNAENKTKKL (SEQ ID NO: 9), KRGINDRNFWRGENGRKTR (SEQ ID NO: 10), RKSGKIAAIVVKRPRK (SEQ ID NO: 11), PKKKRKV (SEQ ID NO: 12), PKKKRKVEGADKRTADGSEFESPKKKRKV (SEQ ID NO: 81), RKSGKIAAIVVKRPRKPKKKRKV (SEQ ID NO: 82), or MDSLLMNRRKFLYQFKNVRWAKGRRETYLC (SEQ ID NO: 13).

In some embodiments, the NLS is a bipartite NLS (BPNLS). A bipartite NLS comprises two basic amino acid clusters, which are separated by a relatively short spacer sequence (hence bipartite—2 parts, while monopartite NLSs are not). The NLS of nucleoplasmin, KR[PAATKKAGQA]KKKK (SEQ ID NO: 8), is the prototype of the ubiquitous bipartite signal: two clusters of basic amino acids, separated by a spacer of about 10 amino acids. In some embodiments, the sequence of a bipartite NLS is PKKKRKVEGADKRTADGSEFESPKKKRKV (SEQ ID NO: 81).

In some embodiments, the NLS comprises an amino acid sequence of any one of the NLS sequences provided or referenced herein. Additional nuclear localization sequences are known in the art and would be apparent to the skilled artisan. For example, NLS sequences are described in Plank et al., PCT/EP2000/011690, the contents of which are incorporated herein by reference for their disclosure of exemplary nuclear localization sequences.

In some embodiments, an NLS comprises an amino acid sequence that facilitates the importation of a protein, that comprises an NLS, into the cell nucleus (e.g., by nuclear transport).

In some embodiments, the NLS is present in a linker or the NLS is flanked by linkers, for example, the linkers described herein.

Components of a prime editor may be connected to each other in any order. In some embodiments, the napDNAbp and the DNA polymerase domain of a prime editor may be fused to form a fusion protein or may be joined by a peptide or protein linker, in any order from the N terminus to the C terminus. In some embodiments, a prime editor comprises a napDNAbp domain fused or linked to the C-terminal end of a DNA polymerase domain. In some embodiments, a prime editor comprises a napDNAbp fused or linked to the N-terminal end of a DNA polymerase domain. In some embodiments, the prime editor comprises a fusion protein comprising the structure NH2-[napDNAbp]-[DNA polymerase]-COOH; or NH2-[DNA polymerase]-[napDNAbp]-COOH, wherein each instance of “]-[” indicates the presence of an optional linker sequence.

In some embodiments, the positioning of the polypeptide domains of a prime editor allows for correct spatial orientation for the polypeptide domains to affect the target polynucleotide with the attributed functional effect. For example, in some embodiments, a DNA polymerase is placed in a spatial orientation which allows it to contact a target polynucleotide sequence and synthesize a new DNA strand templated by the editing template of the chimeric guide polynucleotide. In some embodiments, a prime editor comprises a DNA polymerase inserted into an internal loop of a napDNAbp.

In some embodiments, an NLS of the prime editing composition is localized between a DNA polymerase (e.g., DNA polymerase α, β, γ, δ, or ε) and a nucleic acid programmable DNA binding protein (napDNAbp) having nickase activity (e.g., nCas9). In some embodiments, an NLS of the prime editing composition is localized C-terminal to a napDNAbp. In some embodiments, an NLS of the prime editing composition is localized N-terminal to a napDNAbp. In some embodiments, an NLS is fused to the N-terminus of a prime editor fusion protein. In some embodiments, an NLS is fused to the C-terminus of a prime editor fusion protein. In some embodiments, a prime editor comprises an nCas9 fused to an NLS at the N-terminus of the nCas9 domain. In some embodiments, a prime editor comprises an nCas9 fused to an NLS at the C-terminus of the nCas9 domain. In some embodiments, a prime editor comprises a DNA polymerase (e.g., DNA polymerase α, β, γ, δ, or ε) fused to an NLS at the N-terminus of the DNA polymerase. In some embodiments, a prime editor comprises a DNA polymerase (e.g., DNA polymerase α, β, γ, δ, or ε) fused to an NLS at the C-terminus of the DNA polymerase. In some embodiments, a prime editor comprises a DNA polymerase (e.g., DNA polymerase α, β, γ, δ, or ε) and a napDNAbp (e.g., nCas9) each fused to an NLS at the N-terminus or the N-terminus. In some embodiments, the NLS is fused to the prime editing composition or the prime editor fusion protein via one or more linkers. In some embodiments, the NLS is fused to the prime editor fusion protein without a linker.

### Guide Polynucleotides

The term “prime editing guide polynucleotide”, “PE guide polynucleotide” or “PEg polynucleotide”, refers to a guide polynucleotide that comprises one or more intended nucleotide edits for incorporation into the target DNA. In some embodiments, the PEg polynucleotide associates with and directs a prime editor to incorporate the one or more intended nucleotide edits into the target gene via prime editing. “Nucleotide edit” or “intended nucleotide edit” refers to a specified deletion of one or more nucleotides at one specific position, insertion of one or more nucleotides at one specific position, substitution of a single nucleotide, or other alterations at one specific position to be incorporated into the sequence of the target gene. Intended nucleotide edit may refer to the edit on the editing template as compared to the sequence on the target strand of the target gene or may refer to the edit encoded by the editing template on the newly synthesized single stranded DNA that replaces the editing target sequence, as compared to the editing target sequence.

In some embodiments, a PEg polynucleotide comprises a spacer sequence that is complementary or substantially complementary to a search target sequence on a target strand of the double stranded target DNA. In some embodiments, the spacer sequence is capable of annealing to the search target sequence in the target strand. In some embodiments, the search target sequence is complementary to a protospacer sequence on the edit strand of the double stranded target DNA. A protospacer sequence refers to a specific sequence in the edit strand (the PAM strand) of the target gene that is adjacent to a protospacer adjacent motif (PAM) or a PFS (protospacer flanking sequence or site). In some embodiments, the protospacer sequence is immediately adjacent to a PAM sequence on the edit strand of a double stranded DNA molecule. In some embodiments, the sequence of a PAM is specific and may be specifically recognized by a napDNAbp of a prime editor, e.g., a Cas9 nickase. In a PE guide polynucleotide, a spacer sequence may have substantially identical sequence as the protospacer sequence on the edit strand of a target gene, except that, in some embodiments wherein the spacer sequence comprises RNA, the spacer sequence may comprise Uracil (U) and the protospacer sequence may comprise Thymine (T). In some embodiments, the search target sequence is selected such that its complementary sequence, the protospacer sequence, is upstream or downstream of the PAM. In some embodiments, a napDNAbp with nickase activity is fused to a DNA polymerase, and the protospacer sequence is downstream or 3′ of the PAM. In some embodiments, the protospacer sequence is upstream or 3′ of the PAM. The precise sequence and length requirements for the PAM differ depending on the napDNAbp protein used, but PAMs are typically 2-5 base pair sequences adjacent the protospacer. Examples of the natural PAM sequences for different napDNAbps are provided herein below and the skilled person will be able to identify further PAM sequences for use with a given napDNAbp.

In some embodiments, the PEg polynucleotide comprises an invariable region or a scaffold that associates with a DNA binding domain, e.g., a CRISPR-Cas protein domain, of a prime editor. In some embodiments, the PEg polynucleotide comprises a scaffold that associates with a Cas9 domain of a prime editor, e.g., a SpCas9 nickase. An exemplary RNA sequence of a PEg polynucleotide scaffold is provided below:

(SEQ ID NO: 73)
GTTTTAGAGCTAGAAATAGCAAGTTAAAATAAGGCTAGTCCGTTATCAA
CTTGAAAAAGTGGCACCGAGTCGGTGC

In some embodiments, one or more uracil nucleotides in the exemplary scaffold sequence may be replaced with one or more thymine nucleotides.

In some embodiments, the PEg polynucleotide further comprises an extended nucleotide sequence comprising one or more intended nucleotide edits compared to the endogenous sequence of the target gene, wherein the extended nucleotide sequence may be referred to as an extension arm.

In some embodiments, the extension arm comprises a primer binding site sequence (PBS, or primer binding site) that can initiate target-primed DNA synthesis. In some embodiments, the PBS is complementary or substantially complementary to a sequence on the edit strand of the double stranded target DNA that is immediately upstream of the nick site. In some embodiments, during prime editing, the PBS is capable of anneal to a free 3′ end on the edit strand of the double stranded target DNA at a nick site generated by the prime editor.

In some embodiments, the extension arm further comprises an editing template (also referred to as DNA synthesis template, DNA polymerization template, or DNAP) that comprises one or more intended nucleotide edits to be incorporated in the target gene by prime editing. In some embodiments, the editing template is a synthesis template for a DNA polymerase domain of the prime editor. In some embodiments, the editing template comprises partial or substantial complementarity to a sequence in the edit strand of the double stranded target DNA. In some embodiments, the editing template is complementary or substantially complementary to a sequence on the PAM strand that is immediately downstream of the nick site, except for one or more non-complementary nucleotides at the intended nucleotide edit positions. The endogenous, e.g., genomic, sequence that is complementary or substantially complementary to the editing template, except for the one or more non-complementary nucleotides at the position corresponding to the intended nucleotide edit, may be referred to as an “editing target sequence”. In some embodiments, the editing template of a PEg polynucleotide is complementary to an editing target sequence in the edit strand of the double stranded target DNA except for one or more mismatches at the intended nucleotide edit positions in the editing template. In some embodiments, the editing template encodes a single stranded DNA, wherein the single stranded DNA is identical to the editing target sequence, except for one or more mismatches at positions in the single stranded DNA corresponding to the intended nucleotide edit positions in the editing template. In some embodiments, the editing template has identity or substantial identity to a sequence on the target strand that is complementary to, or having the same position in the genome as, the editing target sequence, except for one or more insertions, deletions, or substitutions at the intended nucleotide edit positions.

In some embodiments, the editing template and the PBS are immediately adjacent to each other. Accordingly, in some embodiments, a chimeric PEg polynucleotide in prime editing comprises a segment that comprises the PBS and the editing template immediately adjacent to each other. In some embodiments, the segment of the chimeric PEg polynucleotide comprising both the PBS and the editing template is complementary or substantially complementary to an endogenous sequence on the PAM strand (i.e., the non-target strand or the edit strand) of the double stranded target DNA except for one or more non-complementary nucleotides at the intended nucleotide edit positions.

PEg polynucleotides provided herein may comprise DNA, RNA, or both DNA and RNA. In some embodiments, a PEg polynucleotide includes only RNA nucleotides. For example, a PEg polynucleotide comprising only RNA may associate with a prime editor that comprises an RNA-dependent DNA polymerase, e.g., a reverse transcriptase. In some embodiments, a PEg polynucleotide is a chimeric PE guide polynucleotide that comprises both RNA and DNA nucleotides. For example, a chimeric PE guide polynucleotides (e.g., DNA-RNA or RNA-DNA guide) may associate with a prime editor that comprises a DNA-dependent DNA polymerase. In some embodiments, a PEg polynucleotide comprising both DNA and RNA nucleotides may be referred to as a “chimeric PE guide polynucleotide”, “chimeric PEg polynucleotide” or “chimeric DNA/RNA priming guide nucleic acid (chipGNA)”. In some embodiments, a PEg polynucleotide comprising both DNA and RNA nucleotides may also be generally referred to as a prime editing guide RNA or PEgRNA.

In some embodiments, a chimeric PE guide polynucleotide has sufficient complementarity with a double-stranded target polynucleotide sequence, e.g., a double stranded target DNA sequence for targeting a specific search target sequence in the double stranded target polynucleotide sequence. In some embodiments, the chimeric PE guide polynucleotide targets a double-stranded target polynucleotide by binding to a search target sequence in the double-stranded target polynucleotide. In some embodiments, the chimeric PE guide polynucleotide is configured to bind a napDNAbp (e.g., nCas9) in a prime editor. In some embodiments, the chimeric PE guide polynucleotide directs sequence specific binding of the prime editor to the double-stranded target polynucleotide.

In certain embodiments, the chimeric PE guide polynucleotide sequence is from 15 to 100 nucleotides. In certain embodiments, the chimeric PE guide polynucleotide is 15, 16, 17, 18, 19, 20, 21, 22, 23, 24, 25, 26, 27, 28, 29, 30, 31, 32, 33, 34, 35, 36, 37, 38, 39 40, 41, 42, 43, 44, 45, 46, 47 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83 84, 85, 86, 87, 88, 89, 90, 91, 92, 93, 94, 95, 96, 97, 98, 99, or 100 nucleotides. hi some embodiments, the chimeric PE guide polynucleotide is greater than 100 nucleotides. In certain embodiments, the chimeric PE guide polynucleotide is at least 100, 110, 120, 130, 140, 150, 160, 170, 180, 190, 200 or more nucleotides. In some embodiments, the chimeric PE guide polynucleotide sequence is selected so as to ensure that it hybridizes to the edit strand of the double stranded target polynucleotide to prime DNA polymerization. Selection can encompass further steps which increase efficacy and specificity of extension.

In some embodiments, a chimeric PE guide polynucleotide comprises a DNA segment and an RNA segment. In some embodiments, the RNA segment is at the 5′ end of the chimeric PE guide polynucleotide and the DNA segment is at the 3′ end of the chimeric PE guide polynucleotide. In some embodiments, the DNA segment is at the 5′ end of the chimeric PE guide polynucleotide and the RNA segment is at the 3′ end of the chimeric PE guide polynucleotide. In some embodiments, the RNA segment is fused to the DNA segment. In some embodiments, a chimeric PE guide polynucleotide is a DNA-RNA guide with a 5′ DNA segment fused to a 3′ RNA segment. In some embodiments, a chimeric PE guide polynucleotide is a RNA-DNA guide with a 3′ DNA segment fused to a 5′ RNA segment. In some embodiments, the chimeric PE guide polynucleotide comprises a DNA segment covalently attached to an RNA segment. In some embodiments, a chimeric PE guide polynucleotide is a DNA-RNA guide with a 5′ DNA segment covalently attached to a 3′ RNA segment. In some embodiments, a chimeric PE guide polynucleotide is a RNA-DNA guide with a 3′ DNA segment covalently attached to a 5′ RNA segment. In some embodiments, the DNA segment and the RNA segment are linked by a linker. In some embodiments, a chimeric PE guide polynucleotide is a DNA-RNA guide with a 5′ DNA segment linked to a 3′ RNA segment.

As used herein, a “segment” refers to a section or region of a molecule. In some embodiments, a segment of a PE guide polynucleotide is a contiguous stretch of nucleotides in the PE guide polynucleotide. In some embodiments, a segment of a PE guide polynucleotide can be a region that comprises more than one molecule. For example, where a guide polynucleotide comprises multiple nucleic acid molecules, an RNA segment can include all or a portion of multiple separate molecules, including, for example, the spacer and the scaffold or a portion of the scaffold. The definition of “segment,” unless otherwise specifically defined in a particular context, is not limited to a specific number of total nucleotides, is not limited to any particular number of nucleotides from a given RNA molecule, is not limited to any particular number of nucleotides from a given DNA molecule, is not limited to a particular number of separate molecules within a complex, and can include regions of RNA and/or DNA molecules that are of any total length and can include regions with complementarity to other molecules.

In some embodiments, a chimeric PE guide polynucleotide comprises a nucleic acid linker. In some embodiments, a DNA segment and an RNA segment of a chimeric PE guide polynucleotide are connected via a linker. In some embodiments, the linker comprises a sequence selected from the group consisting of AAAUUAACAAACUAA (SEQ ID NO: 20), AACAAACUAA (SEQ ID NO: 21), UUUUUUUUUUUUUUU (SEQ ID NO: 22), UUUUUUUUUUU (SEQ ID NO: 23), and UUUUU.

In some embodiments, a chimeric PE guide polynucleotide is an RNA-DNA guide with a 3′ DNA segment linked to a 5′ RNA segment via a linker. In some embodiments, the linker comprises a sequence selected from the group consisting of AAAUUAACAAACUAA (SEQ ID NO: 20), AACAAACUAA (SEQ ID NO: 21), UUUUUUUUUUUUUUU (SEQ ID NO: 22), UUUUUUUUUUU (SEQ ID NO: 23), and UUUUU, or a nucleic acid sequence that is at least 70%, 75%, 80%, 85%, 90%, 95%, 96%, 97%, 98%, 99%, or 99.5% identical thereto.

A prime editing guide polynucleotide may comprise multiple modular components. In some embodiments, the prime editing guide polynucleotide comprises (a) a spacer that is complementary or substantially complementary to a search target sequence on a target strand of a double stranded target polynucleotide (e.g., a target gene), (b) an extension arm that is at least partially complementary to a portion of an edit strand of the double-stranded target polynucleotide, and (c) a scaffold that associates with a napDNAbp. In some embodiments, the extension arm comprises a primer binding site sequence (also referred to as a primer binding site or a PBS). In some embodiments, the extension arm comprises an editing template (or DNA synthesis template). In some embodiments, DNA synthesis by a DNA polymerase of a prime editor may be primed at a free 3′ end generated by the prime editor in the edit strand, using the DNA synthesis template as a template. In some embodiments, the PBS of the PE guide polynucleotide is complementary or substantially complementary to the DNA sequence in the edit strand at the free 3′ end generated by the prime editor.

In various embodiments, a chimeric PE guide polynucleotide targets a double-stranded target polynucleotide. In various embodiments, the chimeric PE guide polynucleotide has sufficient complementarity with a double-stranded DNA target sequence to hybridize with the target nucleic acid sequence and direct sequence-specific binding of a napDNAbp of a prime editor. In some embodiments, a PE guide polynucleotide comprises a spacer sequence that is complementary or substantially complementary to a search target sequence on a target strand of the target gene. By “variable region”, “spacer”, “spacer region”, or “spacer sequence” is meant a polynucleotide sequence of a guide polynucleotide (e.g., chimeric PE guide polynucleotide (e.g., DNA-RNA or RNA-DNA guide) that is at least partially complementary, or substantially complementary to a search target sequence on a target strand of a double stranded a target polynucleotide (e.g., genomic DNA). In some embodiments, the spacer sequence is at least partially complementary to a portion of a target strand of a double-stranded target polynucleotide (e.g., genomic DNA). In some embodiments, the degree of complementarity between the spacer of the chimeric PE guide polynucleotide and a search target sequence is about or more than about 50%, 60%, 75%, 80%, 85%, 90%, 95%, 97.5%, 99%, or 100%.

In some embodiments, the spacer sequence is at least 8-30 nucleotides in length (e.g., 8, 9, 10, 11, 12, 13, 14, 15, 16, 17, 18, 19, 20, 21, 22, 23, 24, 25, 26, 27, 28, 29, 30, or more nucleotides in length). In some embodiments, a spacer sequence is between 10-30 nucleotides in length, between 15-25 nucleotides in length, or between 15-20 nucleotides in length. In some embodiments, the spacer sequence is between 10-120 nucleotides in length (e.g., 10, 20, 30, 40, 50, 60, 70, 80, 90, 100 nucleotides in length). In some embodiments, the spacer sequence is between 15-30 nucleotides in length, between 30-50 nucleotides in length, or between 50-100 nucleotides in length. In some embodiments, the spacer sequence is 10-30 nucleotides in length or 10-25 nucleotides in length. In some embodiments, the spacer sequence is about 17, 18, 19, 20, 21 or 22 nucleotides in length.

A spacer sequence of a PE guide polynucleotide can target a search target sequence in any region or structure of a target polynucleotide, e.g., a target gene. In some embodiments, the spacer sequence is within 50 nucleotides upstream or downstream of a target nucleotide in the editing target sequence of the double stranded target polynucleotide, e.g., a target gene, to be edited.

In some embodiments, the spacer sequence can target a search target sequence in an exon or an intron of a target gene.

In some embodiments, a search target sequence is at least 8-30 nucleotides in length (e.g., 8, 9, 10, 11, 12, 13, 14, 15, 16, 17, 18, 19, 20, 21, 22, 23, 24, 25, 26, 27, 28, 29, 30, or more nucleotides in length). In some embodiments, the search target sequence is between 10-30 nucleotides in length, between 15-25 nucleotides in length, or between 15-20 nucleotides in length. In some embodiments, the search target sequence is between 10-120 nucleotides in length (e.g., 10, 20, 30, 40, 50, 60, 70, 80, 90, 100 nucleotides in length). In some embodiments, the search target sequence is between 15-30 nucleotides in length, between 30-50 nucleotides in length, or between 50-100 nucleotides in length. In some embodiments, the search target acer sequence is 10-30 nucleotides in length or 10-25 nucleotides in length. In some embodiments, the search target sequence is about 17, 18, 19, 20, 21 or 22 nucleotides in length. In some embodiments, the search target sequence is complementary to a protospacer sequence in the target gene.

In some embodiments, the spacer sequence is incorporated into an RNA segment of chimeric PE guide polynucleotide (e.g., DNA-RNA or RNA-DNA guide). In some embodiments, the spacer sequence is at the 5′ end of a chimeric PE guide polynucleotide (e.g., DNA-RNA or RNA-DNA guide). In some embodiments, the spacer sequence is at the 3′ end of a chimeric PE guide polynucleotide (e.g., DNA-RNA or RNA-DNA guide). In some embodiments, the spacer sequence is in between a scaffold and an extension arm of a chimeric PE guide polynucleotide (e.g., DNA-RNA or RNA-DNA guide). In some embodiments, the spacer sequence comprises RNA. In some embodiments, the spacer sequence comprises DNA. In some embodiments, the spacer sequence comprises RNA and DNA. In some embodiments, the spacer sequence consists of RNA. In some embodiments, the spacer sequence consists of DNA.

Methods for selecting and designing PE guide polynucleotides, e.g., spacer sequences for specific targeting of a target polynucleotide, are described herein and known to those skilled in the art. Software tools can be used to optimize the PE guide polynucleotides, e.g., the spacer sequence, corresponding to a target nucleic acid sequence, e.g., to minimize total off-target activity across the genome. For example, for each possible targeting domain choice using S. pyogenes Cas9, all off-target sequences (preceding selected PAMs, e.g., NAG or NGG) may be identified across the genome that contain up to certain number (e.g., 1, 2, 3, 4, 5, 6, 7, 8, 9, or 10) of mismatched base-pairs. All spacer sequences can be ranked according to its total predicted off-target score; the top-ranked targeting domains represent those that are likely to have the greatest on-target and the least off-target activity. Candidate guide polynucleotides can be functionally evaluated by using methods known in the art and/or as set forth herein.

In some embodiments, a reporter system may be used for detecting prime editing activity and testing candidate guide polynucleotides. In some embodiments, a reporter system may comprise a reporter gene-based assay where target DNA editing activity leads to expression of the reporter gene. For example, a reporter system may include a reporter gene comprising a deactivated start codon, e.g., a mutation on the template strand from 3′-TAC-5′ to 3′-CAC-5′. Upon successful editing of the target polynucleotide, the corresponding mRNA will be transcribed as 5′-AUG-3′ instead of 5′-GUG-3′, enabling the translation of the reporter gene. Suitable reporter genes will be apparent to those of skill in the art. Non-limiting examples of reporter genes include gene encoding green fluorescence protein (GFP), red fluorescence protein (RFP), luciferase, secreted alkaline phosphatase (SEAP), or any other gene whose expression are detectable and apparent to those skilled in the art. The reporter system can be used to test many different PE guide polynucleotides. CRISPR-Cas guide RNAs (gRNAs) can also be tested to assess off-target effects of a specific prime editing protein, e.g., a Cas9-DNA polymerase prime editor fusion protein. In some embodiments, such gRNAs can be designed such that the mutated start codon will not be base-paired with the gRNA.

In some embodiments, the chimeric PE guide polynucleotide is configured to bind a napDNAbp (e.g., nCas9). In some embodiments, the PEg polynucleotide comprises a guide polynucleotide core (or a scaffold, an invariable region) that associates with a napDNAbp, e.g., a CRISPR-Cas protein domain, of a prime editor. In some embodiments, the scaffold stabilizes structure of the chimeric PE guide polynucleotide. In some embodiments, the scaffold comprises RNA. In some embodiments, the scaffold comprises DNA. In some embodiments, the scaffold comprises RNA and DNA. In some embodiments, the scaffold is incorporated into an RNA segment of chimeric PE guide polynucleotide (e.g., DNA-RNA or RNA-DNA guide).

In some embodiments, the chimeric PEg polynucleotide further comprises an extended nucleotide sequence comprising one or more intended nucleotide edits compared to the endogenous sequence of the target gene, wherein the extended nucleotide sequence may be referred to as an extension arm. In some embodiments, the extension arm comprises DNA. In some embodiments, the extension arm comprises RNA. In some embodiments, the extension arm comprises both DNA and RNA. In some embodiments, the extension arm is incorporated into a DNA segment of chimeric PE guide polynucleotide (e.g., DNA-RNA or RNA-DNA guide). In some embodiments, the extension arm sequence is at the 3′ end of a chimeric PE guide polynucleotide (e.g., DNA-RNA or RNA-DNA guide). In some embodiments, the extension arm sequence is at the 5′ end of a chimeric PE guide polynucleotide (e.g., DNA-RNA or RNA-DNA guide). In some embodiments, the extension arm: i) comprises one or more intended nucleotide edits to be incorporated into a double-stranded target polynucleotide, ii) is at least partially complementary to a portion of the edit strand of the double-stranded target polynucleotide, and iii) contains a sequence that is capable of priming a DNA polymerase (e.g., DNA polymerase α, β, γ, δ, or ε, or a reverse transcriptase).

In certain embodiments, the extension arm comprises a primer binding site sequence (PBS) that can initiate target-primed DNA synthesis. In some embodiments, the PBS is complementary or substantially complementary to a free 3′ end on the edit strand of the target gene at a nick site generated by the prime editor. In some embodiments, the PBS is incorporated into a DNA segment of chimeric PE guide polynucleotide (e.g., DNA-RNA or RNA-DNA guide). In some embodiments, the PBS sequence is at the 3′ end of a chimeric PE guide polynucleotide (e.g., DNA-RNA or RNA-DNA guide). In some embodiments, the PBS sequence is at the 5′ end of a chimeric PE guide polynucleotide (e.g., DNA-RNA or RNA-DNA guide). In some embodiments, the PBS comprises RNA. In some embodiments, the PBS comprises DNA. In some embodiments, the PBS comprises RNA and DNA.

In some embodiments, the extension arm further comprises an editing template that comprises one or more intended nucleotide edits to be incorporated in the target gene by prime editing. In some embodiments, the editing template is a template for a DNA polymerase domain, e.g., a DNA polymerase α, β, γ, δ, or ε domain, of the prime editor. In some embodiments, the editing template comprises partial complementarity to an editing target sequence in the target gene. In some embodiments, the editing template comprises substantial or partial complementarity to the editing target sequence except at the position of the intended nucleotide edits to be incorporated into the target gene. In some embodiments, the editing template comprises DNA. In some embodiments, the editing template comprises RNA.

In some embodiments, the one or more intended nucleotide edits comprises a substitution, insertion, deletion, modification, or any combination thereof, of one or more bases in a gene or polynucleotide. In some embodiments, the one or more intended nucleotide edits are in consecutive bases. In some embodiments, the one or more intended nucleotide edits are in non-consecutive bases. In some embodiments, the one or more intended nucleotide edits comprise altering one or more adenine bases to a cytosine, guanine, or thymine in the double-stranded target polynucleotide. In some embodiments, the one or more intended nucleotide edits comprises altering one or more cytosine bases to an adenine, guanine, or thymine in the double-stranded target polynucleotide. In some embodiments, the one or more intended nucleotide edits comprises altering one or more guanine bases to an adenine, cytosine, or thymine in the double-stranded target polynucleotide. In various embodiments, the one or more intended nucleotide edits comprises altering one or more thymine bases to an adenine, guanine, or cytosine in the double-stranded target polynucleotide. As a result of incorporation of the one or more intended nucleotide edits, in some embodiments, two or more nucleotides are altered in a double-stranded target polynucleotide. In some embodiments, 1, 2, 3, 4, 5, 6, 7, 8, 9, 10, or more nucleotides are altered in a double-stranded target polynucleotide. In various embodiments, two or more consecutive nucleotides are altered in a double-stranded target polynucleotide. In some embodiments, two or more non-consecutive nucleotides are altered in a double-stranded target polynucleotide. In some embodiments, one or more nucleotides are inserted in a double-stranded target polynucleotide. In various embodiments, 1, 2, 3, 4, 5, 6, 7, 8, 9, 10, 11, 12, 13, 14, 15, 16, 17, 18, 19, 20, 30, 40, 50, 60, 70, 80, 90, 100 or more nucleotides are inserted into a double-stranded target polynucleotide. In some embodiments, one or more nucleotides are deleted in a double-stranded target polynucleotide. In various embodiments, 1, 2, 3, 4, 5, 6, 7, 8, 9, 10, 11, 12, 13, 14, 15, 16, 17, 18, 19, 20, 30, 40, 50, 60, 70, 80, 90, 100 or more nucleotides are deleted in a double-stranded target polynucleotide.

An intended nucleotide edit in an editing template may comprise various types of alterations as compared to the target gene sequence e.g., a double-stranded target polynucleotide. In some embodiments, the nucleotide edit is a single nucleotide substitution as compared to the target gene sequence. In some embodiments, the nucleotide edit is a deletion as compared to the target gene sequence. In some embodiments, the nucleotide edit is an insertion as compared to the target gene sequence. In some embodiments, the editing template comprises one to ten intended nucleotide edits as compared to the target gene sequence. In some embodiments, the editing template comprises one or more intended nucleotide edits as compared to the target gene sequence. In some embodiments, the editing template comprises two or more intended nucleotide edits as compared to the target gene sequence. In some embodiments, a nucleotide substitution comprises an adenine (A)-to-thymine (T) substitution. In some embodiments, a nucleotide substitution comprises an A-to-guanine (G) substitution. In some embodiments, a nucleotide substitution comprises an A-to-cytosine (C) substitution. In some embodiments, a nucleotide substitution comprises a T-A substitution. In some embodiments, a nucleotide substitution comprises a T-G substitution. In some embodiments, a nucleotide substitution comprises a T-C substitution. In some embodiments, a nucleotide substitution comprises a G-to-A substitution. In some embodiments, a nucleotide substitution comprises a G-to-T substitution. In some embodiments, a nucleotide substitution comprises a G-to-C substitution. In some embodiments, a nucleotide substitution comprises a C-to-A substitution. In some embodiments, a nucleotide substitution comprises a C-to-T substitution. In some embodiments, a nucleotide substitution comprises a C-to-G substitution.

The editing template can comprise one or more intended nucleotide edits, compared to the target gene to be edited. Position of the intended nucleotide edit(s) relevant to other components of the PEg polynucleotide (e.g., a chimeric PEg polynucleotide), or to particular nucleotides (e.g., mutations) in the target gene can vary. In some embodiments, the nucleotide edit is in a region of the PEg polynucleotide (e.g., a chimeric PEg polynucleotide) corresponding to or homologous to the protospacer sequence. In some embodiments, the nucleotide edit is in a region of the PEg polynucleotide (e.g., a chimeric PEg polynucleotide) corresponding to a region of the target gene outside of the protospacer sequence.

In some embodiments, the position of a nucleotide edit incorporation in the target gene can be determined based on position of the protospacer adjacent motif (PAM). For instance, the intended nucleotide edit may be installed in a sequence corresponding to the protospacer adjacent motif (PAM) sequence. In some embodiments, a nucleotide edit in the editing template is at a position corresponding to the 5′ most nucleotide of the PAM sequence. In some embodiments, a nucleotide edit in the editing template is at a position corresponding to the 3′ most nucleotide of the PAM sequence. In some embodiments, position of an intended nucleotide edit in the editing template may be referred to by aligning the editing template with the partially complementary edit strand of the target gene and referring to nucleotide positions on the editing strand where the intended nucleotide edit is incorporated.

In some embodiments, the position of a nucleotide edit incorporation in the target gene can be determined based on position of the nick site.

In some embodiments, the chimeric PEg polynucleotide directs a prime editor to generate a nick on an edit strand of a double stranded target polynucleotide. In some embodiments, the nick is upstream or downstream of a protospacer adjacent motif (PAM) site. In some embodiments, the nick is at a specific position relative to a protospacer adjacent motif (PAM) site. For example, in some embodiments, the prime editor is a Cas9-based prime editor, and the chimeric PEg polynucleotide directs the prime editor to generate a nick 3 base pairs upstream of a PAM site on the edit strand of a double stranded target polynucleotide. In some embodiments, the one or more intended nucleotide edits are upstream of a PAM site. In some embodiments, the one or more intended nucleotide edits are 1, 2, 3, 4, 5, 6, 7, 8, 9, 10, 11, 12, 13, 14, 15, 16, 17, 18, 19, or 20 nucleotides upstream of the PAM site. In some embodiments, the one or more intended nucleotide edits are downstream of a PAM site. In some embodiments, the one or more intended nucleotide edits are 1, 2, 3, 4, 5, 6, 7, 8, 9, 10, 11, 12, 13, 14, 15, 16, 17, 18, 19, or 20 nucleotides downstream of the PAM site.

In some embodiments, the editing template comprises DNA. In some embodiments, the editing template comprises RNA. In some embodiments, the editing template comprises both DNA and RNA. In some embodiments, the editing template is incorporated into a DNA segment of chimeric PE guide polynucleotide (e.g., DNA-RNA or RNA-DNA guide). In some embodiments, the editing template is at the 3′ end of a chimeric PE guide polynucleotide (e.g., DNA-RNA or RNA-DNA guide). In some embodiments, the editing template is at the 5′ end of a chimeric PE guide polynucleotide (e.g., DNA-RNA or RNA-DNA guide).

In some embodiments, a PE guide polynucleotide may comprise a DNA segment and an RNA segment.

In some embodiments, the DNA segment of a chimeric PE guide polynucleotide is complementary to a portion of the RNA segment of a chimeric PE guide polynucleotide. In some embodiments, the DNA segment of a chimeric PE guide polynucleotide comprises an extension arm of the chimeric PE guide polynucleotide. In some embodiments, the DNA segment of the chimeric PE guide polynucleotide comprises a primer binding site sequence (PBS) that is capable of priming a DNA polymerase (e.g., DNA polymerase α, β, γ, δ, or ε) of a prime editor. In some embodiments, the DNA segment of a chimeric PE guide polynucleotide comprises an editing template that comprises one or more nucleotide edits to be incorporated into a double stranded target polynucleotide. In some embodiments, the DNA segment of a chimeric PE guide polynucleotide i) comprises one or more nucleotide edits to be incorporated into a double-stranded target polynucleotide, ii) is at least partially complementary to a portion of an edit strand of the double-stranded target polynucleotide, and iii) contains a sequence that is complementary or substantially complementary to, and capable of annealing to a free 3′ end on an edit strand of the double stranded target polynucleotide to initiate DNA synthesis by a DNA polymerase (e.g., DNA polymerase α, β, γ, δ, or ε).

In some embodiments, an RNA segment of a chimeric PE guide polynucleotide is complementary to a portion of the DNA segment of a chimeric PE guide polynucleotide. In some embodiments, an RNA segment of a chimeric PE guide polynucleotide comprises an extension arm of the chimeric PE guide polynucleotide. In some embodiments, an RNA segment of the chimeric PE guide polynucleotide comprises a primer binding site sequence (PBS) that is complementary or substantially complementary to, and capable of annealing to a free 3′ end on an edit strand of the double stranded target polynucleotide to initiate DNA synthesis by a DNA polymerase (e.g., a DNA-dependent DNA polymerase or a reverse transcriptase) of a prime editor. In some embodiments, an RNA segment of a chimeric PE guide polynucleotide comprises an editing template that comprises one or more nucleotide edits to be incorporated into a double stranded target polynucleotide.

In some embodiments, an RNA segment of a chimeric PE guide polynucleotide comprises a spacer sequence that is complementary to a search target sequence in a target strand of a double stranded target polynucleotide. In some embodiments, an RNA segment of a chimeric PE guide polynucleotide comprises a scaffold that associates with a napDNAbp of a prime editor. In some embodiments, an RNA segment of a chimeric PE guide polynucleotide i) is at least partially complementary to a search target sequence of a target strand of the double-stranded target polynucleotide, and ii) is capable of binding to the napDNAbp. In some embodiments, the RNA segment includes i) a spacer that is at least partially complementary to a portion of the target strand of the double-stranded target polynucleotide, and ii) an invariable (constant) region that is capable of binding to the napDNAbp.

In some embodiments, the chimeric PE guide polynucleotide comprises RNA in the spacer and DNA in the extension arm. In some embodiments, the chimeric PE guide polynucleotide comprises RNA in the spacer and the scaffold and comprises DNA in the extension arm. The chimeric PE guide polynucleotide may comprise DNA in the PBS or the editing template of the extension arm. In certain embodiments, the chimeric PE guide polynucleotide may also comprise DNA in the spacer region. In certain embodiments, the chimeric PE guide polynucleotide may also comprise DNA in the scaffold region. In some embodiments, the extension arm comprises DNA. In some embodiments, all nucleotides in the extension arm are DNA nucleotides. In some embodiments, the extension arm is a DNA/RNA hybrid. For example, the extension arm may comprise a PBS that comprises RNA and an editing template that comprises DNA.

In some embodiments, the chimeric PE guide polynucleotide comprises from 5′ to 3′: i) an RNA segment including a) a spacer that is complementary to a search target sequence of a target strand of a double-stranded target polynucleotide; and b) a scaffold that is capable of binding to a nucleic acid programmable DNA binding protein (napDNAbp); and ii) a deoxyribonucleic acid (DNA) segment comprising a) an editing template that comprises one or more intended nucleotide edits to be incorporated into the double-stranded target polynucleotide; and b) a primer binding site that is at least partially complementary to a portion of the edit strand of the double-stranded target polynucleotide. In some embodiments, the chimeric PE guide polynucleotide comprises from 5′ to 3′: i) a DNA segment comprising a) an editing template that comprises one or more intended nucleotide edits to be incorporated into the double-stranded target polynucleotide; and b) a primer binding site that is at least partially complementary to a portion of the edit strand of the double-stranded target polynucleotide; and ii) an RNA segment comprising a) a spacer that is at least partially complementary to a portion of the target strand of a double-stranded target polynucleotide; and b) a scaffold that is capable of binding to a nucleic acid programmable DNA binding protein (napDNAbp). In some embodiments, the chimeric PE guide polynucleotide is used to alter a double-stranded target polynucleotide as shown in FIG. 1. In some embodiments, the chimeric PE guide polynucleotide is used to alter a double-stranded target polynucleotide as shown in FIG. 2.

In some embodiments, a PE guide polynucleotide comprises a single polynucleotide molecule that comprises the spacer sequence, the scaffold, and the extension arm. In some embodiments, a PE guide polynucleotide comprises multiple polynucleotide molecules, for example, two polynucleotide molecules. In some embodiments, a PE guide polynucleotide comprise a first polynucleotide molecule that comprises the spacer and a portion of the scaffold, and a second polynucleotide molecule that comprises the rest of the scaffold and the extension arm. In some embodiments, the scaffold in the first polynucleotide molecule and the scaffold portion in the second polynucleotide molecule are at least partly complementary to each other. In some embodiments, the first polynucleotide comprises an RNA spacer and a first portion of an RNA scaffold, which may also be referred to as a crRNA. In some embodiments, the second polynucleotide comprises a second portion of an RNA scaffold the extension arm, wherein the second portion of the RNA scaffold may also be referred to as a trans-activating crRNA, or tracr RNA.

In some embodiments, the crRNA portion and the tracr RNA portion of the gRNA core are at least partially complementary to each other.

Regions or segments of a PE guide polynucleotide can be arranged in any order. In some embodiments, the spacer, the scaffold, and the extension arm are arranged from 5′ to 3′. In some embodiments, the spacer, the scaffold, and the extension arm are arranged from 3′ to 5′. In some embodiments, the extension arm, the spacer, and the scaffold are arranged from 5′ to 3′. In some embodiments, the scaffold is in between the spacer and the extension arm.

In some embodiments, the PE guide polynucleotide comprises the structure: 5′-RNA Segment-DNA Segment-3′. In some embodiments, the PE guide polynucleotide comprises the structure: 5′-DNA Segment-RNA Segment-3′. In some embodiments, the PE guide polynucleotide comprises a DNA segment and a chimeric segment comprising both DNA and RNA. In some embodiments, the PE guide polynucleotide comprises an RNA segment and a chimeric segment comprising both DNA and RNA. In some embodiments, the DNA segment is complementary to a portion of the RNA segment or the chimeric segment. In some embodiments, the DNA segment i) comprises one or more intended nucleotide edits to be incorporated into a double-stranded target polynucleotide, ii) is at least partially complementary to a portion of an edit strand of the double-stranded target polynucleotide, and iii) contains a sequence that is complementary or substantially complementary to, and capable of annealing to a free 3′ end on an edit strand of the double stranded target polynucleotide to initiate DNA synthesis by a DNA polymerase (e.g., DNA polymerase α, β, γ, δ, or ε). In some embodiments, the chimeric segment i) comprises one or more intended nucleotide edits to be incorporated into a double-stranded target polynucleotide, ii) is at least partially complementary to a portion of an edit strand of the double-stranded target polynucleotide, and iii) contains a sequence that is complementary or substantially complementary to, and capable of annealing to a free 3′ end on an edit strand of the double stranded target polynucleotide to initiate DNA synthesis by a DNA polymerase (e.g., DNA polymerase α, β, γ, δ, or ε). In some embodiments, the RNA segment i) is at least partially complementary to a portion of a non-edit strand of the double-stranded target polynucleotide, and ii) is capable of binding to the napDNAbp. In some embodiments, the chimeric segment i) is at least partially complementary to a portion of a non-edit strand of the double-stranded target polynucleotide, and ii) is capable of binding to the napDNAbp.

In some embodiments, the DNA segment comprises i) a primer binding site that is at least partially complementary to a portion of the edit strand of the double-stranded target polynucleotide, and ii) an editing template capable of priming the DNA polymerase and comprises one or more intended nucleotide edits to be incorporated into the double-stranded target polynucleotide. In some embodiments, the chimeric segment comprises i) a primer binding site that is at least partially complementary to a portion of the edit strand of the double-stranded target polynucleotide, and ii) an editing template capable of priming the DNA polymerase and comprises one or more intended nucleotide edits to be incorporated into the double-stranded target polynucleotide. In some embodiments, the RNA segment comprises i) a variable (spacer) region that is at least partially complementary to a portion of the target strand of the double-stranded target polynucleotide, and ii) an invariable (constant or scaffold) region that is capable of binding to the napDNAbp. In some embodiments, the chimeric segment comprises i) a variable (spacer) region that is at least partially complementary to a portion of the target strand of the double-stranded target polynucleotide, and ii) an invariable (constant or scaffold) region that is capable of binding to the napDNAbp.

In some embodiments, the editing template of a PEg polynucleotide is 5′ of the PBS of the PEg polynucleotide. As used herein, regardless of relative 5′-3′ positioning in other context, the relative positions as between the PBS and the editing template, and the relative positions as among elements of a chimeric PEg polynucleotide in embodiments wherein the PEg polynucleotide is a single molecule PEg polynucleotide, are determined by the 5′ to 3′ order of the chimeric PEg polynucleotide as a single molecule regardless of the position of sequences in the double stranded target DNA that may have complementarity or identity to elements of the chimeric PEg polynucleotide. In some embodiments, the PE guide polynucleotide comprises the structure: 5′-spacer-scaffold-Editing template-Primer Binding Site-3′. In some embodiments, the PE guide polynucleotide comprises the structure: 5′-Editing template-Primer Binding Site-spacer-scaffold-3′. In some embodiments, the spacer comprises RNA. In some embodiments, the spacer comprises DNA. In some embodiments, all nucleotides in the spacer are RNA nucleotides. In some embodiments, all nucleotides in the spacer are DNA nucleotides. In some embodiments, the primer binding site comprises RNA. In some embodiments, the primer binding site comprises DNA. In some embodiments, all nucleotides in the primer binding site are RNA nucleotides. In some embodiments, all nucleotides in the primer binding site are DNA nucleotides. In some embodiments, the editing template comprises RNA. In some embodiments, the editing template comprises DNA. In some embodiments, all nucleotides in the editing template are RNA nucleotides. In some embodiments, all nucleotides in the editing template are DNA nucleotides. In some embodiments, the scaffold comprises RNA. In some embodiments, the scaffold comprises DNA. In some embodiments, all nucleotides in the spacer are RNA nucleotides.

In some embodiments, a PE guide polynucleotide comprises a region that forms a secondary structure. In some embodiments, a scaffold of the PE guide polynucleotide can form a secondary structure. In some embodiments, the secondary structure comprises a stem, a hairpin, and/or a loop. The length of a loop and a stem can vary. For example, a loop can range from or from about 3 to 10 nucleotides in length, and a stem can range from or from about 6 to 20 base pairs in length. In some embodiments, a stem can comprise one or more bulges of 1 to 10 or about 10 nucleotides. The overall length of the region that forms a secondary structure can range from or from about 16 to 60 nucleotides in length. For example, a loop can be or can be about 4 nucleotides in length and a stem can be or can be about 12 base pairs.

In some embodiments, the PE guide polynucleotide forms a stem loop with a separate non-covalently linked sequence, which can be DNA or RNA. In some embodiments, the stem-loop forming sequences can be chemically synthesized. In some embodiments, the chemical synthesis uses automated, solid-phase oligonucleotide synthesis machines with 2′-acetoxyethyl orthoester (2′-ACE) (Scaringe et al., J. Am. Chem. Soc. (1998) 120: 11820-11821: Scaringe, Methods Enzymol. (2000) 317: 3-18) or 2′-thionocarbamate 2′-TC) chemistry (Dellinger et al., J. Am. Chem. Soc. (2011) 133: 11540-11546; Hendel et al., Nat. Biotechnol. (2015) 33:985-989).

In some embodiments, the sequence of PE guide polynucleotide, is selected to reduce the degree secondary structure within the PE guide polynucleotide. In some embodiments, the sequence of PE guide polynucleotide is selected for optimal folding of the PE guide polynucleotide. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and P A Carr and GM Church, 2009, Nature Biotechnology 27(12): 1151-62). In some embodiments, the PE guide polynucleotide is adjusted to avoid cleavage DNA- or RNA-cleaving enzymes (e.g., RNase H).

In some embodiments, the chimeric PE guide polynucleotide comprises natural nucleotides (e.g., adenosine). In some embodiments, the chimeric PE guide polynucleotide comprises non-natural (or unnatural) nucleotides (e.g., peptide nucleic acid or nucleotide analogs). In some embodiments, the chimeric PE guide polynucleotide is modified. In some embodiments, the RNA segment of the chimeric PE guide polynucleotide comprises modified RNA. In some embodiments, the DNA segment of the chimeric PE guide polynucleotide comprises modified DNA. In some embodiments, the chimeric PE guide polynucleotide comprises one or more modified nucleotides. In some embodiments, the chimeric PE guide polynucleotide comprises one or more modified nucleotides that are resistant to RNase cleavage. In some embodiments, the chimeric PE guide polynucleotide comprises a modified ribonucleotide that is resistant to RNase cleavage. In some embodiments, the one or more modified ribonucleotides are selected from 2′-O-methyl modified, 2′-ribo 3′-phosphorothioate modified, 2′O-methy 3′-phosphorothioate modified, deoxy modified, 2′-O-methoxyethyl (MOE) modified, 2′-fluoro, or methylphosphonate modified. In some embodiments, the chimeric PE guide polynucleotide comprises one or more peptide nucleic acids (PNAs), morpholino nucleic acids, bridged nucleic acids (BNA), or locked nucleic acids (LNAs). In some embodiments, the chimeric PE guide polynucleotide further comprises a blocking oligonucleotide. In some embodiments, the blocking oligonucleotide is a DNA. In some embodiments, the blocking oligonucleotide is a modified RNA. In some embodiments, the blocking oligonucleotide is at least 12 nucleotides or ribonucleotides in length. In some embodiments, the chimeric PE guide polynucleotide comprises a hydrolysable protecting group. The guide polynucleotides can comprise standard ribonucleotides, modified ribonucleotides (e.g., pseudouridine), ribonucleotide isomers, and/or ribonucleotide analogs. In some embodiments, the PE guide polynucleotide can comprise at least one detectable label. The detectable label can be a fluorophore (e.g., FAM, TMR, Cy3, Cy5, Texas Red, Oregon Green, Alexa Fluors, Halo tags, or suitable fluorescent dye), a detection tag (e.g., biotin, digoxigenin, and the like), quantum dots, or gold particles.

In some embodiments, a chimeric PE guide polynucleotide may be modified with a 5′ phosphate. In some embodiments, the 5′ phosphate may be incorporated either chemically or with a kinase. Other methods known by one skilled in the art may be used to incorporate a 5′ phosphate. In some embodiments, a chimeric PE guide polynucleotide can be modified by 5′adenylate, 5′ guanosine-triphosphate cap, 5′N7-Methylguanosine-triphosphate cap, 5′triphosphate cap, 3′phosphate, 3′thiophosphate, 5′phosphate, 5′thiophosphate, Cis-Syn thymidine dimer, trimers, C12 spacer, C3 spacer, C6 spacer, dSpacer, PC spacer, rSpacer, Spacer 18, Spacer 9,3′-3′ modifications, 5′-5′ modifications, abasic, acridine, azobenzene, biotin, biotin BB, biotin TEG, cholesteryl TEG, desthiobiotin TEG, DNP TEG, DNP-X, DOTA, dT-Biotin, dual biotin, PC biotin, psoralen C2, psoralen C6, TINA, 3′DABCYL, black hole quencher 1, black hole quencher 2, DABCYL SE, dT-DABCYL, IRDye QC-1, QSY-21, QSY-35, QSY-7, QSY-9, carboxyl linker, thiol linkers, 2′-deoxyribonucleoside analog purine, 2′-deoxyribonucleoside analog pyrimidine, ribonucleoside analog, 2′-O-methyl ribonucleoside analog, sugar modified analogs, wobble/universal bases, fluorescent dye label, 2′-fluoro RNA, 2′-O-methyl RNA, methylphosphonate, phosphodiester DNA, phosphodiester RNA, phosphothioate DNA, phosphorothioate RNA, UNA, pseudouridine-5′-triphosphate, 5′-methylcytidine-5′-triphosphate, or any combination thereof.

A modification can also be a phosphorothioate substitute. In some embodiments, a natural phosphodiester bond can be susceptible to rapid degradation by cellular nucleases and; a modification of internucleotide linkage using phosphorothioate (PS) bond substitutes can be more stable towards hydrolysis by cellular degradation. A modification can increase stability in a gRNA or a guide polynucleotide. A modification can also enhance biological activity. In some embodiments, a phosphorothioate enhanced PE guide polynucleotide can inhibit RNase A, RNase T1, calf serum nucleases, or any combinations thereof. These properties can allow the use of PE guide polynucleotide to be used in applications where exposure to nucleases is of high probability in vivo or in vitro. For example, phosphorothioate (PS) bonds can be introduced between the last 3-5 nucleotides at the 5′- or ″-end of a PE guide polynucleotide which can inhibit exonuclease degradation. In some embodiments, phosphorothioate bonds can be added throughout an entire gRNA to reduce attack by endonucleases.

In some embodiments, a chimeric PE guide polynucleotide can comprise a nucleic acid affinity tag. A chimeric PE guide polynucleotide can comprise synthetic nucleotide, synthetic nucleotide analog, nucleotide derivatives, and/or modified nucleotides. In some embodiments, quality control can include PAGE, HPLC, MS, or any combination thereof.

In some embodiments, a modification to the chimeric PE guide polynucleotide is permanent. In other embodiments, a modification to the chimeric PE guide polynucleotide is transient. In some embodiments, multiple modifications are made to the chimeric PE guide polynucleotide. In some embodiments, a modification can alter physiochemical properties of a nucleotide, such as their conformation, polarity, hydrophobicity, chemical reactivity, base-pairing interactions of the chimeric PE guide polynucleotide, or any combination thereof.

In particular embodiments, the susceptibility of the chimeric PE guide polynucleotide to RNases can be reduced by slight modifications of the sequence of the guide molecule which do not affect its function. For instance, in particular embodiments, premature termination of transcription, such as premature transcription of U6 Pol-III, can be removed by modifying a putative Pol-Ill terminator (4 consecutive U's) in the guide molecules sequence. Where such sequence modification is required, it is preferably ensured by a base pair flip. In some embodiments, ribonucleic acids are added in a DNA portion of the chimeric PE guide polynucleotide. In some embodiments, the chimeric PE guide polynucleotide comprises modified ribonucleotides that are resistant to RNase cleavage. In some embodiments, one or more nucleotides of the DNA segment of a guide polynucleotide comprise a hydrolysable protecting group to prevent RNase cleavage. In some embodiments, a blocking oligonucleotide (e.g., DNA or modified RNA) is used to prevent RNase cleavage. In some embodiments, the blocking oligonucleotide is at least 12 nucleotides or ribonucleotides in length.

In some embodiments, the sequence of the chimeric PE guide polynucleotide is selected to reduce the degree secondary structure within the guide molecule. In some embodiments, about or less than about 75%, 50%, 40%, 30%, 25%, 20%, 15%, 10%, 5%), 1%), or fewer of the nucleotides of the chimeric PE guide polynucleotide participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and PA Carr and GM Church, 2009, Nature Biotechnology 27(12): 1151-62).

In some embodiments, it is of interest to reduce the susceptibility of the guide molecule to RNA or DNA cleavage. Accordingly, in particular embodiments, the chimeric PE guide polynucleotide is adjusted to avoid cleavage DNA- or RNA-cleaving enzymes (e.g., RNase H).

The PE guide polynucleotides described herein can be synthesized chemically, synthesized enzymatically, or a combination thereof.

In some embodiments, the chimeric PE guide polynucleotide sequences can be chemically synthesized. In some embodiments, the chemical synthesis uses automated, solid-phase oligonucleotide synthesis machines with 2′-acetoxyethyl orthoester (2′-ACE) (Scaringe et al., J. Am. Chem. Soc. (1998) 120: 11820-11821; Scaringe, Methods Enzymol. (2000) 317: 3-18) or 2′-thionocarbamate (2′-TC) chemistry (Dellinger et al., J. Am Chem. Soc. (2011) 133: 11540-11546; Hendel et al., Nat. Biotechnol. (2015) 33:985-989). In some embodiments, the PE guide polynucleotide can be synthesized using standard phosphoramidite-based solid-phase synthesis methods.

In particular embodiments, the sequences forming the chimeric PE guide polynucleotide are first synthesized using the standard phosphoramidite synthetic protocol (Herdewijn, P., ed., Methods in Molecular Biology Col 288, Oligonucleotide Synthesis: Methods and Applications, Humana Press, New Jersey (2012)). In some embodiments, these sequences can be functionalized to contain an appropriate functional group using the standard protocol known in the art (Hermanson, G. T., Bioconjugate Techniques, Academic Press (2013)). Examples of functional groups include, but are not limited to, hydroxyl, amine, carboxylic acid, carboxylic acid halide, carboxylic acid active ester, aldehyde, carbonyl, chlorocarbonyl, imidazolylcarbonyl, hydrozide, semicarbazide, thio semicarbazide, thiol, maleimide, haloalkyl, sufonyl, ally, propargyl, diene, alkyne, and azide. Once this sequence is functionalized, covalent chemical bond or linkage can be formed between this sequence and the direct repeat sequence. Examples of chemical bonds include, but are not limited to, those based on carbamates, ethers, esters, amides, imines, amidines, aminotrizines, hydrazone, disulfides, thioethers, thioesters, phosphorothioates, phosphorodithioates, sulfonamides, sulfonates, fulfones, sulfoxides, ureas, thioureas, hydrazide, oxime, triazole, photolabile linkages, C—C bond forming groups such as Diels-Alder cyclo-addition pairs or ring-closing metathesis pairs, and Michael reaction pairs.

In some embodiments, the chimeric PE guide polynucleotide sequences can be enzymatically synthesized. In some embodiments, the chimeric PE guide polynucleotide can be synthesized in vitro by operably linking DNA encoding the guide RNA to a promoter control sequence that is recognized by a phage RNA polymerase. Examples of suitable phage promoter sequences include T7, T3, SP6 promoter sequences, or variations thereof. In embodiments in which the PE guide polynucleotide comprises two separate molecules (e.g., the scaffold separated in two molecules), one of the two molecules can be chemically synthesized and the other can be enzymatically synthesized. For example, one of the two guide polynucleotide molecules may comprise only RNA (e.g., an RNA spacer and an RNA scaffold) and may be enzymatically synthesized, while the other one of the two guide polynucleotide molecules may comprise both RNA and DNA (e.g., an RNA PBS and DNA editing template) and may be chemically synthesized.

### Prime Editing Compositions

The term “prime editing composition” or “prime editing system” refers to compositions involved in the method of prime editing as described herein. A prime editing composition may include a prime editor, e.g., a prime editor fusion protein, and a PE guide nucleotide. Components of a prime editing composition may form a complex for prime editing, or may be placed separately, e.g., for administration purposes.

Prime editing compositions as provided herein provide an approach to genome editing that uses a prime editor and a chimeric PE guide polynucleotide to incorporate changes in target double stranded polynucleotides, e.g., target DNA, without generating double-strand DNA breaks and having increased efficiency. In some embodiments, the prime editing composition comprises i) a prime editor comprising the nucleic acid programmable DNA binding protein (napDNAbp) with nickase activity (e.g., nCas9) and a DNA polymerase (e.g., DNA polymerase α, β, γ, δ, or ε), and ii) a chimeric PE guide polynucleotide. In some embodiments, the prime editor comprises a fusion protein comprising the napDNAbp and the DNA polymerase. In some embodiments, the prime editor forms a complex with the chimeric PE guide polynucleotide and binds an edit strand of a double-stranded target polynucleotide at a target locus of interest.

Provided herein are prime editing compositions, complexes and methods for editing a double-stranded target polynucleotide (e.g., DNA). In various embodiments, the prime editing composition is capable of modifying one or more nucleotides within a double-stranded target polynucleotide (e.g., DNA). In various embodiments, the prime editing composition is capable of substituting one or more nucleotides within a double-stranded target polynucleotide (e.g., DNA). In various embodiments, the prime editing composition is capable of inserting one or more nucleotides within a double-stranded target polynucleotide (e.g., DNA). In various embodiments, the prime editing composition is capable of deleting one or more nucleotides within a double-stranded target polynucleotide (e.g., DNA). In various embodiments, the prime editing composition is capable of modifying one or more nucleotides within a double-stranded target polynucleotide (e.g., DNA). In some embodiments, the double-stranded target polynucleotide is in the genome of a cell. In some embodiments, cell is a bacterial cell, plant cell, insect cell, or mammalian cell. In some embodiments, the cell is a human cell. In some embodiments, the chimeric PE guide polynucleotide is used to incorporate one or more intended nucleotide edits in a double-stranded target polynucleotide as shown in FIG. 1. In some embodiments, the chimeric PE guide polynucleotide is used to incorporate one or more intended nucleotide edits in a double-stranded target polynucleotide as shown in FIG. 2.

In some embodiments, a prime editing composition can comprise one or more PE guide polynucleotides. In some embodiments, a prime editing composition can comprise multiple PE guide polynucleotides (for example, 1, 2, 3, 4, 5, 6, 7, 8, 9, 10, 20, 30, or 50 PE guide polynucleotides). In some embodiments, the multiple PE polynucleotides each comprises a spacer sequence complementary to a same search target sequence. In some embodiments, the multiple PE polynucleotides each comprises a spacer sequence complementary to a different search target sequence. In some embodiments, the different search target sequences are in a same target gene. In some embodiments, the different search target sequences are in a same region, e.g., an exon, of a target gene. In some embodiments, the different search target sequences are in different genes on a same chromosome. In some embodiments, the different search target sequences are in different genes in a same cell.

In some embodiments, a prime editing composition comprises one or more polynucleotides encoding one or more prime editor polypeptides or one or more PE guide polynucleotides. In some embodiments, the polynucleotides encoding one or more prime editing composition components are DNA sequences. In some embodiments, the polynucleotides encoding prime editing composition components can be DNA, RNA, or any combination thereof. In some embodiments, the polynucleotides encoding prime editing composition components are mRNA sequences.

In some embodiments, a polynucleotide encoding a prime editor component or a PE guide polynucleotide, e.g., a DNA or RNA, is linear. In some embodiments, a polynucleotide encoding a prime editor component or a PE guide polynucleotide, e.g., a DNA or RNA, is circular.

Polynucleotides encoding a prime editor or a PE guide polynucleotide described herein may be harbored and delivered by any delivery approach known in the art, including lipid nanoparticles (LNPs), viral vectors, non-viral vectors, and physical techniques such as cell membrane disruption by a microfluidics device. In some embodiments, a polynucleotide encoding a prime editor polypeptide or a PE guide polynucleotide is in an expression construct. In some embodiments, a polynucleotide encoding a prime editing composition component is a vector. In some embodiments, the vector is a DNA vector. In some embodiments, the vector is a plasmid. In some embodiments, the vector is a virus vector, e.g., a retroviral vector, adenoviral vector, lentiviral vector, herpesvirus vector, or an adeno-associated virus vector (AAV). In some embodiments, a vector can comprise additional expression control sequences (e.g., enhancer sequences, Kozak sequences, polyadenylation sequences, transcriptional termination sequences, etc.), selectable marker sequences (e.g., GFP or antibiotic resistance genes such as puromycin), origins of replication, and the like. In some embodiments, a polynucleotide encoding a prime editor polypeptide or a PE guide polynucleotide is delivered by an LNP. In some embodiments, a polynucleotide encoding a prime editor polypeptide or a PE guide polynucleotide is delivered by a physical technique such as such as cell membrane disruption by a microfluidics device.

In some embodiments, a polynucleotide encoding a prime editor polypeptide or a PE guide polynucleotide comprises RNA. In some embodiments, the polynucleotide encoding a prime editor polypeptide or a PE guide polynucleotide is a mRNA.

In some embodiments, polynucleotides encoding polypeptide components of a prime editing composition are codon optimized by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. In some embodiments, a polynucleotide encoding a polypeptide component of a prime editing composition are operably linked to one or more expression regulatory elements, for example, a promoter, a 3′ UTR, a 5′ UTR, or any combination thereof. In some embodiments, a polynucleotide encoding a prime editing composition component is a messenger RNA (mRNA). In some embodiments, the mRNA comprises a Cap at the 5′ end and/or a poly A tail at the 3′ end.

Polynucleotides encoding prime editor or PE guide polynucleotide components may be introduced into an expression system, e.g., a cell, together or separately. For example, DNA sequences encoding a napDNAbp of a prime editor (e.g., a nCas9) and a guide RNA may be introduced into a cell, each DNA sequence can be part of a separate molecule (e.g., one vector containing the napDNAbp coding sequence and a second vector containing the guide polynucleotide coding sequence) or both can be part of a same molecule.

In some embodiments, a DNA that encodes a PE guide polynucleotide or a portion or segment thereof can be in a vector. In some embodiments, the vector encoding a PE guide polynucleotide or a portion or segment thereof can be introduced into a cell by transfecting the cell or transferred by any other delivery approach descried herein or known in the art, such as using virus-mediated gene delivery.

In some embodiments, a PE guide polynucleotide in a prime editing composition can be isolated. For example, a PE guide polynucleotide can be transfected in the form of an isolated RNA into a cell or organism. In some embodiments, a PE guide polynucleotide can be prepared by in vitro transcription using any in vitro transcription system known in the art. A guide RNA can be transferred to a cell in the form of isolated polynucleotide rather than in the form of plasmid comprising encoding sequence.

In some embodiments, a PE guide polynucleotide and/or a polynucleotide encoding a prime editor polypeptide can be introduced into a cell, tissue, organ or subject as an RNA molecule. For example, an RNA molecule can be transcribed in vitro and/or can be chemically synthesized. An RNA can be transcribed from a synthetic DNA molecule, e.g., a gBlocks® gene fragment. In some embodiments, a polynucleotide encoding a prime editor polypeptide or a PE guide polynucleotide is delivered by mRNA delivery.

In some embodiments, a PE guide polynucleotide and/or a polynucleotide encoding a prime editor polypeptide can be introduced into a cell, tissue, organ or subject in the form of a non-RNA nucleic acid molecule, e.g., DNA molecule. For example, a DNA can be operably linked to promoter control sequence for expression of the PE guide polynucleotide and/or the prime editor polypeptide in a cell, tissue, organ or subject of interest. A RNA coding sequence can be operably linked to a promoter sequence that is recognized by RNA polymerase III (Pol III). Plasmid vectors that can be used to express RNA include, but are not limited to, px330 vectors and px333 vectors. In some embodiments, a plasmid vector (e.g., px333 vector) can comprise at least two PE guide polynucleotides. In some embodiments, a prime editor polypeptide and a PE guide polynucleotide can be introduced into a cell, tissue, organ or subject as a ribonucleoprotein (RNP).

### Methods of Editing

The methods and compositions disclosed herein can be used to edit a target gene of interest by prime editing.

In some embodiments, the prime editing method comprises contacting a double stranded target polynucleotide with a PE guide polynucleotide and a prime editor (PE) polypeptide described herein. In some embodiments, the contacting with a PE guide polynucleotide and the contacting with a prime editor are performed sequentially. In some embodiments, the contacting with a prime editor is performed after the contacting with a PE guide polynucleotide. In some embodiments, the contacting with a PE guide polynucleotide is performed after the contacting with a prime editor. In some embodiments, the contacting with a PE guide polynucleotide, and the contacting with a prime editor are performed simultaneously. In some embodiments, the PE guide polynucleotide and the prime editor are associated in a complex prior to contacting a target gene.

In some embodiments, contacting the target gene with the prime editing composition results in binding of the PE guide polynucleotide to a target strand of the target gene. In some embodiments, contacting the target gene with the prime editing composition results in binding of the PE guide polynucleotide to a search target sequence on the target strand of the target gene upon contacting with the PE guide polynucleotide. In some embodiments, contacting the target gene with the prime editing composition results in binding of a spacer sequence of the PE guide polynucleotide to a search target sequence with the search target sequence on the target strand of the target gene upon said contacting of the PE guide polynucleotide.

In some embodiments, contacting the double stranded target polynucleotide with the prime editing composition results in binding of the prime editor to the double stranded target polynucleotide, upon the contacting of the PE composition with the double stranded target polynucleotide. In some embodiments, the napDNAbp of the PE associates with the PE guide polynucleotide. In some embodiments, the PE binds the double stranded target polynucleotide, directed by the PE guide polynucleotide. Accordingly, in some embodiments, the contacting of the double stranded target polynucleotide result in binding of a DNA binding domain of a prime editor of the double stranded target polynucleotide directed by the PE guide polynucleotide.

In some embodiments, contacting the double stranded target polynucleotide with the prime editing composition results in a nick in an edit strand of the double stranded target polynucleotide by the prime editor upon contacting with the double stranded target polynucleotide, thereby generating a nicked on the edit strand of the double stranded target polynucleotide. In some embodiments, contacting the double stranded target polynucleotide with the prime editing composition results in a single-stranded DNA comprising a free 3′ end at the nick site of the edit strand of the double stranded target polynucleotide. In some embodiments, contacting the double stranded target polynucleotide with the prime editing composition results in a nick in the edit strand of the double stranded target polynucleotide by a DNA binding domain of the prime editor, thereby generating a single-stranded DNA comprising a free 3′ end at the nick site. In some embodiments, the DNA binding domain of the prime editor is a Cas domain. In some embodiments, the DNA binding domain of the prime editor is a Cas9. In some embodiments, the DNA binding domain of the prime editor is a Cas9 nickase.

In some embodiments, contacting the double stranded target polynucleotide with the prime editing composition results in hybridization of the PE guide polynucleotide with the 3′ end of the nicked single-stranded DNA, thereby priming DNA polymerization by a DNA polymerase domain of the prime editor. In some embodiments, the free 3′ end of the single-stranded DNA generated at the nick site hybridizes to a primer binding site sequence (PBS) of the contacted PE guide polynucleotide, thereby priming DNA polymerization.

In some embodiments, contacting the double stranded target polynucleotide with the prime editing composition generates an edited single stranded DNA that is coded by the editing template of the PE guide polynucleotide by DNA polymerase mediated polymerization from the 3′ free end of the single-stranded DNA at the nick site. In some embodiments, the editing template of the PE guide polynucleotide comprises one or more intended nucleotide edits compared to endogenous sequence of the double stranded target polynucleotide. In some embodiments, the intended nucleotide edits are incorporated in the double stranded target polynucleotide, by excision of the 5′ single stranded DNA of the edit strand of the double stranded target polynucleotide generated at the nick site and DNA repair. in some embodiments, the intended nucleotide edits are incorporated in the double stranded target polynucleotide by excision of the editing target sequence and DNA repair. hi some embodiments, excision of the 5′ single stranded DNA of the edit strand generated at the nick site is by a flap endonuclease. In some embodiments, the flap nuclease is FEN1. In some embodiments, the method further comprises contacting the double stranded target polynucleotide with a flap endonuclease. In some embodiments, the flap endonuclease is provided as a part of a prime editor fusion protein. In some embodiments, the flap endonuclease is provided in trans.

In some embodiments, contacting the double stranded target polynucleotide with the prime editing composition generates a mismatched heteroduplex comprising the edit strand of the double stranded target polynucleotide that comprises the edited single stranded DNA, and the unedited target strand of the double stranded target polynucleotide. Without being bound by theory, the endogenous DNA repair and replication may resolve the mismatched edited DNA to incorporate the nucleotide change(s) to form the desired edited double stranded target polynucleotide. In some embodiments, contacting the double stranded target polynucleotide with the prime editing composition results in incorporation of one or more intended nucleotide edits in the double stranded target polynucleotide.

In some embodiments, an intended nucleotide edit is upstream of a PAM site in the edit strand of the double stranded target polynucleotide. hi some embodiments, an intended nucleotide edit is 1, 2, 3, 4, 5, 6, 7, 8, 9, 10, 11, 12, 13, 14, 15, 16, 17, 18, 19, or 20 nucleotides upstream of a PAM site in the edit strand of the double stranded target polynucleotide. In some embodiments, an intended nucleotide edit is downstream of a PAM site in the edit strand of the double stranded target polynucleotide. In some embodiments an intended nucleotide edit is 1, 2, 3, 4, 5, 6, 7, 8, 9, 10, 11, 12, 13, 14, 15, 16, 17, 18, 19, or 20 nucleotides downstream of a PAM site in the edit strand of the double stranded target polynucleotide.

In some embodiments, the method comprises recruiting a RNase to the edit site of the double stranded target polynucleotide. In some embodiments, the method comprises using RNase H II to cleave the RNA-DNA junction. In some embodiments, the method comprises an RNase H tethered to the prime editing composition with a linker. In some embodiments, the method does not require RNase H. In some embodiments, the method comprises dissociating the DNA polymerase from the edit strand of the double stranded target polynucleotide to allow repair of the double stranded target polynucleotide with DNA repair proteins (e.g., FEN1 and DNA ligase).

In some embodiments, the double stranded target polynucleotide, is in a cell. Accordingly, also provided herein are methods of modifying a cell. In some embodiments, the PE guide polynucleotide and the prime editor polypeptide form a complex prior to the introduction into the cell. In some embodiments, the PE guide polynucleotide and the prime editor polypeptide form a complex after the introduction into the cell. The prime editors, PE guide polynucleotide, or the prime editing complexes may be introduced into the cell by any delivery approaches described herein or any delivery approach known in the art, including ribonucleoprotein (RNPs), lipid nanoparticles (LNPs), viral vectors, non-viral vectors, snRNA delivery, and physical techniques such as cell membrane disruption by a microfluidics device. The prime editors, PE guide polynucleotide, and/or prime editing complexes may be introduced into the cell simultaneously or sequentially.

In some embodiments, the cell is a prokaryotic cell. In some embodiments, the cell is a eukaryotic cell. In some embodiments, the cell is a mammalian cell. in some embodiments, the cell is a non-human primate cell, bovine cell, porcine cell, rodent or mouse cell. In some embodiments, the cell is a human cell.

In some embodiments, the double stranded target polynucleotide edited by prime editing is in a chromosome of the cell. In some embodiments, the intended nucleotide edits incorporate in the chromosome of the cell and are inheritable by progeny cells. In some embodiments, the intended nucleotide edits introduced to the cell by the prime editing compositions and methods are such that the cell and progeny of the cell also include the intended nucleotide edits. In some embodiments, the cell is autologous, allogeneic, or xenogeneic to a subject. In some embodiments, the cell is from or derived from a subject. In some embodiments, the cell is from or derived from a human subject. In some embodiments, the cell is introduced back into the subject, e.g., a human subject, after incorporation of the intended nucleotide edits by prime editing.

In some embodiments, the method provided herein comprises introducing the prime editor polypeptide or the polynucleotide encoding the prime editor polypeptide, and the PE guide polynucleotide or the polynucleotide encoding the PE guide polynucleotide, into a plurality or a population of cells that comprise the double stranded target polynucleotide. In some embodiments, the population of cells is of the same cell type. In some embodiments, the population of cells is of the same tissue or organ. In some embodiments, the population of cells is heterogeneous. In some embodiments, the population of cells is homogeneous. In some embodiments, the population of cells is from a single tissue or organ, and the cells are heterogeneous. In some embodiments, the introduction into the population of cells is ex vivo. In some embodiments, the introduction into the population of cells is in vivo, e.g., into a human subject.

In some embodiments, the double stranded target polynucleotide is in a genome of each cell of the population. In some embodiments, introduction of the prime editor polypeptide or the polynucleotide encoding the prime editor polypeptide and the PE guide polynucleotide or the polynucleotide encoding the PE guide polynucleotide results in incorporation of one or more intended nucleotide edits in the double stranded target polynucleotide in at least one of the cells in the population of cells. In some embodiments, introduction of the prime editor polypeptide or the polynucleotide encoding the prime editor polypeptide and the PE guide polynucleotide or the polynucleotide encoding the PE guide polynucleotide results in incorporation of the one or more intended nucleotide edits in the double stranded target polynucleotide in a plurality of the population of cells. In some embodiments, introduction of the prime editor polypeptide or the polynucleotide encoding the prime editor polypeptide and the PE guide polynucleotide or the polynucleotide encoding the PE guide polynucleotide results in incorporation of the one or more intended nucleotide edits in the double stranded target polynucleotide in each cell of the population of cells.

In some embodiments, editing efficiency of the prime editing compositions and method described herein can be measured by calculating the percentage of edited target genes in a population of cells introduced with the prime editing composition. In some embodiments, the editing efficiency is determined after 1 hour, 2 hours, 6 hours, 12 hours. 24 hours, 36 hours, 48 hours, 3 days. 4 days, 5 days, 7 days, 10 days, or 14 days of exposing a target gene (e.g., a ATP7B gene within the genome of a cell) to a prime editing composition. In some embodiments, the population of cells introduced with the prime editing composition is ex vivo. In some embodiments, the population of cells introduced with the prime editing composition is in vitro. In some embodiments, the population of cells introduced with the prime editing composition is in vivo.

In some embodiments, the prime editing composition, guide polynucleotides and prime editor proteins as described herein can modify a double-stranded target polynucleotide without generating a significant proportion of indels. An “indel,” as used herein, refers to the insertion or deletion of a nucleotide base(s) within a nucleic acid. Such insertions or deletions can lead to frame shift mutations within a coding region of a gene. In certain embodiments, any of the prime editing compositions provided herein are capable of generating a greater proportion of intended modifications (e.g., mutations) versus indels.

In some embodiments, any prime editing composition provided herein result in less than 50%, less than 40%, less than 30%, less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, less than 1%, less than 0.9%, less than 0.8%, less than 0.7%, less than 0.6%, less than 0.5%, less than 0.4%, less than 0.3%, less than 0.2%, less than 0.1%, less than 0.09%, less than 0.08%, less than 0.07%, less than 0.06%, less than 0.05%, less than 0.04%, less than 0.03%, less than 0.02%, or less than 0.01% indel formation in the target polynucleotide sequence.

Some aspects of the disclosure are based on the recognition that any of the prime editing composition provided herein are capable of efficiently incorporating one or more intended nucleotide edits, such as a point mutation, in a nucleic acid (e.g., a nucleic acid within a genome of a subject) without generating a significant number of unintended mutations, such as unintended point mutations. In some embodiments, any of the prime editing composition provided herein are capable of generating at least 0.01% of intended nucleotide edits (i.e. at least 0.01% editing efficiency). In some embodiments, any of the prime editing composition provided herein are capable of generating at least 0.01%, 1%, 2%, 3%, 4%, 5%, 10%, 15%, 20%, 25%, 30%, 40%, 45%, 50%, 60%, 70%, 80%, 90%, 95%, or 99% of intended nucleotide edits.

In some embodiments, the prime editing composition provided herein are capable of generating a ratio of intended nucleotide edits to indels that is greater than 1:1. In some embodiments, the prime editing composition provided herein are capable of generating a ratio of intended nucleotide edits to indels that is at least 1.5:1, at least 2:1, at least 2.5:1, at least 3:1, at least 3.5:1, at least 4:1, at least 4.5:1, at least 5:1, at least 5.5:1, at least 6:1, at least 6.5:1, at least 7:1, at least 7.5:1, at least 8:1, at least 10:1, at least 12:1, at least 15:1, at least 20:1, at least 25:1, at least 30:1, at least 40:1, at least 50:1, at least 100:1, at least 200:1, at least 300:1, at least 400:1, at least 500:1, at least 600:1, at least 700:1, at least 800:1, at least 900:1, or at least 1000:1, or more.

The number of intended nucleotide edits and indels can be determined using any suitable method, for example, as described in International PCT Application Nos. PCT/2017/045381 (WO2018/027078) and PCT/US2016/058344 (WO2017/070632); Komor, A. C., et al., “Programmable editing of a target base in genomic DNA without double-stranded DNA cleavage” Nature 533, 420-424 (2016); Gaudelli, N. M., et al., “Programmable base editing of A⋅T to G⋅C in genomic DNA without DNA cleavage” Nature 551, 464-471 (2017); and Komor, A. C., et al., “Improved base excision repair inhibition and bacteriophage Mu Gam protein yields C:G-to-T:A prime editing composition with higher efficiency and product purity” Science Advances 3:eaao4774 (2017); the entire contents of which are hereby incorporated by reference.

In some embodiments, to calculate indel frequencies, sequencing reads are scanned for exact matches to two 10-bp sequences that flank both sides of a window in which indels can occur. If no exact matches are located, the read is excluded from analysis. If the length of this indel window exactly matches the reference sequence the read is classified as not containing an indel. If the indel window is two or more bases longer or shorter than the reference sequence, then the sequencing read is classified as an insertion or deletion, respectively. In some embodiments, the prime editing composition provided herein can limit formation of indels in a region of a nucleic acid. In some embodiments, the region is at a nucleotide targeted by a prime editing composition or a region within 2, 3, 4, 5, 6, 7, 8, 9, or 10 nucleotides of a nucleotide targeted by a prime editing composition.

The number of indels formed at a target polynucleotide region can depend on the amount of time a nucleic acid (e.g., a nucleic acid within the genome of a cell) is exposed to a prime editing composition. In some embodiments, the number or proportion of indels is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing the target nucleotide sequence (e.g., a nucleic acid within the genome of a cell) to a prime editing composition. It should be appreciated that the characteristics of the prime editing composition as described herein can be applied to any of the prime editor fusion proteins, or methods of using the prime editor fusion proteins provided herein.

In some embodiments, the prime editing composition provided herein are capable of limiting formation of indels in a region of a nucleic acid. In some embodiments, the region is at a nucleotide targeted by a prime editing composition or a region within 2, 3, 4, 5, 6, 7, 8, 9, or 10 nucleotides of a nucleotide targeted by a prime editing composition. In some embodiments, any of the prime editing composition provided herein are capable of limiting the formation of indels at a region of a nucleic acid to less than 1%, less than 1.5%, less than 2%, less than 2.5%, less than 3%, less than 3.5%, less than 4%, less than 4.5%, less than 5%, less than 6%, less than 7%, less than 8%, less than 9%, less than 10%, less than 12%, less than 15%, or less than 20%. The number of indels formed at a nucleic acid region may depend on the amount of time a nucleic acid (e.g., a nucleic acid within the genome of a cell) is exposed to a prime editing composition. In some embodiments, any number or proportion of indels is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing a nucleic acid (e.g., a nucleic acid within the genome of a cell) to a prime editing composition.

Some aspects of the disclosure are based on the recognition that any of the prime editing composition provided herein are capable of efficiently incorporating intended nucleotide edits in a target polynucleotide (e.g., a nucleic acid within a genome of a subject) without generating a significant number of unintended mutations.

In some embodiments, any of the prime editing composition provided herein are capable of generating a ratio of intended nucleotide edits to unintended mutation (e.g., intended nucleotide edits: unintended mutation) that is greater than 1:1. In some embodiments, any of the prime editing composition provided herein are capable of generating a ratio of intended nucleotide edits to unintended mutation that is at least 1.5:1, at least 2:1, at least 2.5:1, at least 3:1, at least 3.5:1, at least 4:1, at least 4.5:1, at least 5:1, at least 5.5:1, at least 6:1, at least 6.5:1, at least 7:1, at least 7.5:1, at least 8:1, at least 10:1, at least 12:1, at least 15:1, at least 20:1, at least 25:1, at least 30:1, at least 40:1, at least 50:1, at least 100:1, at least 150:1, at least 200:1, at least 250:1, at least 500:1, or at least 1000:1, or more. It should be appreciated that the characteristics of the prime editing composition described herein may be applied to any of the prime editor fusion proteins, or methods of using the prime editor fusion proteins provided herein.

The suitability of prime editing compositions that target and edit a double-stranded DNA target sequence is evaluated as described herein. In one embodiment, a single cell of interest is transduced with a prime editing composition together with a small amount of a vector encoding a reporter (e.g., GFP). These cells can be any cell line known in the art, including immortalized human cell lines, such as 293T, K562 or U20S. Alternatively, primary cells (e.g., human) may be used. Such cells may be relevant to the eventual cell target.

The activity of the prime editing composition is assessed as described herein, i.e., by sequencing the genome of the cells to detect alterations in a target sequence. For Sanger sequencing, purified PCR amplicons are cloned into a plasmid backbone, transformed, miniprepped and sequenced with a single primer. Sequencing may also be performed using next generation sequencing techniques. When using next generation sequencing, amplicons may be 300-500 bp with the intended cut site placed asymmetrically. Following PCR, next generation sequencing adapters and barcodes (for example Illumina multiplex adapters and indexes) may be added to the ends of the amplicon, e.g., for use in high throughput sequencing (for example on an Illumina MiSeq). The prime editing composition that induces the greatest levels of target specific alterations in initial tests can be selected for further evaluation.

### Delivery and Expression

Prime editor proteins and may be delivered to and expressed in virtually any host cell of interest, including but not limited to bacteria, yeast, fungi, insects, plants, and animal cells using routine methods known to the skilled artisan.

In some embodiments, a prime editor polypeptide or a PE guide polynucleotide is encoded by DNA. For example, a DNA encoding a DNA polymerase (e.g., DNA polymerase α, β, γ, δ, or ε) and a napDNAbp with nickase activity (e.g., nCas9) can be cloned by designing suitable primers for the upstream and downstream of CDS based on the cDNA sequence. The cloned DNA may be directly, or after digestion with a restriction enzyme, or after addition of a suitable linker and/or a nuclear localization signal, ligated with a DNA encoding one or more additional components of a prime editing composition. The prime editor may be translated in a host cell to form a complex with a PE guide polynucleotide.

A DNA encoding a protein domain described herein can be obtained by chemically synthesizing the DNA, or by connecting synthesized partly overlapping oligo DNA short chains by utilizing the PCR method and the Gibson Assembly method to construct a DNA encoding the full length thereof. The advantage of constructing a full-length DNA by chemical synthesis or a combination of PCR method or Gibson Assembly method is that the codon to be used can be designed in CDS full-length according to the host into which the DNA is introduced.

In some embodiments, a polynucleotide encoding a prime editor or polypeptide component thereof is codon optimized for expression the desired cell type (e.g., bacterial cell, plant cell, insect cell, or mammalian cell). In some embodiments, the cell is a eukaryotic cell, preferably a mammalian cell or a human cell. In some embodiments, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. In some embodiments, the expression level of the prime editor or polypeptide component thereof may be increased by codon optimization. Codon usage tables are readily available. See, Nakamura, Y., et al. “Codon usage tabulated from the international DNA sequence databases: status for the year 2000” Nucl. Acids Res. 28:292 (2000). In some embodiments, one or more codons (e.g., 1, 2, 3, 4, 5, 10, 15, 20, 25, 50, or more, or all codons) in a sequence encoding a prime editor or polypeptide component thereof correspond to the most frequently used codon for a particular amino acid.

In some embodiments, a polynucleotide encoding a prime editor or polypeptide component thereof comprises one or more nucleic acid sequences encoding an internal ribosome entry site (IRES) or 2A self-cleaving peptide. Introducing an IRES or 2A into an RNA permits a single RNA molecule to be translated into two or more discrete and distinct polypeptides. In some embodiments, an mRNA molecule can be translated into two or more peptides through insertion of nucleic acid sequences encoding an IRES or a 2A self-cleaving peptide between the nucleic acid sequences encoding the discrete polypeptides. Any suitable type of 2A peptide sequences may be used, including P2A, T2A, E2A, F2A, BmCPV 2A, and BmIFV 2A.

An expression vector containing a DNA encoding a nucleic acid sequence-recognizing module and/or a nucleic acid base converting enzyme can be produced, for example, by linking the DNA to the downstream of a promoter in a suitable expression vector.

In some embodiments, the expression vectors are Escherichia coli-derived plasmids (e.g., pBR322, pBR325, pUC12, pUC13); Bacillus subtilis-derived plasmids (e.g., pUB110, pTP5, pC194); yeast-derived plasmids (e.g., pSH19, pSH15); insect cell expression plasmids (e.g., pFast-Bac); animal cell expression plasmids (e.g., pA1-11, pXT1, pRc/CMV, pRc/RSV, pcDNAI/Neo); bacteriophages such as lambda phage and the like; insect virus vectors such as baculovirus and the like (e.g., BmNPV, AcNPV); or animal virus vectors such as retrovirus, vaccinia virus, adenovirus.

In some embodiments, any promoter appropriate for a host to be used for gene expression can be used. For example, when the host is an animal cell, an EFla promoter, SRa promoter, SV40 promoter, LTR promoter, CMV (cytomegalovirus) promoter, RSV (Rous sarcoma virus) promoter, MoMuLV (Moloney mouse leukemia virus) LTR, HSV-TK (simple herpes virus thymidine kinase) promoter and the like may be used. In some embodiments, when the host is Escherichia coli, a trp promoter, lac promoter, recA promoter, lamda.P.sub.L promoter, lpp promoter, T7 promoter and the like are preferable. In some embodiments, when the host is genus Bacillus, an SPO1 promoter, SPO2 promoter, penP promoter and the like are preferable. In some embodiments, when the host is a yeast, a Gal l/10 promoter, PHOS promoter, PGK promoter, GAP promoter, ADH promoter and the like are preferable. In some embodiments, when the host is an insect cell, a polyhedrin promoter, P10 promoter and the like are preferable. In some embodiments, when the host is a plant cell, a CaMV35S promoter, CaMV19S promoter, NOS promoter and the like are preferable.

In some embodiments, the expression vector comprises an enhancer, splicing signal, terminator, polyA addition signal, a selection marker such as drug resistance gene, auxotrophic complementary gene, and/or replication origin. In some embodiments, a prime editor polypeptide or a PE guide polynucleotide is encoded by RNA. An RNA encoding a protein domain or a PE guide polynucleotide described herein can be prepared by, for example, transcription to mRNA in an in vitro transcription system. In some embodiments, a polynucleotide encoding a prime editor polypeptide or a PE guide polynucleotide is modified. In some embodiments, the polynucleotide encoding a prime editor polypeptide or a PE guide polynucleotide is modified to include one or more modified nucleosides e.g., using pseudo-U or 5-Methyl-C.

A host cell for expressing one or more prime editing composition components can be any cell type or derived from any organism known in the art. In some embodiments, the host cell is a Escherichia cell, a cell from genus Bacillus, a yeast cell, an insect cell, an animal cell.

An expression vector can be introduced by a known method (e.g., lysozyme method, competent method, PEG method, CaCl₂) coprecipitation method, electroporation method, the microinjection method, the particle gun method, lipofection method, Agrobacterium method and the like) according to the kind of the host cell.

Polynucleotides encoding prime editor composition components according to the present disclosure can be administered to subjects or delivered into cells in vitro or in vivo by art-known methods or as described herein. In one embodiment, polynucleotides encoding prime editor composition components can be delivered by, e.g., vectors (e.g., viral or non-viral vectors), non-vector based methods (e.g., using naked DNA, DNA complexes, lipid nanoparticles), or a combination thereof.

Polynucleotides encoding prime editor composition components can be delivered directly to cells (e.g., hematopoietic cells or their progenitors, hematopoietic stem cells, and/or induced pluripotent stem cells) as naked DNA or RNA, for instance by means of transfection or electroporation, or can be conjugated to molecules (e.g., N-acetylgalactosamine) promoting uptake by the target cells. Nucleic acid vectors, such as the vectors described herein can also be used.

Nucleic acid vectors can comprise one or more polynucleotides encoding prime editor composition components described herein. A vector can also comprise a sequence encoding a signal peptide (e.g., for nuclear localization, nucleolar localization, or mitochondrial localization), associated with (e.g., inserted into or fused to) a sequence coding for a protein. As one example, nucleic acid vectors can include a napDNAbp, e.g., nCas9 coding sequence that includes one or more nuclear localization sequences (e.g., a BPNLS) and a DNA polymerase (e.g., DNA polymerase α, β, γ, δ, or ε).

The nucleic acid vector can also include any suitable number of regulatory/control elements, e.g., promoters, enhancers, introns, polyadenylation signals, or Kozak consensus sequences. In some embodiments, the promoter used to drive expression of a guide polynucleotide can include a Pol III promoter such as U6 or H1, or a Pol II promoter and intronic cassettes to express gRNA Adeno Associated Virus (AAV). In some embodiments, a promoter can be cell or tissue type specific. For hematopoietic cells suitable promoters can include IFNbeta or CD45. In some embodiments, the promoter is an AAV ITR. In some embodiments, the promoter is a CMV promoter, a CAG promoter, a CBh promoter, a PGK promoter, a SV40 promoter, or a Ferritin heavy or light chains promoter for ubiquitous expression. For brain or other CNS cell expression, suitable promoters can include: SynapsinI promoter for all neurons, CaMKIIα promoter for excitatory neurons, GAD67 promoter, GAD65 promoter, or VGAT promoter for GABAergic neurons. For liver cell expression, suitable promoters include the albumin promoter. For lung cell expression, suitable promoters can include SP-B promoter. For endothelial cells, suitable promoters can include ICAM promoter. For hematopoietic cells suitable promoters can include the IFNβ promoter or the CD45 promoter. For osteoblasts, suitable promoters can include OG-2 promoter. Nucleic acid vectors according to this disclosure include recombinant viral vectors. Exemplary viral vectors are set forth herein. Other viral vectors known in the art can also be used. In addition, viral particles can be used to deliver editor complex components in nucleic acid and/or peptide form. For example, “empty” viral particles can be assembled to contain any suitable cargo. Viral vectors and viral particles can also be engineered to incorporate targeting ligands to alter target tissue specificity.

In addition to viral vectors, non-viral vectors can be used to deliver nucleic acids encoding editor complexes according to the present disclosure. One important category of non-viral nucleic acid vectors are nanoparticles, which can be organic or inorganic. Nanoparticles are well known in the art. Any suitable nanoparticle design can be used to deliver genome editing system components or nucleic acids encoding such components. For instance, organic (e.g., lipid and/or polymer) nanoparticles can be suitable for use as delivery vehicles in certain embodiments of this disclosure. Exemplary lipids for use in nanoparticle formulations, and/or gene transfer are shown in Table 5 (below).

TABLE 5
Lipids Used for Gene Transfer

| Lipid | Abbreviation | Feature |
|---|---|---|
| 1,2-Dioleoyl-sn-glycero-3-phosphatidylcholine | DOPC | Helper |
| 1,2-Dioleoyl-sn-glycero-3- | DOPE | Helper |
| phosphatidylethanolamine | | |
| Cholesterol | | Helper |
| N-[1-(2,3-Dioleyloxy)prophyl]N,N,N- | DOTMA | Cationic |
| trimethylammonium chloride | | |
| 1,2-Dioleoyloxy-3-trimethylammonium- | DOTAP | Cationic |
| propane | | |
| Dioctadecylamidoglycylspermine | DOGS | Cationic |
| N-(3-Aminopropyl)-N,N-dimethyl-2,3- | GAP- | Cationic |
| bis(dodecyloxy)-1-propanaminium bromide | DLRIE | |
| Cetyltrimethylammonium bromide | CTAB | Cationic |
| 6-Lauroxyhexyl ornithinate | LHON | Cationic |
| 1-(2,3-Dioleoyloxypropyl)-2,4,6- | 2Oc | Cationic |
| trimethylpyridinium | | |
| 2,3-Dioleyloxy-N-[2(sperminecarboxamido- | DOSPA | Cationic |
| ethyl]-N,N-dimethyl-1-propanaminium | | |
| trifluoroacetate | | |
| 1,2-Dioleyl-3-trimethylammonium-propane | DOPA | Cationic |
| N-(2-Hydroxyethyl)-N,N-dimethyl-2,3- | MDRIE | Cationic |
| bis(tetradecyloxy)-1-propanaminium bromide | | |
| Dimyristooxypropyl dimethyl hydroxyethyl | DMRI | Cationic |
| ammonium bromide | | |
| 3β-[N-(N′,N′-Dimethylaminoethane)- | DC-Chol | Cationic |
| carbamoyl]cholesterol | | |
| Bis-guanidium-tren-cholesterol | BGTC | Cationic |
| 1,3-Diodeoxy-2-(6-carboxy-spermyl)- | DOSPER | Cationic |
| propylamide | | |
| Dimethyloctadecylammonium bromide | DDAB | Cationic |
| Dioctadecylamidoglicylspermidin | DSL | Cationic |
| rac-[(2,3-Dioctadecyloxypropyl)(2- | CLIP-1 | Cationic |
| hydroxyethyl)]-dimethylammonium chloride | | |
| rac-[2(2,3-Dihexadecyloxypropyl- | CLIP-6 | Cationic |
| oxymethyloxy)ethyl]trimethylammoniun | | |
| bromide | | |
| Ethyldimyristoylphosphatidylcholine | EDMPC | Cationic |
| 1,2-Distearyloxy-N,N-dimethyl-3-aminopropane | DSDMA | Cationic |
| 1,2-Dimyristoyl-trimethylammonium propane | DMTAP | Cationic |
| O,O′-Dimyristyl-N-lysyl aspartate | DMKE | Cationic |
| 1,2-Distearoyl-sn-glycero-3-ethylpho | DSEPC | Cationic |
| sphocholine | | |
| N-Palmitoyl D-erythro-sphingosyl carbamoyl- | CCS | Cationic |
| spermine | | |
| N-t-Butyl-N0-tetradecyl-3- | diC14- | Cationic |
| tetradecylaminopropionamidine | amidine | |
| Octadecenolyoxy[ethyl-2-heptadecenyl-3 | DOTIM | Cationic |
| hydroxyethyl] imidazolinium chloride | | |
| N1-Cholesteryloxycarbonyl-3,7-diazanonane- | CDAN | Cationic |
| 1,9-diamine | | |
| 2-(3-[Bis(3-amino-propyl)-amino] | RPR209120 | Cationic |
| propylamino)-N-ditetradecylcarbamoylme- | | |
| ethyl-acetamide | | |
| 1,2-dilinoleyloxy-3-dimethylaminopropane | DLinDMA | Cationic |
| 2,2-dilinoleyl-4-dimethylaminoethyl-[1,3]- | DLin-KC2- | Cationic |
| dioxolane | DMA | |
| dilinoleyl-methyl-4-dimethylaminobutyrate | DLin-MC3- | Cationic |
| | DMA | |

Table 6 lists exemplary polymers for use in gene transfer and/or nanoparticle formulations.

TABLE 6
Polymers Used for Gene Transfer

| Polymer | Abbreviation |
|---|---|
| Poly(ethylene)glycol | PEG |
| Polyethylenimine | PEI |
| Dithiobis (succinimidylpropionate) | DSP |
| Dimethyl-3,3′-dithiobispropionimidate | DTBP |
| Poly(ethylene imine)biscarbamate | PEIC |
| Poly(L-lysine) | PLL |
| Histidine modified PLL | |
| Poly(N-vinylpyrrolidone) | PVP |
| Poly(propylenimine) | PPI |
| Poly(amidoamine) | PAMAM |
| Poly(amidoethylenimine) | SS-PAEI |
| Triethylenetetramine | TETA |
| Poly(β-aminoester) | |
| Poly(4-hydroxy-1-proline ester) | PHP |
| Poly(allylamine) | |
| Poly(α-[4-aminobutyl]-1-glycolic acid) | PAGA |
| Poly(D,L-lactic-co-glycolic acid) | PLGA |
| Poly(N-ethyl-4-vinylpyridinium bromide) | |
| Poly(phosphazene)s | PPZ |
| Poly(phosphoester)s | PPE |
| Poly(phosphoramidate)s | PPA |
| Poly(N-2-hydroxypropylmethacrylamide) | pHPMA |
| Poly(2-(dimethylamino)ethyl methacrylate) | pDMAEMA |
| Poly(2-aminoethyl propylene phosphate) | PPE-EA |
| Chitosan | |
| Galactosylated chitosan | |
| N-Dodacylated chitosan | |
| Histone | |
| Collagen | |
| Dextran-spermine | D-SPM |

Table 7 summarizes delivery methods for a polynucleotide encoding a prime editor fusion protein described herein.

TABLE 7

| | | Delivery | | | |
|---|---|---|---|---|---|
| | | into | | | |
| | | Non- | Duration | | Type of |
| | | Dividing | of | Genome | Molecule |
| Delivery | Vector/Mode | Cells | Expression | Integration | Delivered |
| Physical | (e.g., | YES | Transient | NO | Nucleic |
| | electro- | | | | Acids and |
| | poration, | | | | Proteins |
| | particle gun, | | | | |
| | Calcium | | | | |
| | Phosphate | | | | |
| | transfection | | | | |
| Viral | Retrovirus | NO | Stable | YES | RNA |
| | Lentivirus | YES | Stable | YES/NO | RNA |
| | | | | with | |
| | | | | modification | |
| | Adenovirus | YES | Transient | NO | DNA |
| | Adeno- | YES | Stable | NO | DNA |
| | Associated | | | | |
| | Virus | | | | |
| | (AAV) | | | | |
| | Vaccinia | YES | Very | NO | DNA |
| | Virus | | Transient | | |
| | Herpes | YES | Stable | NO | DNA |
| | Simplex | | | | |
| | Virus | | | | |
| Non- | Cationic | YES | Transient | Depends on | Nucleic |
| Viral | Liposomes | | | what is | Acids and |
| | | | | delivered | Proteins |
| | Polymeric | YES | Transient | Depends on | Nucleic |
| | Nanoparticles | | | what is | Acids and |
| | | | | delivered | Proteins |
| Biological | Attenuated | YES | Transient | NO | Nucleic |
| Non-Viral | Bacteria | | | | Acids |
| Delivery | Engineered | YES | Transient | NO | Nucleic |
| Vehicles | Bacteriophages | | | | Acids |
| | Mammalian | YES | Transient | NO | Nucleic |
| | Virus-like | | | | Acids |
| | Particles | | | | |
| | Biological | YES | Transient | NO | Nucleic |
| | liposomes: | | | | Acids |
| | Erythrocyte | | | | |
| | Ghosts and | | | | |
| | Exosomes | | | | |

In another aspect, the delivery of prime editing composition components or polynucleotides encoding such components, for example, a Cas9 nickase, a DNA polymerase (e.g., DNA polymerase α, β, γ, δ, or ε), and a chimeric PE guide polynucleotide targeting a genomic nucleic acid sequence of interest, may be accomplished by delivering a ribonucleoprotein (RNP) to cells. The RNP comprises the nucleic acid binding protein, e.g., nCas9, in complex with the DNA polymerase and targeting chimeric PE guide polynucleotide. RNPs may be delivered to cells using known methods, such as electroporation, nucleofection, or cationic lipid-mediated methods, for example, as reported by Zuris, J. A. et al., 2015, Nat. Biotechnology, 33(1):73-80.

In some embodiments, a prime editing composition of the present disclosure is of small enough size to allow separate promoters to drive expression of the prime editor and a compatible chimeric PE guide polynucleotide within the same nucleic acid molecule. For instance, a vector or viral vector can comprise a first promoter operably linked to a nucleic acid encoding the prime editing composition and a second promoter operably linked to the chimeric PE guide polynucleotide.

A prime editing composition or components thereof described herein can be delivered with viral vectors. In some embodiments, a prime editing composition component, e.g., a prime editor, disclosed herein can be encoded on a nucleic acid that is contained in a viral vector. In some embodiments, one or more components of the prime editing composition can be encoded on one or more viral vectors. For example, a prime editing composition comprising a DNA polymerase, a napDNAbp, and chimeric PE guide polynucleotide can be encoded on a single viral vector. In some embodiments, the prime editing composition comprising a DNA polymerase, a napDNAbp, and chimeric PE guide polynucleotide can be encoded on different viral vectors. In either case, the prime editing composition components, e.g., a DNA polymerase, a napDNAbp, and chimeric PE guide polynucleotide can each be operably linked to a promoter and terminator. The combination of prime editing composition components encoded on a viral vector can be determined by the cargo size constraints of the chosen viral vector.

The use of RNA or DNA viral based systems for the delivery of a prime editing composition or a component thereof takes advantage of highly evolved processes for targeting a virus to specific cells in culture or in the host and trafficking the viral payload to the nucleus or host cell genome. Viral vectors can be administered directly to cells in culture, patients (in vivo), or they can be used to treat cells in vitro, and the modified cells can optionally be administered to patients (ex vivo). Conventional viral based systems could include retroviral, lentivirus, adenoviral, adeno-associated and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

Methods of generating vectors for viral delivery are known in the art. In some embodiments, packaging cells are used to form virus particles that are capable of infecting a host cell. In some embodiments, the packaging cell is a HEK293T cell, (e.g., for packaging of adenovirus) In some embodiments, the packaging cell is a ψ2 cell or a PA317 cell (e.g., for packaging of retrovirus). In some embodiments, the viral vectors contain the minimal viral sequences required for packaging and/or subsequent integration into a host, other viral sequences being replaced by an expression cassette for the polynucleotide(s) to be expressed. In some embodiments, the missing viral functions are supplied in trans by the packaging cell line. In some embodiments, the packaging cell line includes helper plasmids that encode proteins required for viral functions.

Viral vectors can include those derived from lentivirus (e.g., HIV and FIV-based vectors), Adenovirus (e.g., AD100), Retrovirus (e.g., gamma retrovirus, Maloney murine leukemia virus, MML-V), herpesvirus vectors (e.g., HSV-2), and Adeno-associated viruses (AAVs), or other plasmid or viral vector types, in particular, using formulations and doses from, for example, U.S. Pat. No. 8,454,972 (formulations, doses for adenovirus), U.S. Pat. No. 8,404,658 (formulations, doses for AAV) and U.S. Pat. No. 5,846,946 (formulations, doses for DNA plasmids) and from clinical trials and publications regarding the clinical trials involving lentivirus, AAV and adenovirus. For example, for AAV, the route of administration, formulation and dose can be as in U.S. Pat. No. 8,454,972 and as in clinical trials involving AAV. For Adenovirus, the route of administration, formulation and dose can be as in U.S. Pat. No. 8,404,658 and as in clinical trials involving adenovirus. For plasmid delivery, the route of administration, formulation and dose can be as in U.S. Pat. No. 5,846,946 and as in clinical studies involving plasmids. Doses can be based on or extrapolated to an average 70 kg individual (e.g., a male adult human), and can be adjusted for patients, subjects, mammals of different weight and species. Frequency of administration is within the ambit of the medical or veterinary practitioner (e.g., physician, veterinarian), depending on usual factors including the age, sex, general health, other conditions of the patient or subject and the particular condition or symptoms being addressed. The viral vectors can be injected into the tissue of interest. For cell-type specific editing, the expression of the prime editor and/or PE guide polynucleotide can be driven by a cell-type specific promoter.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SIV), human immuno deficiency virus (HIV), and combinations thereof (See, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); PCT/US94/05700).

Retroviral vectors, especially lentiviral vectors, can require polynucleotide sequences smaller than a given length for efficient integration into a target cell. For example, retroviral vectors of length greater than 9 kb can result in low viral titers compared with those of smaller size. In some embodiments, all or some components of a prime editing composition of the present disclosure is of sufficient size so as to enable efficient packaging and delivery into a target cell via a retroviral vector. In some embodiments, a prime editor and/or a PE guide polynucleotide are of a size so as to allow efficient packing and delivery even when expressed together with a guide nucleic acid and/or other components of a targetable nuclease system.

In applications where transient expression is preferred, adenoviral based systems can be used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors can also be used to transduce cells with target nucleic acids, e.g., in the in vitro production of nucleic acids and peptides, and for in vivo and ex vivo gene therapy procedures (See, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). The construction of recombinant AAV vectors is described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989).

AAV is a small, single-stranded DNA dependent virus belonging to the parvovirus family. The 4.7 kb wild-type (wt) AAV genome is made up of two genes that encode four replication proteins and three capsid proteins, respectively, and is flanked on either side by 145-bp inverted terminal repeats (ITRs). The virion is composed of three capsid proteins, Vp1, Vp2, and Vp3, produced in a 1:1:10 ratio from the same open reading frame but from differential splicing (Vp1) and alternative translational start sites (Vp2 and Vp3, respectively). Vp3 is the most abundant subunit in the virion and participates in receptor recognition at the cell surface defining the tropism of the virus. A phospholipase domain, which functions in viral infectivity, has been identified in the unique N terminus of Vp1.

Similar to wt AAV, recombinant AAV (rAAV) utilizes the cis-acting 145-bp ITRs to flank vector transgene cassettes, providing up to 4.5 kb for packaging of foreign DNA. Subsequent to infection, rAAV can express a prime editor fusion protein of the disclosure and persist without integration into the host genome by existing episomally in circular head-to-tail concatemers. Although there are numerous examples of rAAV success using this system, in vitro and in vivo, the limited packaging capacity has limited the use of AAV-mediated gene delivery when the length of the coding sequence of the gene is equal or greater in size than the wt AAV genome.

Viral vectors can be selected based on the application. For example, for in vivo gene delivery, AAV can be advantageous over other viral vectors. In some embodiments, AAV allows low toxicity, which can be due to the purification method not requiring ultra-centrifugation of cell particles that can activate the immune response. In some embodiments, AAV allows low probability of causing insertional mutagenesis because it doesn't integrate into the host genome. Adenoviruses are commonly used as vaccines because of the strong immunogenic response they induce.

In some embodiments, a polynucleotide encoding a prime editor and/or a PE guide polynucleotide is less than 4 kb. In some embodiments, a polynucleotide encoding a prime editor and/or a PE guide polynucleotide is less than 4.5 kb, 4.4 kb, 4.3 kb, 4.2 kb, 4.1 kb, 4 kb, 3.9 kb, 3.8 kb, 3.7 kb, 3.6 kb, 3.5 kb, 3.4 kb, 3.3 kb, 3.2 kb, 3.1 kb, 3 kb, 2.9 kb, 2.8 kb, 2.7 kb, 2.6 kb, 2.5 kb, 2 kb, or 1.5 kb. In some embodiments, a polynucleotide encoding a prime editor and/or a PE guide polynucleotide is 4.5 kb or less in length.

An AAV can be AAV1, AAV2, AAV5 or any combination thereof. One can select the type of AAV with regard to the cells to be targeted; e.g., one can select AAV serotypes 1, 2, 5 or a hybrid capsid AAV1, AAV2, AAV5 or any combination thereof for targeting brain or neuronal cells; and one can select AAV4 for targeting cardiac tissue. AAV8 is useful for delivery to the liver. A tabulation of certain AAV serotypes as to these cells can be found in Grimm, D. et al, J. Virol. 82: 5887-5911 (2008)).

In some embodiments, a prim editor polypeptide, e.g., a prime editor fusion protein, is delivered into two or more fragments, wherein the N-terminal fragment is fused to a split intein-N and the C-terminal fragment is fused to a split intein-C. In some embodiments, the N-terminal fragment fused to split intein-N and the C terminal fragment fused to split intein-C are then packaged into two or more AAV vectors. As used herein, “intein” refers to a self-splicing protein intron (e.g., peptide) that ligates flanking N-terminal and C-terminal exteins (e.g., fragments to be joined). The use of certain inteins for joining heterologous protein fragments is described, for example, in Wood et al., J. Biol. Chem. 289(21); 14512-9 (2014). For example, when fused to separate protein fragments, the inteins IntN and IntC recognize each other, splice themselves out and simultaneously ligate the flanking N- and C-terminal exteins of the protein fragments to which they were fused, thereby reconstituting a full-length protein from the two protein fragments. Other suitable inteins will be apparent to a person of skill in the art.

An N-terminal fragment or C-terminal fragment of a prime editor fusion protein can vary in length. In some embodiments, an N-terminal fragment or C-terminal fragment of a prime editor fusion protein ranges from 2 amino acids to about 1000 amino acids in length. In some embodiments, the N-terminal fragment or the C-terminal fragment of a prime editor fusion protein ranges from about 5 amino acids to about 500 amino acids in length, from about 20 amino acids to about 200 amino acids in length, or from about 10 amino acids to about 100 amino acids in length. Suitable protein fragments of other lengths will be apparent to a person of skill in the art.

In some embodiments, dual AAV vectors are generated by splitting a large transgene expression cassette, e.g., an expression cassette that encodes a prime editor fusion protein disclosed herein that comprises a DNA binding domain and a DNA polymerase domain, in two separate portions (5′ and 3′ ends, or head and tail), where each portion of the cassette is packaged in a single AAV vector (of <5 kb). The re-assembly of the full-length transgene expression cassette can then be achieved upon co-infection of the same cell by both dual AAV vectors followed by: (1) homologous recombination (HR) between 5′ and 3′ genomes (dual AAV overlapping vectors); (2) ITR-mediated tail-to-head concatemerization of 5′ and 3′ genomes (dual AAV trans-splicing vectors); or (3) a combination of these two mechanisms (dual AAV hybrid vectors). The use of dual AAV vectors in vivo results in the expression of full-length proteins. The use of the dual AAV vector platform represents an efficient and viable gene transfer strategy for transgenes of >4.7 kb in size.

Lentiviruses are complex retroviruses that have the ability to infect and express their genes in both mitotic and post-mitotic cells. The most commonly known lentivirus is the human immunodeficiency virus (HIV), which uses the envelope glycoproteins of other viruses to target a broad range of cell types.

Lentiviruses can be prepared as follows. After cloning pCasES10 (which contains a lentiviral transfer plasmid backbone), HEK293FT at low passage (p=5) were seeded in a T-75 flask to 50% confluence the day before transfection in DMEM with 10% fetal bovine serum and without antibiotics. After 20 hours, media is changed to OptiMEM (serum-free) media and transfection was done 4 hours later. Cells are transfected with 10 μg of lentiviral transfer plasmid (pCasES10) and the following packaging plasmids: 5 μg of pMD2.G (VSV-g pseudotype), and 7.5 μg of psPAX2 (gag/pol/rev/tat). Transfection can be done in 4 mL OptiMEM with a cationic lipid delivery agent (50 μl Lipofectamine 2000 and 100 ul Plus reagent). After 6 hours, the media is changed to antibiotic-free DMEM with 10% fetal bovine serum. These methods use serum during cell culture, but serum-free methods are preferred.

Lentivirus can be purified as follows. Viral supernatants are harvested after 48 hours. Supernatants are first cleared of debris and filtered through a 0.45 μm low protein binding (PVDF) filter. They are then spun in an ultracentrifuge for 2 hours at 24,000 rpm. Viral pellets are resuspended in 50 μl of DMEM overnight at 4° C. They are then aliquoted and immediately frozen at −80° C.

In another embodiment, minimal non-primate lentiviral vectors based on the equine infectious anemia virus (EIAV) are also contemplated. In another embodiment, RetinoStat®, an equine infectious anemia virus-based lentiviral gene therapy vector that expresses angiostatic proteins endostatin and angiostatin that is contemplated to be delivered via a subretinal injection. In another embodiment, use of self-inactivating lentiviral vectors are contemplated.

Any RNA of the prime editing composition, for example a PE guide RNA, a chimeric PE guide polynucleotide, or a prime editor-encoding mRNA, can be delivered in the form of RNA. Prime editor-encoding mRNA can be generated using in vitro transcription. For example, nuclease mRNA can be synthesized using a PCR cassette containing the following elements: T7 promoter, optional kozak sequence (GCCACC), nuclease sequence, and 3′ UTR such as a 3′ UTR from beta globin-polyA tail. The cassette can be used for transcription by T7 polymerase. Guide polynucleotides (e.g., gRNA) can also be transcribed using in vitro transcription from a cassette containing a T7 promoter, followed by the sequence “GG”, and guide polynucleotide sequence.

### Pharmaceutical Compositions

Other aspects of the present disclosure relate to pharmaceutical compositions comprising any of the prime editing compositions, prime editor or prime editor fusion proteins, or the prime editor-PE guide polynucleotide complexes described herein. The term “pharmaceutical composition,” as used herein, refers to a composition formulated for pharmaceutical use. In some embodiments, the pharmaceutical composition further comprises a pharmaceutically acceptable carrier. In some embodiments, the pharmaceutical composition comprises additional agents (e.g., for specific delivery, increasing half-life, or other therapeutic compounds).

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the compound from one site (e.g., the delivery site) of the body, to another site (e.g., organ, tissue or portion of the body). A pharmaceutically acceptable carrier is “acceptable” in the sense of being compatible with the other ingredients of the formulation and not injurious to the tissue of the subject (e.g., physiologically compatible, sterile, physiologic pH, etc.).

Some nonlimiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient,” “carrier,” “pharmaceutically acceptable carrier,” “vehicle” or the like are used interchangeably herein.

Pharmaceutical compositions can comprise one or more pH buffering compounds to maintain the pH of the formulation at a predetermined level that reflects physiological pH, such as in the range of about 5.0 to about 8.0. The pH buffering compound used in the aqueous liquid formulation can be an amino acid or mixture of amino acids, such as histidine or a mixture of amino acids such as histidine and glycine. Alternatively, the pH buffering compound is preferably an agent which maintains the pH of the formulation at a predetermined level, such as in the range of about 5.0 to about 8.0, and which does not chelate calcium ions. Illustrative examples of such pH buffering compounds include, but are not limited to, imidazole and acetate ions. The pH buffering compound may be present in any amount suitable to maintain the pH of the formulation at a predetermined level.

Pharmaceutical compositions can also contain one or more osmotic modulating agents, i.e., a compound that modulates the osmotic properties (e.g., tonicity, osmolality, and/or osmotic pressure) of the formulation to a level that is acceptable to the blood stream and blood cells of recipient individuals. The osmotic modulating agent can be an agent that does not chelate calcium ions. The osmotic modulating agent can be any compound known or available to those skilled in the art that modulates the osmotic properties of the formulation. One skilled in the art may empirically determine the suitability of a given osmotic modulating agent for use in the inventive formulation. Illustrative examples of suitable types of osmotic modulating agents include, but are not limited to: salts, such as sodium chloride and sodium acetate; sugars, such as sucrose, dextrose, and mannitol; amino acids, such as glycine; and mixtures of one or more of these agents and/or types of agents. The osmotic modulating agent(s) may be present in any concentration sufficient to modulate the osmotic properties of the formulation.

In some embodiments, the pharmaceutical composition is formulated for delivery to a subject, e.g., for gene editing. Suitable routes of administrating the pharmaceutical composition described herein include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration.

In some embodiments, the pharmaceutical composition described herein is administered locally to a diseased site (e.g., tumor site). In some embodiments, the pharmaceutical composition described herein is administered to a subject by injection, by means of a catheter, by means of a suppository, or by means of an implant, the implant being of a porous, non-porous, or gelatinous material, including a membrane, such as a sialastic membrane, or a fiber.

In other embodiments, the pharmaceutical composition described herein is delivered in a controlled release system. In one embodiment, a pump can be used (see, e.g., Langer, 1990, Science 249: 1527-1533; Sefton, 1989, CRC Crit. Ref. Biomed. Eng. 14:201; Buchwald et al., 1980, Surgery 88:507; Saudek et al, 1989, N. Engl. J. Med. 321:574). In another embodiment, polymeric materials can be used. (See, e.g., Medical Applications of Controlled Release (Langer and Wise eds., CRC Press, Boca Raton, Fla., 1974); Controlled Drug Bioavailability, Drug Product Design and Performance (Smolen and Ball eds., Wiley, New York, 1984); Ranger and Peppas, 1983, Macromol. Sci. Rev. Macromol. Chem. 23:61. See also Levy et al., 1985, Science 228: 190; During et al., 1989, Ann. Neurol. 25:351; Howard et ah, 1989, J. Neurosurg. 71: 105.) Other controlled release systems are discussed, for example, in Langer, supra.

In some embodiments, the pharmaceutical composition is formulated in accordance with routine procedures as a composition adapted for intravenous or subcutaneous administration to a subject, e.g., a human. In some embodiments, pharmaceutical composition for administration by injection are solutions in sterile isotonic use as solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachet indicating the quantity of active agent. Where the pharmaceutical is to be administered by infusion, it can be dispensed with an infusion bottle containing sterile pharmaceutical grade water or saline. Where the pharmaceutical composition is administered by injection, an ampoule of sterile water for injection or saline can be provided so that the ingredients can be mixed prior to administration.

A pharmaceutical composition for systemic administration can be a liquid, e.g., sterile saline, lactated Ringer's or Hank's solution. In addition, the pharmaceutical composition can be in solid forms and re-dissolved or suspended immediately prior to use. Lyophilized forms are also contemplated. The pharmaceutical composition can be contained within a lipid particle or vesicle, such as a liposome or microcrystal, which is also suitable for parenteral administration. The particles can be of any suitable structure, such as unilamellar or plurilamellar, so long as compositions are contained therein. Compounds can be entrapped in “stabilized plasmid-lipid particles” (SPLP) containing the fusogenic lipid dioleoylphosphatidylethanolamine (DOPE), low levels (5-10 mol %) of cationic lipid, and stabilized by a polyethyleneglycol (PEG) coating (Zhang Y. P. et ah, Gene Ther. 1999, 6: 1438-47). Positively charged lipids such as N-[1-(2,3-dioleoyloxi)propyl]-N,N,N-trimethyl-amoniummethylsulfate, or “DOTAP,” are particularly preferred for such particles and vesicles. The preparation of such lipid particles is well known. See, e.g., U.S. Pat. Nos. 4,880,635; 4,906,477; 4,911,928; 4,917,951; 4,920,016; and 4,921,757; each of which is incorporated herein by reference.

The pharmaceutical composition described herein can be administered or packaged as a unit dose, for example. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent; i.e., carrier, or vehicle.

Further, the pharmaceutical composition can be provided as a pharmaceutical kit comprising (a) a container containing a compound of the disclosure in lyophilized form and (b) a second container containing a pharmaceutically acceptable diluent (e.g., sterile used for reconstitution or dilution of the lyophilized compound of the disclosure. Optionally associated with such container(s) can be a notice in the form prescribed by a governmental agency regulating the manufacture, use or sale of pharmaceuticals or biological products, which notice reflects approval by the agency of manufacture, use or sale for human administration.

In another aspect, an article of manufacture containing materials useful for the treatment of the diseases described above is included. In some embodiments, the article of manufacture comprises a container and a label. Suitable containers include, for example, bottles, vials, syringes, and test tubes. The containers can be formed from a variety of materials such as glass or plastic. In some embodiments, the container holds a composition that is effective for treating a disease described herein and can have a sterile access port. For example, the container can be an intravenous solution bag or a vial having a stopper pierceable by a hypodermic injection needle. The active agent in the composition is a compound of the disclosure. In some embodiments, the label on or associated with the container indicates that the composition is used for treating the disease of choice. The article of manufacture can further comprise a second container comprising a pharmaceutically-acceptable buffer, such as phosphate-buffered saline, Ringer's solution, or dextrose solution. It can further include other materials desirable from a commercial and user standpoint, including other buffers, diluents, filters, needles, syringes, and package inserts with instructions for use.

In some embodiments, any of the prime editors, chimeric PE guide polynucleotides, and/or prime editing compositions or complexes described herein are provided as part of a pharmaceutical composition. In some embodiments, the pharmaceutical composition comprises any of the prime editor fusion proteins provided herein. In some embodiments, the pharmaceutical composition comprises any of the prime editing composition provided herein. In some embodiments, the pharmaceutical composition comprises any of the chimeric PE guide polynucleotides provided herein. In some embodiments, the pharmaceutical composition comprises one or more polynucleotides, e.g., DNA or RNA, that encodes any of the prime editor polypeptide or prime editor fusion protein, or chimeric PE guide polynucleotide provided herein. In some embodiments, the pharmaceutical composition comprises any of the chimeric PE guide polynucleotides provided herein. In some embodiments, the pharmaceutical composition comprises a nucleic acid programmable DNA binding protein (napDNAbp) with nickase activity (e.g., nCas9) fused to, covalently or non-covalently attached to, or linked to a DNA polymerase (e.g., DNA polymerase α, β, γ, δ, or ε) and forms a complex with a chimeric PE guide polynucleotide (e.g., DNA-RNA or RNA-DNA guide). In some embodiments pharmaceutical composition comprises a guide polynucleotide, a napDNAbp, a DNA polymerase, and a pharmaceutically acceptable excipient. Pharmaceutical compositions can optionally comprise one or more additional therapeutically active substances.

In some embodiments, compositions provided herein are administered to a subject, for example, to a human subject, in order to effect a targeted genomic modification within the subject. In some embodiments, cells are obtained from the subject and contacted with any of the pharmaceutical compositions provided herein. In some embodiments, cells removed from a subject and contacted ex vivo with a pharmaceutical composition are re-introduced into the subject, optionally after the desired genomic modification has been effected or detected in the cells. Methods of delivering pharmaceutical compositions comprising nucleases are known, and are described, for example, in U.S. Pat. Nos. 6,453,242; 6,503,717; 6,534,261; 6,599,692; 6,607,882; 6,689,558; 6,824,978; 6,933,113; 6,979,539; 7,013,219; and 7,163,824, the disclosures of all of which are incorporated by reference herein in their entireties. Although the descriptions of pharmaceutical compositions provided herein are principally directed to pharmaceutical compositions which are suitable for administration to humans, it will be understood by the skilled artisan that such compositions are generally suitable for administration to animals or organisms of all sorts, for example, for veterinary use.

Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, domesticated animals, pets, and commercially relevant mammals such as cattle, pigs, horses, sheep, cats, dogs, mice, and/or rats; and/or birds, including commercially relevant birds such as chickens, ducks, geese, and/or turkeys.

Formulations of the pharmaceutical compositions described herein can be prepared by any method known or hereafter developed in the art of pharmacology. In general, such preparatory methods include the step of bringing the active ingredient(s) into association with an excipient and/or one or more other accessory ingredients, and then, if necessary and/or desirable, shaping and/or packaging the product into a desired single- or multi-dose unit. Pharmaceutical formulations can additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21st Edition, A. R. Gennaro (Lippincott, Williams & Wilkins, Baltimore, Md., 2006; incorporated in its entirety herein by reference) discloses various excipients used in formulating pharmaceutical compositions and known techniques for the preparation thereof. See also PCT application PCT/US2010/055131 (Publication number WO2011/053982 A8, filed Nov. 2, 2010), incorporated in its entirety herein by reference, for additional suitable methods, reagents, excipients and solvents for producing pharmaceutical compositions comprising a nuclease.

Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this disclosure.

The compositions, as described above, can be administered in effective amounts. The effective amount will depend upon the mode of administration, the particular condition being treated, and the desired outcome. It may also depend upon the stage of the condition, the age and physical condition of the subject, the nature of concurrent therapy, if any, and like factors well-known to the medical practitioner. For therapeutic applications, it is that amount sufficient to achieve a medically desirable result.

In some embodiments, compositions in accordance with the present disclosure can be used for treatment of any of a variety of diseases, disorders, and/or conditions.

### Kits

Various aspects of this disclosure provide kits comprising a prime editing composition. In some embodiments, the kit comprises a prime editor comprising a DNA polymerase (e.g., DNA polymerase α, β, γ, δ, or ε) and a nucleic acid programmable DNA binding protein (napDNAbp) having nickase activity (e.g., nCas9). In some embodiments, the kit comprises a prime editor fusion protein comprising a DNA polymerase (e.g., DNA polymerase α, β, γ, δ, or ε) and a nucleic acid programmable DNA binding protein (napDNAbp) having nickase activity (e.g., nCas9). In some embodiments, the kit comprises a vector encoding a prime editor fusion protein of a DNA polymerase (e.g., DNA polymerase α, β, γ, δ, or ε) and a nucleic acid programmable DNA binding protein (napDNAbp) having nickase activity (e.g., nCas9). In some embodiments, the kit comprises a vector encoding a prime editing composition or a component thereof as provided herein. In some embodiments, the kit comprises at least one chimeric PE guide polynucleotide capable of targeting a double-stranded target polynucleotide of interest. In some embodiments, the kit comprises a nucleic acid construct comprising a polynucleotide sequence encoding at least one chimeric PE guide polynucleotide. In some embodiments, the kit comprises a cell comprising a prime editing composition or a component thereof as provided herein. In some embodiments, the kit comprises a pharmaceutical composition comprising a prime editing composition as provided herein.

The kit provides, in some embodiments, instructions for using the kit to edit one or more polynucleotides. The instructions will generally include information about the use of the kit for editing nucleic acid molecules. In other embodiments, the instructions include at least one of the following: precautions; warnings; clinical studies; and/or references. The instructions may be printed directly on the container (when present), or as a label applied to the container, or as a separate sheet, pamphlet, card, or folder supplied in or with the container. In a further embodiment, a kit can comprise instructions in the form of a label or separate insert (package insert) for suitable operational parameters. In yet another embodiment, the kit can comprise one or more containers with appropriate positive and negative controls or control samples, to be used as standard(s) for detection, calibration, or normalization. The kit can further comprise a second container comprising a pharmaceutically-acceptable buffer, such as (sterile) phosphate-buffered saline, Ringer's solution, or dextrose solution. It can further include other materials desirable from a commercial and user standpoint, including other buffers, diluents, filters, needles, syringes, and package inserts with instructions for use.

The practice of the present disclosure employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the disclosure, and, as such, may be considered in making and practicing the disclosure. Particularly useful techniques for particular embodiments will be discussed in the sections that follow.

The following examples are put forth so as to provide those of ordinary skill in the art with a complete disclosure and description of how to make and use the assay, screening, and therapeutic methods of the disclosure and are not intended to limit the scope of what the inventors regard as their invention.

### EXAMPLES

### Example 1: Identification of Polymerase Editor Complexes

An expression vector is designed to encode a prime editor fusion protein comprising a DNA polymerase fused to a napDNAbp having nickase activity. The DNA polymerase can be a DNA Pol α, β, γ, δ, or ε. The napDNAbp can be a Cas9 nickase (nCas9). The expression vector is co-transformed into competent host cells with a selection plasmid. The host cells can be of a known cell type, for example, mammalian cells, yeast cells, insect cells, or bacteria cells. As an example, the expression vector is co-transformed into HEK293T cells with a selection plasmid. After transformation, the host cells are selected for restoration of expression of a selection marker of the selection plasmid, which is indicative of polymerase activity.

A host cell line is established to harbor an antibiotic resistance gene, which contains a nucleotide mutation that renders the gene non-functional. A chimeric prime editing guide polynucleotide, or chipGNA, is designed to incorporate a nucleotide edit in the target antibiotic resistance gene to correct the nucleotide mutation and restore expression of the target gene to have antibiotic resistance. After polymerase activity is confirmed, the expression vector that encodes the prime editor fusion protein described above is co-transformed into the host cell line with the chipGNA. The cells are then plated onto a series of agarose plates with increasing selection concentration. Prime editors having efficient editing activity are able to correct the mutation in the antibiotic resistance gene and result in antibiotic resistance are selected for further analysis.

Lentiviral expression vectors encoding the prime editor fusion protein described above are generated. To examine prime editing in mammalian cells, HEK293T cells expressing a gene of interest that that contain a nucleotide polymorphism and encodes a protein is are used. A chipGNA is designed to target the nucleotide polymorphism in the gene of interest. The HEK293T cells are transduced with the chipGNA and the lentiviral vector expressing the prime editor fusion protein described above. After transduction, the HEK293T cells are cultured, and genomic DNA is harvested after 24, 48, 72, and 96 hours and sequenced with next generation sequencing. The prime editor editing efficiency and average indel frequency are assessed. To evaluate the specificity of editing, target and unintended or bystander edits are monitored.

Further analysis of selected editors is carried out in fibroblast cells expressing the protein of interest comprising one or more mutations. The prime editor editing efficiency and average indel frequency are assessed. To evaluate the specificity of editing, target and unintended or bystander edits are monitored.

### Example 2: Editing Double-Stranded Target Polynucleotide Target Sequences with Prime Editing Compositions

A prime editor comprising an nCas9 napDNAbp and a DNA polymerase is made as described in Example 1. The prime editor interacts with a chimeric DNA/RNA priming guide nucleic acid (chipGNA) capable of recognizing a double-stranded DNA target polynucleotide and programmed to edit one or more bases in the double-stranded DNA target sequence.

As shown in FIG. 1, the prime editor and chipGNA incorporates an edit in genomic DNA. The nCas9 napDNAbp binds to the chipGNA scaffold (e.g., in an RNA segment) to form a riboprotein complex, as shown in FIG. 1(i). The chipGNA directs the nCas9 to a specific location of a double-stranded DNA target polynucleotide (e.g., genomic DNA) to be edited through annealing of the spacer sequence in the chipGNA to the search target sequence on a target strand of the double stranded DNA target polynucleotide. The nCas9 then nicks the edit strand (the opposite strand of the target strand) of the double-stranded DNA target polynucleotide and generates a free 3′ end on the edit strand. Subsequently, the primer binding site sequence (PBS) in the extension arm of the chipGNA (e.g., in a DNA segment) binds to a complementary portion of the nicked strand, as shown in FIG. 1(ii). Next, the DNA polymerase of the prime editor synthesizes a new strand of DNA, primed by the sequence in the edit strand annealed with the PBS, using the editing template sequence of the DNA segment of the chipGNA as a synthesis template, as shown in FIG. 1(iii). Subsequently, the edit is incorporated incorporate the edit in the genomic DNA permanently (e.g., via DNA repair mechanism involving DNA repair proteins such as FEN1 and DNA ligase) FIG. 1(iv)). FIG. 1(v) shows the resulting stably edited genomic DNA sequence.

A chipGNA may be bound to a prime editor comprising a napDNA nickase (e.g., Cas9 nickase or nCas9) and a DNA polymerase not fused to the programmable nickase, as shown in FIG. 2. The prime editor may be made essentially as described in Example 1 except that the expression vector encodes the nCas9 and, optionally, the DNA polymerase. If the expression vector is used to express both nCas9 and a DNA polymerase, the nucleotide sequences encoding the two proteins are separated by an IRES or P2A site. In some embodiments, the DNA polymerase is endogenously expressed. As described above, the chipGNA directs the nCas9 to a specific location of a double-stranded DNA target polynucleotide, nCas9 then nicks the edit strand, the DNA polymerase synthesizes a new strand of DNA primed by the sequence in the edit strand annealed with the PBS. Subsequently, the edit is incorporated incorporate the edit in the genomic DNA permanently (e.g., via DNA repair mechanism involving DNA repair proteins such as FEN1 and DNA ligase).

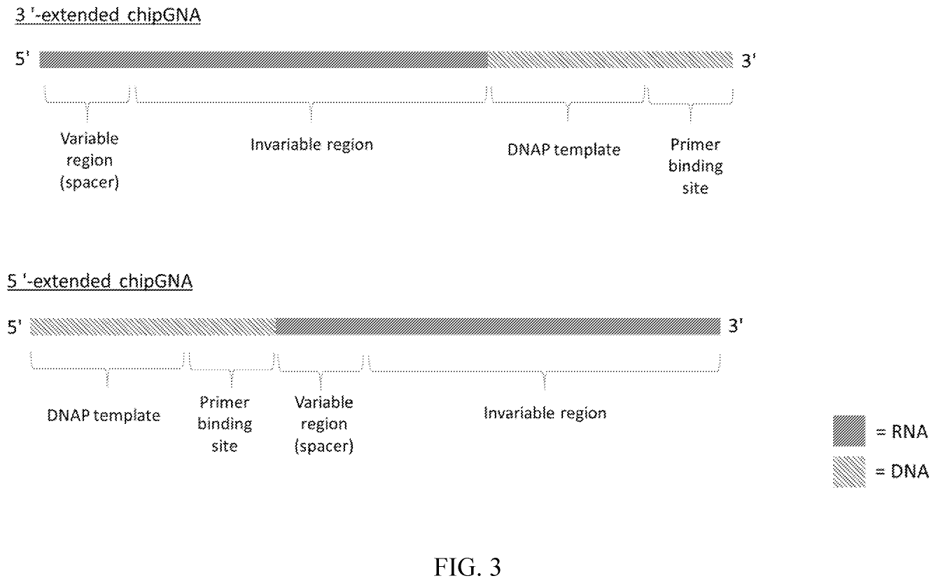

Different types of chipGNAs were designed for editing the double-stranded DNA target sequence. An RNA-DNA PE guide polynucleotide with a 5′ RNA segment and a 3′ DNA segment (FIG. 3, Top) was designed to edit a double-stranded DNA target sequence. A DNA-RNA PE guide polynucleotide with a 5′ DNA segment and a 3′ RNA segment (FIG. 3, Bottom) was also designed to edit a double-stranded DNA target sequence. The DNA segment may contain a primer binding site that is at least partially complementary to a portion of an edit strand of a double-stranded target DNA. The DNA segment may also contain an editing template capable of priming the DNA polymerase and contains one or more intended nucleotide edits to be incorporated into the DNA target sequence. The RNA segment may contain a variable (spacer) region that is at least partially complementary to a portion of a non-edit strand of the double-stranded target polynucleotide. The RNA segment may also a constant (invariable) region that is capable of binding to the napDNAbp (e.g., nCas9). The RNA and/or DNA bases of the chipGNA may also be modified (e.g., 2′ O-Me modifications).

As shown in FIG. 3 (Top) an RNA-DNA guide was designed from 5′ to 3′ to include i) an RNA segment comprising a) a variable (spacer) region; and b) a scaffold region; and ii) a DNA segment comprising a) an editing template; and b) a primer binding site. As shown in FIG. 3 (Bottom) an DNA-RNA guide was designed from 5′ to 3′ to include i) a DNA segment comprising a) an editing template; and b) a primer binding site; and ii) an RNA segment comprising a) a variable (spacer) region; and b) a constant (invariable) region.

From the foregoing description, it will be apparent that variations and modifications may be made to the disclosure described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All publications, patents, and patent applications mentioned in this specification are herein incorporated by reference to the same extent as if each individual publication, patent, or patent application was specifically and individually indicated to be incorporated by reference. Absent any indication otherwise, publications, patents, and patent applications mentioned in this specification are incorporated herein by reference in their entireties.

1. A method of editing a double-stranded target polynucleotide comprising
contacting the double-stranded target polynucleotide with a chimeric prime editing guide polynucleotide (chimeric PEg polynucleotide), a nucleic acid programmable DNA binding protein (napDNAbp) having nickase activity, and a DNA polymerase;
wherein the double-stranded target polynucleotide comprises a target strand and an edit strand;
wherein the chimeric PEg polynucleotide comprises i) a deoxyribonucleic acid (DNA) segment comprising one or more intended nucleotide edits to be incorporated into the double-stranded target polynucleotide and is at least partially complementary to a portion of the edit strand of the double-stranded target polynucleotide, and ii) a ribonucleic acid (RNA) segment capable of binding to the napDNAbp and is at least partially complementary to a portion of the target strand of the double-stranded target polynucleotide;
wherein the napDNAbp results in a nick in the edit strand of the double-stranded target polynucleotide;
wherein the RNA segment hybridizes to the nicked edit strand of the double-stranded target polynucleotide; and wherein the DNA polymerase synthesizes a single stranded DNA that replaces an editing target sequence in the edit strand of the double-stranded target polynucleotide, thereby editing the double-stranded target polynucleotide.

2. A method of editing a double-stranded target polynucleotide comprising
(a) contacting the double-stranded target polynucleotide with a chimeric prime editing guide polynucleotide (chimeric PEg polynucleotide), a nucleic acid programmable DNA binding protein (napDNAbp) having nickase activity, and a DNA polymerase;
wherein the double-stranded target polynucleotide comprises a target strand and an edit strand;
wherein the chimeric PEg polynucleotide comprises a ribonucleic acid (RNA) segment comprising a variable region and an invariable region, and a deoxyribonucleic acid (DNA) segment comprising an editing template and a primer binding site (PBS);
wherein the variable region is at least partially complementary to a portion of the target strand of the double-stranded target polynucleotide;
wherein the invariable region is capable of binding to the napDNAbp;
wherein the primer binding site is at least partially complementary to a portion of the edit strand of the double-stranded target polynucleotide;
wherein the editing template comprises one or more intended nucleotide edits to be incorporated into the double-stranded target polynucleotide;
wherein the napDNAbp results in a nick in the edit strand of the double-stranded target polynucleotide;
wherein the PBS hybridizes to the nicked edit strand of the double-stranded target polynucleotide; and wherein the DNA polymerase synthesizes a single stranded DNA encoded by the editing template; wherein the single stranded DNA replaces an editing target sequence in the edit strand of the double-stranded target polynucleotide, thereby altering the double-stranded target polynucleotide.

3. The method of claim 2, wherein the nicking the edit strand of the double-stranded target polynucleotide with the napDNAbp forms 5′ and 3′ ends.

4. The method of claim 3, wherein the PBS hybridizes to the 3′ end of the nicked edit strand of the double-stranded target polynucleotide.

5. The method of claim 4, wherein the DNA polymerase extends the 3′ end of the nicked edit strand thereby altering the double-stranded target polynucleotide.

6. The method of claim 5, further comprising repairing the double-stranded target polynucleotide with a DNA repair protein to.

7. The method of claim 6, wherein the DNA repair protein is FEN1 or a DNA ligase.

8. The method of claim 7, further comprising nicking the target strand of the double-stranded target polynucleotide with a nickase to promote incorporation of the one or more intended nucleotide edits.

9. The method of claim 8, wherein the RNA segment is at the 5′ end of the chimeric PEg polynucleotide and the DNA segment is at the 3′ end of the chimeric PE guide polynucleotide.

10. The method of claim 9, wherein the DNA segment is at the 5′ end of the chimeric PEg polynucleotide and the RNA segment is at the 3′ end of the chimeric PEg polynucleotide.

11. The method of claim 10, wherein the chimeric PEg polynucleotide comprises from 5′ to 3′:
i) the RNA segment comprising
a) the variable region; and
b) the invariable region; and
ii) the DNA segment comprising
a) the editing template; and
b) the primer binding site.

12. The method of claim 11, wherein the chimeric PEg polynucleotide comprises from 5′ to 3′:
i) the DNA segment comprising
a) the editing template; and
b) the primer binding site; and
ii) the RNA segment comprising
a) the variable region; and
b) the invariable region.

13. The method of claim 12, wherein the one or more intended nucleotide edits comprises one or more substitution, an insertion, a deletion, and/or a modification.

14. The method of claim 13, wherein an edited double-stranded target polynucleotide comprises 1, 2, 3, 4, 5, 6, 7, 8, 9, 10, or more nucleotides that differ from a double-stranded target polynucleotide that is not edited.

15. The method of claim 14, wherein the edited double-stranded target polynucleotide comprises two or more nucleotides that differ from the double-stranded target polynucleotide that is not edited.

16. The method of claim 15, wherein any of the two or more nucleotides of the edited double-stranded target polynucleotide are consecutive nucleotides.

17. The method of claim 15, wherein any of the two or more nucleotides of the edited double-stranded target polynucleotide are non-consecutive nucleotides.

18. The method of claim 17, wherein the DNA segment comprises 1, 2, 3, 4, 5, 6, 7, 8, 9, 10, 11, 12, 13, 14, 15, 16, 17, 18, 19, 20, 30, 40, 50, 60, 70, 80, 90, 100 or more nucleotides to be inserted into the double-stranded target polynucleotide.

19. The method of claim 18, wherein the edited double-stranded target polynucleotide comprises a deletion of one or more nucleotides compared to the double-stranded target polynucleotide that is not edited.

20. The method of claim 19, wherein the napDNAbp or the DNA polymerase is associated with at least one nuclear localization sequence (NLS), or wherein the napDNAbp and the DNA polymerase are each bound to at least one nuclear localization sequence (NLS).

21. The method of any one of claims 1-19, wherein the napDNAbp and the DNA polymerase are each bound to at least one nuclear localization sequence (NLS).

21-135. (canceled)


SEQUENCE LISTING (82 sequences)

SEQ ID NO: 1 - DNA, 6 bp, Artificial Sequence
  Description of Artificial Sequence Synthetic oligonucleotide
  modified_base (1)..(2): a, c, t, g, unknown or other
nngrrt

SEQ ID NO: 2 - DNA, 6 bp, Artificial Sequence
  Description of Artificial Sequence Synthetic oligonucleotide
  modified_base (1)..(2): a, c, t, g, unknown or other
  modified_base (6)..(6): a, c, t, g, unknown or other
nngrrn

SEQ ID NO: 3 - DNA, 6 bp, Artificial Sequence
  Description of Artificial Sequence Synthetic oligonucleotide
  modified_base (1)..(3): a, c, t, g, unknown or other
nnnrrt

SEQ ID NO: 4 - DNA, 8 bp, Artificial Sequence
  Description of Artificial Sequence Synthetic oligonucleotide
  modified_base (1)..(4): a, c, t, g, unknown or other
nnnngatt

SEQ ID NO: 5 - DNA, 7 bp, Artificial Sequence
  Description of Artificial Sequence Synthetic oligonucleotide
  modified_base (1)..(2): a, c, t, g, unknown or other
nnagaaw

SEQ ID NO: 6 - DNA, 6 bp, Artificial Sequence
  Description of Artificial Sequence Synthetic oligonucleotide
  modified_base (1)..(1): a, c, t, g, unknown or other
naaaac

SEQ ID NO: 7 - protein, 18 aa, Artificial Sequence
  Description of Artificial Sequence Synthetic peptide
KRTADGSEFESPKKKRKV

SEQ ID NO: 8 - protein, 16 aa, Artificial Sequence
  Description of Artificial Sequence Synthetic peptide
KRPAATKKAGQAKKKK

SEQ ID NO: 9 - protein, 17 aa, Artificial Sequence
  Description of Artificial Sequence Synthetic peptide
KKTELQTTNAENKTKKL

SEQ ID NO: 10 - protein, 19 aa, Artificial Sequence
  Description of Artificial Sequence Synthetic peptide
KRGINDRNFWRGENGRKTR

SEQ ID NO: 11 - protein, 16 aa, Artificial Sequence
  Description of Artificial Sequence Synthetic peptide
RKSGKIAAIVVKRPRK

SEQ ID NO: 12 - protein, 7 aa, Artificial Sequence
  Description of Artificial Sequence Synthetic peptide
PKKKRKV

SEQ ID NO: 13 - protein, 30 aa, Artificial Sequence
  Description of Artificial Sequence Synthetic polypeptide
MDSLLMNRRKFLYQFKNVRWAKGRRETYLC

SEQ ID NO: 14 - protein, 4 aa, Artificial Sequence
  Description of Artificial Sequence Synthetic peptide
SGGS

SEQ ID NO: 15 - protein, 4 aa, Artificial Sequence
  Description of Artificial Sequence Synthetic peptide
GGGS

SEQ ID NO: 16 - protein, 5 aa, Artificial Sequence
  Description of Artificial Sequence Synthetic peptide
GGGGS

SEQ ID NO: 17 - protein, 5 aa, Artificial Sequence
  Description of Artificial Sequence Synthetic peptide
EAAAK

SEQ ID NO: 18 - protein, 16 aa, Artificial Sequence
  Description of Artificial Sequence Synthetic peptide
SGSETPGTSESATPES

SEQ ID NO: 19 - protein, 24 aa, Artificial Sequence
  Description of Artificial Sequence Synthetic peptide
SGGSSGGSSGSETPGTSESATPES

SEQ ID NO: 20 - RNA, 15 nt, Artificial Sequence
  Description of Artificial Sequence Synthetic oligonucleotide
aaauuaacaaacuaa

SEQ ID NO: 21 - RNA, 10 nt, Artificial Sequence
  Description of Artificial Sequence Synthetic oligonucleotide
aacaaacuaa

SEQ ID NO: 22 - RNA, 15 nt, Artificial Sequence
  Description of Artificial Sequence Synthetic oligonucleotide
uuuuuuuuuuuuuuu

SEQ ID NO: 23 - RNA, 11 nt, Artificial Sequence
  Description of Artificial Sequence Synthetic oligonucleotide
uuuuuuuuuuu

SEQ ID NO: 24 - DNA, 4104 bp, Streptococcus pyogenes
atggataagaaatactcaataggcttagatatcggcacaaatagcgtcggatgggcggtgatcactgatgattataaggttccgtctaaaaagttcaaggttctgggaaatacagaccgccacagtatcaaaaaaaatcttataggggctcttttatttggcagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggcagattctactgataaagcggatttgcgcttaatctatttggccttagcgcatatgattaagtttcgtggtcattttttgattgagggagatttaaatcctgataatagtgatgtggacaaactatttatccagttggtacaaatctacaatcaattatttgaagaaaaccctattaacgcaagtagagtagatgctaaagcgattctttctgcacgattgagtaaatcaagacgattagaaaatctcattgctcagctccccggtgagaagagaaatggcttgtttgggaatctcattgctttgtcattgggattgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatagtgaaataactaaggctcccctatcagcttcaatgattaagcgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgagggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggcgcctaccatgatttgctaaaaattattaaagataaagattttttggataatgaagaaaatgaagatatcttagaggatattgttttaacattgaccttatttgaagatagggggatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagatattcaaaaagcacaggtgtctggacaaggccatagtttacatgaacagattgctaacttagctggcagtcctgctattaaaaaaggtattttacagactgtaaaaattgttgatgaactggtcaaagtaatggggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctacaaaatggaagagacatgtatgtggaccaagaattagatattaatcgtttaagtgattatgatgtcgatcacattgttccacaaagtttcattaaagacgattcaatagacaataaggtactaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactga

SEQ ID NO: 25 - protein, 1367 aa, Streptococcus pyogenes
MDKKYSIGLDIGTNSVGWAVITDDYKVPSKKFKVLGNTDRHSIKKNLIGALLFGSGETAEATRLKRTARRRYTRRKNRICYLQEIFSNEMAKVDDSFFHRLEESFLVEEDKKHERHPIFGNIVDEVAYHEKYPTIYHLRKKLADSTDKADLRLIYLALAHMIKFRGHFLIEGDLNPDNSDVDKLFIQLVQIYNQLFEENPINASRVDAKAILSARLSKSRRLENLIAQLPGEKRNGLFGNLIALSLGLTPNFKSNFDLAEDAKLQLSKDTYDDDLDNLLAQIGDQYADLFLAAKNLSDAILLSDILRVNSEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGAYHDLLKIIKDKDFLDNEENEDILEDIVLTLTLFEDRGMIEERLKTYAHLFDDKVMKQLKRRRYTGWGRLSRKLINGIRDKQSGKTILDFLKSDGFANRNFMQLIHDDSLTFKEDIQKAQVSGQGHSLHEQIANLAGSPAIKKGILQTVKIVDELVKVMGHKPENIVIEMARENQTTQKGQKNSRERMKRIEEGIKELGSQILKEHPVENTQLQNEKLYLYYLQNGRDMYVDQELDINRLSDYDVDHIVPQSFIKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 26 - DNA, 4107 bp, Streptococcus pyogenes
atggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactga

SEQ ID NO: 27 - protein, 1368 aa, Streptococcus pyogenes [sequence omitted]

SEQ ID NO: 28 - protein, 1368 aa, Streptococcus pyogenes
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 29 - protein, 1367 aa, Streptococcus pyogenes
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 30 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 31 - protein, 1052 aa, Staphylococcus aureus
KRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 32 - protein, 1368 aa, Artificial Sequence
  Description of Artificial Sequence Synthetic polypeptide
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 33 - protein, 1410 aa, Artificial Sequence
  Description of Artificial Sequence Synthetic polypeptide
EIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFMQPTVAYSVLVVAKVEKGKSKKLKSVKELLGITIMERSSFEKNPIDFLEAKGYKEVKKDLIIKLPKYSLFELENGRKRMLASAKFLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPRAFKYFDTTIARKEYRSTKEVLDATLIHQSITGLYETRIDLSQLGGDGGSGGSGGSGGSGGSGGSGGMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEGADKRTADGSEFESPKKKRKV

SEQ ID NO: 34 - protein, 1367 aa, Artificial Sequence
  Description of Artificial Sequence Synthetic polypeptide
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTAFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGALSRKLINGIRDKQSGKTILDFLKSDGFANRNFMALIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRAITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 35 - protein, 1300 aa, Francisella novicida
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 36 - protein, 1299 aa, Francisella novicida
SIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 37 - protein, 1300 aa, Artificial Sequence
  Description of Artificial Sequence Synthetic polypeptide
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIARGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 38 - protein, 1300 aa, Artificial Sequence
  Description of Artificial Sequence Synthetic polypeptide
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFADLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 39 - protein, 1300 aa, Artificial Sequence
  Description of Artificial Sequence Synthetic polypeptide
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDAAANGAYHIGLKGLMLLGRIKNNQEGKKLNLVIKNEEYFEFVQNRNN

SEQ ID NO: 40 - protein, 1300 aa, Artificial Sequence
  Description of Artificial Sequence Synthetic polypeptide
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIARGERHLAYYTLVDGKGNIIKQDTFNIIGNDRMKTNYHDKLAAIEKDRDSARKDWKKINNIKEMKEGYLSQVVHEIAKLVIEYNAIVVFADLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 41 - protein, 1300 aa, Artificial Sequence
  Description of Artificial Sequence Synthetic polypeptide
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIARGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDAAANGAYHIGLKGLMLLGRIKNNQEGKKLNLVIKNEEYFEFVQNRNN

SEQ ID NO: 42 - protein, 1300 aa, Artificial Sequence
  Description of Artificial Sequence Synthetic polypeptide
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFADLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDAAANGAYHIGLKGLMLLGRIKNNQEGKKLNLVIKNEEYFEFVQNRNN

SEQ ID NO: 43 - protein, 1300 aa, Artificial Sequence
  Description of Artificial Sequence Synthetic polypeptide
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIARGERHLAYYTLVDGKGNIIKQDTFNIIGNDRMKTNYHDKLAAIEKDRDSARKDWKKINNIKEMKEGYLSQVVHEIAKLVIEYNAIVVFADLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDAAANGAYHIGLKGLMLLGRIKNNQEGKKLNLVIKNEEYFEFVQNRNN

SEQ ID NO: 44 - protein, 1129 aa, Alicyclobacillus acidoterrestris
MAVKSIKVKLRLDDMPEIRAGLWKLHKEVNAGVRYYTEWLSLLRQENLYRRSPNGDGEQECDKTAEECKAELLERLRARQVENGHRGPAGSDDELLQLARQLYELLVPQAIGAKGDAQQIARKFLSPLADKDAVGGLGIAKAGNKPRWVRMREAGEPGWEEEKEKAETRKSADRTADVLRALADFGLKPLMRVYTDSEMSSVEWKPLRKGQAVRTWDRDMFQQAIERMMSWESWNQRVGQEYAKLVEQKNRFEQKNFVGQEHLVHLVNQLQQDMKEASPGLESKEQTAHYVTGRALRGSDKVFEKWGKLAPDAPFDLYDAEIKNVQRRNTRRFGSHDLFAKLAEPEYQALWREDASFLTRYAVYNSILRKLNHAKMFATFTLPDATAHPIWTRFDKLGGNLHQYTFLFNEFGERRHAIRFHKLLKVENGVAREVDDVTVPISMSEQLDNLLPRDPNEPIALYFRDYGAEQHFTGEFGGAKIQCRRDQLAHMHRRRGARDVYLNVSVRVQSQSEARGERRPPYAAVFRLVGDNHRAFVHFDKLSDYLAEHPDDGKLGSEGLLSGLRVMSVDLGLRTSASISVFRVARKDELKPNSKGRVPFFFPIKGNDNLVAVHERSQLLKLPGETESKDLRAIREERQRTLRQLRTQLAYLRLLVRCGSEDVGRRERSWAKLIEQPVDAANHMTPDWREAFENELQKLKSLHGICSDKEWMDAVYESVRRVWRHMGKQVRDWRKDVRSGERPKIRGYAKDVVGGNSIEQIEYLERQYKFLKSWSFFGKVSGQVIRAEKGSRFAITLREHIDHAKEDRLKKLADRIIMEALGYVYALDERGKGKWVAKYPPCQLILLEELSEYQFNNDRPPSENNQLMQWSHRGVFQELINQAQVHDLLVGTMYAAFSSRFDARTGAPGIRCRRVPARCTQEHNPEPFPWWLNKFVVEHTLDACPLRADDLIPTGEGEIFVSPFSAEEGDFHQIHADLNAAQNLQQRLWSDFDISQIRLRCDWGEVDGELVLIPRLTGKRTADSYSNKVFYTNTGVTYYERERGKKRRKVFAQEKLSEEEAELLVEADEAREKSVVLMRDPSGIINRGNWTRQKEFWSMVNQRIEGYLVKQIRSRVPLQDSACENTGDI

SEQ ID NO: 45 - protein, 1108 aa, Bacillus hisashii
MATRSFILKIEPNEEVKKGLWKTHEVLNHGIAYYMNILKLIRQEAIYEHHEQDPKNPKKVSKAEIQAELWDFVLKMQKCNSFTHEVDKDEVFNILRELYEELVPSSVEKKGEANQLSNKFLYPLVDPNSQSGKGTASSGRKPRWYNLKIAGDPSWEEEKKKWEEDKKKDPLAKILGKLAEYGLIPLFIPYTDSNEPIVKEIKWMEKSRNQSVRRLDKDMFIQALERFLSWESWNLKVKEEYEKVEKEYKTLEERIKEDIQALKALEQYEKERQEQLLRDTLNTNEYRLSKRGLRGWREIIQKWLKMDENEPSEKYLEVFKDYQRKHPREAGDYSVYEFLSKKENHFIWRNHPEYPYLYATFCEIDKKKKDAKQQATFTLADPINHPLWVRFEERSGSNLNKYRILTEQLHTEKLKKKLTVQLDRLIYPTESGGWEEKGKVDIVLLPSRQFYNQIFLDIEEKGKHAFTYKDESIKFPLKGTLGGARVQFDRDHLRRYPHKVESGNVGRIYFNMTVNIEPTESPVSKSLKIHRDDFPKVVNFKPKELTEWIKDSKGKKLKSGIESLEIGLRVMSIDLGQRQAAAASIFEVVDQKPDIEGKLFFPIKGTELYAVHRASFNIKLPGETLVKSREVLRKAREDNLKLMNQKLNFLRNVLHFQQFEDITEREKRVTKWISRQENSDVPLVYQDELIQIRELMYKPYKDWVAFLKQLHKRLEVEIGKEVKHWRKSLSDGRKGLYGISLKNIDEIDRTRKFLLRWSLRPTEPGEVRRLEPGQRFAIDQLNHLNALKEDRLKKMANTIIMHALGYCYDVRKKKWQAKNPACQIILFEDLSNYNPYEERSRFENSKLMKWSRREIPRQVALQGEIYGLQVGEVGAQFSSRFHAKTGSPGIRCSVVTKEKLQDNRFFKNLQREGRLTLDKIAVLKEGDLYPDKGGEKFISLSKDRKCVTTHADINAAQNLQKRFWTRTHGFYKVYCKAYQVDGQTVYIPESKDQKQKIIEEFGEGYFILKDGVYEWVNAGKLKIKKGSSKQSSSELVDSDILKDSFDLASELKGEKLMLYRDPSGNVFPSDKWMAAGVFFGKLERILISKLTNQYSISTIEDDSSKQSM

SEQ ID NO: 46 - protein, 1112 aa, Bacillus sp.
MAIRSIKLKMKTNSGTDSIYLRKALWRTHQLINEGIAYYMNLLTLYRQEAIGDKTKEAYQAELINIIRNQQRNNGSSEEHGSDQEILALLRQLYELIIPSSIGESGDANQLGNKFLYPLVDPNSQSGKGTSNAGRKPRWKRLKEEGNPDWELEKKKDEERKAKDPTVKIFDNLNKYGLLPLFPLFTNIQKDIEWLPLGKRQSVRKWDKDMFIQAIERLLSWESWNRRVADEYKQLKEKTESYYKEHLTGGEEWIEKIRKFEKERNMELEKNAFAPNDGYFITSRQIRGWDRVYEKWSKLPESASPEELWKVVAEQQNKMSEGFGDPKVFSFLANRENRDIWRGHSERIYHIAAYNGLQKKLSRTKEQATFTLPDAIEHPLWIRYESPGGTNLNLFKLEEKQKKNYYVTLSKIIWPSEEKWIEKENIEIPLAPSIQFNRQIKLKQHVKGKQEISFSDYSSRISLDGVLGGSRIQFNRKYIKNHKELLGEGDIGPVFFNLVVDVAPLQETRNGRLQSPIGKALKVISSDFSKVIDYKPKELMDWMNTGSASNSFGVASLLEGMRVMSIDMGQRTSASVSIFEVVKELPKDQEQKLFYSINDTELFAIHKRSFLLNLPGEVVTKNNKQQRQERRKKRQFVRSQIRMLANVLRLETKKTPDERKKAIHKLMEIVQSYDSWTASQKEVWEKELNLLTNMAAFNDEIWKESLVELHHRIEPYVGQIVSKWRKGLSEGRKNLAGISMWNIDELEDTRRLLISWSKRSRTPGEANRIETDEPFGSSLLQHIQNVKDDRLKQMANLIIMTALGFKYDKEEKDRYKRWKETYPACQIILFENLNRYLFNLDRSRRENSRLMKWAHRSIPRTVSMQGEMFGLQVGDVRSEYSSRFHAKTGAPGIRCHALTEEDLKAGSNTLKRLIEDGFINESELAYLKKGDIIPSQGGELFVTLSKRYKKDSDNNELTVIHADINAAQNLQKRFWQQNSEVYRVPCQLARMGEDKLYIPKSQTETIKKYFGKGSFVKNNTEQEVYKWEKSEKMKIKTDTTFDLQDLDGFEDISKTIELAQEQQKKYLTMFRDPSGYFFNNETWRPQKEYWSIVNNIIKSCLKKKILSNKVEL

SEQ ID NO: 47 - protein, 1367 aa, Artificial Sequence
  Description of Artificial Sequence Synthetic polypeptide
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFESPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGD

SEQ ID NO: 48 - protein, 1368 aa, Artificial Sequence
  Description of Artificial Sequence Synthetic polypeptide
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGD

SEQ ID NO: 49 - protein, 1368 aa, Artificial Sequence
  Description of Artificial Sequence Synthetic polypeptide
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKEYRSTKEVLDATLIHQSITGLYETRIDLSQLGGD

SEQ ID NO: 50 - protein, 1052 aa, Artificial Sequence
  Description of Artificial Sequence Synthetic polypeptide
KRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRKLINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYKNDLIKINGELYRVIGVNNDLLNRIEVNMIDITYREYLENMNDKRPPHIIKTIASKTQSIKKYSTDILGNLYEVKSKKHPQIIKKG

SEQ ID NO: 51 - protein, 1359 aa, Artificial Sequence
  Description of Artificial Sequence Synthetic polypeptide
MDKKYSIGLDIGTNSVGWAVITDDYKVPSKKFKVLGNTDRHSIKKNLIGALLFGSGETAEATRLKRTARRRYTRRKNRICYLQEIFSNEMAKVDDSFFHRLEESFLVEEDKKHERHPIFGNIVDEVAYHEKYPTIYHLRKKLADSTDKADLRLIYLALAHMIKFRGHFLIEGDLNPDNSDVDKLFIQLVQIYNQLFEENPINASRVDAKAILSARLSKSRRLENLIAQLPGEKRNGLFGNLIALSLGLTPNFKSNFDLAEDAKLQLSKDTYDDDLDNLLAQIGDQYADLFLAAKNLSDAILLSDILRVNSEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGAYHDLLKIIKDKDFLDNEENEDILEDIVLTLTLFEDRGMIEERLKTYAHLFDDKVMKQLKRRRYTGWGRLSRKLINGIRDKQSGKTILDFLKSDGFANRNFMQLIHDDSLTFKEDIQKAQVSGQGHSLHEQIANLAGSPAIKKGILQTVKIVDELVKVMGHKPENIVIEMARENQTTQKGQKNSRERMKRIEEGIKELGSQILKEHPVENTQLQNEKLYLYYLQNGRDMYVDQELDINRLSDYDVDHIVPQSFIKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEIQTVGQNGGLFDDNPKSPLEVTPSKLVPLKKELNPKKYGGYQKPTTAYPVLLITDTKQLIPISVMNKKQFEQNPVKFLRDRGYQQVGKNDFIKLPKYTLVDIGDGIKRLWASSKEIHKGNQLVVSKKSQILLYHAHHLDSDLSNDYLQNHNQQFDVLFNEIISFSKKCKLGKEHIQKIENVYSNKKNSASIEELAESFIKLLGFTQLGATSPFNFLGVKLNQKQYKGKKDYILPCTEGTLIRQSITGLYETRVDLSKIGED

SEQ ID NO: 52 - protein, 598 aa, Homo sapiens
MSASAQQLAEELQIFGLDCEEALIEKLVELCVQYGQNEEGMVGELIAFCTSTHKVGLTSEILNSFEHEFLSKRLSKARHSTCKDSGHAGARDIVSIQELIEVEEEEEILLNSYTTPSKGSQKRAISTPETPLTKRSVSTRSPHQLLSPSSFSPSATPSQKYNSRSNRGEVVTSFGLAQGVSWSGRGGAGNISLKVLGCPEALTGSYKSMFQKLPDIREVLTCKIEELGSELKEHYKIEAFTPLLAPAQEPVTLLGQIGCDSNGKLNNKSVILEGDREHSSGAQIPVDLSELKEYSLFPGQVVIMEGINTTGRKLVATKLYEGVPLPFYQPTEEDADFEQSMVLVACGPYTTSDSITYDPLLDLIAVINHDRPDVCILFGPFLESKHEQVENCLLTSPFEDIFKQCLRTIIEGTRSSGSHLVFVPSLRDVHHEPVYPQPPFSYSDLSREDKKQVQFVSEPCSLSINGVIFGLTSTDLLFHLGAEEISSSSGTSDRFSRILKHILTQRSYYPLYPPQEDMAIDYESFYVYAQLPVTPDVLIIPSELRYFVKDVLGCVCVNPGRLTKGQVGGTFARLYLRRPAADGAERQSPCIAVQVVRI

SEQ ID NO: 53 - protein, 335 aa, Homo sapiens
MSKRKAPQETLNGGITDMLTELANFEKNVSQAIHKYNAYRKAASVIAKYPHKIKSGAEAKKLPGVGTKIAEKIDEFLATGKLRKLEKIRQDDTSSSINFLTRVSGIGPSAARKFVDEGIKTLEDLRKNEDKLNHHQRIGLKYFGDFEKRIPREEMLQMQDIVLNEVKKVDSEYIATVCGSFRRGAESSGDMDVLLTHPSFTSESTKQPKLLHQVVEQLQKVHFITDTLSKGETKFMGVCQLPSKNDEKEYPHRRIDIRLIPKDQYYCGVLYFTGSDIFNKNMRAHALEKGFTINEYTIRPLGVTGVAGEPLPVDSEKDIFDYIQWKYREPKDRSE

SEQ ID NO: 54 - protein, 1107 aa, Homo sapiens [sequence omitted]

SEQ ID NO: 55 - protein, 2259 aa, Artificial Sequence
  Description of Artificial Sequence Synthetic polypeptide
MSLRSGGRRRADPGADGEASRDDGATSSVSALKRLERSQWTDKMDLRFGFERLKEPGEKTGWLINMHPVALPYKPYFYIATRKGCEREVSSFLSKKFQGKIAKVETVPKEDLDLPNHLVGLKRNYIRLSFHTVEDLVKVRKEISPAVKKNREQDHASDAYTALLSSVLQRGGVITDEEETSKKIADQLDNIVDMREYDVPYHIRLSIDLKIHVAHWYNVRYRGNAFPVEITRRDDLVERPDPVVLAFDIETTKLPLKFPDAETDQIMMISYMIDGQGYLITNREIVSEDIEDFEFTPKPEYEGPFCVFNEPDEAHLIQRWFEHVQETKPTIMVTYNGDFFDWPFVEARAAVHGLSMQQEIGFQKDSQGEYKAPQCIHMDCLRWVKRDSYLPVGSHNLKAAAKAKLGYDPVELDPEDMCRMATEQPQTLATYSVSDAVATYYLYMKYVHPFIFALCTIIPMEPDEVLRKGSGTLCEALLMVQAFHANIIFPNKQEQEFNKLTDDGHVLDSETYVGGHVEALESGVFRSDIPCRFRMNPAAFDFLLQRVEKTLRHALEEEEKVPVEQVTNFEEVCDEIKSKLASLKDVPSRIECPLIYHLDVGAMYPNIILTNRLQPSAMVDEATCAACDFNKPGANCQRKMAWQWRGEFMPASRSEYHRIQHQLESEKFPPLFPEGPARAFHELSREEQAKYEKRRLADYCRKAYKKIHITKVEERLTTICQRENSFYVDTVRAFRDRRYEFKGLHKVWKKKLSAAVEVGDAAEVKRCKNMEVLYDSLQLAHKCILNSFYGYVMRKGARWYSMEMAGIVCFTGANIITQARELIEQIGRPLELDTDGIWCVLPNSFPENFVFKTTNVKKPKVTISYPGAMLNIMVKEGFTNDQYQELAEPSSLTYVTRSENSIFFEVDGPYLAMILPASKEEGKKLKKRYAVFNEDGSLAELKGFEVKRRGELQLIKIFQSSVFEAFLKGSTLEEVYGSVAKVADYWLDVLYSKAANMPDSELFELISENRSMSRKLEDYGEQKSTSISTAKRLAEFLGDQMVKDAGLSCRYIISRKPEGSPVTERAIPLAIFQAEPTVRKHFLRKWLKSSSLQDFDIRAILDWDYYIERLGSAIQKIITIPAALQQVKNPVPRVKHPDWLHKKLLEKNDVYKQKKISELFTLEGRRQVTMAEASEDSPRPSAPDMEDFGLVKLPHPAAPVTVKRKRVLWESQEESQDLTPTVPWQEILGQPPALGTSQEEWLVWLRFHKKKWQLQARQRLARRKRQRLESAEGVLRPGAIRDGPATGLGSFLRRTARSILDLPWQIVQISETSQAGLFRLWALVGSDLHCIRLSIPRVFYVNQRVAKAEEGASYRKVNRVLPRSNMVYNLYEYSVPEDMYQEHINEINAELSAPDIEGVYETQVPLLFRALVHLGCVCVVNKQLVRHLSGWEAETFALEHLEMRSLAQFSYLEPGSIRHIYLYHHAQAHKALFGIFIPSQRRASVFVLDTVRSNQMPSLGALYSAEHGLLLEKVGPELLPPPKHTFEVRAETDLKTICRAIQRFLLAYKEERRGPTLIAVQSSWELKRLASEIPVLEEFPLVPICVADKINYGVLDWQRHGARRMIRHYLNLDTCLSQAFEMSRYFHIPIGNLPEDISTFGSDLFFARHLQRHNHLLWLSPTARPDLGGKEADDNCLVMEFDDQATVEINSSGCYSTVCVELDLQNLAVNTILQSHHVNDMEGADSMGISFDVIQQASLEDMITGGQAASAPASYDETALCSNTFRILKSMVVGWVKEITQYHNIYADNQVMHFYRWLRSPSSLLHDPALHRTLHNMMKKLFLQLIAEFKRLGSSVIYANFNRIILCTKKRRVEDAIAYVEYITSSIHSKETFHSLTISFSRCWEFLLWMDPSNYGGIKGKVSSRIHCGLQDSQKAGGAEDEQENEDDEEERDGEEEEEAEESNVEDLLENNWNILQFLPQAASCQNYFLMIVSAYIVAVYHCMKDGLRRSAPGSTPVRRRGASQLSQEAEGAVGALPGMITFSQDYVANELTQSFFTITQKIQKKVTGSRNSTELSEMFPVLPGSHLLLNNPALEFIKYVCKVLSLDTNITNQVNKLNRDLLRLVDVGEFSEEAQFRDPCRSYVLPEVICRSCNFCRDLDLCKDSSFSEDGAVLPQWLCSNCQAPYDSSAIEMTLVEVLQKKLMAFTLQDLVCLKCRGVKETSMPVYCSCAGDFALTIHTQVFMEQIGIFRNIAQHYGMSYLLETLEWLLQKNPQLGH

SEQ ID NO: 56 - protein, 257 aa, Artificial Sequence
  Description of Artificial Sequence Synthetic polypeptide
MPSLKISPSEAEKIQNYLVSSGFRKINAPYTLWALEGNGVKVYYYKTGSLLIQGKNSEKVLKEVLNLLEKKKLPGCDESGKGDIFGSLVLCCVCIPEENYLKVSSLNPRDTKRLSDKRVERLYLALKPLVKAYCYEIKPEEYNKLYRKFRNLNKMMTHFYKLLIERVKEECGVSEVVVDKYQPSNPFGEDVIFETEAERNLAVAVASIFARYKFLQSLKEVERELGIKIPKGTSKEVKELAKSLKNPERFIKLNFNV

SEQ ID NO: 57 - protein, 198 aa, Artificial Sequence
  Description of Artificial Sequence Synthetic polypeptide
MIEFVYPHTQLVAGVDEVGRGPLVGAVVTAAVILDPARPIAGLNDSKKLSEKRRLALYEEIKEKALSWSLGRAEPHEIDELNILHATMLAMQRAVAGLHIAPEYVLIDGNRCPKLPMPAMAVVKGDSRVPEISAASILAKVTRDAEMAALDIVFPQYGFAQHKGYPTAFHLEKLAEHGATEHHRRSFGPVKRALGLAS

SEQ ID NO: 58 - protein, 120 aa, Artificial Sequence
  Description of Artificial Sequence Synthetic polypeptide
  SITE (1)..(120): This sequence may encompass 1-30 “Ser Gly Gly Ser” repeating units
SGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGS

SEQ ID NO: 59 - protein, 120 aa, Artificial Sequence
  Description of Artificial Sequence Synthetic polypeptide
  SITE (1)..(120): This sequence may encompass 1-30 “Gly Gly Gly Ser” repeating units
GGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGS

SEQ ID NO: 60 - protein, 150 aa, Artificial Sequence
  Description of Artificial Sequence Synthetic polypeptide
  SITE (1)..(150): This sequence may encompass 1-30 “Gly Gly Gly Gly Ser” repeating units
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 61 - protein, 30 aa, Artificial Sequence
  Description of Artificial Sequence Synthetic polypeptide
  SITE (1)..(30): This sequence may encompass 1-30 residues
GGGGGGGGGGGGGGGGGGGGGGGGGGGGGG

SEQ ID NO: 62 - protein, 150 aa, Artificial Sequence
  Description of Artificial Sequence Synthetic polypeptide
  SITE (1)..(150): This sequence may encompass 1-30 “Glu Ala Ala Ala Lys” repeating units
EAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAK

SEQ ID NO: 63 - protein, 90 aa, Artificial Sequence
  Description of Artificial Sequence Synthetic polypeptide
  SITE (1)..(90): This sequence may encompass 1-30 “Gly Gly Ser” repeating units
GGSGGSGGSGGSGGSGGSGGSGGSGGSGGSGGSGGSGGSGGSGGSGGSGGSGGSGGSGGSGGSGGSGGSGGSGGSGGSGGSGGSGGSGGS

SEQ ID NO: 64 - protein, 60 aa, Artificial Sequence
  Description of Artificial Sequence Synthetic polypeptide
  MOD_RES (1)..(1): Any amino acid
  SITE (1)..(60): This sequence may encompass 1-30 “Xaa Pro” repeating units
  MOD_RES (3)..(3): Any amino acid
  MOD_RES (5)..(5): Any amino acid
  MOD_RES (7)..(7): Any amino acid
  MOD_RES (9)..(9): Any amino acid
  MOD_RES (11)..(11): Any amino acid
  MOD_RES (13)..(13): Any amino acid
  MOD_RES (15)..(15): Any amino acid
  MOD_RES (17)..(17): Any amino acid
  MOD_RES (19)..(19): Any amino acid
  MOD_RES (21)..(21): Any amino acid
  MOD_RES (23)..(23): Any amino acid
  MOD_RES (25)..(25): Any amino acid
  MOD_RES (27)..(27): Any amino acid
  MOD_RES (29)..(29): Any amino acid
  MOD_RES (31)..(31): Any amino acid
  MOD_RES (33)..(33): Any amino acid
  MOD_RES (35)..(35): Any amino acid
  MOD_RES (37)..(37): Any amino acid
  MOD_RES (39)..(39): Any amino acid
  MOD_RES (41)..(41): Any amino acid
  MOD_RES (43)..(43): Any amino acid
  MOD_RES (45)..(45): Any amino acid
  MOD_RES (47)..(47): Any amino acid
  MOD_RES (49)..(49): Any amino acid
  MOD_RES (51)..(51): Any amino acid
  MOD_RES (53)..(53): Any amino acid
  MOD_RES (55)..(55): Any amino acid
  MOD_RES (57)..(57): Any amino acid
  MOD_RES (59)..(59): Any amino acid
XPXPXPXPXPXPXPXPXPXPXPXPXPXPXPXPXPXPXPXPXPXPXPXPXPXPXPXPXPXP

SEQ ID NO: 65 - protein, 24 aa, Artificial Sequence
  Description of Artificial Sequence Synthetic peptide
SGGSSGSETPGTSESATPESSGGS

SEQ ID NO: 66 - protein, 32 aa, Artificial Sequence
  Description of Artificial Sequence Synthetic polypeptide
SGGSSGGSSGSETPGTSESATPESSGGSSGGS

SEQ ID NO: 67 - protein, 104 aa, Artificial Sequence
  Description of Artificial Sequence Synthetic polypeptide
GGSGGSPGSPAGSPTSTEEGTSESATPESGPGTSTEPSEGSAPGSPAGSPTSTEEGTSTEPSEGSAPGTSTEPSEGSAPGTSESATPESGPGSEPATSGGSGGS

SEQ ID NO: 68 - protein, 4 aa, Artificial Sequence
  Description of Artificial Sequence Synthetic peptide
SGGS

SEQ ID NO: 69 - protein, 21 aa, Artificial Sequence
  Description of Artificial Sequence Synthetic peptide
  SITE (1)..(21): This sequence may encompass 1, 3, or 7 “Gly Gly Ser” repeating units
GGSGGSGGSGGSGGSGGSGGS

SEQ ID NO: 70 - protein, 40 aa, Artificial Sequence
  Description of Artificial Sequence Synthetic polypeptide
SGGSSGGSSGSETPGTSESATPESSGGSSGGSSGGSSGGS

SEQ ID NO: 71 - protein, 64 aa, Artificial Sequence
  Description of Artificial Sequence Synthetic polypeptide
SGGSSGGSSGSETPGTSESATPESSGGSSGGSSGGSSGGSSGSETPGTSESATPESSGGSSGGS

SEQ ID NO: 72 - protein, 92 aa, Artificial Sequence
  Description of Artificial Sequence Synthetic polypeptide
PGSPAGSPTSTEEGTSESATPESGPGTSTEPSEGSAPGSPAGSPTSTEEGTSTEPSEGSAPGTSTEPSEGSAPGTSESATPESGPGSEPATS

SEQ ID NO: 73 - DNA, 76 bp, Artificial Sequence
  Description of Artificial Sequence Synthetic oligonucleotide
gttttagagctagaaatagcaagttaaaataaggctagtccgttatcaacttgaaaaagtggcaccgagtcggtgc

SEQ ID NO: 74 - protein, 5 aa, Artificial Sequence
  Description of Artificial Sequence Synthetic peptide
PAPAP

SEQ ID NO: 75 - protein, 6 aa, Artificial Sequence
  Description of Artificial Sequence Synthetic peptide
PAPAPA

SEQ ID NO: 76 - protein, 7 aa, Artificial Sequence
  Description of Artificial Sequence Synthetic peptide
PAPAPAP

SEQ ID NO: 77 - protein, 8 aa, Artificial Sequence
  Description of Artificial Sequence Synthetic peptide
PAPAPAPA

SEQ ID NO: 78 - protein, 9 aa, Artificial Sequence
  Description of Artificial Sequence Synthetic peptide
PAPAPAPAP

SEQ ID NO: 79 - protein, 15 aa, Artificial Sequence
  Description of Artificial Sequence Synthetic peptide
PAPAPAPAPAPAPAP

SEQ ID NO: 80 - protein, 21 aa, Artificial Sequence
  Description of Artificial Sequence Synthetic peptide
PAPAPAPAPAPAPAPAPAPAP

SEQ ID NO: 81 - protein, 29 aa, Artificial Sequence
  Description of Artificial Sequence Synthetic peptide
PKKKRKVEGADKRTADGSEFESPKKKRKV

SEQ ID NO: 82 - protein, 23 aa, Artificial Sequence
  Description of Artificial Sequence Synthetic peptide
RKSGKIAAIVVKRPRKPKKKRKV